(19) United States
(12) Patent Application Publication
(10) Pub. No.: US 2022/0062243 A1
(43) Pub. Date: Mar. 3, 2022

(54) P14-Kinase Inhibitors with Anti-Cancer Activity

(71) Applicants: Jeffrey S. GLENN, Palo Alto, CA (US); Mark SMITH, San Francisco,, CA (US); Edward PHAM, Palo Alto, CA (US); The Board of Trustees of the Leland Stanford Junior University, Stanford, CA (US)

(72) Inventors: Jeffrey S. GLENN, Palo Alto, CA (US); Mark SMITH, Stanford, CA (US); Edward PHAM, Stanford, CA (US)

(21) Appl. No.: 17/420,252

(22) PCT Filed: Jan. 9, 2020
(86) PCT No.: PCT/US2020/012965

§ 371 Date: Jul. 1, 2021

Related U.S. Application Data
(60) Provisional application No. 62/791,301, filed on Jan. 11, 2019.
(60) Provisional application No. 62/821,853, filed on Mar. 21, 2019.

(51) Int. Cl. A61K 31/426; A61P 35/00; A61K 45/06; A61K 31/427; A61K 31/4439

(52) U.S. Cl. CPC A61K 31/426; A61P 35/00; A61K 31/4439; A61K 31/427; A61K 45/06

(57) ABSTRACT

Methods of treating a subject for cancer using a PI4-kinase inhibitor are provided. Also provided are methods of inhibiting PI4-kinase in a cancer cell to reduce cellular proliferation. The PI4-kinase inhibitor can be a compound that is a 5-aryl or heteroaryl-thiazole, e.g., as described herein. In certain embodiments, the PI4-kinase inhibitor is a substituted 2-amino-5-phenylthiazole or substituted 2-amino-5-pyridylthiazole compound. The subject compounds may be formulated or provided to a subject in combination with one or more additional anti-cancer agents. Use of PI4-kinase inhibitors in methods of reducing cellular proliferation and methods of treatment is provided in a variety of cancer cells and cancer subjects.

### CROSS-REFERENCE TO RELATED APPLICATION

Pursuant to 35 U.S.C. § 119 (e), this application claims priority to the filing date of U.S. Provisional Patent Application Ser. No. 62/791,301, filed Jan. 11, 2019, and to the filing date of U.S. Provisional Patent Application Ser. No. 62/821,853 filed Mar. 21, 2019, the disclosures of which application are incorporated herein by reference.

### GOVERNMENT SUPPORT

This invention was made with Government support under contracts AI109662 and AI099245 awarded by the National Institutes of Health. The Government has certain rights in the invention.

### INTRODUCTION

Many cancers are dependent on PI4-kinase for growth and metastasis. In many cases this reflects a tumor “addiction” for PI4-kinase activity. Among the ways that this can be readily identified is the presence of increased PI4-kinase activity in target cancer cells. This increased activity can be directly measured, or reliably predicted by the presence of increased levels of factors known to enhance PI4-kinase activity (e.g., Eukaryotic protein translation elongation factor 1 alpha 2 (eEF1A2)), or chromosomal amplifications that increase the PI4-kinase gene copy number. For example, high levels of eEF1A2 protein and mRNA can be detected in 30-60% of ovarian, breast, and lung tumors among others. Similarly, amplification of PI4-kinase is readily detected in a significant percentage of most human tumor types (see e.g., Cancer Genome Atlas (TCGA) available through cbioportal.org). Other cancer cells are also more sensitive to selective PI-4 kinase inhibition as compared to normal cells. Thus, pharmacologic inhibitors of PI-4kinase are useful for treating cancer, including human cancers and/or their metastases.

### SUMMARY

Methods of treating a subject for cancer using a PI4-kinase inhibitor are provided. Also provided are methods of inhibiting PI4-kinase in a cancer cell to reduce cellular proliferation. The PI4-kinase inhibitor can be a compound that is a 5-aryl or heteroaryl-thiazole, e.g., as described herein. In certain embodiments, the PI4-kinase inhibitor is a substituted 2-amino-5-phenylthiazole or substituted 2-amino-5-pyridylthiazole compound. The subject compounds may be formulated or provided to a subject in combination with one or more additional anti-cancer agents. Use of PI4-kinase inhibitors in methods of reducing cellular proliferation and methods of treatment is provided in a variety of cancer cells and cancer subjects.

These and other advantages and features of the present disclosure will become apparent to those persons skilled in the art upon reading the details of the compositions and methods of use, which are more fully described below.

### BRIEF DESCRIPTION OF THE DRAWINGS

FIGS. 1A-1C illustrate that PI4-kinase is a target in cancer cells of interest (FIG. 1A) The PI4-kinase IIIα, PI4-kinase IIIβα, and eEF1A2 (which increases PI4-kinase activity) genes are amplified across cancer types. (FIG. 1B) The eEF1A2 gene, which increases PI4-kinase activity, is overexpressed across cancer types. (FIG. 1C) The median eEF1A2 mRNA expression is significantly higher in cancer cells compared to the corresponding normal tissue types. A significant number of K-ras mutant cancers contain one of the amplifications that increase PI4-kinase activity. Data obtained from cbioportal.org as well as the human protein atlas (proteinatlas.org).

FIG. 2A. Intracellular PI4P concentrations in H2122 lung cancer cells treated with compound B (PI4-kinase inhibitor) or vehicle DMSO.

FIG. 2B Left panel: Relative densities of PI4KIIIβ-amplified (red) and diploid (black) human lung adenocarcinoma cell lines by WST-1 assays after 5 days of compound B treatment. Results expressed relative to the lowest dose, which was set at 100%. Right panel: Half maximal inhibitory (IC50) concentrations of compound B determined from left panel data.

FIG. 2C. Migrated and invaded H23 human lung cancer cells in Transwell chambers were photographed (images) and counted (bar graphs) after treatment with compound B. Results expressed relative to DMSO-treated cells, which were set at 1.0.

FIG. 2D-2E. Colonies formed by H2122 human lung cancer cells in soft agarose (FIG. 2D) and on plastic (FIG. 2E) were photographed (images) and counted (bar graphs) after 7 days of treatment with the indicated doses of compound B or vehicle DMSO (0 μM). Results expressed relative to DMSO control, which were set at 1.0.

FIGS. 2F and 2G. Schema of compound B treatment: Day 0, H2122 human lung cancer cell injection; day 7-27 compound B treatment; tumor imaging day 26 and necropsy day 27. (FIG. 2F) Mice subjected to micro-computed tomography after 19 days of treatment to determine tumor areas (left dot plot). Tumor diameters determined at necropsy (right dot plot). (FIG. 2G) Mice grouped on the basis of lung tumor measurements determined at necropsy, which showed a shift toward smaller tumor diameters in compound B-treated mice.

FIGS. 3A-3C. Schema of compound A treatment: Day 0, H2122 human lung cancer cell injection; day 7-15 compound A treatment; tumor imaging day 14 and necropsy day 15. (FIG. 3A) Mouse body weight changes after 8 days treatment with vehicle (left panel) or vehicle plus 100 mg/kg/day compound A (right panel). (FIG. 3B) Mice subjected to micro-computed tomography before and after treatment to determine tumor areas after 7 days treatment with vehicle or vehicle plus 100 mg/kg/day compound A. Left panel: tumor area as measured before and after treatment. Right panel: tumor area expressed as percent of baseline measurement. (FIG. 3C) Tumor diameters determined at necropsy (left panel), and number of tumor metastases (right panel).

FIG. 4. Breast tumors were established by injecting human MDA-MB-468 cells into the mammary fat pads of nude mice. After the tumors were established, the mice were treated with an exemplary 5-aryl-thiazole compound (Compound A).

FIG. 5A-5D illustrates the overall survival rates of cancer patients having tumors characterized as having no PI4Kβ amplification versus PI4Kβ amplification. Data was obtained from the Cancer Genome Atlas (TCGA) database for patients having lung adenocarcinoma (FIG. 5A), liver (FIG. 5B), pancreatic (FIG. 5C) or liver (FIG. 5D) cancer.

### DEFINITIONS

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Phosphoinositides, such as phosphatidylinositol-4-phosphate (PI(4)P) and PI-4,5-bisphosphate (PI(4,5)P₂, or “PIP2”), are enriched in various specific plasma membrane and intracellular locations. The steady state location and abundance of specific PI isoform pools within the cell is regulated by a family of phosphatidylinositol (PI)-kinases and phosphatases. There are least four human phosphatidylinositol 4-kinases (PI4-kinases), with family members PI4KIIIα and PI4KIIIβ being primarily localized to ER and Golgi-derived membranes where they contribute to the PI(4)P and PI(4,5)P₂ pools associated with these membranes, and with family members PI4KIIα and PI4KIIβ contributing primarily to other pools.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction in tumor burden).

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to affect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “substituted alkyl” is meant to include an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C1-C6 alkoxy” or “lower alkoxy” herein may, for example, may contain 1 to 3 carbon atoms, and as a further example, such substituents may contain 1 or 2 carbon atoms (i.e., methoxy and ethoxy).

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C3-C14 moieties, exemplified but not limited to phenyl, biphenyl, naphthyl, pyridyl, furyl, thiophenyl, imidazoyl, pyrimidinyl, and oxazoyl; which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” as used herein refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

Similarly, the terms “alkenylene,” “alkynylene,” “arylene,” “aralkylene,” and “alkarylene” as used herein refer to di-radical alkenyl, alkynyl, aryl, aralkyl, and alkaryl groups, respectively.

The term “amino” is used herein to refer to the group —NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

“Carboxyl,” “carboxy” or “carboxylate” refers to —CO₂H or salts thereof.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO— alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

As used herein, the terms “Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. A hydrocarbyl may be substituted with one or more substituent groups. The term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C₆ alkenyl), C2-C24 alkynyl (including C2-C18 alkynyl, further including C2-C12 alkynyl, and further including C2-C₆ alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂-, phenyl-SO₂—, and 4-methylphenyl-SO₂—.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH2), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH2), carbamido (—NH—(CO)—NH2), cyano (—C≡N), isocyano (—N+C—), cyanato (—O—C≡N), isocyanato (—O—N+C—), isothiocyanato (—S—C≡N), azido (—N═N+═N—), formyl (—(CO)—H), thioformyl (—(CS)—H), amino (—NH2), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO2), nitroso (—NO), sulfo (—SO₂—OH), sulfonato (—SO₂—O—, C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO₂-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

By “linking” or “linker” as in “linking group,” “linker moiety,” etc., is meant a bivalent radical moiety that connects two groups via covalent bonds. Examples of such linking groups include alkylene, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), carbonyldioxy (—O—CO—O—), alkyldioxy (—O—(CH2)n-O—), epoxyimino (—O—NH—), epimino (—NH—), carbonyl (—CO—), etc. Any convenient orientation and/or connections of the linkers to the linked groups may be used.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻ M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰ 's, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —OR⁷⁰, —NR⁸⁰R⁸⁰, trihalomethyl, —CF₃, —CN, —OCN, —SCN, —NO, —NO₂, —N₃, —SO₂R⁷⁰, —SO₃⁻ M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —OR⁷⁰, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰) R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus, the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

Definitions of other terms and concepts appear throughout the detailed description below.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### DETAILED DESCRIPTION OF THE EMBODIMENTS

As summarized above, methods are provided for the treatment of cancer using a PI4-kinase inhibitor. Also provided are methods of inhibiting PI4-kinase in a cancer cell to reduce cellular proliferation. The methods of the present disclosure can target cancer cells. The target cancer cells and their metastases can be considered “addicted” to increased PI4-kinase activity. The latter can result from amplification of chromosomal segments that harbor a PI4-kinase gene, such as PI4-III-kinase α or PI4-III-kinase β, or eukaryotic protein translation elongation factor 1 alpha 2 (eEF1A2). eEF1A2 is a translation factor that is involved in internal ribosome entry site (IRES) mediated translation. eEF1A2 also stimulates PI4-kinase activity and is overexpressed in many cancers. IRESs are often used by viruses as a means to ensure that viral translation is active when host translation is inhibited. IRES-mediated translation can contribute to the translation of certain cellular RNAs, particularly under abnormal cellular states. The target cancer cells can have the above chromosomal amplifications, or increased expression of eEF1A2 without chromosomal amplifications, any of which can lead to increased PI4 kinase activity. The inventors discovered that anti-viral PI4 kinase inhibitors that potently target IRES containing viruses are also effective in reducing proliferation of cancer cells and find use in the treatment of cancer.

### PI4-Kinase Inhibitor Compounds

As summarized above, aspects of the disclosure include use of PI4-kinase inhibitor compounds. Any convenient PI4-kinase inhibitors can be utilized in the subject methods. In certain instances, the PI4-kinase inhibitor is a class III PI4-kinase inhibitor. In certain instances, the PI4-kinase inhibitor is a PI4IIIβ kinase inhibitor. In certain cases, the PI4-kinase inhibitor has specific inhibition activity for a PI4-kinase over PI3-kinases.

In some cases, the PI4-kinase inhibitor is a pyrazolopyridine compound, such as a compound described by Chatterjee A K, et al. in WO2014078802A1, the disclosure of which is herein incorporated by reference in its entirety. In some cases, the PI4-kinase inhibitor is KDU731.

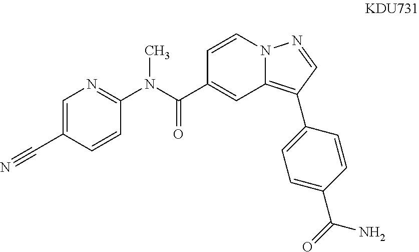 [SMILES: CN(C(=O)c1ccn2ncc(-c3ccc(C(N)=O)cc3)c2c1)c1ccc(C#N)cn1]

In some cases, the PI4-kinase inhibitor is an aminoimidazole inhibitor compound, such as a compound described by M. J. Lamarche et al. (“Anti-hepatitis C virus activity and toxicity of type III phosphatidylinositol-4-kinase beta inhibitors”, Antimicrob Agents Chemother, 56 (2012), pp. 5149-5156), the disclosure of which is herein incorporated by reference in its entirety, such as one of the following compounds 1-6 of Table 1:

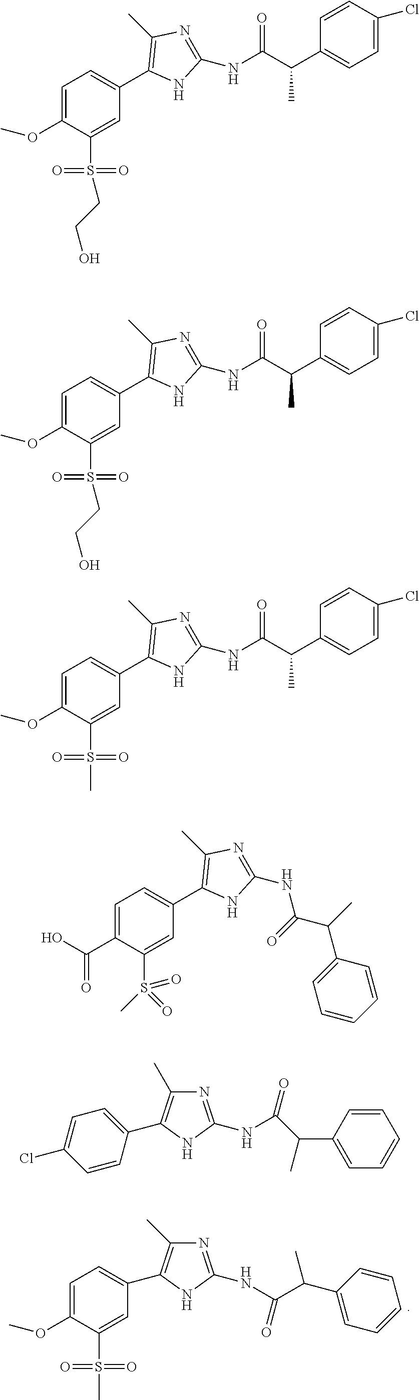 [SMILES: COc1ccc(-c2[nH]c(NC(=O)[C@@H](C)c3ccc(Cl)cc3)nc2C)cc1S(=O)(=O)CCO ; COc1ccc(-c2[nH]c(NC(=O)[C@H](C)c3ccc(Cl)cc3)nc2C)cc1S(=O)(=O)CCO ; COc1ccc(-c2[nH]c(NC(=O)[C@@H](C)c3ccc(Cl)cc3)nc2C)cc1S(C)(=O)=O ; Cc1nc(NC(=O)C(C)c2ccccc2)[nH]c1-c1ccc(C(=O)O)c(S(C)(=O)=O)c1 ; Cc1nc(NC(=O)C(C)c2ccccc2)[nH]c1-c1ccc(Cl)cc1 ; COc1ccc(-c2[nH]c(NC(=O)C(C)c3ccccc3)nc2C)cc1S(C)(=O)=O]

In some cases, the PI4-kinase inhibitor is an aminoquinoline or quinazolinone inhibitor compound, such as a compound described by Banka et al. in WO 2012037108 or by Leivers et al. (“Discovery of selective small molecule type III phosphatidylinositol 4-kinase alpha (PI4KIIIa) inhibitors as anti hepatitis C (HCV) agents”, J. Med. Chem. 2014, 57, 2091-2106), the disclosures of which are herein incorporated by reference in their entirety, such as one of the following compounds 20-27:

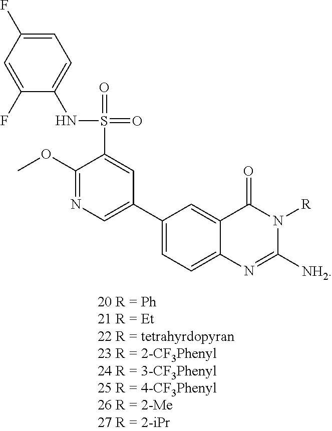

In some cases, the PI4-kinase inhibitor is an imidazo[1,2-a]pyrazine inhibitor compound, such as a compound described by van der Schaar et al. (“A Novel, Broad-Spectrum Inhibitor of Enterovirus Replication That Targets Host Cell Factor Phosphatidylinositol 4-Kinase IIIβ”, Antimicrobial Agents and Chemotherapy p. 4971-4981, 2013, 57(10), the disclosure of which is herein incorporated by reference in its entirety, such as one of the following compounds:

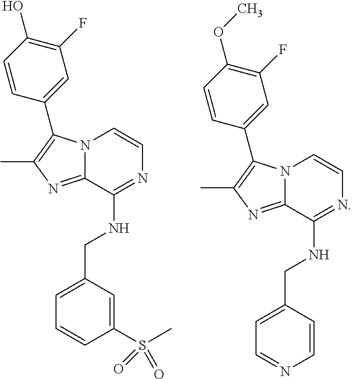

In some cases, the PI4-kinase inhibitor is a pyrazolo[1,5-a]pyrimidine inhibitor compound, such as a compound described by Sala et al. (“Purine analogs as phosphatidylinositol 4-kinase Mb inhibitors”, Bioorg. Med. Chem. Lett. 26 (2016) 2706-2712), the disclosure of which is herein incorporated by reference in its entirety, such as T-00127-HEV1 (3-(3,4-Dimethoxyphenyl)-2,5-dimethyl-N-(2-morpholinoethyl)pyrazolo[1,5-a]pyrimidin-7-amine)

Other PI4-kinase inhibitors of interest include, but are not limited to, wortmannin, quercetin, 2-(4-morpholinyl)-8-phenyl-1(4H)-benzopyran-4-one (LY-294,002), 4-anilino-quinazoline inhibitor compounds (e.g., AL-9),

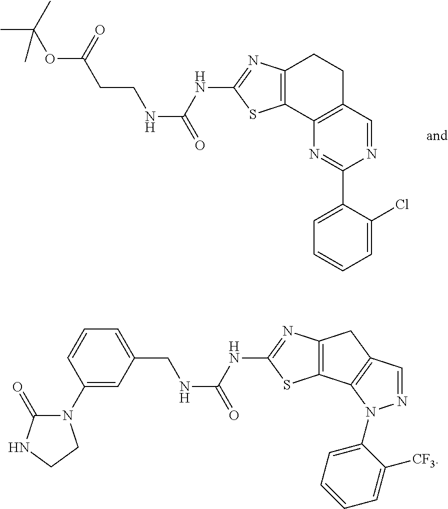 [SMILES: CC(C)(C)OC(=O)CCNC(=O)Nc1nc2c(s1)-c1nc(-c3ccccc3Cl)ncc1CC2 ; O=C(NCc1cccc(N2CCNC2=O)c1)Nc1nc2c(s1)-c1c(cnn1-c1ccccc1C(F)(F)F)C2]

Other PI4-kinase inhibitors of interest include, but are not limited to, those PI4-kinase inhibitors described by: Glenn et al. in WO2013/052845, U.S. Pat. Nos. 9,309,236, 9,926,309, 10,428,060 and WO2017/147526; Rutaganira et al. (Design and Structural Characterization of Potent and Selective Inhibitors of Phosphatidylinositol 4 Kinase IIIbeta. J. Med. Chem. 59, 1830-1839, 2016); Toth et al. (inhibitors wortmannin and PIK93) (“Phosphatidylinositol 4-Kinase IIIβ Regulates the Transport of Ceramide between the Endoplasmic Reticulum and Golgi”, J. Biol. Chem., 281, 36369-36377, 2006), the disclosures of which are herein incorporated by reference in their entirety.

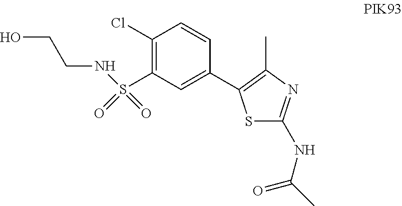 [SMILES: CC(=O)Nc1nc(C)c(-c2ccc(Cl)c(S(=O)(=O)NCCO)c2)s1]

The PI4-kinase inhibitor can be of the class of compounds having a 5-aryl-thiazole or a 5-heteroaryl-thiazole core structure, see e.g., PIK93 and compounds described by Glenn et al. in WO 2017/147526. The thiazole ring of the compounds can include a substituted amino at the 2-position. The 5-aryl or 5-heteroaryl ring may be a 6-membered heteroaryl (e.g., pyridyl) or phenyl ring that includes at least a further substituent meta to the thiazole ring substituent. The thiazole ring of the core structure may include further substituents at the 2- and/or 4-positions of the ring, such as a 2-amino group and a 4-alkyl group, each optionally further substituted. In some embodiments, the PI4-kinase inhibitor compounds are substituted 2-amino-5-phenylthiazole compounds that include a thiazole ring having an amino at the 2-position of the ring, and a phenyl substituent at the 5-position of the ring. In some embodiments, the PI4-kinase inhibitor compounds are substituted 2-amino-5-pyridyl-thiazole compounds that include a thiazole ring having an amino at the 2-position of the ring, and a pyridyl substituent at the 5-position of the ring. In some embodiments, the compound includes further substituents, such as a substituent at either the 4 or 5-position of the thiazole ring. The aryl ring of the core structure (e.g., 5-phenyl or pyridyl ring) may be further substituted with any convenient substituents including but not limited to alkyl, acyl, acyloxy, aminoalkoxy, cyano, halogen, hydroxyl, nitro, —NHCOR, —SO₂R, —SO₂NHR, —COR, —CONHR or —NHSO₂R, where R is alkyl, heteroalkyl, heterocycle or aryl. Exemplary 5-aryl-thiazole compounds are set forth in the following structures and formulae I-XLIX.

In some cases, the PI4-kinase inhibitor is described by the structure of formula (Ia):

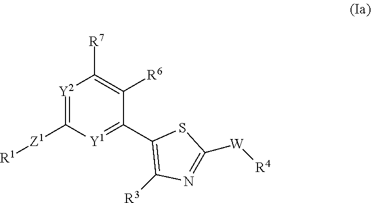

where:

Z¹ and W are each independently a covalent bond or a linking functional group;

Y¹ and Y² are each independently CR² or N;

R¹ is selected from hydrogen, an alkyl, a substituted alkyl, an aryl, a substituted aryl, an alkyl-heterocycle, a substituted heterocycle, a heterocycle and a substituted heterocycle;

R³ is selected from hydrogen and an alkyl;

R⁴ is selected from an alkyl, a substituted alkyl, an aralkyl, a substituted aralkyl, an aryl, a substituted aryl, an alkyl-cycloalkyl, a substituted alkyl-cyclohexyl, a cycloalkyl, a substituted cycloalkyl, an alkyl-heterocycle, a substituted alkyl-heterocycle, a heterocycle, a substituted heterocycle, an amino, a substituted amino, an alkoxy and a substituted alkoxy; and

R², R⁶ and R⁷ are independently selected from hydrogen, an alkyl, a substituted alkyl, an aryl, a substituted aryl, a hydroxy, an alkoxy, a substituted alkoxy, an aryloxy, a substituted aryloxy, a heterocycle, a substituted heterocycle, a cyano, a halogen, an amino, a substituted amino, an acyl, an acyloxy, an amido, and a nitro.

In some cases, the PI4-kinase inhibitor is described by the structure of formula (Ib):

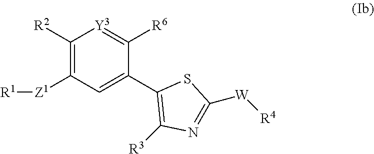

where:

Z¹ and W are each independently a covalent bond or a linking functional group;

Y³ is CR⁷ or N;

R¹ is selected from hydrogen, an alkyl, a substituted alkyl, an aryl, a substituted aryl, an alkyl-heterocycle, a substituted heterocycle, a heterocycle and a substituted heterocycle;

R³ is selected from hydrogen and an alkyl;

R⁴ is selected from an alkyl, a substituted alkyl, an aralkyl, a substituted aralkyl, an aryl, a substituted aryl, an alkyl-cycloalkyl, a substituted alkyl-cyclohexyl, a cycloalkyl, a substituted cycloalkyl, an alkyl-heterocycle, a substituted alkyl-heterocycle, a heterocycle, a substituted heterocycle, an amino, a substituted amino, an alkoxy and a substituted alkoxy; and

R², R⁶ and R⁷ are independently selected from hydrogen, an alkyl, a substituted alkyl, an aryl, a substituted aryl, a hydroxy, an alkoxy, a substituted alkoxy, an aryloxy, a substituted aryloxy, a heterocycle, a substituted heterocycle, a cyano, a halogen, an amino, a substituted amino, an acyl, an acyloxy, an amido, and a nitro.

In certain embodiments, in formula (Ia) or (Ib), R¹ to R⁷ are independently selected from corresponding groups as depicted in any of the structures of Table 1 or 2.

In some embodiments, in formula (Ia), Y¹ is CH and Y² is CR², such that the compound is described by the formula (IIa):

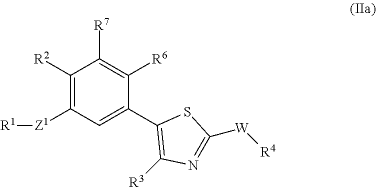

where:

Z¹ and W are each independently a covalent bond or a linking functional group;

R¹ is selected from an alkyl, an aryl, an alkyl-heterocycle and a heterocycle;

R² is selected from hydrogen, a halogen, an alkyl, a substituted alkyl, an alkoxy and a substituted alkoxy;

R³ is selected from hydrogen and an alkyl;

R⁴ is selected from an alkyl, an aralkyl, an aryl, an alkyl-cycloalkyl, a cycloalkyl, an alkyl-heterocycle, a heterocycle; and

R⁶ and R⁷ are independently selected from hydrogen, an alkyl, an aryl, a hydroxy, an alkoxy, an aryloxy, a heterocycle, a cyano, a halogen, an amino, an acyl, an acyloxy, an amido and nitro.

In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In some embodiments, R³ and R⁶ are selected such that they form a 6-membered ring as part of a fused tricyclic aryl-thiazole core structure.

In some embodiments, R¹ is not a hydroxy-substituted alkyl group, such as —(CH₂)₂—OH.

In some embodiments, R¹ is selected from hydrogen, an alkyl, an aryl (e.g., a phenyl), an alkyl-heterocycle and a heterocycle (e.g., pyridyl, pyrimidinyl, pyrrolyl, pyrrolidinyl, quinolinyl, indolyl, furyl, imidazolyl, oxazolyl, thiazolyl, 1,2,4-triazolyl, tetrazolyl, pyrrolidino, morpholino, piperazino, piperidino, tetrahydrofuranyl). In some embodiments, R¹ is selected from hydrogen, a substituted lower alkyl (e.g., a substituted methyl or ethyl), a phenyl, a cycloalkyl, a pyridyl and a pyrimidinyl.

In some instances, R⁴ is —(CH₂)ₙ—R¹⁰, where n is 0, 1, 2 or 3; and R¹⁰ is a cycloalkyl or a heterocycle (e.g., a 5- or 6-membered saturated N-containing heterocycle). In certain cases, R¹⁰ is selected from a cyclohexyl, a cyclopentyl, a cyclopropyl, a lower alkyl, a pyrrolidinyl and a piperidinyl.

In some embodiments, the PI4-kinase inhibitor is described by the structure of formula (IIb):

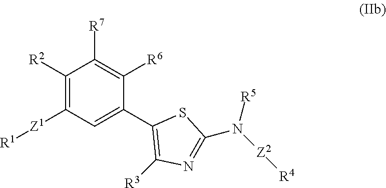

where:

Z¹ and Z² are each independently a covalent bond or a linking functional group;

R¹ is selected from hydrogen, an alkyl, a substituted alkyl (e.g., a substituted ethyl, or a heterocycle-substituted lower alkyl), an aryl (e.g., a phenyl), a substituted aryl, a heterocycle (e.g., a pyridyl, a pyrimidinyl) and a substituted heterocycle;

R² is selected from hydrogen, a halogen, an alkyl, a substituted alkyl, an alkoxy and a substituted alkoxy;

R³ and R⁵ are selected from hydrogen and an alkyl (e.g., a lower alkyl such as a methyl);

R⁴ is selected from an alkyl (e.g., a cycloalkyl such as cycloheptyl, cyclohexyl, cyclopentyl, cyclopropyl or a lower alkyl such as methyl, ethyl or tert-butyl), an aralkyl (e.g., a benzyl or a phenylethyl), an aryl, an alkyl-heterocycle, a heterocycle, an amino and an alkoxy; and

R⁶ and R⁷ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro.

In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments, in formula (IIb), R¹ to R⁴ are independently selected from corresponding groups as depicted in any of the structures of Table 1 or 2.

The linking functional group may be any convenient bivalent group. Linking functional groups of interest include, but are not limited to, an amino, an amido, an ester, a carbonyloxy, an ether, a carbamate, a sulfonamide, a carbonyl, a sulfonyl, a sulfinyl, or the like. In some embodiments, the linking functional group is described by one of the following formulas: —SO₂NR—, —NR—, —NRC(═O)—, or —NRC(═O)NR— where each R is independently H, an alkyl, a cycloalkyl, a heterocycle, a heterocycloalkyl, an aryl or a heteroaryl; —O—; —C(═O)—; —C(═O)X— where X is NR, O or S and where R is H or an alkyl; —S(═O)— or —SO₂—; where for each of the formulae depicted it is understood that both possible orientations of a functional group are included. In some embodiments, in formula (I), Z¹ is —SO₂NH— or —CONH— and W is a covalent bond, —NR— or —NRC(═O)—, where R is H or an alkyl.

In some embodiments, in formula (II), Z¹ is —NHSO₂— or —SO₂NH—; and Z² is a covalent bond or —C(═O)—.

In some embodiments, R¹ is described by the formula —(CH₂)ₙ—CH(R⁸)—CHR⁹, where R⁸ is hydrogen or a lower alkyl (e.g., methyl) and R⁹ is hydrogen, an aryl (e.g., a phenyl) or a heterocycle (e.g., pyridyl (e.g., 3-pyridyl), pyrimidinyl, pyrrolyl, pyrrolidinyl, quinolinyl, indolyl, furyl, imidazolyl, oxazolyl, thiazolyl, 1,2,4-triazolyl, tetrazolyl, pyrrolidino, morpholino, piperazino, piperidino, tetrahydrofuranyl); and n is 0, 1, 2 or 3. In some embodiments, n is 0. In certain embodiments, R¹ is a substituted ethyl group, for example, a group described by one of the following structures:

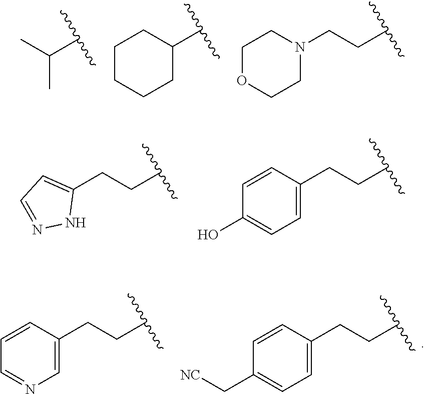

In other embodiments, R¹ is described by the formula:

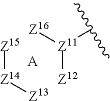

where A is a 6-membered aryl, heteroaryl, heterocyclyl, or cycloalkyl, where Z¹¹-Z¹⁶ are independently selected from N, CR′, NR and CR′R″, where R is H or alkyl, and R′ and R″ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro.

In some embodiments, R¹ is described by the following formula:

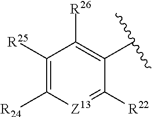

where Z¹³ is CR²³ or N, where R²²-R²⁶ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro.

In certain embodiments, R¹ is described by the following formula:

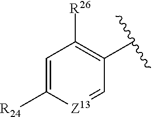

where Z¹³ is CR²³ or N, where R²³, R²⁴ and R²⁶ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro.

In some embodiments, R¹ is described by the following formula:

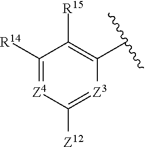

where Z³ is N or CR¹¹; Z⁴ is N or CR¹³; and R¹¹ to R¹⁵ are each independently selected from where R²²-R²⁶ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro. In certain embodiments, R¹¹ to R¹⁵ are each independently selected from hydrogen, an alkyl, an alkoxy, an acyloxy, a cyano, a halogen, and hydroxyl. In certain embodiments, Z³ is CR¹¹, Z⁴ is CR¹³; R¹¹, R¹⁴ and R¹⁵ are each hydrogen; R¹² is hydrogen, an alkoxy (e.g., methoxy) or a halogen (e.g., fluoro); and R¹³ is selected from hydrogen, acetyloxy, hydroxy, methoxy, cyano-methyl and halogen (e.g., fluoro). In certain embodiments, Z⁴ is N. In certain embodiments, Z³ and Z⁴ are each N.

In some instances, R¹ is described by the following formula:

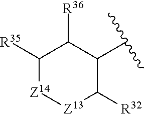

where Z¹³ and Z¹⁴ are each independently CR′R″ or NR, where R is H or alkyl, and R³², R³⁵, R³⁶, R′ and R″ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro. In certain embodiments, R³², R³⁵, R³⁶, R′ and R″ are each independently selected from hydrogen, an alkyl, an alkoxy, an acyloxy, a cyano, a halogen, and hydroxyl.

In certain embodiments, R¹ is described by one of the following formulas:

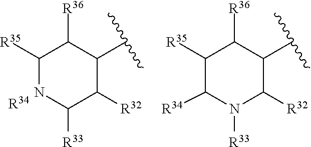

In some embodiments, in formulae (Ia), (Ib), (IIa) or (IIb), R² is methoxy. In some embodiments, in formulae (Ia), (Ib), (IIa) or (IIb), R³ is methyl.

In some embodiments, in formula (IIb), Z² is a covalent bond or —C(═O)—, and R⁴ is a lower alkyl (trifluoromethyl, tert-butyl, methyl, ethyl), a cycloalkyl (e.g., cyclopentyl, 1-fluoro-cyclopentyl or cyclohexyl) or —CH₂-cycloalkyl, a heterocycle (e.g., a N-linked saturated heterocycle such as N-pyrollidinyl, N-morpholino), or an amino (e.g., an amino-alkyl such as N-amino-cyclopentyl). In some embodiments, in formula (IIb), R⁴ is described by the formula —NR¹⁶R¹⁷, wherein R¹⁶ and R¹⁷ are each independently selected from hydrogen, an alkyl, a cycloalkyl, and wherein optionally R¹⁶ and R¹⁷ are cyclically linked (e.g., to form a N-heterocyclyl). In some embodiments, in formula (IIb), Z² is a covalent bond; and R⁴ is an alkyl or an alkyl-cycloalkyl (e.g., 1-cyclopentyl-methyl-). In some embodiments, in formula (IIb), R⁴ is selected from methyl, trifluoromethyl, ethyl, tert-butyl, cyclopentyl, N-pyrrolidinyl, N-morpholinyl, N-amino-cyclopentyl and 1-fluoro-cyclopentyl. In certain embodiments, in formula (IIb), R⁵ is hydrogen. In certain embodiments, in formula (II), R⁶ and R⁷ are each hydrogen.

In some instances, the compound is described by the structure of formula (III):

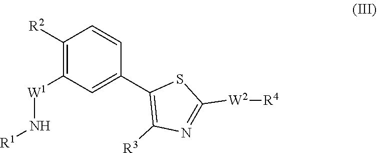

where R¹ is selected from hydrogen, an alkyl, an aryl, an alkyl-heterocycle and a heterocycle; R² is selected from hydrogen, a halogen, an alkyl, a substituted alkyl, an alkoxy and a substituted alkoxy; R³ is alkyl; R⁵ is H or alkyl; R⁴ is lower alkyl, cycloalkyl, -alkyl-cycloalkyl, heterocyclyl or alkyl-heterocyclyl (e.g., —(CH₂)ₙ-cycloalkyl or —(CH₂)ₙ-heterocycyl, where n is 0, 1 or 2); W¹ is —SO₂— or —C(═O)—; and W² is a covalent bond, —NH—, or —NHCO—. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments, in formula (III), R¹ is described by the following structure:

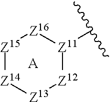

where A is a 6-membered aryl, heteroaryl, heterocyclyl, or cycloalkyl, where Z¹¹-Z¹⁶ are independently selected from N, CR′, NR and CR′R″, where R is H or alkyl, and R′ and R″ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro.

In some embodiments, in formula (III), R¹ is described by one of the following structures:

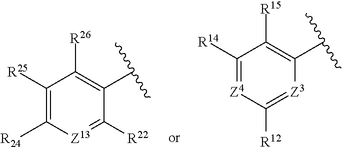

where Z¹³ is CR²³ or N, where Z³ is N or CR¹¹; Z⁴ is N or CR¹³; and R¹¹ to R¹⁵ and R²³-R²⁶ are each independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro.

In certain embodiments, in formula (III), R¹ is described by the following:

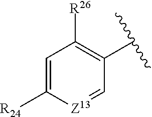

where Z¹³ is CR²³ or N, and R²³, R²⁴ and R²⁶ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro. In certain embodiments, R²³, R²⁴ and R²⁶ are independently selected from H, alkyl (e.g., methyl or ethyl), alkoxy (e.g., methoxy or ethoxy) and halo (e.g., fluoro or chloro). In certain cases, Z¹³ is selected from CH and N.

In certain embodiments, in formula (III), R¹ to R⁴ are independently selected from corresponding groups as depicted in any of the structures of Table 1 or 2.

In some cases, the compound is described by the structure of formula (IV):

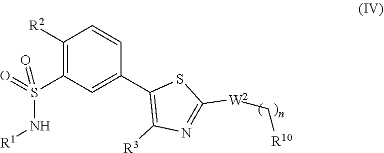

where: R¹ is —(CH₂)ₙ-R²⁰, where R²⁰ is an aryl, a cycloalkyl or a heterocycle and n is 0, 1 or 2; R² is selected from hydrogen, a halogen, an alkyl, a substituted alkyl, an alkoxy and a substituted alkoxy; R³ is H or alkyl; W² is a covalent bond, —NH—, or —NHCO—; n is 0, 1, 2 or 3; and R¹⁰ is a cycloalkyl or a heterocycle. In certain cases, in formula (IV), R¹ is a phenyl, a pyridyl, a diazinyl, a piperidinyl, a piperazinyl, or a pyrriloidinyl. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain cases, in formula (IV), R¹ is described by the following structure:

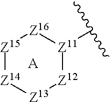

where A is a 6-membered aryl, heteroaryl, heterocyclyl, or cycloalkyl, where Z¹¹-Z¹⁶ are independently selected from N, CR′, NR and CR′R″, where R is H or alkyl, and R′ and R″ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro.

In some cases, the compound is described by the structure of formula (V):

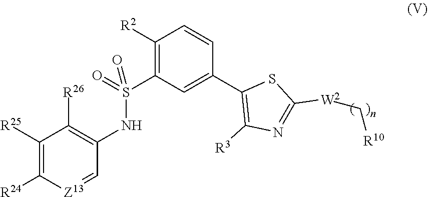

where: R² is selected from hydrogen, a halogen, an alkyl, a substituted alkyl, an alkoxy and a substituted alkoxy; R³ is lower alkyl, W² is a covalent bond, —NH—, or —NHCO—; n is 0, 1 or 2; Z¹³ is N or CR²³, R¹⁰ is a cycloalkyl or a heterocycle; and R²³-R²⁶ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro. In certain embodiments, R²³-R²⁶ are independently selected from hydrogen, halo, alkyl, and alkoxy. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In some instances, the compound is described by the structure of one of formulae (VI), (VII) or (VIII):

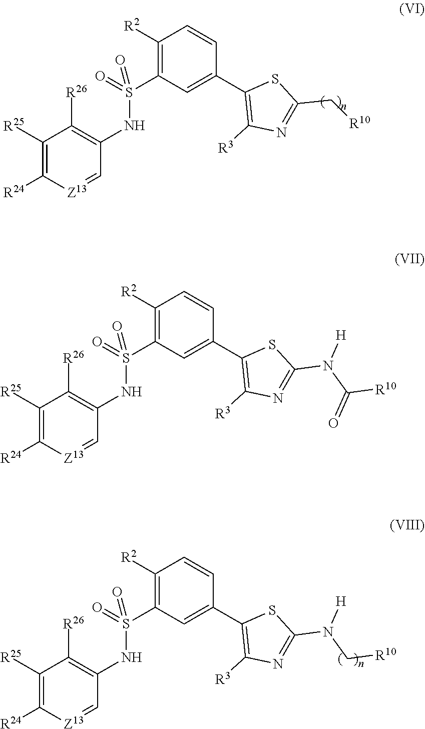

where: R² is selected from hydrogen, a halogen, an alkyl, a substituted alkyl, an alkoxy and a substituted alkoxy; R³ is lower alkyl, n is 0, 1 or 2; Z¹³ is N or CR²³, R¹⁰ is a cycloalkyl or a heterocycle; and R²³-R²⁶ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro. In certain embodiments, R²³-R²⁶ are independently selected from hydrogen, halo, alkyl, and alkoxy. In certain instances, R¹⁰ is a cyclopentyl, a cyclohexyl, a piperidinyl or a pyrrolidinyl.

In certain embodiments, in formulae (IV)-(VIII), R² is methoxy. In certain embodiments, in formulae (IV)-(VIII), R³ is methyl. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments, in formulae (IV)-(VIII), Z¹³, R²³-R²⁶, R², R³ and R¹⁰ are independently selected from corresponding groups as depicted in any of the structures of Table 1 or 2.

In some cases, the compound is described by the structure of formula (IX):

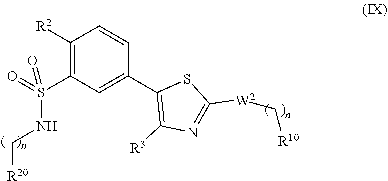

where: R² is selected from hydrogen, a halogen, an alkyl, a substituted alkyl, an alkoxy and a substituted alkoxy; R³ is lower alkyl; W² is a covalent bond, —NH—, or —NHCO—; each n is independently 0, 1 or 2; Z¹³ is N or CR²³; R¹⁰ is a cycloalkyl or a heterocycle; and R²⁰ is an aryl, a cycloalkyl or a heterocycle. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain cases, R²⁰ and R¹⁰ are independently described by the following structure:

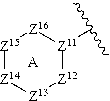

where A is a 6-membered aryl, heteroaryl, heterocyclyl, or cycloalkyl, where Z¹¹-Z¹⁶ are independently selected from N, CR′, NR and CR′R″, where R is H, an alkyl, a cycloalkyl, a heterocycloalkyl, an aryl or a heteroaryl; and R′ and R″ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro. In certain embodiments, R²⁰ is a phenyl, a pyridyl, a diazinyl, a piperidinyl, a piperazinyl, or a pyrrolidinyl.

In some instances, the PI4-kinase inhibitor is described by the structure of one of formulae (X), (XI) or (XII):

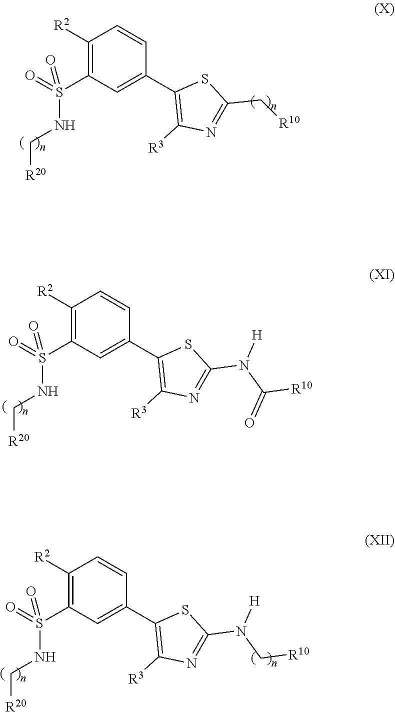

where R² is selected from hydrogen, a halogen, an alkyl, a substituted alkyl, an alkoxy and a substituted alkoxy; R³ is a lower alkyl, each n is independently 0, 1 or 2; R¹⁰ is a cycloalkyl or a heterocycle; and R²⁰ is an aryl, a cycloalkyl or a heterocycle. In certain embodiments, R²⁰ is a phenyl, a pyridyl, a diazinyl, a piperidinyl, a piperazinyl, or a pyrrolidinyl.

In certain instances, R¹⁰ is a cyclopentyl, a cyclohexyl, a piperidinyl or a pyrrolidinyl. In certain instances, R²⁰ is a phenyl, or a pyridyl. In certain embodiments, in formulae (IX)-(XII), R² is methoxy. In certain embodiments, in formulae (IX)-(XII), R³ is methyl. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In some embodiments, the PI4-kinase inhibitor is described by the structure of formula (XIII):

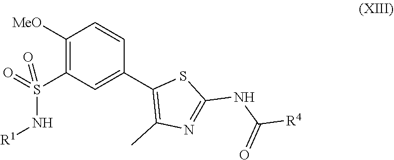

where R¹ is an alkyl, an aryl, an alkyl-heterocycle or a heterocycle; and R⁴ is an alkyl, an aralkyl, an aryl, an alkyl-cycloalkyl, a cycloalkyl, an alkyl-heterocycle, or a heterocycle. In certain instances, R⁴ is a cyclopentyl, a cyclohexyl, a piperidinyl or a pyrrolidinyl. In certain instances, R¹ is a phenyl or a pyridiyl.

In certain embodiments, in formulae (I)-(XIII), R¹ or R²⁰ is described by one of the following structures:

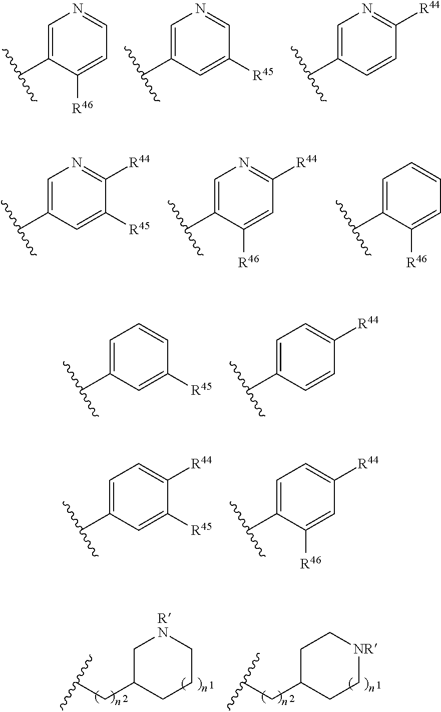

where R⁴⁴-R⁴⁶ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro; R′ is hydrogen, an alkyl, an aryl or a heterocycle; n² is 0, 1, 2 or 3, and n¹ is 0, 1 or 2; and

R⁴ is —(CH₂)ₙ-cycloalkyl (e.g., cyclopropyl, cyclopentyl or cyclohexyl), —(CH₂)ₙ-heterocycle (e.g., piperidinyl, a piperazinyl, or a pyrriloidinyl), or lower alkyl, where each n is independently 0, 1, 2 or 3. In certain embodiments, in formula (XIII), n is 1.

In certain embodiments, in formula (XIII), R¹ and R⁴ are independently selected from corresponding groups as depicted in any of the structures of Table 1 or 2.

In certain embodiments, the PI4-kinase inhibitor is described by one of the following structures:

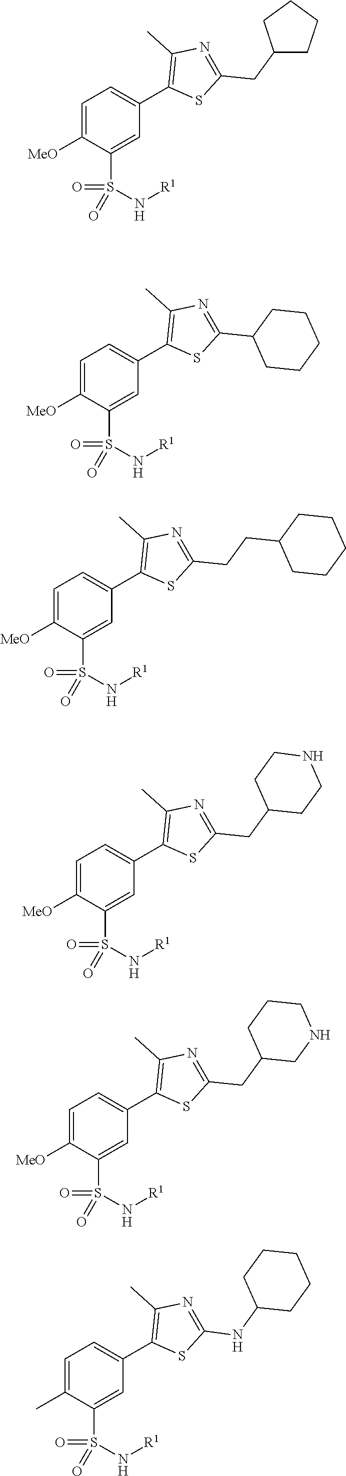

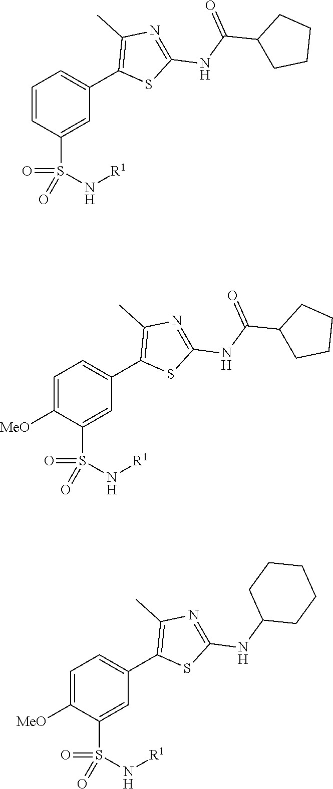

where R¹ is selected from a phenyl, a pyridyl, a diazinyl, a piperidinyl, a piperazinyl, a pyrriloidinyl and —(CH₂)ₙ-R²⁰ where R²⁰ is an aryl, a cycloalkyl or a heterocycle and n is 0, 1 or 2.

In certain embodiments, in the nine structures depicted above, R¹ is described by one of the following structures:

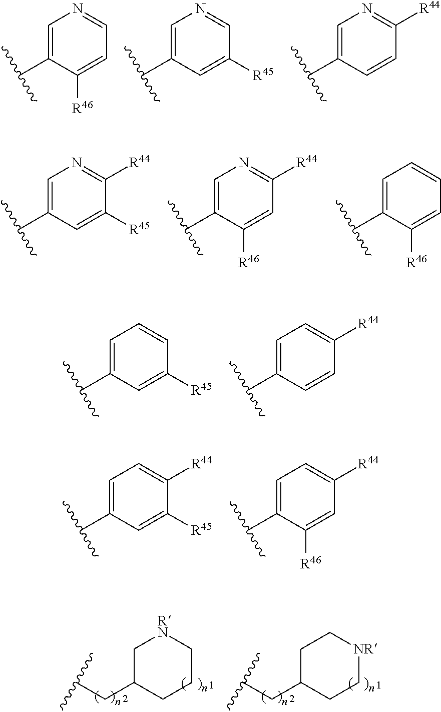

where R⁴⁴-R⁴⁶ are independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro; R′ is hydrogen, an alkyl, an aryl or a heterocycle; n² is 0, 1, 2 or 3, and n¹ is 0, 1 or 2. In certain embodiments, R⁴⁴-R⁴⁶ are independently selected from H, an alkyl, an alkoxy, hydroxyl, and a halo (e.g., fluoro or chloro).

In some embodiments, the PI4-kinase inhibitor is described by the structure of formula (XIV):

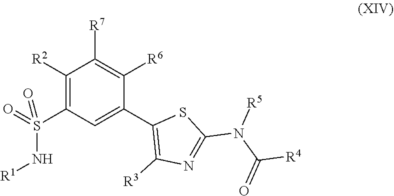

where R¹ is a phenyl, a pyridyl (e.g., 4-pyridyl or 3-pyridyl) or a pyrimidinyl (e.g., a 4-pyrimidinyl or 3-pyrimidinyl). In some embodiments, in formula (XIV), R⁵-R⁷ are each hydrogen. In certain embodiments, in formula (XIV), R¹ to R⁵ are independently selected from corresponding groups as depicted in any of the structures of Table 1 or 2. In some embodiments, in formula (XIV), R¹ is a phenyl, and R⁵-R⁷ are each hydrogen.

In some embodiments, the PI4-kinase inhibitor is described by the structure of formula (XIVb):

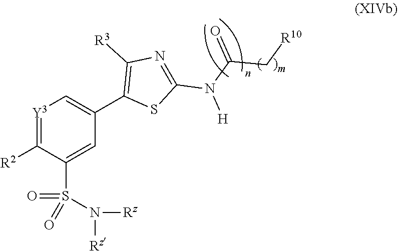

wherein n is 0 or 1; m is 0 or 1; R², R³, Y³ and R¹⁰ are as defined herein; R^{z} and R^{z′} are cyclically linked and together form a 5- or 6-membered saturated heterocycle with the N to which they are attached (e.g., a pyrrolidinyl ring or a piperidinyl ring), where the heterocycle is optionally substituted (e.g., substituted with an amino, a hydroxy, a substituted alkoxy, an alkoxy, a substituted alkyl or an alkyl group). In certain instances, n is 1 and m is 1 and R¹⁰ is a cycloalkyl or a substituted cycloalkyl. In certain instances, n is 1 and m is 0 and R¹⁰ is a cycloalkyl or a substituted cycloalkyl. In certain instances, n is 0 and m is 0 and R¹⁰ is a cycloalkyl or a substituted cycloalkyl. In certain instances, n is 0 and m is 1 and R¹⁰ is a cycloalkyl or a substituted cycloalkyl.

In certain embodiments, the PI4-kinase inhibitor is described by the structure of formula (XV):

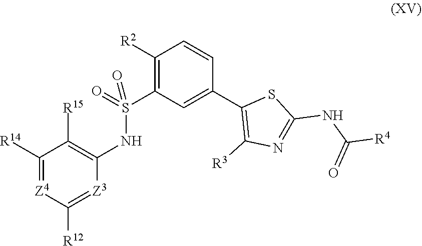

where Z³ is N or CR¹¹; Z⁴ is N or CR¹³; and R¹¹-R¹⁵ are each independently selected from hydrogen, an alkyl (e.g., a lower alkyl such as methyl or trifluoromethyl), an aryl, a hydroxy, an alkoxy, an aryloxy, a heterocycle, a cyano, a halogen (e.g., fluoro, chloro or bromo), an amino (e.g., —NMe₂), an acyl, an acyloxy, an amido, or a nitro. In some embodiments, in formula (XV), R² is selected from hydrogen, a halogen, an alkyl, a substituted alkyl, an alkoxy and a substituted alkoxy (e.g., methoxy).

In some embodiments, in formula (XV), R³ is an alkyl (e.g., methyl). In some embodiments, in formula (XV), Z⁴ is CR¹³ and Z³ is CR¹¹. In some embodiments, in formula (XV), Z⁴ is N and Z³ is CR¹¹. In some embodiments, in formula (XV), Z³ and Z⁴ are each N. In some embodiments, in formula (XV), R¹¹-R¹⁵ are each independently selected from hydrogen, an alkoxy (e.g., methoxy), a halogen (e.g., fluoro), acyloxy (e.g., acetyloxy), hydroxy and cyano-alkyl (e.g., cyano-methyl). In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In some embodiments, the PI4-kinase inhibitor is described by the structure of formula (XVI):

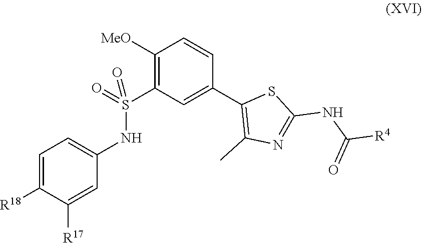

where R¹⁷ is hydrogen, an alkoxy (e.g., methoxy) or a halogen (e.g., fluoro); and R¹⁸ is selected from hydrogen, acetyloxy, hydroxy, methoxy, cyano-methyl and halogen (e.g., fluoro). In some embodiments, in formula (XVI), R⁴ is selected from methyl, trifluoromethyl, ethyl, tert-butyl, cyclopentyl, N-pyrrolidinyl, N-morpholinyl, N-amino-cyclopentyl and 1-fluoro-cyclopentyl; and R¹⁷ and R¹⁸ are independently selected from hydrogen, methoxy, fluoro, acetyloxy, hydroxy, and cyano-methyl.

In some embodiments, the PI4-kinase inhibitor is described by the structure of formula (XVII):

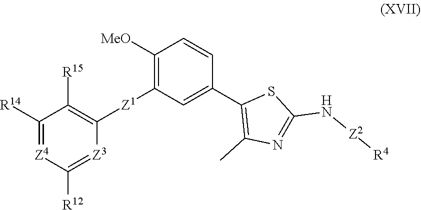

wherein: Z¹ is —NHSO₂— or —SO₂NH—; Z² is a covalent bond or —C(═O)—; Z³ is N or CR¹¹; Z⁴ is N or CR¹³; and R¹¹ to R¹⁵ are each independently selected from hydrogen, an alkyl, an aryl, a hydroxy, an alkoxy, an aryloxy, a heterocycle, a cyano, a halogen (e.g., fluoro, chloro or bromo), an amino, an acyl, an acyloxy, an amido, and a nitro. In certain embodiments, in formula (XVII), R¹¹ to R¹⁵ are each independently selected from hydrogen, an alkoxy, an acyloxy, a halogen, and hydroxyl. In some embodiments, in formula (XVII), Z³ is CR¹¹, Z⁴ is CR¹³; R¹¹, R¹², R¹⁴ and R¹⁵ are each hydrogen; and R¹³ is selected from hydrogen, acetyloxy, hydroxy, methoxy and halogen (e.g., fluoro). In some embodiments, in formula (XVII), Z³ is CR¹¹, Z⁴ is N, and R¹¹, R¹², R¹⁴ and R¹⁵ are each hydrogen. In some embodiments, in formula (XVII), Z³ and Z⁴ are each N, and R¹², R¹⁴ and R¹⁵ are each hydrogen. In some embodiments, in formula (XVII), Z² is a covalent bond or —C(═O)—, and R⁴ is a lower alkyl (e.g., trifluoromethyl, tert-butyl), a cycloalkyl (e.g., cyclopentyl or 1-fluoro-cyclopentyl) or —CH₂-cycloalkyl (e.g., —CH₂-cyclopentyl), a heterocycle (e.g., a N-linked saturated heterocycle such as N-pyrrolidino or N-morpholino), or an amino (e.g., an amino-alkyl such as N-amino-cyclopentyl). In some embodiments, in formula (XVII), Z² is —C(═O)—, and R⁴ is described by the formula —NR¹⁶R¹⁷, where R¹⁶ and R¹⁷ are each independently selected from hydrogen, an alkyl, and a cycloalkyl, wherein optionally R¹⁶ and R¹⁷ are cyclically linked (e.g., to form a N-heterocyclyl). In some embodiments, in formula (XVII), Z² is a single bond; and R⁴ is an alkyl or a cycloalkyl-alkyl (e.g., 1-cyclopentyl-methyl). In some embodiments, in formula (XVII), Z² is —C(═O)—, and R⁴ is selected from trifluoromethyl, ethyl, tert-butyl, N-pyrrolidinyl, N-morpholinyl, N-amino-cyclopentyl and 1-fluoro-cyclopentyl.

In some embodiments, the PI4-kinase inhibitor is described by the structure of formula (XVIII):

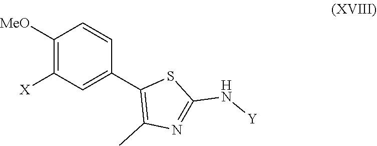

where X and Y are independently selected from the substituents groups shown below:

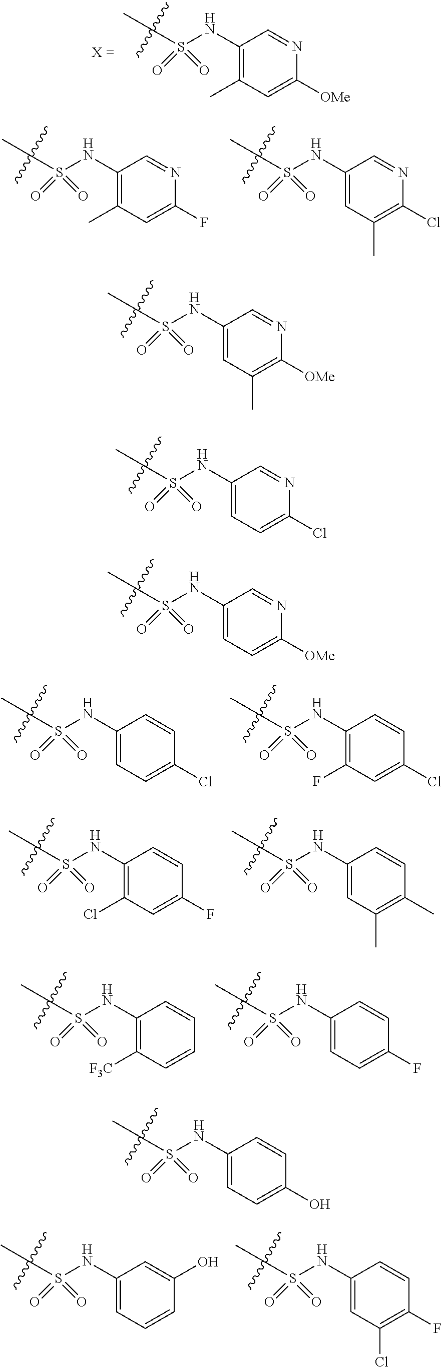

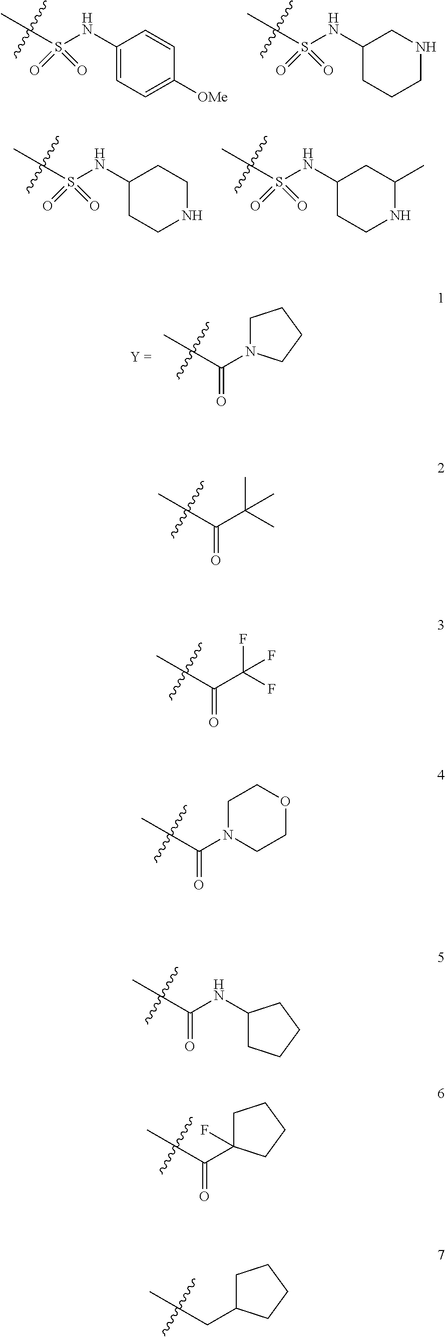

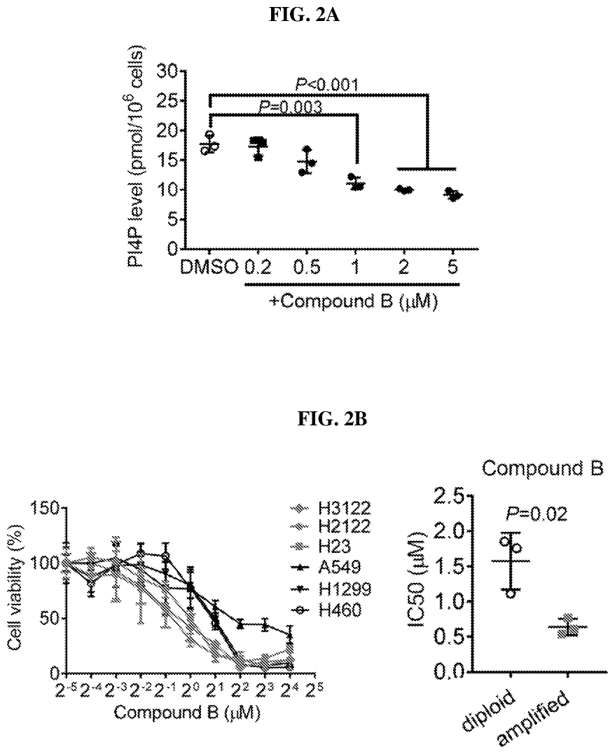

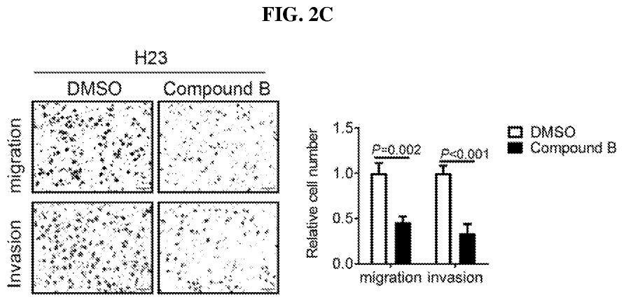

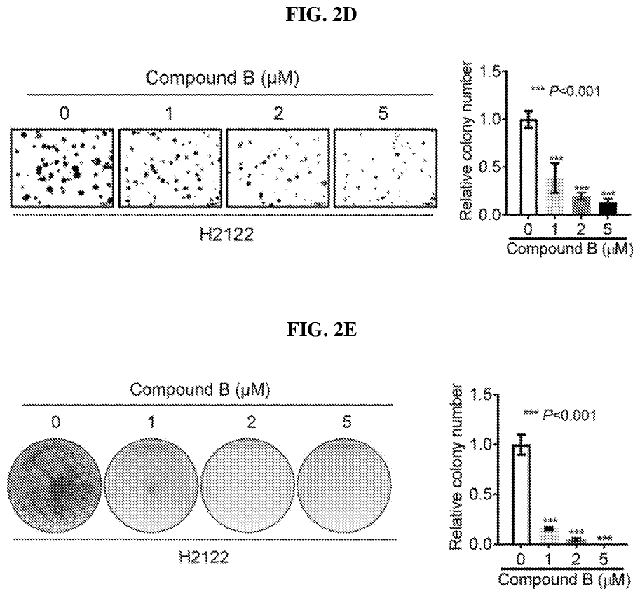

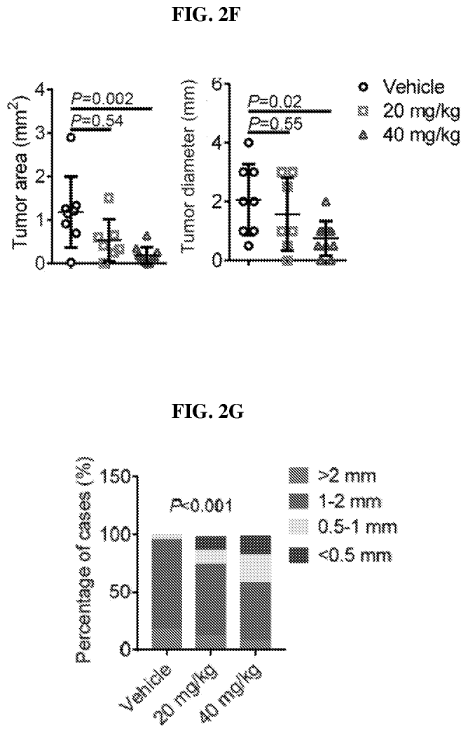

In certain embodiments, in formula (XVIII), R¹ and R⁴ are independently selected from corresponding groups as depicted in any of the structures of Table 1 or 2. In some embodiments, the subject compound is described by the structure of compound PT423, shown in Table 1. In some embodiments, the subject compound is described by one of the structures labeled M1, M2, and M3 in FIG. 2.

In some instances of formula (Ib), the PI4-kinase inhibitor has the formula (XXI):

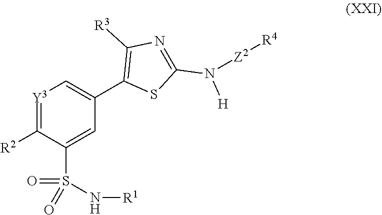

wherein: R² is selected from hydrogen, a halogen, an alkyl, a substituted alkyl, an alkoxy and a substituted alkoxy; R³ is hydrogen, a lower alkyl (e.g., methyl) or a substituted lower alkyl; Y³ is CH or N; Z² is absent, CO or SO₂; R¹ is an aryl, a substituted aryl (e.g., a substituted phenyl), a heteroaryl, a substituted heteroaryl (e.g., a substituted pyridyl), an alkyl, a substituted alkyl, a cycloalkyl, a substituted cycloalkyl (e.g., a substituted cyclohexyl), a heterocycle (e.g., a tetrahydropyran or a piperidinyl) or a substituted heterocycle; and R⁴ is selected from alkyl, substituted alkyl, cycloalkyl, substituted cycloalkyl, alkyl-cycloalkyl, substituted alkyl-cycloalkyl, aryl, substituted aryl, heterocycle, substituted heterocycle, alkyl-heterocycle (e.g., —CH₂-(4-tetrahydropyran)) and substituted alkyl-heterocycle; or a prodrug thereof, or a pharmaceutically acceptable salt thereof. In certain instances, Z² is absent. In certain instances, Z² is —C(═O)—. In certain instances, Z² is —S(═O)₂—. In certain instances, R¹ is a phenyl, a substituted phenyl, a cyclohexyl, a substituted cyclohexyl, a piperidinyl (e.g., a 3-piperidinyl or a 4-piperidinyl) or a substituted piperidinyl (e.g., a substituted 3-piperidinyl or substituted 4-piperidinyl). In certain instances, R¹ is a substituted phenyl. In certain instances, R¹ is a substituted alkyl, e.g., a substituted lower alkyl. In certain instances, R⁴ is a cyclohexyl, a substituted cyclohexyl, a tetrahydropyran, a benzyl, a substituted benzyl, a phenyl, a substituted phenyl, a methylene-cyclohexane and a substituted methylene-cyclohexane. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments of formula (XXI), the PI4-kinase inhibitor has formula (XXII):

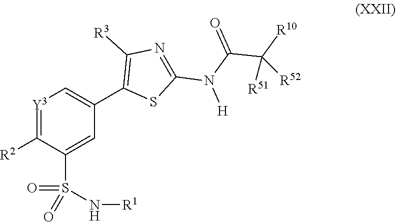

wherein: R² is selected from hydrogen, a halogen, an alkyl, a substituted alkyl, an alkoxy and a substituted alkoxy; R¹⁰ is selected from cycloalkyl, substituted cycloalkyl, heterocycle (e.g., 4-tetrahydropyran) and substituted heterocycle; and R⁵¹ and R⁵² are independently selected from H, halogen (e.g., fluoro), alkyl (e.g., lower alkyl) and substituted alkyl. In certain instances, R¹ is a phenyl, a substituted phenyl, a cyclohexyl, a substituted cyclohexyl, a piperidinyl (e.g., a 3-piperidinyl or a 4-piperidinyl) or a substituted piperidinyl (e.g., a substituted 3-piperidinyl or substituted 4-piperidinyl). In certain instances, R¹ is a substituted phenyl. In certain instances, R¹ is a substituted alkyl, e.g., a substituted lower alkyl. In certain instances, R¹⁰ is a cyclohexyl, a substituted cyclohexyl, a tetrahydropyran, a benzyl, a substituted benzyl, a phenyl, a substituted phenyl, a methylene-cyclohexane and a substituted methylene-cyclohexane. In certain instances, R⁵¹ and R⁵² are each hydrogen. In certain instances, R⁵¹ is hydrogen and R⁵² is alkyl or substituted alkyl (e.g., methyl). In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments of formula (XXII), the PI4-kinase inhibitor has formula (XXIII):

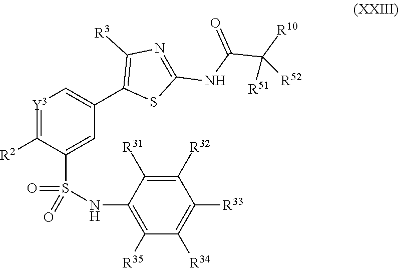

wherein: R² is selected from hydrogen, a halogen, an alkyl, a substituted alkyl, an alkoxy and a substituted alkoxy; R³¹-R³⁵ are independently selected from hydrogen, halogen (e.g., fluoro), alkyl, substituted alkyl, hydroxy, alkoxy, substituted alkoxy, acyl, substituted acyl and —CO₂R, wherein R is H, alkyl or substituted alkyl. In certain instances, R³¹ and R³³ are halogen (e.g., fluoro or chloro). In certain instances, R³¹ and R³⁵ are halogen (e.g., fluoro or chloro). In certain instances, R³¹ is halogen (e.g., fluoro or chloro). In certain instances, 3 or 4 of R³¹-R³⁵ are hydrogen. In certain instances, R⁵¹ and R⁵² are each hydrogen. In certain instances, R⁵¹ is hydrogen and R⁵² is alkyl or substituted alkyl (e.g., methyl). In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments of formulae (XXII) or (XXIII), the PI4-kinase inhibitor has the formula (XXIV) or formula (XXV):

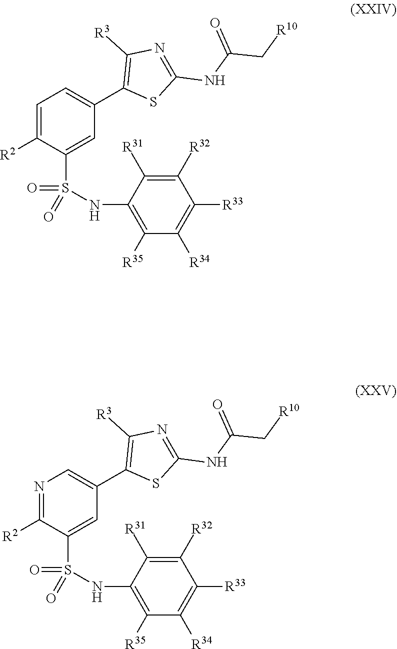

wherein: one and only one of R³³ and R³⁴ is hydroxy; and R² is selected from hydrogen, a halogen, an alkyl, a substituted alkyl, an alkoxy and a substituted alkoxy. In certain instances, R³³ is hydroxy. In certain instances, R³⁴ is hydroxy. In certain instances, 3, 4 or 5 of R³¹-R³⁵ are hydrogen. In certain cases, R¹⁰ is a cyclohexyl or substituted cyclohexyl. In certain cases, R¹⁰ is tetrahydropyran (e.g., 4-tetrahydropyranyl) or substituted tetrahydropyran (e.g., substituted 4-tetrahydropyranyl). In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments of formula (XXIV) and formula (XXV), the PI4-kinase inhibitor has one of the formulae (XXVI)-(XXVIII):

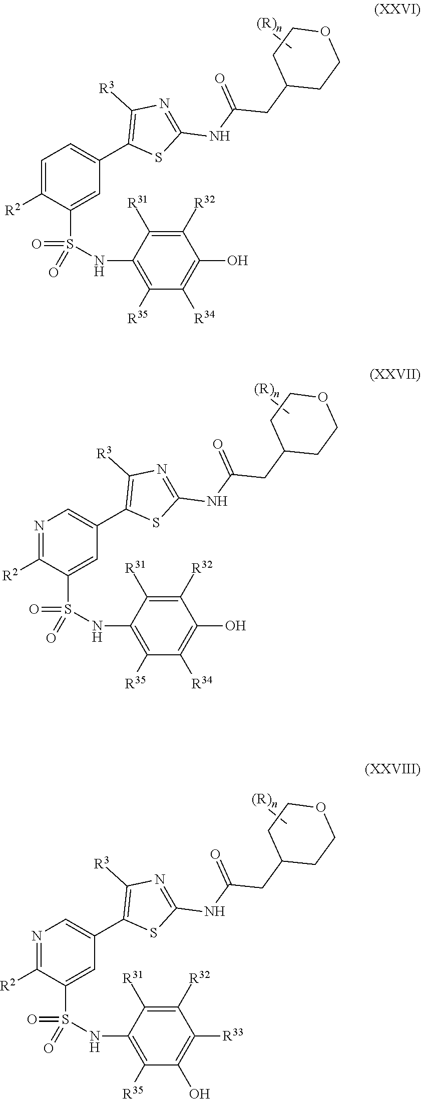

wherein: R² is selected from hydrogen, a halogen, an alkyl, a substituted alkyl, an alkoxy and a substituted alkoxy; R³¹, R³², R³⁴ and R³⁵ are independently selected from hydrogen and halogen (e.g., fluoro); and (R)ₙ is one or more optional substituents each independently selected from alkyl, substituted alkyl, hydroxyl, alkoxy, substituted alkoxy, halogen (e.g., fluoro or chloro), and CO₂R″ where R″ is hydrogen, alkyl or substituted alkyl. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe. In certain embodiments, the PI4-kinase inhibitor has one of the following structures:

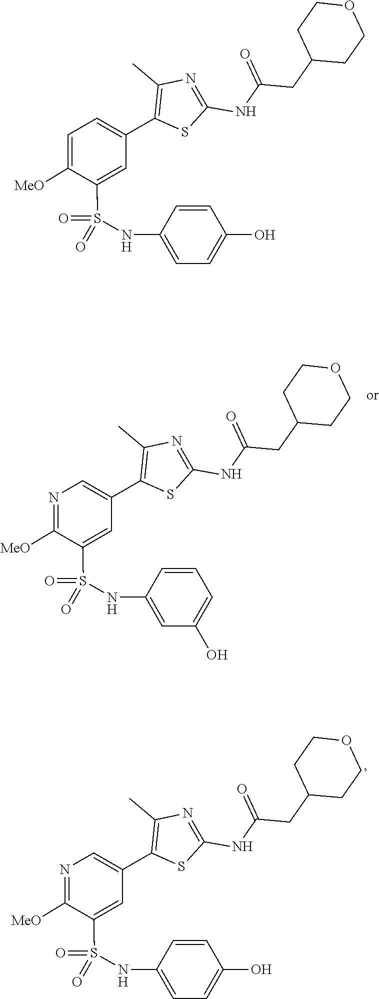 [SMILES: COc1ccc(-c2sc(NC(=O)CC3CCOCC3)nc2C)cc1S(=O)(=O)Nc1ccc(O)cc1 ; COc1ncc(-c2sc(NC(=O)CC3CCOCC3)nc2C)cc1S(=O)(=O)Nc1cccc(O)c1 ; COc1ncc(-c2sc(NC(=O)CC3CCOCC3)nc2C)cc1S(=O)(=O)Nc1ccc(O)cc1]

or a prodrug thereof, or a pharmaceutically acceptable salt thereof.

In certain embodiments of formula (XXIII), the PI4-kinase inhibitor has formula (XXIX):

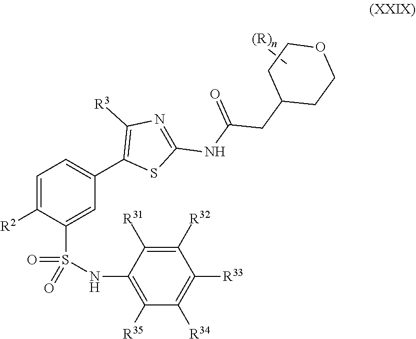

wherein: R² is selected from hydrogen, a halogen, an alkyl, a substituted alkyl, an alkoxy and a substituted alkoxy; R³¹-R³⁵ are independently selected from hydrogen and halogen (e.g., fluoro or chloro), wherein 0, 1 or 2 of R³¹-R³⁵ are halogen; and (R)ₙ is one or more optional substituents each independently selected from alkyl, substituted alkyl, hydroxyl, alkoxy, substituted alkoxy, halogen (e.g., fluoro or chloro) and CO₂R″ wherein R″ is hydrogen, alkyl or substituted alkyl. In certain instances, R³¹ and R³³ are halogen (e.g., fluoro or chloro). In certain instances, R³¹ and R³⁵ are halogen (e.g., fluoro or chloro). In certain instances, R³¹ is halogen (e.g., fluoro or chloro). In certain instances, 3 or 4 of R³¹-R³⁵ are hydrogen. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments of formula (XXII), the PI4-kinase inhibitor has formula (XXX):

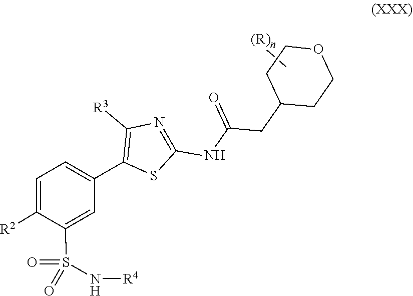

wherein: R² is selected from hydrogen, a halogen, an alkyl, a substituted alkyl, an alkoxy and a substituted alkoxy; R⁴ is a lower alkyl or a substituted lower alkyl (e.g., an isopropyl); and (R)ₙ is one or more optional substituents each independently selected from alkyl, substituted alkyl, hydroxyl, alkoxy, substituted alkoxy, halogen (e.g., fluoro or chloro), and CO₂R″ where R″ is hydrogen, alkyl or substituted alkyl. In certain instances, n is 0. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments of formula (XXII), the PI4-kinase inhibitor has the formula (XXXI):

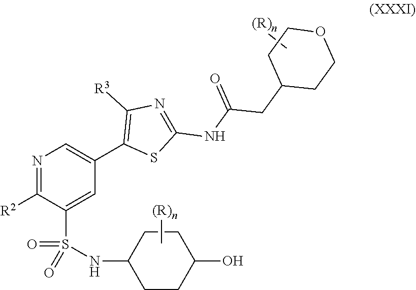

wherein: R² is selected from hydrogen, a halogen, an alkyl, a substituted alkyl, an alkoxy and a substituted alkoxy; each (R)ₙ is one or more optional substituents each independently selected from alkyl, substituted alkyl, hydroxyl, alkoxy, substituted alkoxy, halogen (e.g., fluoro or chloro) and CO₂R″ where R″ is hydrogen, alkyl or substituted alkyl. In certain instances, each n is 0. In certain instances, each n is 0. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments, the PI4-kinase inhibitor has the structure:

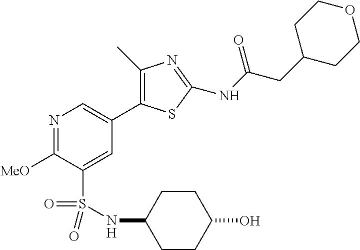

or a prodrug thereof, or a pharmaceutically acceptable salt thereof.

In certain embodiments of formula (XXIII), the PI4-kinase inhibitor has the formula (XXXII):

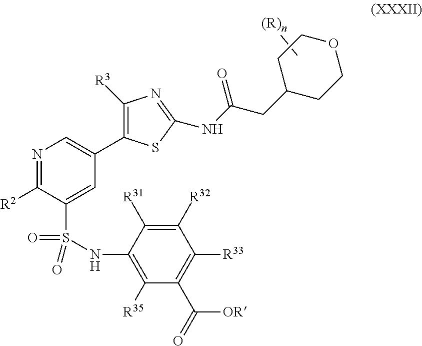

wherein: R² is selected from hydrogen, a halogen, an alkyl, a substituted alkyl, an alkoxy and a substituted alkoxy; R³¹-R³³ and R³⁵ are independently selected from hydrogen and halogen (e.g., fluoro); (R)ₙ is one or more optional substituents each independently selected from alkyl, substituted alkyl, hydroxyl, alkoxy, substituted alkoxy, halogen (e.g., fluoro or chloro) and CO₂R″ where R″ is hydrogen, alkyl or substituted alkyl; and R′ is H, alkyl or substituted alkyl. In certain embodiments, R′ is a lower alkyl. In certain cases, R′ is ethyl. In certain cases, R′ is methyl. In certain embodiments, n is 0. In certain embodiments, R³¹-R³³ and R³⁵ are each hydrogen. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe. In certain embodiments, the PI4-kinase inhibitor has the structure:

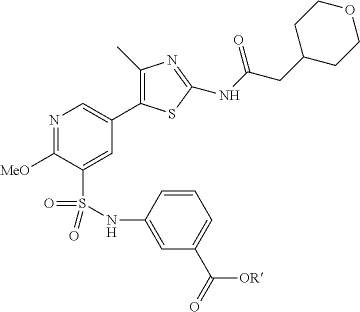

wherein R′ is H, a lower alkyl or a substituted lower alkyl; or a prodrug thereof, or a pharmaceutically acceptable salt thereof. In certain cases, R′ is hydrogen. In certain cases, R′ is lower alkyl. In certain cases, R′ is ethyl. In certain cases, R′ is methyl.

In some embodiments, the PI4-kinase inhibitor has the following structure:

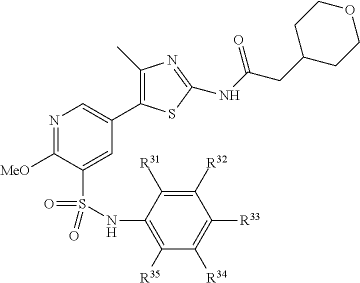

where R³¹-R³⁵ are selected from one of the following embodiments:

| Embodiment | R³¹ | R³² | R³³ | R³⁴ | R³⁵ |
|---|---|---|---|---|---|
| 1 | H | H | H | H | H |
| 2 | F | H | F | H | H |
| 3 | Cl | H | H | H | H |
| 4 | Cl | H | Cl | H | H |
| 5 | Cl | H | H | H | Cl |

In certain embodiments of formula (XXI), the PI4-kinase inhibitor has the formula (XXXIII) or formula (XXXIV)

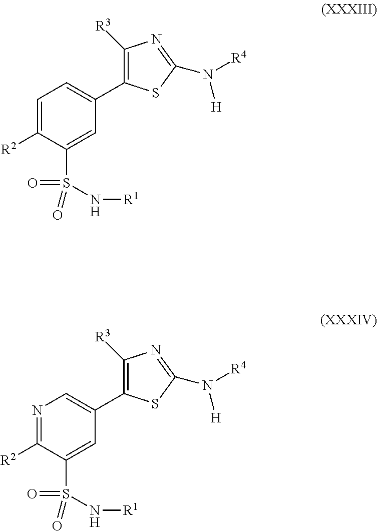

wherein: R² is selected from hydrogen, a halogen, an alkyl, a substituted alkyl, an alkoxy and a substituted alkoxy; R³ is hydrogen, a lower alkyl (e.g., methyl) or a substituted lower alkyl; R¹ is an aryl, a substituted aryl, (e.g., a substituted phenyl), a heteroaryl, a substituted heteroaryl, (e.g., a substituted pyridyl), a cycloalkyl, a substituted cycloalkyl (e.g., a substituted cyclohexyl), a heterocycle (e.g., a tetrahydropyran or a piperidinyl) or a substituted heterocycle; and R⁴ is selected from alkyl, substituted alkyl, cycloalkyl, substituted cycloalkyl, alkyl-cycloalkyl, substituted alkyl-cycloalkyl, heterocycle, substituted heterocycle, alkyl-heterocycle (e.g., —CH₂-(4-tetrahydropyran)) and substituted alkyl-heterocycle. In certain instances, R¹ is a phenyl, a substituted phenyl, a cyclohexyl, a substituted cyclohexyl, a piperidinyl (e.g., a 3-piperidinyl or a 4-piperidinyl) or a substituted piperidinyl (e.g., a substituted 3-piperidinyl or substituted 4-piperidinyl). In certain instances, R¹ is a substituted phenyl. In certain instances, R⁴ is a cyclohexyl, a substituted cyclohexyl, a tetrahydropyran, a methylene-tetrahydropyran, a substituted tetrahydropyran, a substituted methylene-tetrahydropyran, a benzyl, a substituted benzyl, a phenyl, a substituted phenyl, a methylene-cyclohexane and a substituted methylene-cyclohexane. In certain instances, R⁴ is cycloheptyl or a substituted cycloheptyl. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe.

In certain embodiments of formulae (XXXIII) or (XXXIV), the PI4-kinase inhibitor has one of the formula (XXXV) and formula (XXXVI):

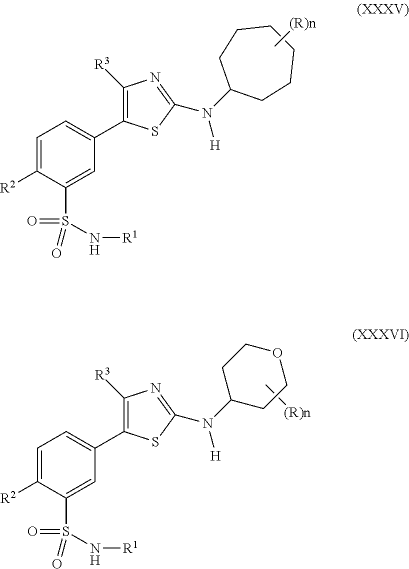

wherein: R² is selected from hydrogen, a halogen, an alkyl, a substituted alkyl, an alkoxy and a substituted alkoxy; (R)ₙ is one or more optional substituents each independently selected from alkyl, substituted alkyl, hydroxyl, alkoxy, substituted alkoxy, halogen (e.g., fluoro or chloro), and CO₂R″ where R″ is hydrogen, alkyl and substituted alkyl. In certain instances, n is 0. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe. In certain embodiments, R¹ is phenyl or substituted phenyl (e.g., as described in any of the compounds of Tables 1-2). In certain embodiments, R¹ is pyridyl (e.g., 2-pyridyl, 3-pyridyl or 4-pyridyl) or substituted pyridyl (e.g., as described in compounds of Tables 1-2). In certain embodiments, R¹ is a saturated heterocycle or a substituted saturated heterocycle.

In some embodiments of formula (XXXV) and formula (XXXVI), the PI4-kinase inhibitor has one of the formula (XXXVII) and formula (XXXVIII):

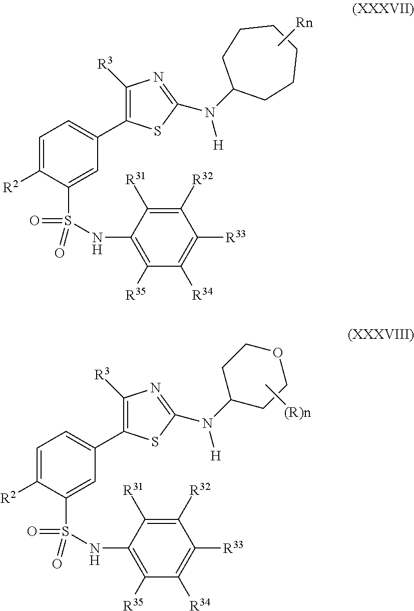

wherein: R² is selected from hydrogen, a halogen, an alkyl, a substituted alkyl, an alkoxy and a substituted alkoxy; R³¹-R³⁵ are independently selected from hydrogen, halogen (e.g., fluoro), alkyl, substituted alkyl, hydroxy, alkoxy, substituted alkoxy, acyl, substituted acyl and —CO₂R, wherein R is H, alkyl or substituted alkyl. In certain embodiments of formula (XXXVII) and formula (XXXVIII), R³¹-R³⁵ are independently selected from hydrogen, methyl, halogen (e.g., fluoro or chloro) and hydroxy. In certain instances, R³¹ and R³³ are halogen (e.g., fluoro or chloro). In certain instances, R³¹ and R³⁵ are halogen (e.g., fluoro or chloro). In certain instances, R³¹ is halogen (e.g., fluoro or chloro). In certain instances, 3 or 4 of R³¹-R³⁵ are hydrogen. In certain embodiments of formula (XXXVII) and formula (XXXVIII), R³¹ and R³⁵ are independently lower alkyl or substituted lower alkyl (e.g., methyl). In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe. In certain instances, the PI4-kinase inhibitor has the structure:

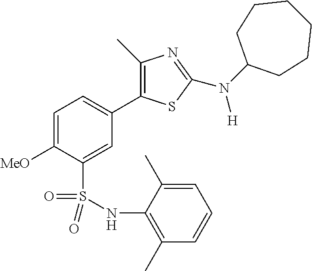 [SMILES: COc1ccc(-c2sc(NC3CCCCCC3)nc2C)cc1S(=O)(=O)Nc1c(C)cccc1C]

or a prodrug thereof, or a pharmaceutically acceptable salt thereof.

In some embodiments, the PI4-kinase inhibitor has one of the following structures:

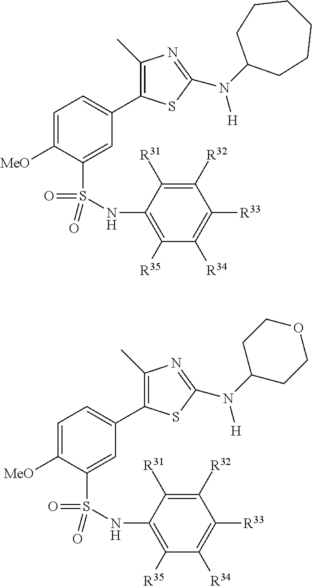

where R³¹-R³⁵ are selected from one of the following embodiments:

| Embodiment | R³¹ | R³² | R³³ | R³⁴ | R³⁵ |
|---|---|---|---|---|---|
| 1 | Cl | H | H | H | Cl |
| 2 | Cl | H | H | H | F |
| 3 | Cl | H | Cl | H | H |
| 4 | Cl | H | H | H | H |
| 5 | Me | H | OH | H | H |
| 6 | Cl | H | OH | H | H |

In some embodiments of formula (XXI), the PI4-kinase inhibitor has the formula (XXXIX):

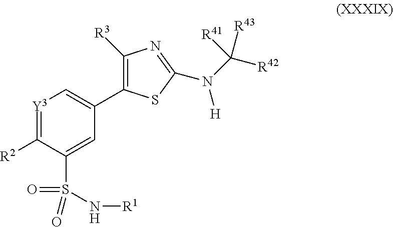

wherein: R² is selected from hydrogen, a halogen, an alkyl, a substituted alkyl, an alkoxy and a substituted alkoxy; R⁴¹ and R⁴³ are independently hydrogen, a lower alkyl or a substituted lower alkyl (e.g., methyl); and R⁴² is selected from alkyl, substituted alkyl, cycloalkyl, substituted cycloalkyl, alkyl-cycloalkyl, substituted alkyl-cycloalkyl, heterocycle, substituted heterocycle, alkyl-heterocycle (e.g., —CH₂-(4-tetrahydropyran)) and substituted alkyl-heterocycle. In certain cases, Y³ is N. In certain cases, Y³ is CH. In certain embodiments, R¹ is phenyl or substituted phenyl (e.g., as described in any of the compounds of Tables 1-2). In certain embodiments, R¹ is pyridyl (e.g., 2-pyridyl, 3-pyridyl or 4-pyridyl) or substituted pyridyl (e.g., as described in compounds of Tables 1-2). In certain embodiments, R¹ is a saturated heterocycle or a substituted saturated heterocycle. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe. In certain embodiments, R⁴³ is H. In certain embodiments, R⁴¹ is alkyl or substituted alkyl. In certain embodiments, R⁴¹ and R⁴³ are independently alkyl or substituted alkyl. In certain embodiments of formula (XXXIX), the PI4-kinase inhibitor has the formula (XL) or (XLI):

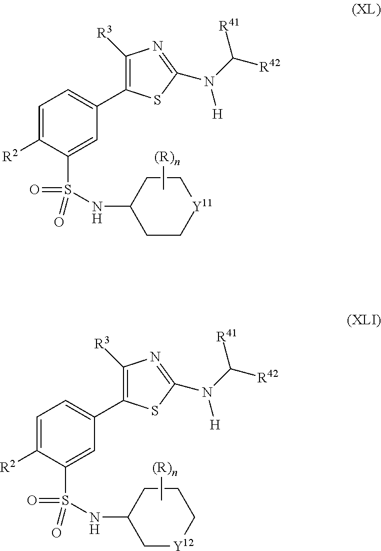

wherein: R² is selected from hydrogen, a halogen, an alkyl, a substituted alkyl, an alkoxy and a substituted alkoxy; Y¹¹ and Y¹² are selected from CR″₂, NR″ and O, wherein each R″ is independently H, R, an acyl or a substituted acyl; each R is independently H, an alkyl, a substituted alkyl, an alkoxy or a halogen (e.g., a fluoro); and n is 0, 1, 2, 3 or 4. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe. In certain embodiments, Y¹¹ is CH(OH). In certain embodiments, Y¹¹ is CH(OR), where R is acyl (e.g., acetyl) or substituted acyl. In certain embodiments, Y¹¹ is O. In certain embodiments, Y¹¹ is NH.

In certain embodiments of formula (XL) or (XLI), the PI4-kinase inhibitor has the formulae (XLII) or (XLIII):

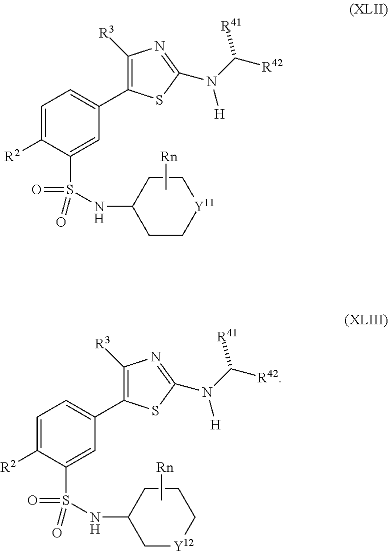

In some instances, Y¹¹ and Y¹² are each NH. In certain cases, n is 0 (e.g., there are no R groups present). In some instances of formulae (XL)-(XLIII), (R)ₙ is 4-CO₂R′, wherein R′ is hydrogen or lower alkyl (e.g., ethyl). In certain embodiments of formulae (XXXIX)-(XLIII), R⁴¹ is methyl; and R⁴² is selected from cyclohexyl, substituted cyclohexyl, —CH₂-cyclohexyl and substituted —CH₂— cyclohexyl. In certain embodiments, Y¹¹ is CH(OH). In certain embodiments, Y¹¹ is CH(OR), where R is acyl (e.g., acetyl) or substituted acyl. In certain embodiments, Y¹¹ is O. In certain embodiments, Y¹¹ is NH. In certain embodiments, the PI4-kinase inhibitor has one of the following structures:

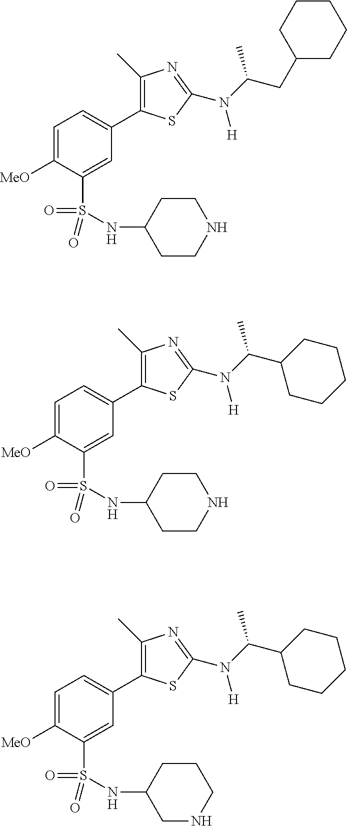 [SMILES: COc1ccc(-c2sc(N[C@H](C)CC3CCCCC3)nc2C)cc1S(=O)(=O)NC1CCNCC1 ; COc1ccc(-c2sc(N[C@H](C)C3CCCCC3)nc2C)cc1S(=O)(=O)NC1CCNCC1 ; COc1ccc(-c2sc(N[C@H](C)C3CCCCC3)nc2C)cc1S(=O)(=O)NC1CCCNC1]

or a prodrug thereof, or a pharmaceutically acceptable salt thereof.

In certain embodiments of formula (XXXIX), the PI4-kinase inhibitor has one of the formulae (XLIVa)-(XLIVh) and (XLVa)-(XLVh):

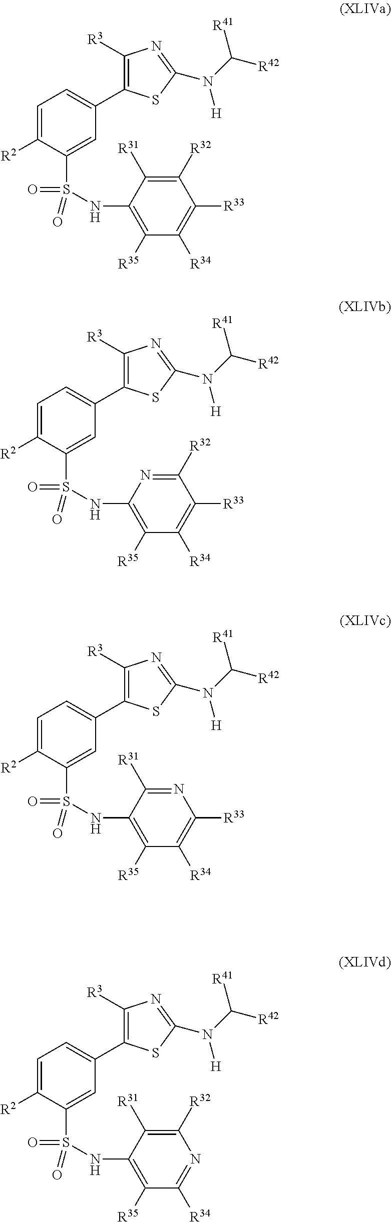

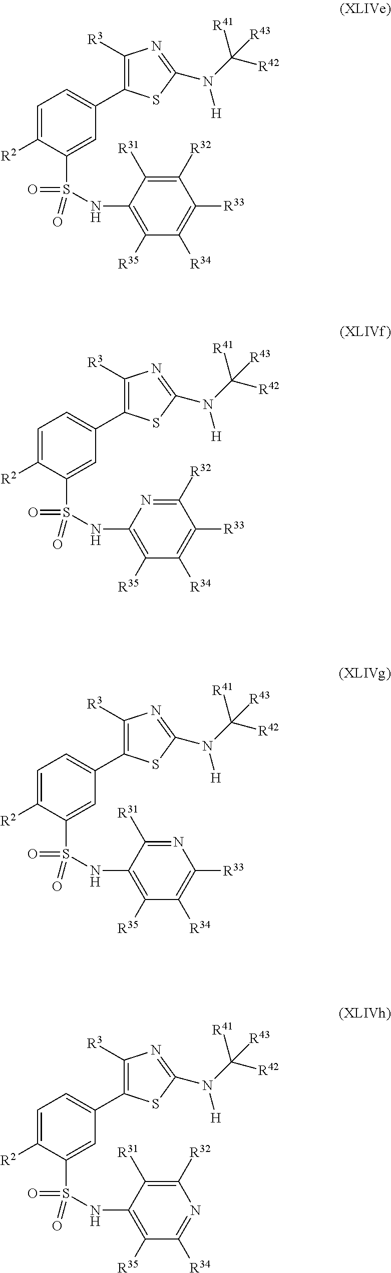

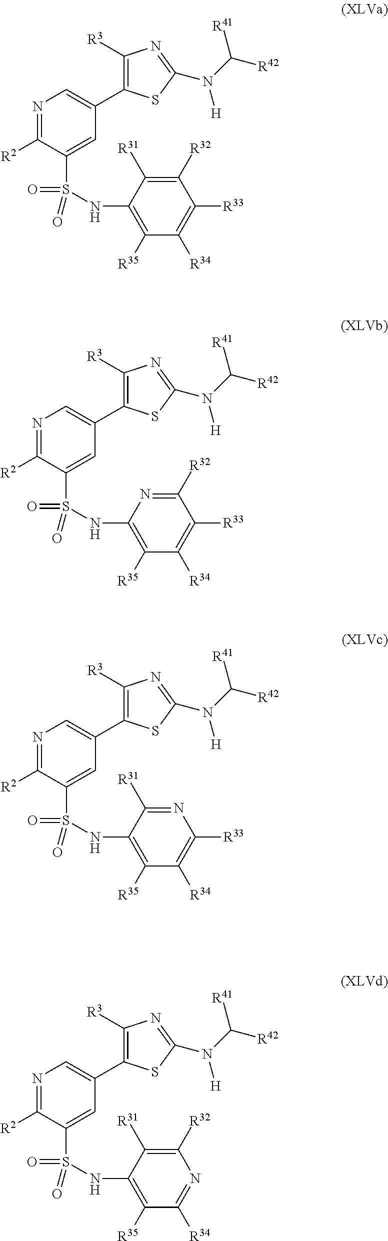

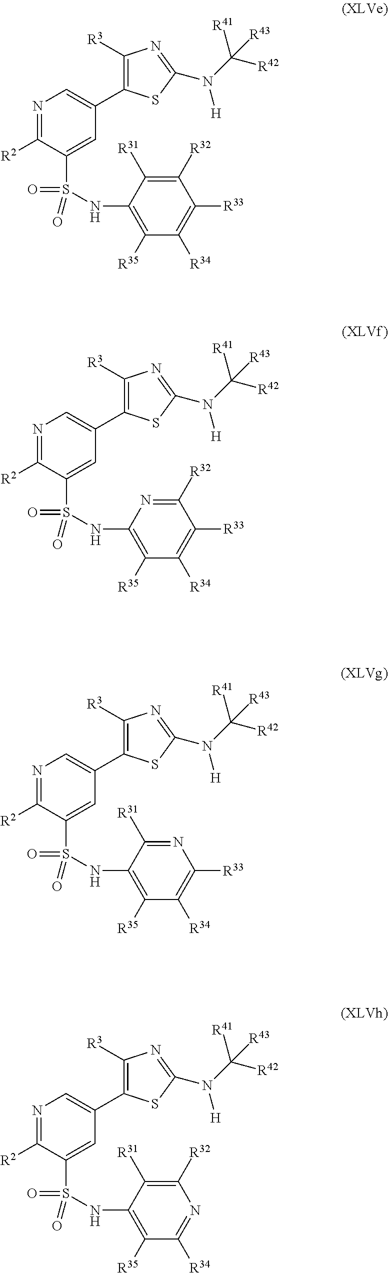

wherein R³¹-R³⁵ are independently selected from hydrogen, halogen (e.g., fluoro), alkyl, substituted alkyl, hydroxy, alkoxy, substituted alkoxy, acyl, substituted acyl and —CO₂R, wherein R is H, alkyl or substituted alkyl, R⁴¹ is H, alkyl or substituted alkyl, and R⁴³ is alkyl or substituted alkyl. In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe. In certain embodiments, R⁴¹ and R⁴³ are each independently alkyl or substituted alkyl. In certain embodiments, R⁴¹ and R⁴³ are each methyl. In certain embodiments, R⁴² is alkyl or substituted alkyl. In certain embodiments, R⁴² is methyl, ethyl, isopropyl, propyl. In certain embodiments, R⁴¹ and R⁴³ are each methyl and R⁴² is a lower alkyl or substituted lower alkyl (e.g., methyl, ethyl, isopropyl, propyl). In certain embodiments, R⁴² is a saturated heterocycle (e.g., 4-tetrahydropyran) or substituted saturated heterocycle. In certain embodiments, R⁴² is a cycloalkyl or substituted cycloalkyl. In certain embodiments of formula (XLIVa-d), R⁴¹ is alkyl or substituted alkyl and R⁴² is a saturated heterocycle (e.g., 4-tetrahydropyran) or substituted saturated heterocycle. In certain embodiments of formula (XLIVe-h), R⁴¹-R⁴³ are independently alkyl or substituted alkyl. In certain embodiments of formula (XLIVe-h), R⁴¹ and R⁴³ are each lower alkyl and R⁴² is alkyl or substituted alkyl. In certain embodiments of formula (XLVa-d), R⁴¹ is alkyl or substituted alkyl and R⁴² is a saturated heterocycle (e.g., 4-tetrahydropyran) or substituted saturated heterocycle. In certain embodiments of formula (XLVe-h), R⁴¹-R⁴³ are independently alkyl or substituted alkyl. In certain embodiments of formula (XLVe-h), R⁴¹ and R⁴³ are each lower alkyl and R⁴² is alkyl or substituted alkyl.

In certain embodiments of formulae (XLIVa-h) and formulae (XLVa-h), the PI4-kinase inhibitor has one of the formulae (XLVIa)-(XLVId) and (XLVIIa)-(XLVIId):

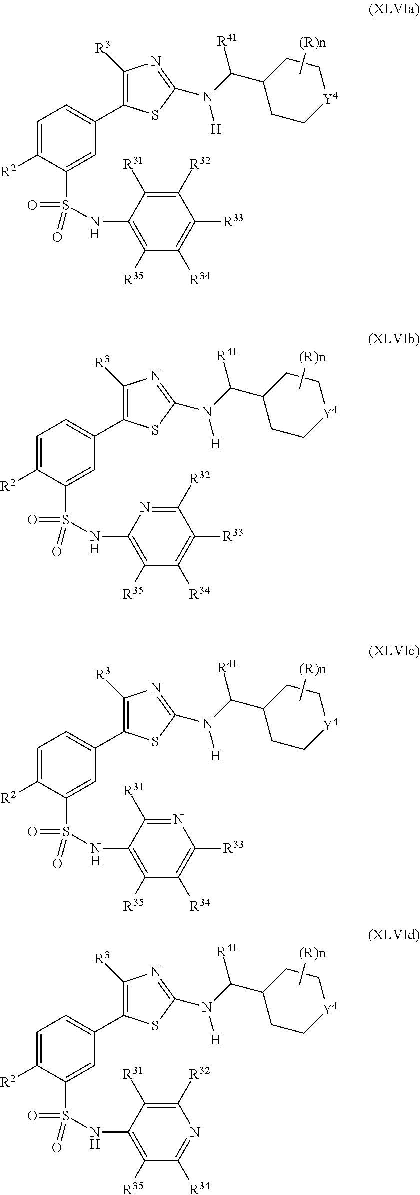

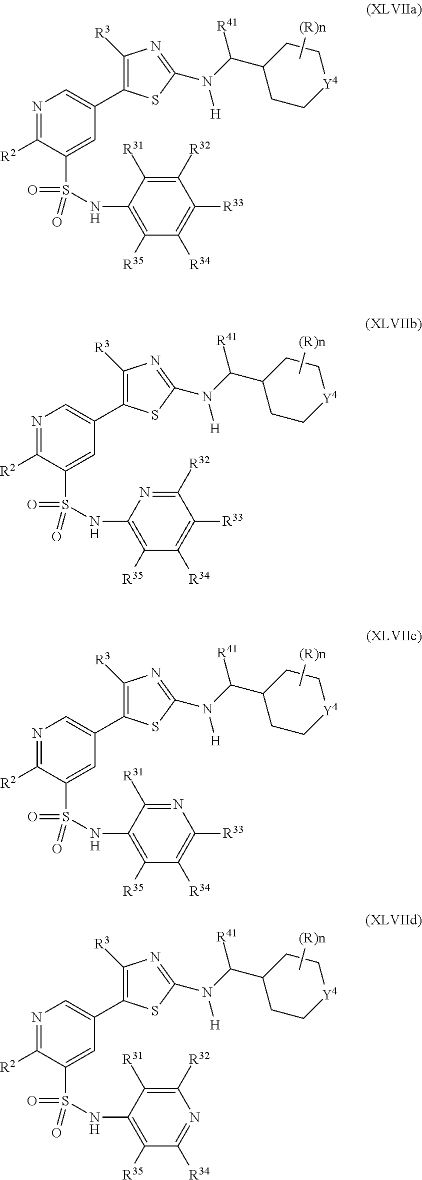

wherein: (R)ₙ is one or more optional substituents (i.e., n is 0, 1, 2, 3, 4 or 5) each independently selected from alkyl, substituted alkyl, hydroxyl, alkoxy, substituted alkoxy, halogen (e.g., fluoro or chloro) and CO₂R″ where R″ is hydrogen, alkyl or substituted alkyl; Y⁴ is CH, CR or O; and R⁴¹ is H, lower alkyl or substituted lower alkyl. In certain instances of formula (XLVIa-d) and formula (XLVIIa-d), R³¹-R³⁵ are independently selected from hydrogen, methyl, halogen (e.g., fluoro or chloro) and hydroxy. In certain embodiments, R² is selected from hydrogen, a halogen and an alkoxy. In certain embodiments, R³¹-R³⁵ are selected as described in one of the compounds of Tables 1-2. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe. In certain embodiments, Y⁴ is CH. In certain embodiments, Y⁴ is CR, wherein R is not H. In certain embodiments, Y⁴ is O. In certain embodiments, R⁴¹ is lower alkyl or substituted lower alkyl. In certain embodiments, R⁴¹ is H. In certain instances, R³¹-R³⁵ are independently selected from hydrogen, methyl, halogen and hydroxy, R⁴¹ is lower alkyl (e.g., methyl) and Y⁴ is O.

In some embodiments, the PI4-kinase inhibitor has the following structure:

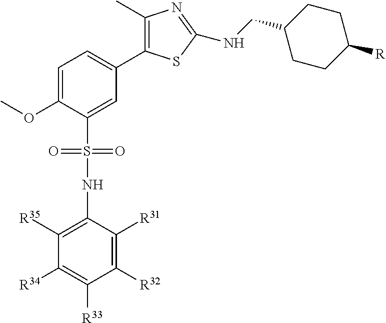

where R and R³¹-R³⁵ are selected from one of the following embodiments:

| Embodiment | R | R³¹ | R³² | R³³ | R³⁴ | R³⁵ |
|---|---|---|---|---|---|---|
| 1 | CO₂Et | H | H | OH | H | H |
| 2 | CO₂Et | H | H | H | H | H |
| 3 | CO₂Et | F | H | F | H | H |
| 4 | CO₂Et | Cl | H | H | H | H |
| 5 | CO₂Et | Cl | H | Cl | H | H |
| 6 | CO₂Et | Cl | H | H | H | Cl |
| 7 | H | H | H | OH | H | H |
| 8 | H | H | H | H | H | H |
| 9 | H | F | H | F | H | H |
| 10 | H | Cl | H | H | H | H |
| 11 | H | Cl | H | Cl | H | H |
| 12 | H | Cl | H | H | H | Cl |

In some embodiments, the PI4-kinase inhibitor has the following structure:

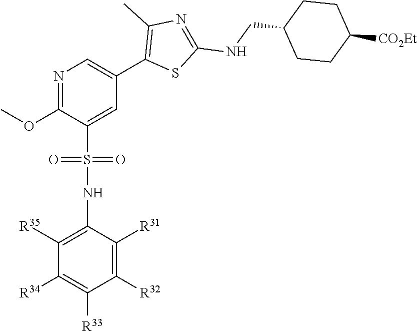

where R³¹-R³⁵ are selected from one of the following embodiments:

| Embodiment | R³¹ | R³² | R³³ | R³⁴ | R³⁵ |
|---|---|---|---|---|---|
| 1 | H | H | OH | H | H |
| 2 | H | H | H | H | H |
| 3 | F | H | F | H | H |
| 4 | Cl | H | H | H | H |
| 5 | Cl | H | Cl | H | H |
| 6 | Cl | H | H | H | Cl |

In certain instances of formula (XXI), the PI4-kinase inhibitor has one of the formula (XLVIII) or formula (XLIX):

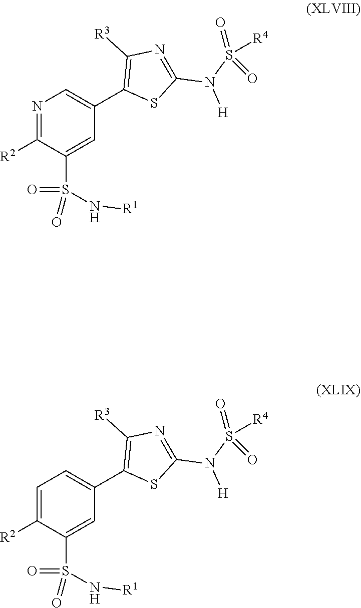

wherein R¹ is selected from aryl, substituted aryl, heteroaryl and substituted heteroaryl. In certain instances of formula (XLVIII) or formula (XLIX): R⁴ is methyl, isopropyl, cyclohexyl, substituted cyclohexyl, phenyl, substituted phenyl, benzyl, substituted benzyl or —CH₂-4-tetrahydropyran. In certain instances of formula (XLVIII) or formula (XLIX): R¹ is a substituted phenyl (e.g., 4-hydroxy-phenyl). In certain embodiments, R² is selected from hydrogen, a halogen and an alkoxy. In certain embodiments, R² is selected from hydrogen, a halogen, an alkyl, a substituted alkyl and an alkoxy. In certain embodiments, R² is selected from a lower alkyl, a halogen, a substituted lower alkyl, and a lower alkoxy. In certain embodiments, R² is selected from Me, Cl, Br, CHF₂, CF₃, CH₂F and OMe. Also provided are compounds based on any of the structure and formulae depicted herein having OMe at the R² position, which have an analogous structure but with one Me, Cl, Br, CHF₂, CF₃ and CH₂F appearing at the R² position instead of this OMe group.

In some embodiments, the PI4-kinase inhibitor has the following structure:

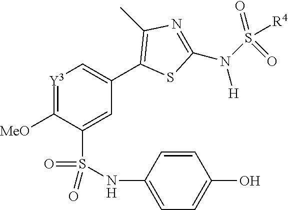

where Y³ and R⁴ are selected from one of the following embodiments:

| Embodiment | Y³ | R⁴ |
|---|---|---|
| 1 | CH | methyl |
| 2 | CH | isopropyl |
| 3 | CH | cyclohexyl |
| 4 | CH | phenyl |
| 5 | CH | benzyl |
| 6 | CH | —CH₂-(4-tetrahydropyran) |
| 7 | N | methyl |
| 8 | N | isopropyl |
| 9 | N | cyclohexyl |
| 10 | N | phenyl |
| 11 | N | benzyl |
| 12 | N | —CH₂-(4-tetrahydropyran) |

In some embodiments of any one of the formulae (Ia)-(XLIX), R² is methoxy. In some embodiments of any one of the formulae (Ia)-(XLIX), R³ is methyl. In some embodiments of any one of the formulae (Ia)-(XLIX), R² is methoxy and R³ is methyl. In some embodiments of any one of the formulae (Ia)-(XLIX), R² is a halogen (e.g., Cl or Br). In some embodiments of any one of the formulae (Ia)-(XLIX), R² is a substituted lower alkyl. In some embodiments of any one of the formulae (Ia)-(XLIX), R² is CF₃. In some embodiments of any one of the formulae (Ia)-(XLIX), R² is CHF₂. In some embodiments of any one of the formulae (Ia)-(XLIX), R² is CH₂F. In some embodiments of any one of the formulae (Ia)-(XLIX), R² is a lower alkyl. In some embodiments of any one of the formulae (Ia)-(XLIX), R² is methyl.

In certain embodiments, the PI4-kinase inhibitor is described by the structure of one of the compounds of Table 1 or Table 2. It is understood that any of the compounds shown in Table 1 or 2 may be present in a salt form, such as a trifluoroacetate salt (e.g., CF₃COOH salt). In some cases, the salt form of the compound is a pharmaceutically acceptable salt.

TABLE 1
Compounds

| Cmp | Structure |
|---|---|
| S-1 | 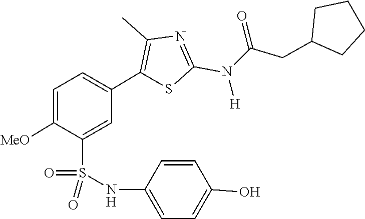 [SMILES: COc1ccc(-c2sc(NC(=O)CC3CCCC3)nc2C)cc1S(=O)(=O)Nc1ccc(O)cc1] |
| | 2-cyclopentyl-N-[5-[3-[(4- |
| | hydroxyphenyl)sulfamoyl]-4-methoxy- |
| | phenyl]-4-methyl-thiazol-2-yl]acetamide |
| S-2 | 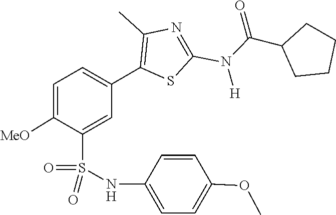 [SMILES: COc1ccc(NS(=O)(=O)c2cc(-c3sc(NC(=O)C4CCCC4)nc3C)ccc2OC)cc1] |
| | N-[5-[4-methoxy-3-[(4- |
| | methoxyphenyl)sulfamoyl]phenyl]-4- |
| | methyl-thiazol-2- |
| | yl]cyclopentanecarboxamide |
| S-3 | 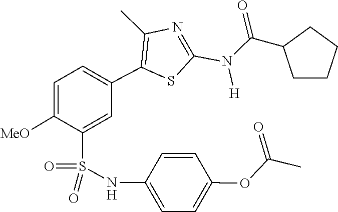 [SMILES: COc1ccc(-c2sc(NC(=O)C3CCCC3)nc2C)cc1S(=O)(=O)Nc1ccc(OC(C)=O)cc1] |
| | [4-[[5-[2-(cyclopentanecarbonylamino)-4- |
| | methyl-thiazol-5-yl]-2-methoxy- |
| | phenyl]sulfonylamino]phenyl]acetate |
| S-4 | 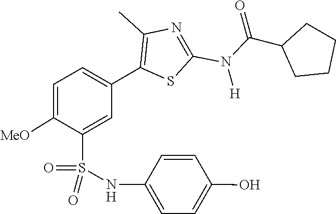 [SMILES: COc1ccc(-c2sc(NC(=O)C3CCCC3)nc2C)cc1S(=O)(=O)Nc1ccc(O)cc1] |
| | N-[5-[3-[(4-hydroxyphenyl)sulfamoyl]- |
| | 4-methoxy-phenyl]-4-methyl-thiazol-2- |
| | yl]cyclopentanecarboxamide |
| S-5 | 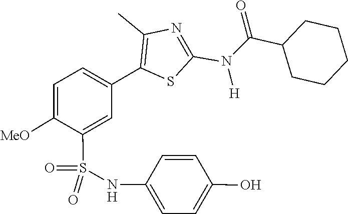 [SMILES: COc1ccc(-c2sc(NC(=O)C3CCCCC3)nc2C)cc1S(=O)(=O)Nc1ccc(O)cc1] |
| | N-[5-[3-[(4-hydroxyphenyl)sulfamoyl]-4- |
| | methoxy-phenyl]-4-methyl-thiazol-2- |
| | yl]cyclohexanecarboxamide |
| S-6 | 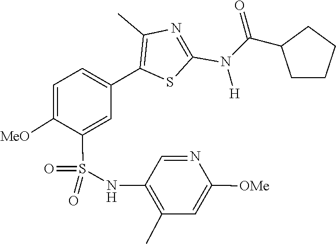 [SMILES: COc1cc(C)c(NS(=O)(=O)c2cc(-c3sc(NC(=O)C4CCCC4)nc3C)ccc2OC)cn1] |
| | N-[5-[4-methoxy-3-[(6-methoxy-4- |
| | methyl-3-pyridyl)sulfamoyl]phenyl]-4- |
| | methyl-thiazol-2- |
| | yl]cyclopentanecarboxamide |
| S-7 | 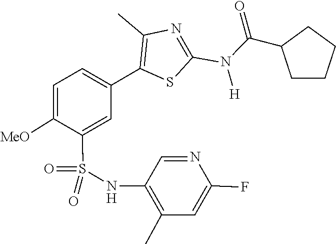 [SMILES: COc1ccc(-c2sc(NC(=O)C3CCCC3)nc2C)cc1S(=O)(=O)Nc1cnc(F)cc1C] |
| | N-[5-[3-[(6-fluoro-4-methyl-3- |
| | pyridyl)sulfamoyl]-4-methoxy-phenyl]-4- |
| | methyl-thiazol-2- |
| | yl]cyclopentanecarboxamide |
| S-8 | 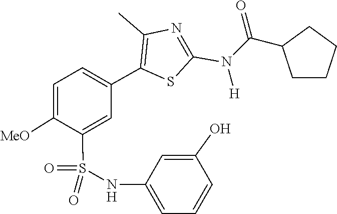 [SMILES: COc1ccc(-c2sc(NC(=O)C3CCCC3)nc2C)cc1S(=O)(=O)Nc1cccc(O)c1] |
| | N-[5-[3-[(3-hydroxyphenyl)sulfamoyl]- |
| | 4-methoxy-phenyl]-4-methyl-thiazol-2- |
| | yl]cyclopentanecarboxamide |
| S-9 | 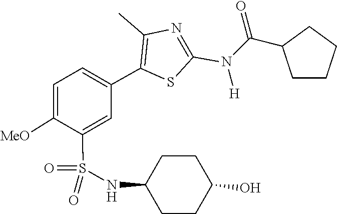 [SMILES: COc1ccc(-c2sc(NC(=O)C3CCCC3)nc2C)cc1S(=O)(=O)N[C@H]1CC[C@H](O)CC1] |
| | N-[5-[3-[(4- |
| | hydroxycyclohexyl)sulfamoyl]-4-methoxy- |
| | phenyl]-4-methyl-thiazol-2- |
| | yl]cyclopentanecarboxamide |
| S-10 | 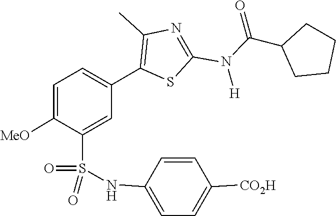 [SMILES: COc1ccc(-c2sc(NC(=O)C3CCCC3)nc2C)cc1S(=O)(=O)Nc1ccc(C(=O)O)cc1] |
| | 4-[[5-[2-(cyclopentanecarbonylamino)- |
| | 4-methyl-thiazol-5-yl]-2-methoxy- |
| | phenyl]sulfonylamino]benzoic acid |
| S-11 | 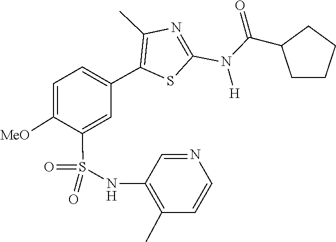 [SMILES: COc1ccc(-c2sc(NC(=O)C3CCCC3)nc2C)cc1S(=O)(=O)Nc1cnccc1C] |
| | N-[5-[4-methoxy-3-[(4-methyl-3- |
| | pyridyl)sulfamoyl]phenyl]-4-methyl- |
| | thiazol-2-yl]cyclopentanecarboxamide |
| S-12 | 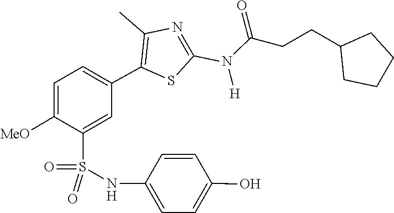 [SMILES: COc1ccc(-c2sc(NC(=O)CCC3CCCC3)nc2C)cc1S(=O)(=O)Nc1ccc(O)cc1] |
| | 3-cyclopentyl-N-[5-[3-[(4- |
| | hydroxyphenyl)sulfamoyl]-4-methoxy- |
| | phenyl]-4-methyl-thiazol-2- |
| | yl]propanamide |
| S-13 | 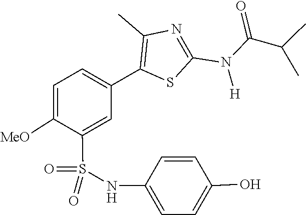 [SMILES: COc1ccc(-c2sc(NC(=O)C(C)C)nc2C)cc1S(=O)(=O)Nc1ccc(O)cc1] |
| | N-[5-[3-[(4-hydroxyphenyl)sulfamoyl]-4- |
| | methoxy-phenyl]-4-methyl-thiazol-2-yl]-2- |
| | methyl-propanamide |
| S-14 | 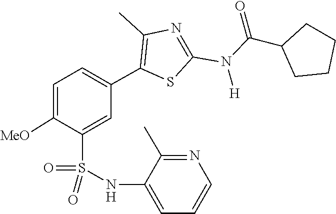 [SMILES: COc1ccc(-c2sc(NC(=O)C3CCCC3)nc2C)cc1S(=O)(=O)Nc1cccnc1C] |
| | N-[5-[4-methoxy-3-[(2-methyl-3- |
| | pyridyl)sulfamoyl]phenyl]-4-methyl- |
| | thiazol-2-yl]cyclopentanecarboxamide |
| S-15 | 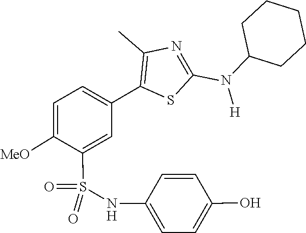 [SMILES: COc1ccc(-c2sc(NC3CCCCC3)nc2C)cc1S(=O)(=O)Nc1ccc(O)cc1] |
| | 5-[2-(cyclohexylamino)-4-methyl-thiazol- |
| | 5-yl]-N-(4-hydroxyphenyl)-2-methoxy- |
| | benzenesulfonamide |
| S-16 | 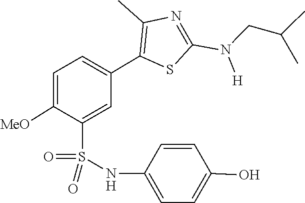 [SMILES: COc1ccc(-c2sc(NCC(C)C)nc2C)cc1S(=O)(=O)Nc1ccc(O)cc1] |
| | N-(4-hydroxyphenyl)-5-[2- |
| | (isobutylamino)-4-methyl-thiazol-5-yl]- |
| | 2-methoxy-benzenesulfonamide |
| S-17 | 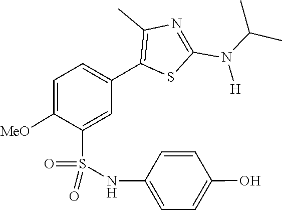 [SMILES: COc1ccc(-c2sc(NC(C)C)nc2C)cc1S(=O)(=O)Nc1ccc(O)cc1] |
| | N-(4-hydroxyphenyl)-5-[2- |
| | (isopropylamino)-4-methyl-thiazol-5-yl]-2- |
| | methoxy-benzenesulfonamide |
| S-18 | 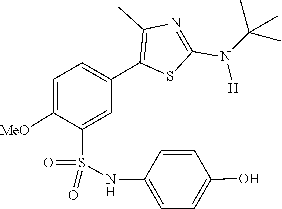 [SMILES: COc1ccc(-c2sc(NC(C)(C)C)nc2C)cc1S(=O)(=O)Nc1ccc(O)cc1] |
| | 5-[2-(tert-butylamino)-4-methyl-thiazol- |
| | 5-yl]-N-(4-hydroxyphenyl)-2-methoxy- |
| | benzenesulfonamide |
| S-19 | 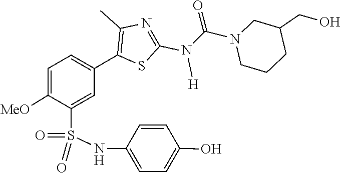 [SMILES: COc1ccc(-c2sc(NC(=O)N3CCCC(CO)C3)nc2C)cc1S(=O)(=O)Nc1ccc(O)cc1] |
| | 3-(hydroxymethyl)-N-[5-[3-[(4- |
| | hydroxyphenyl)sulfamoyl]-4-methoxy- |
| | phenyl]-4-methyl-thiazol-2-yl]piperidine- |
| | 1-carboxamide |
| S-20 | 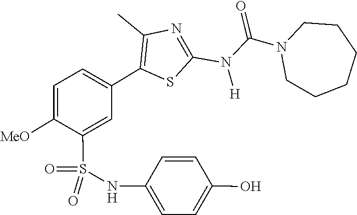 [SMILES: COc1ccc(-c2sc(NC(=O)N3CCCCCC3)nc2C)cc1S(=O)(=O)Nc1ccc(O)cc1] |
| | N-[5-[3-[(4-hydroxyphenyl)sulfamoyl]- |
| | 4-methoxy-phenyl]-4-methyl-thiazol-2- |
| | yl]azepane-l-carboxamide |
| S-21 | 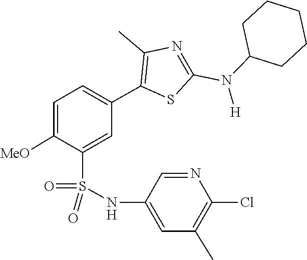 [SMILES: COc1ccc(-c2sc(NC3CCCCC3)nc2C)cc1S(=O)(=O)Nc1cnc(Cl)c(C)c1] |
| | N-(6-chloro-5-methyl-3-pyridyl)-5-[2- |
| | (cyclohexylamino)-4-methyl-thiazol-5-yl]- |
| | 2-methoxy-benzenesulfonamide |
| S-22 | 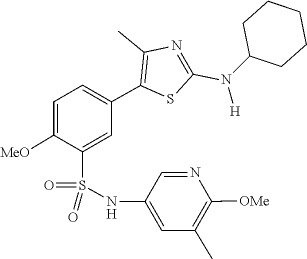 [SMILES: COc1ccc(-c2sc(NC3CCCCC3)nc2C)cc1S(=O)(=O)Nc1cnc(OC)c(C)c1] |
| | 5-[2-(cyclohexylamino)-4-methyl- |
| | thiazol-5-yl]-2-methoxy-N-(6-methoxy- |
| | 5-methyl-3-pyridyl)benzenesulfonamide |
| S-23 | 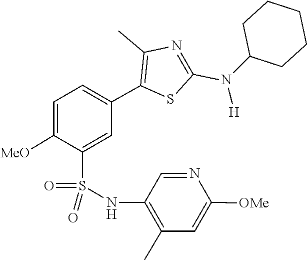 [SMILES: COc1cc(C)c(NS(=O)(=O)c2cc(-c3sc(NC4CCCCC4)nc3C)ccc2OC)cn1] |
| | 5-[2-(cyclohexylamino)-4-methyl-thiazol- |
| | 5-yl]-2-methoxy-N-(6-methoxy-N-(6-methoxy-4-methyl- |
| | 3-pyridyl)benzenesulfonamide |
| S-24 | 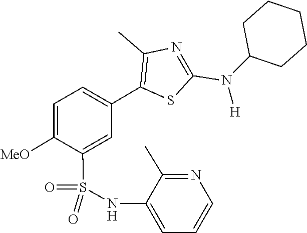 [SMILES: COc1ccc(-c2sc(NC3CCCCC3)nc2C)cc1S(=O)(=O)Nc1cccnc1C] |
| | 5-[2-(cyclohexylamino)-4-methyl- |
| | thiazol-5-yl]-2-methoxy-N-(2-methyl-3- |
| | pyridyl)benzenesulfonamide |
| S-25 | 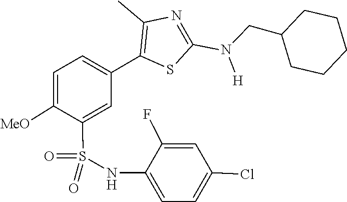 [SMILES: COc1ccc(-c2sc(NCC3CCCCC3)nc2C)cc1S(=O)(=O)Nc1ccc(Cl)cc1F] |
| | N-(4-chloro-2-fluoro-phenyl)-5-[2- |
| | (cyclohexylmethylamino)-4-methyl- |
| | thiazol-5-yl]-2-methoxy- |
| | benzenesulfonamide |
| S S-26 | 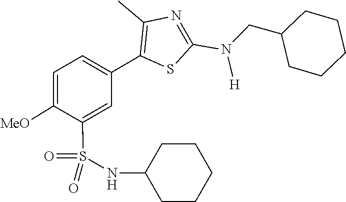 [SMILES: COc1ccc(-c2sc(NCC3CCCCC3)nc2C)cc1S(=O)(=O)NC1CCCCC1] |
| | N-cyclohexyl-5-[2- |
| | (cyclohexylmethylamino)-4-methyl- |
| | thiazol-5-yl]-2-methoxy- |
| | benzenesulfonamide |
| S-27 | 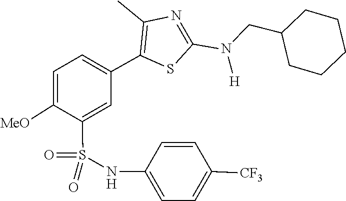 [SMILES: COc1ccc(-c2sc(NCC3CCCCC3)nc2C)cc1S(=O)(=O)Nc1ccc(C(F)(F)F)cc1] |
| | 5-[2-(cyclohexylmethylamino)-4-methyl- |
| | thiazol-5-yl]-2-methoxy-N-[4- |
| | (trifluoromethyl)phenyl]benzenesulfonamide |
| S-28 | 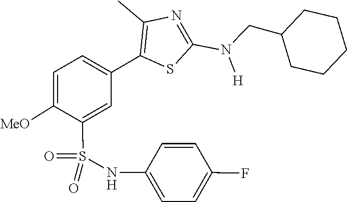 [SMILES: COc1ccc(-c2sc(NCC3CCCCC3)nc2C)cc1S(=O)(=O)Nc1ccc(F)cc1] |
| | 5-[2-(cyclohexylmethylamino)-4- |
| | methyl-thiazol-5-yl]-N-(4-fluorophenyl)- |
| | 2-methoxy-benzenesulfonamide |
| S-29 | 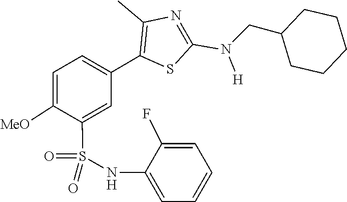 [SMILES: COc1ccc(-c2sc(NCC3CCCCC3)nc2C)cc1S(=O)(=O)Nc1ccccc1F] |
| | 5-[2-(cyclohexylmethylamino)-4-methyl- |
| | thiazol-5-yl]-N-(2-fluorophenyl)-2- |
| | methoxy-benzenesulfonamide |
| S-30 | 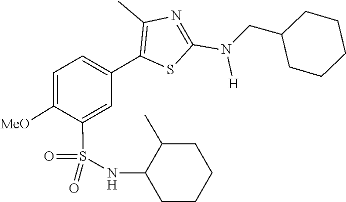 [SMILES: COc1ccc(-c2sc(NCC3CCCCC3)nc2C)cc1S(=O)(=O)NC1CCCCC1C] |
| | 5-[2-(cyclohexylmethylamino)-4- |
| | methyl-thiazol-5-yl]-2-methoxy-N-(2- |
| | methylcyclohexyl)benzenesulfonamide |
| S-31 | 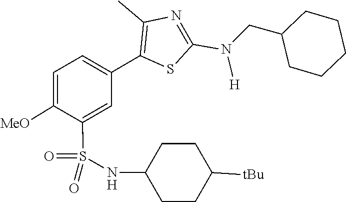 [SMILES: COc1ccc(-c2sc(NCC3CCCCC3)nc2C)cc1S(=O)(=O)NC1CCC(C(C)(C)C)CC1] |
| | N-(4-tert-butylphenyl)-5-[2- |
| | (cyclohexylmethylamino)-4-methyl- |
| | thiazol-5-yl]-2-methoxy- |
| | benzenesulfonamide;2,2,2-trifluoroacetic |
| | acid |
| S-32 | 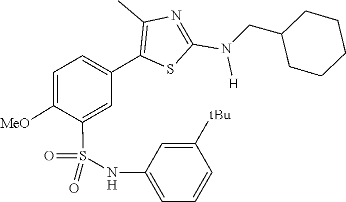 [SMILES: COc1ccc(-c2sc(NCC3CCCCC3)nc2C)cc1S(=O)(=O)Nc1cccc(C(C)(C)C)c1] |
| | N-(3-tert-butylphenyl)-5-[2- |
| | (cyclohexylmethylamino)-4-methyl- |
| | thiazol-5-yl]-2-methoxy- |
| | benzenesulfonamide;2,2,2-trifluoroacetic |
| | acid |
| S-33 | 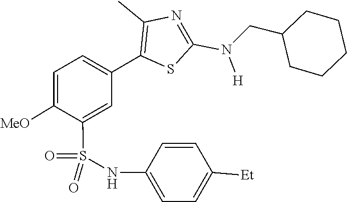 [SMILES: CCc1ccc(NS(=O)(=O)c2cc(-c3sc(NCC4CCCCC4)nc3C)ccc2OC)cc1] |
| | 5-[2-(cyclohexylmethylamino)-4-methyl- |
| | thiazol-5-yl]-N-(4-ethylphenyl)-2- |
| | methoxy-benzenesulfonamide;2,2,2- |
| | trifluoroacetic acid |
| S-34 | 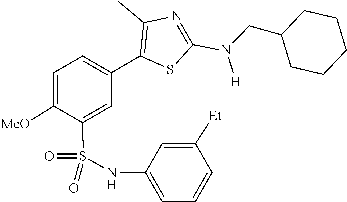 [SMILES: CCc1cccc(NS(=O)(=O)c2cc(-c3sc(NCC4CCCCC4)nc3C)ccc2OC)c1] |
| | 5-[2-(cyclohexylmethylamino)-4- |
| | methyl-thiazol-5-yl]-N-(3-ethylphenyl)- |
| | 2-methoxy-benzenesulfonamide |
| S-35 | 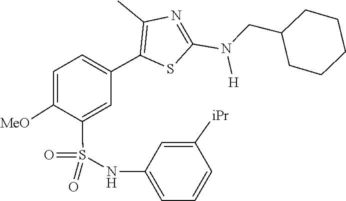 [SMILES: COc1ccc(-c2sc(NCC3CCCCC3)nc2C)cc1S(=O)(=O)Nc1cccc(C(C)C)c1] |
| | 5-[2-(cyclohexylmethylamino)-4-methyl- |
| | thiazol-5-yl]-N-(3-isopropylphenyl)-2- |
| | methoxy-benzenesulfonamide |
| S-36 | 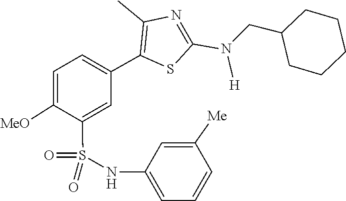 [SMILES: COc1ccc(-c2sc(NCC3CCCCC3)nc2C)cc1S(=O)(=O)Nc1cccc(C)c1] |
| | 5-[2-(cyclohexylmethylamino)-4- |
| | methyl-thiazol-5-yl]-2-methoxy-N-(m- |
| | tolyl)benzenesulfonamide |
| S-37 | 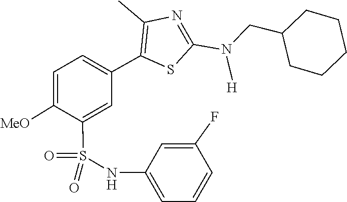 [SMILES: COc1ccc(-c2sc(NCC3CCCCC3)nc2C)cc1S(=O)(=O)Nc1cccc(F)c1] |
| | 5-[2-(cyclohexylmethylamino)-4-methyl- |
| | thiazol-5-yl]-N-(3-fluorophenyl)-2- |
| | methoxy-benzenesulfonamide |
| S-38 | 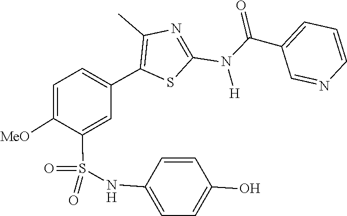 [SMILES: COc1ccc(-c2sc(NC(=O)c3cccnc3)nc2C)cc1S(=O)(=O)Nc1ccc(O)cc1] |
| | N-[5-[3-[(4-hydroxyphenyl)sulfamoyl]- |
| | 4-methoxy-phenyl]-4-methyl-thiazol-2- |
| | yl]pyridine-3-carboxamide |
| S-39 | 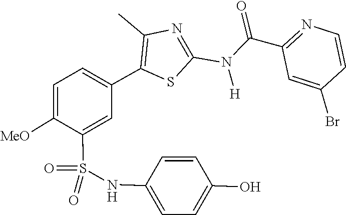 [SMILES: COc1ccc(-c2sc(NC(=O)c3cc(Br)ccn3)nc2C)cc1S(=O)(=O)Nc1ccc(O)cc1] |
| | 4-bromo-N-[5-[3-[(4- |
| | hydroxyphenyl)sulfamoyl]-4-methoxy- |
| | phenyl]-4-methyl-thiazol-2-yl]pyridine-2- |
| | carboxamide |
| S-40 | 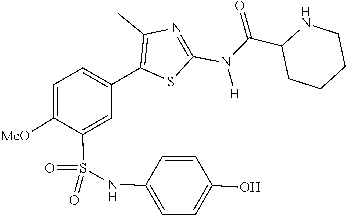 [SMILES: COc1ccc(-c2sc(NC(=O)C3CCCCN3)nc2C)cc1S(=O)(=O)Nc1ccc(O)cc1] |
| | N-[5-[3-[(4-hydroxyphenyl)sulfamoyl]- |
| | 4-methoxy-phenyl]-4-methyl-thiazol-2- |
| | yl]piperidine-2-carboxamide |
| S-41 | 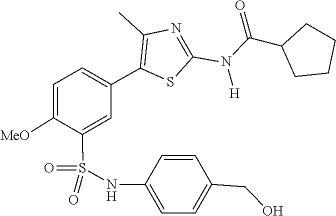 [SMILES: COc1ccc(-c2sc(NC(=O)C3CCCC3)nc2C)cc1S(=O)(=O)Nc1ccc(CO)cc1] |
| | N-[5-[3-[[4- |
| | (hydroxymethyl)phenyl]sulfamoyl]-4- |
| | methoxy-phenyl]-4-methyl-thiazol-2- |
| | yl]cyclopentanecarboxamide |
| S-42 | 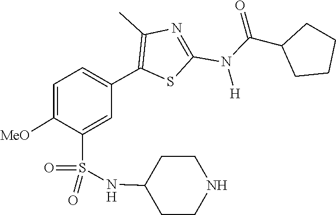 [SMILES: COc1ccc(-c2sc(NC(=O)C3CCCC3)nc2C)cc1S(=O)(=O)NC1CCNCC1] |
| | N-[5-[4-methoxy-3-(4- |
| | piperidylsulfamoyl)phenyl]-4-methyl- |
| | thiazol-2-yl]cyclopentanecarboxamide |
| S-43 | 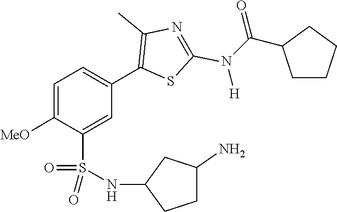 [SMILES: COc1ccc(-c2sc(NC(=O)C3CCCC3)nc2C)cc1S(=O)(=O)NC1CCC(N)C1] |
| | N-[5-[3-(3-aminopyrrolidin-1-yl)sulfonyl- |
| | 4-methoxy-phenyl]-4-methyl-thiazol-2- |
| | yl]cyclopentanecarboxamide |
| S-44 | 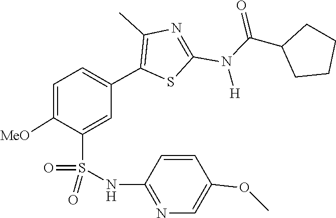 [SMILES: COc1ccc(NS(=O)(=O)c2cc(-c3sc(NC(=O)C4CCCC4)nc3C)ccc2OC)nc1] |
| | N-[5-[4-methoxy-3-[(5-methoxy-2- |
| | pyridyl)sulfamoyl]phenyl]-4-methyl- |
| | thiazol-2-yl]cyclopentanecarboxamide |
| S-45 | 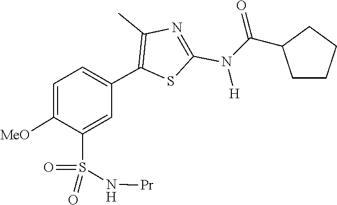 |
| | N-[5-[4-methoxy-3- |
| | (propylsulfamoyl)phenyl]-4-methyl- |
| | thiazol-2-yl]cyclopentanecarboxamide |
| S-46 | 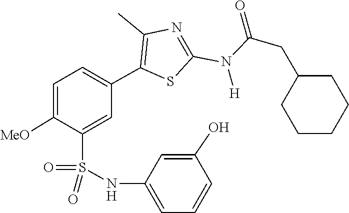 [SMILES: COc1ccc(-c2sc(NC(=O)CC3CCCCC3)nc2C)cc1S(=O)(=O)Nc1cccc(O)c1] |
| | 2-cyclohexyl-N-[5-[3-[(3- |
| | hydroxyphenyl)sulfamoyl]-4-methoxy- |
| | phenyl]-4-methyl-thiazol-2-yl]acetamide |
| S-47 | 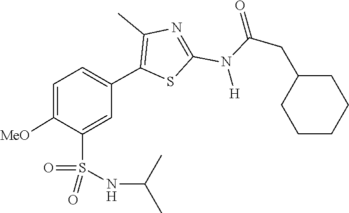 [SMILES: COc1ccc(-c2sc(NC(=O)CC3CCCCC3)nc2C)cc1S(=O)(=O)NC(C)C] |
| | 2-cyclohexyl-N-[5-[3- |
| | (isopropylsulfamoyl)-4-methoxy-phenyl]- |
| | 4-methyl-thiazol-2-yl]acetamide |
| S-48 | 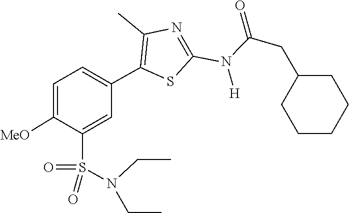 [SMILES: CCN(CC)S(=O)(=O)c1cc(-c2sc(NC(=O)CC3CCCCC3)nc2C)ccc1OC] |
| | 2-cyclohexyl-N-[5-[3- |
| | (diethylsulfamoyl)-4-methoxy-phenyl]- |
| | 4-methyl-thiazol-2-yl]acetamide |
| S-49 | 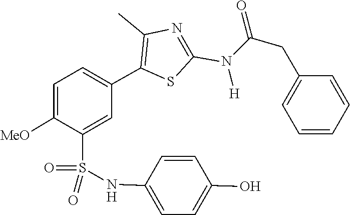 [SMILES: COc1ccc(-c2sc(NC(=O)Cc3ccccc3)nc2C)cc1S(=O)(=O)Nc1ccc(O)cc1] |
| | N-[5-[3-[(4-hydroxyphenyl)sulfamoyl]-4- |
| | methoxy-phenyl]-4-methyl-thiazol-2-yl]-2- |
| | phenyl-acetamide |
| S-50 | 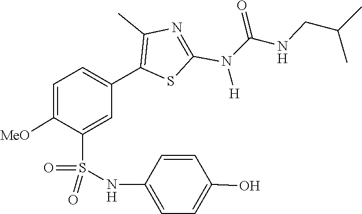 [SMILES: COc1ccc(-c2sc(NC(=O)NCC(C)C)nc2C)cc1S(=O)(=O)Nc1ccc(O)cc1] |
| | 1-[5-[3-[(4-hydroxyphenyl)sulfamoyl]- |
| | 4-methoxy-phenyl]-4-methyl-thiazol-2- |
| | yl]-3-isobutyl-urea |
| S-51 | 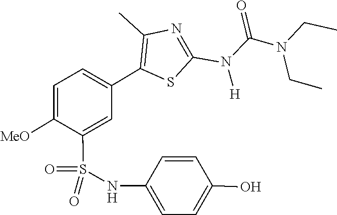 [SMILES: CCN(CC)C(=O)Nc1nc(C)c(-c2ccc(OC)c(S(=O)(=O)Nc3ccc(O)cc3)c2)s1] |
| | 1,1-diethyl-3-[5-[3-[(4- |
| | hydroxyphenyl)sulfamoyl]-4-methoxy- |
| | phenyl]-4-methyl-thiazol-2-yl]urea |
| S-52 | 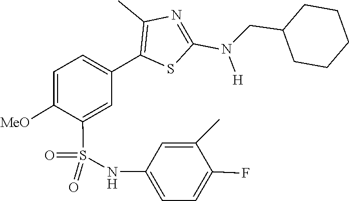 [SMILES: COc1ccc(-c2sc(NCC3CCCCC3)nc2C)cc1S(=O)(=O)Nc1ccc(F)c(C)c1] |
| | 5-[2-(cyclohexylmethylamino)-4- |
| | methyl-thiazol-5-yl]-N-(4-fluoro-3- |
| | methyl-phenyl)-2-methoxy- |
| | benzenesulfonamide;2,2,2-trifluoroacetic |
| | acid |
| S-53 | 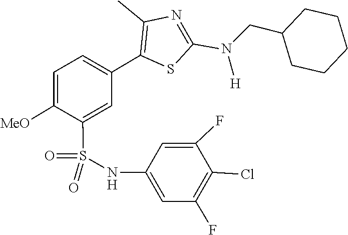 [SMILES: COc1ccc(-c2sc(NCC3CCCCC3)nc2C)cc1S(=O)(=O)Nc1cc(F)c(Cl)c(F)c1] |
| | N-(4-chloro-3,5-difluoro-phenyl)-5-[2- |
| | (cyclohexylmethylamino)-4-methyl- |
| | thiazol-5-yl]-2-methoxy- |
| | benzenesulfonamide |
| S-54 | 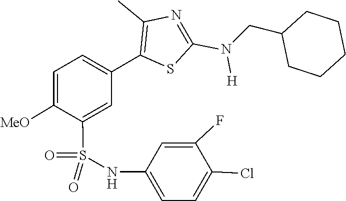 [SMILES: COc1ccc(-c2sc(NCC3CCCCC3)nc2C)cc1S(=O)(=O)Nc1ccc(Cl)c(F)c1] |
| | N-(4-chloro-3-fluoro-phenyl)-5-[2- |
| | (cyclohexylmethylamino)-4-methyl- |
| | thiazol-5-yl]-2-methoxy- |
| | benzenesulfonamide |
| S-55 | 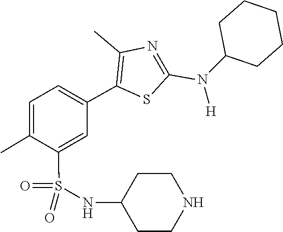 [SMILES: Cc1ccc(-c2sc(NC3CCCCC3)nc2C)cc1S(=O)(=O)NC1CCNCC1] |
| | 5-[2-(cyclohexylamino)-4-methyl-thiazol- |
| | 5-yl]-2-methyl-N-(4- |
| | piperidyl)benzenesulfonamide |
| S-56 | 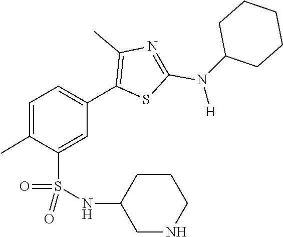 [SMILES: Cc1ccc(-c2sc(NC3CCCCC3)nc2C)cc1S(=O)(=O)NC1CCCNC1] |
| | 5-[2-(cyclohexylamino)-4-methyl- |
| | thiazol-5-yl]-2-methyl-N-(3- |
| | piperidyl)benzenesulfonamide |
| S-57 | 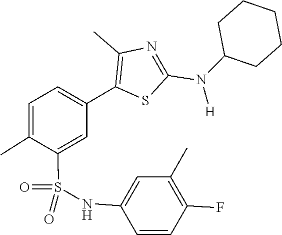 [SMILES: Cc1cc(NS(=O)(=O)c2cc(-c3sc(NC4CCCCC4)nc3C)ccc2C)ccc1F] |
| | 5-[2-(cyclohexylamino)-4-methyl-thiazol- |
| | 5-yl]-N-(4-fluoro-3-methyl-phenyl)-2- |
| | methyl-benzenesulfonamide;2,2,2- |
| | trifluoroacetic acid |
| S-58 | 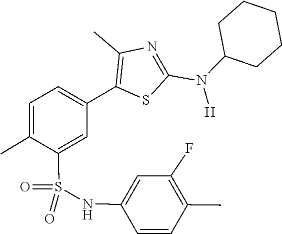 [SMILES: Cc1ccc(NS(=O)(=O)c2cc(-c3sc(NC4CCCCC4)nc3C)ccc2C)cc1F] |
| | 5-[2-(cyclohexylamino)-4-methyl- |
| | thiazol-5-yl]-N-(3-fluoro-4-methyl- |
| | phenyl)-2-methyl-benzenesulfonamide |
| S-59 | 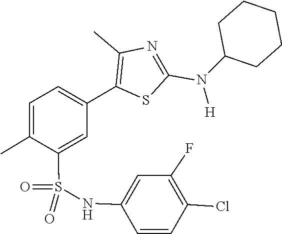 [SMILES: Cc1ccc(-c2sc(NC3CCCCC3)nc2C)cc1S(=O)(=O)Nc1ccc(Cl)c(F)c1] |
| | N-(4-chloro-3-fluoro-phenyl)-5-[2- |
| | (cyclohexylamino)-4-methyl-thiazol-5-yl]- |
| | 2-methyl-benzenesulfonamide |
| S-60 | 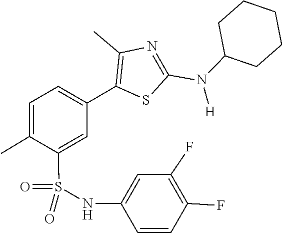 [SMILES: Cc1ccc(-c2sc(NC3CCCCC3)nc2C)cc1S(=O)(=O)Nc1ccc(F)c(F)c1] |
| | 5-[2-(cyclohexylamino)-4-methyl- |
| | thiazol-5-yl]-N-(3,4-difluorophenyl)-2- |
| | methyl-benzenesulfonamide |
| S-61 | 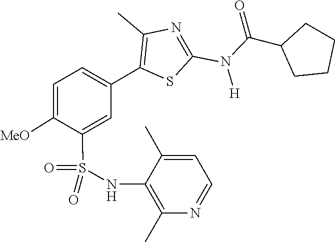 [SMILES: COc1ccc(-c2sc(NC(=O)C3CCCC3)nc2C)cc1S(=O)(=O)Nc1c(C)ccnc1C] |
| | N-[5-[3-[(2,4-dimethyl-3- |
| | pyridyl)sulfamoyl]-4-methoxy-phenyl]-4- |
| | methyl-thiazol-2- |
| | yl]cyclopentanecarboxamide |
| S-62 | 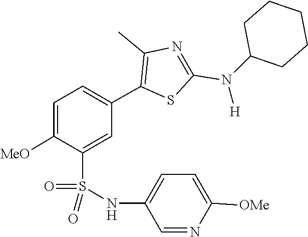 [SMILES: COc1ccc(NS(=O)(=O)c2cc(-c3sc(NC4CCCCC4)nc3C)ccc2OC)cn1] |
| | 5-[2-(cyclohexylamino)-4-methyl- |
| | thiazol-5-yl]-2-methoxy-N-(6-methoxy- |
| | 3-pyridyl)benzenesulfonamide |
| S-63 | 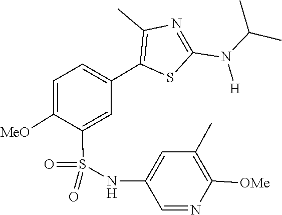 [SMILES: COc1ccc(-c2sc(NC(C)C)nc2C)cc1S(=O)(=O)Nc1cnc(OC)c(C)c1] |
| | 5-[2-(isopropylamino)-4-methyl-thiazol-5- |
| | yl]-2-methoxy-N-(6-methoxy-5-methyl-3- |
| | pyridyl)benzenesulfonamide |
| S-64 | 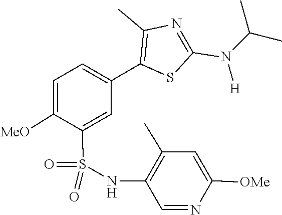 [SMILES: COc1cc(C)c(NS(=O)(=O)c2cc(-c3sc(NC(C)C)nc3C)ccc2OC)cn1] |
| | 5-[2-(isopropylamino)-4-methyl-thiazol- |
| | 5-yl]-2-methoxy-N-(6-methoxy-4- |
| | methyl-3-pyridyl)benzenesulfonamide |
| S-65 | 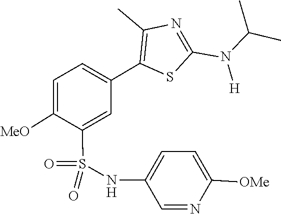 [SMILES: COc1ccc(NS(=O)(=O)c2cc(-c3sc(NC(C)C)nc3C)ccc2OC)cn1] |
| | 5-[2-(isopropylamino)-4-methyl-thiazol-5- |
| | yl]-2-methoxy-N-(6-methoxy-3- |
| | pyridyl)benzenesulfonamide;2,2,2- |
| | trifluoroacetic acid |
| S-66 | 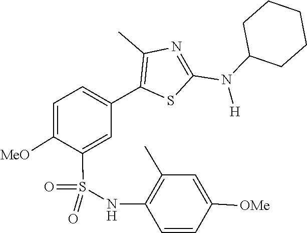 [SMILES: COc1ccc(NS(=O)(=O)c2cc(-c3sc(NC4CCCCC4)nc3C)ccc2OC)c(C)c1] |
| | 5-[2-(cyclohexylamino)-4-methyl- |
| | thiazol-5-yl]-2-methoxy-N-(4-methoxy- |
| | 2-methyl- |
| | phenyl)benzenesulfonamide;2,2,2- |
| | trifluoroacetic acid |
| S-67 | 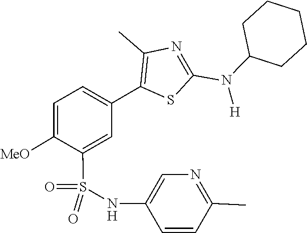 [SMILES: COc1ccc(-c2sc(NC3CCCCC3)nc2C)cc1S(=O)(=O)Nc1ccc(C)nc1] |
| | 5-[2-(cyclohexylamino)-4-methyl-thiazol- |
| | 5-yl]-2-methoxy-N-(6-methyl-3- |
| | pyridyl)benzenesulfonamide;2,2,2- |
| | trifluoroacetic acid |
| S-68 | 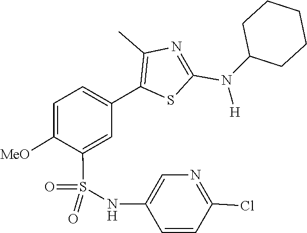 [SMILES: COc1ccc(-c2sc(NC3CCCCC3)nc2C)cc1S(=O)(=O)Nc1ccc(Cl)nc1] |
| | N-(6-chloro-3-pyridyl)-5-[2- |
| | (cyclohexylamino)-4-methyl-thiazol-5- |
| | yl]-2-methoxy- |
| | benzenesulfonamide;2,2,2-trifluoroacetic |
| | acid |
| S-69 | 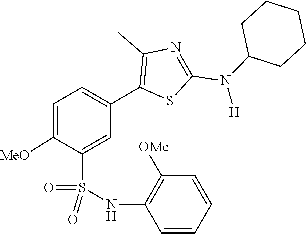 [SMILES: COc1ccccc1NS(=O)(=O)c1cc(-c2sc(NC3CCCCC3)nc2C)ccc1OC] |
| | 5-[2-(cyclohexylamino)-4-methyl-thiazol- |
| | 5-yl]-2-methoxy-N-(2- |
| | methoxyphenyl)benzenesulfonamide;2,2,2- |
| | trifluoroacetic acid |
| S-70 | 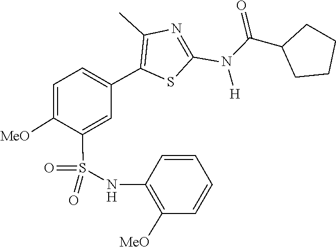 [SMILES: COc1ccccc1NS(=O)(=O)c1cc(-c2sc(NC(=O)C3CCCC3)nc2C)ccc1OC] |
| | N-[5-[4-methoxy-3-[(2- |
| | methoxyphenyl)sulfamoyl]phenyl]-4- |
| | methyl-thiazol-2- |
| | yl]cyclopentanecarboxamide |
| S-71 | 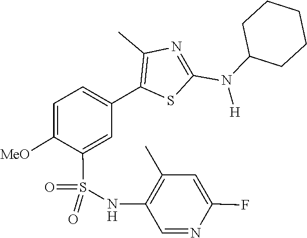 [SMILES: COc1ccc(-c2sc(NC3CCCCC3)nc2C)cc1S(=O)(=O)Nc1cnc(F)cc1C] |
| | 5-[2-(cyclohexylamino)-4-methyl-thiazol- |
| | 5-yl]-N-(6-fluoro-4-methyl-3-pyridyl)-2- |
| | methoxy-benzenesulfonamide |
| S-72 | 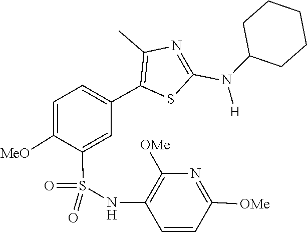 [SMILES: COc1ccc(NS(=O)(=O)c2cc(-c3sc(NC4CCCCC4)nc3C)ccc2OC)c(OC)n1] |
| | 5-[2-(cyclohexylamino)-4-methyl- |
| | thiazol-5-yl]-N-(2,6-dimethoxy-3- |
| | pyridyl)-2-methoxy-benzenesulfonamide |
| S-73 | 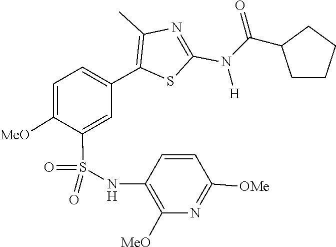 [SMILES: COc1ccc(NS(=O)(=O)c2cc(-c3sc(NC(=O)C4CCCC4)nc3C)ccc2OC)c(OC)n1] |
| | N-[5-[3-[(2,6-dimethoxy-3- |
| | pyridyl)sulfamoyl]-4-methoxy-phenyl]-4- |
| | methyl-thiazol-2- |
| | yl]cyclopentanecarboxamide |
| S-74 | 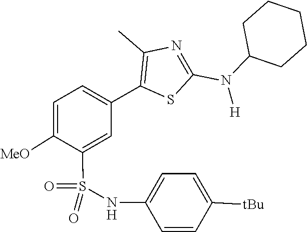 [SMILES: COc1ccc(-c2sc(NC3CCCCC3)nc2C)cc1S(=O)(=O)Nc1ccc(C(C)(C)C)cc1] |
| | N-(4-tert-butylphenyl)-5-[2- |
| | (cyclohexylamino)-4-methyl-thiazol-5- |
| | yl]-2-methoxy-benzenesulfonamide |
| S-75 | 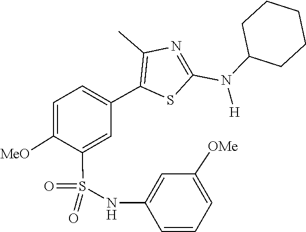 [SMILES: COc1cccc(NS(=O)(=O)c2cc(-c3sc(NC4CCCCC4)nc3C)ccc2OC)c1] |
| | 5-[2-(cyclohexylamino)-4-methyl-thiazol- |
| | 5-yl]-2-methoxy-N-(3- |
| | methoxyphenyl)benzenesulfonamide |
| S-76 | 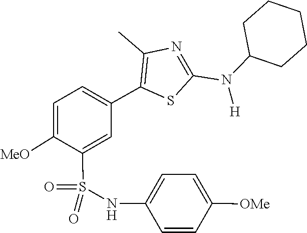 [SMILES: COc1ccc(NS(=O)(=O)c2cc(-c3sc(NC4CCCCC4)nc3C)ccc2OC)cc1] |
| | 5-[2-(cyclohexylamino)-4-methyl- |
| | thiazol-5-yl]-2-methoxy-N-(4- |
| | methoxyphenyl)benzenesulfonamide |
| S-77 | 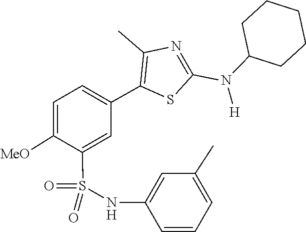 [SMILES: COc1ccc(-c2sc(NC3CCCCC3)nc2C)cc1S(=O)(=O)Nc1cccc(C)c1] |
| | 5-[2-(cyclohexylamino)-4-methyl-thiazol- |
| | 5-yl]-2-methoxy-N-(m- |
| | tolyl)benzenesulfonamide |
| S-78 | 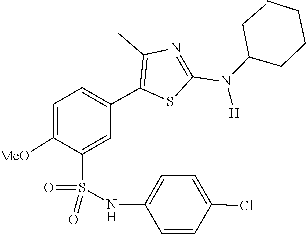 [SMILES: CCCCC(CC)Nc1nc(C)c(-c2ccc(OC)c(S(=O)(=O)Nc3ccc(Cl)cc3)c2)s1] |
| | N-(4-chlorophenyl)-5-[2- |
| | (cyclohexylamino)-4-methyl-thiazol-5- |
| | yl]-2-methoxy-benzenesulfonamide |
| S-79 | 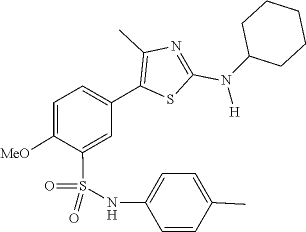 [SMILES: COc1ccc(-c2sc(NC3CCCCC3)nc2C)cc1S(=O)(=O)Nc1ccc(C)cc1] |
| | 5-[2-(cyclohexylamino)-4-methyl-thiazol- |
| | 5-yl]-2-methoxy-N-(p- |
| | tolyl)benzenesulfonamide |
| S-80 | 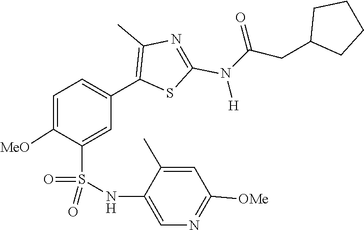 [SMILES: COc1cc(C)c(NS(=O)(=O)c2cc(-c3sc(NC(=O)CC4CCCC4)nc3C)ccc2OC)cn1] |
| | 2-cyclopentyl-N-[5-[4-methoxy-3-[(6- |
| | methoxy-4-methyl-3- |
| | pyridyl)sulfamoyl]phenyl]-4-methyl- |
| | thiazol-2-yl]acetamide |
| S-81 | 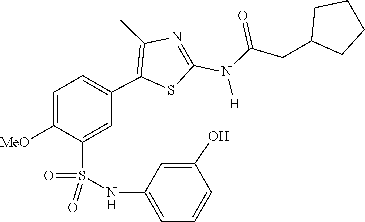 [SMILES: COc1ccc(-c2sc(NC(=O)CC3CCCC3)nc2C)cc1S(=O)(=O)Nc1cccc(O)c1] |
| | 2-cyclopentyl-N-[5-[3-[(3- |
| | hydroxyphenyl)sulfamoyl]-4-methoxy- |
| | phenyl]-4-methyl-thiazol-2-yl]acetamide |
| S-82 | 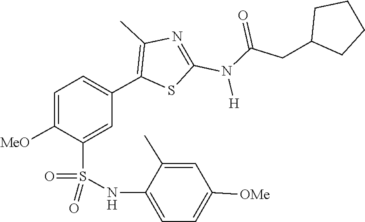 [SMILES: COc1ccc(NS(=O)(=O)c2cc(-c3sc(NC(=O)CC4CCCC4)nc3C)ccc2OC)c(C)c1] |
| | 2-cyclopentyl-N-[5-[4-methoxy-3-[(4- |
| | methoxy-2-methyl- |
| | phenyl)sulfamoyl]phenyl]-4-methyl- |
| | thiazol-2-yl]acetamide |
| S-83 | 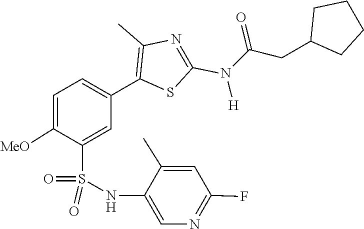 [SMILES: COc1ccc(-c2sc(NC(=O)CC3CCCC3)nc2C)cc1S(=O)(=O)Nc1cnc(F)cc1C] |
| | 2-cyclopentyl-N-[5-[3-[(6-fluoro-4-methyl- |
| | 3-pyridyl)sulfamoyl]-4-methoxy-phenyl]- |
| | 4-methyl-thiazol-2-yl]acetamide;2,2,2- |
| | trifluoroacetic acid |
| S-84 | 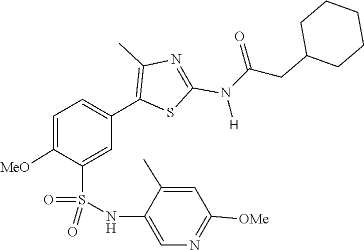 [SMILES: COc1cc(C)c(NS(=O)(=O)c2cc(-c3sc(NC(=O)CC4CCCCC4)nc3C)ccc2OC)cn1] |
| | 2-cyclohexyl-N-[5-[4-methoxy-3-[(6- |
| | methoxy-4-methyl-3- |
| | pyridyl)sulfamoyl]phenyl]-4-methyl- |
| | thiazol-2-yl]acetamide;2,2,2- |
| | trifluoroacetic acid |
| S-85 | 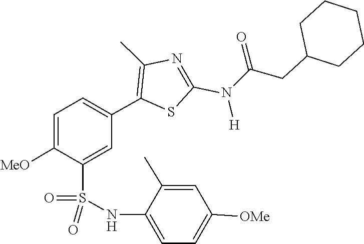 [SMILES: COc1ccc(NS(=O)(=O)c2cc(-c3sc(NC(=O)CC4CCCCC4)nc3C)ccc2OC)c(C)c1] |
| | 2-cyclohexyl-N-[5-[4-methoxy-3-[(4- |
| | methoxy-2-methyl- |
| | phenyl)sulfamoyl]phenyl]-4-methyl- |
| | thiazol-2-yl]acetamide |
| S-86 | 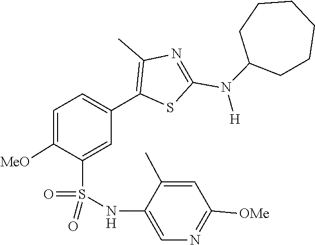 [SMILES: COc1cc(C)c(NS(=O)(=O)c2cc(-c3sc(NC4CCCCCC4)nc3C)ccc2OC)cn1] |
| | 5-[2-(cycloheptylamino)-4-methyl- |
| | thiazol-5-yl]-2-methoxy-N-(6-methoxy- |
| | 4-methyl-3- |
| | pyridyl)benzenesulfonamide;2,2,2- |
| | trifluoroacetic acid |
| S-87 | 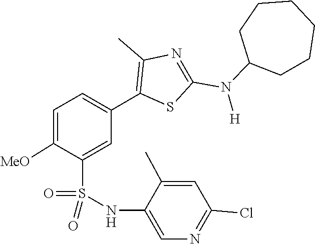 [SMILES: COc1ccc(-c2sc(NC3CCCCCC3)nc2C)cc1S(=O)(=O)Nc1cnc(Cl)cc1C] |
| | N-(6-chloro-5-methyl-3-pyridyl)-5-[-2 |
| | (cycloheptylamino)-4-methyl-thiazol-5-yl]- |
| | 2-methoxy-benzenesulfonamide;2,2,2- |
| | trifluoroacetic acid |
| S-88 | 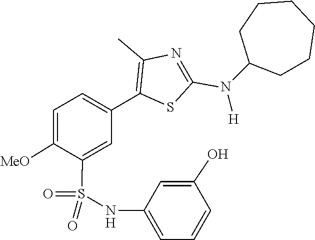 [SMILES: COc1ccc(-c2sc(NC3CCCCCC3)nc2C)cc1S(=O)(=O)Nc1cccc(O)c1] |
| | 5-[2-(cycloheptylamino)-4-methyl- |
| | thiazol-5-yl]-N-(3-hydroxyphenyl)-2- |
| | methoxy-benzenesulfonamide;2,2,2- |
| | trifluoroacetic acid |
| S-89 | 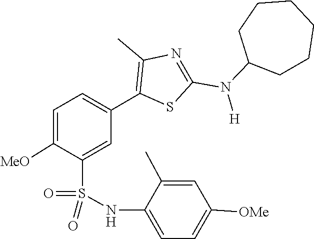 [SMILES: COc1ccc(NS(=O)(=O)c2cc(-c3sc(NC4CCCCCC4)nc3C)ccc2OC)c(C)c1] |
| | 5-[2-(cycloheptylamino)-4-methyl-thiazol- |
| | 5-yl]-2-methoxy-N-(4-methoxy-2-methyl- |
| | phenyl)benzenesulfonamide;2,2,2- |
| | trifluoroacetic acid |
| S-90 | 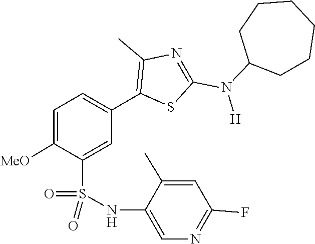 [SMILES: COc1ccc(-c2sc(NC3CCCCCC3)nc2C)cc1S(=O)(=O)Nc1cnc(F)cc1C] |
| | 5-[2-(cycloheptylamino)-4-methyl- |
| | thiazol-5-yl]-N-(6-fluoro-4-methyl-3- |
| | pyridyl)-2-methoxy-benzenesulfonamide |
| S-91 | 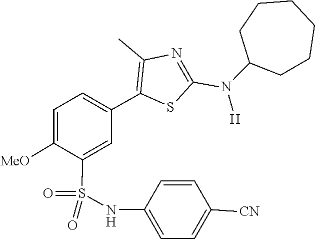 [SMILES: COc1ccc(-c2sc(NC3CCCCCC3)nc2C)cc1S(=O)(=O)Nc1ccc(C#N)cc1] |
| | N-(4-cyanophenyl)-5-[2- |
| | (cycloheptylamino)-4-methyl-thiazol-5-yl]- |
| | 2-methoxy-benzenesulfonamide;2,2,2- |
| | trifluoroacetic acid |
| S-92 | 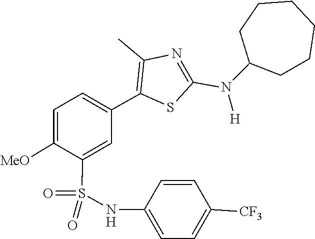 [SMILES: COc1ccc(-c2sc(NC3CCCCCC3)nc2C)cc1S(=O)(=O)Nc1ccc(C(F)(F)F)cc1] |
| | 5-[2-(cycloheptylamino)-4-methyl- |
| | thiazol-5-yl]-2-methoxy-N-[4- |
| | (trifluoromethyl)phenyl]benzenesulfonamide |
| S-93 | 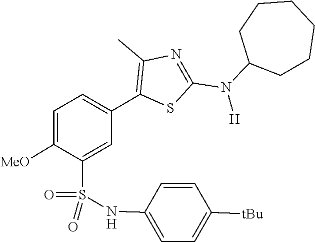 [SMILES: COc1ccc(-c2sc(NC3CCCCCC3)nc2C)cc1S(=O)(=O)Nc1ccc(C(C)(C)C)cc1] |
| | N-(4-tert-butylphenyl)-5-[2- |
| | (cycloheptylamino)-4-methyl-thiazol-5-yl]- |
| | 2-methoxy-benzenesulfonamide |
| S-94 | 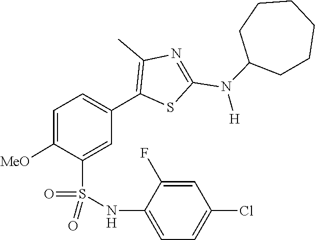 [SMILES: COc1ccc(-c2sc(NC3CCCCCC3)nc2C)cc1S(=O)(=O)Nc1ccc(Cl)cc1F] |
| | N-(4-chloro-2-fluoro-phenyl)-5-[2- |
| | (cycloheptylamino)-4-methyl-thiazol-5- |
| | yl]-2-methoxy- |
| | benzenesulfonamide;2,2,2-trifluoroacetic acid |
| S-95 | 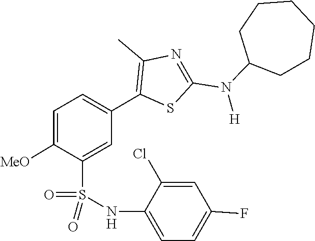 [SMILES: COc1ccc(-c2sc(NC3CCCCCC3)nc2C)cc1S(=O)(=O)Nc1ccc(F)cc1Cl] |
| | N-(2-chloro-4-fluoro-phenyl)-5-[2- |
| | (cycloheptylamino)-4-methyl-thiazol-5-yl]- |
| | 2-methoxy-benzenesulfonamide;2,2,2- |
| | trifluoroacetic acid |
| S-96 | 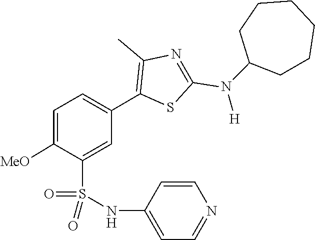 [SMILES: COc1ccc(-c2sc(NC3CCCCCC3)nc2C)cc1S(=O)(=O)Nc1ccncc1] |
| | 5-[2-(cycloheptylamino)-4-methyl- |
| | thiazol-5-yl]-2-methoxy-N-(4- |
| | pyridyl)benzenesulfonamide;2,2,2- |
| | trifluoroacetic acid |
| S-97 | 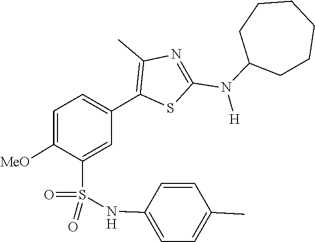 [SMILES: COc1ccc(-c2sc(NC3CCCCCC3)nc2C)cc1S(=O)(=O)Nc1ccc(C)cc1] |
| | 5-[2-(cycloheptylamino)-4-methyl-thiazol- |
| | 5-yl]-2-methoxy-N-(p- |
| | toly)benzenesulfonamide;2,2,2- |
| | trifluoroacetic acid |
| S-98 | 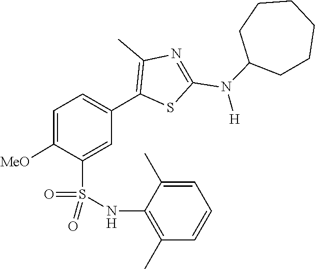 [SMILES: COc1ccc(-c2sc(NC3CCCCCC3)nc2C)cc1S(=O)(=O)Nc1c(C)cccc1C] |
| | 5-[2-(cycloheptylamino)-4-methyl- |
| | thiazol-5-yl]-N-(2,6-dimethylphenyl)-2- |
| | methoxy-benzenesulfonamide;2,2,2- |
| | trifluoroacetic acid |
| S-99 | 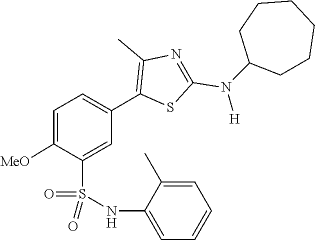 [SMILES: COc1ccc(-c2sc(NC3CCCCCC3)nc2C)cc1S(=O)(=O)Nc1ccccc1C] |
| | 5-[2-(cycloheptylamino)-4-methyl-thiazol- |
| | 5-yl]-2-methoxy-N-(o- |
| | toly)benzenesulfonamide;2,2,2- |
| | trifluoroacetic acid |
| S-100 | 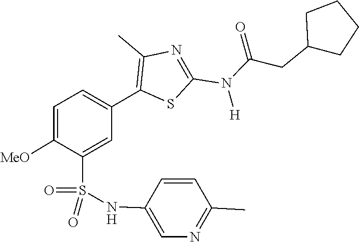 [SMILES: COc1ccc(-c2sc(NC(=O)CC3CCCC3)nc2C)cc1S(=O)(=O)Nc1ccc(C)nc1] |
| | 2-cyclopentyl-N-[5-[4-methoxy-3-[(6- |
| | methyl-3-pyridyl)sulfamoyl]phenyl]-4- |
| | methyl-thiazol-2-yl]acetamide;2,2,2- |
| | trifluoroacetic acid |
| S-101 | 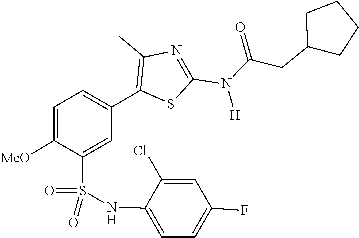 [SMILES: COc1ccc(-c2sc(NC(=O)CC3CCCC3)nc2C)cc1S(=O)(=O)Nc1ccc(F)cc1Cl] |
| | N-[5-[3-[(2-chloro-4-fluoro- |
| | phenyl)sulfamoyl]-4-methoxy-phenyl]-4- |
| | methyl-thiazol-2-yl]-2-cyclopentyl- |
| | acetamide |
| S-102 | 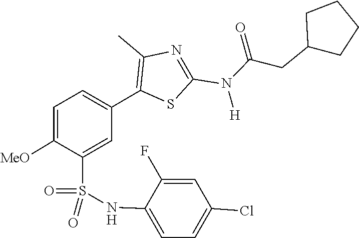 [SMILES: COc1ccc(-c2sc(NC(=O)CC3CCCC3)nc2C)cc1S(=O)(=O)Nc1ccc(Cl)cc1F] |
| | N-[5-[3-[(4-chloro-2-fluoro- |
| | phenyl)sulfamoyl]-4-methoxy-phenyl]- |
| | 4-methyl-thiazol-2-yl]-2-cyclopentyl- |
| | acetamide |
| S-103 | 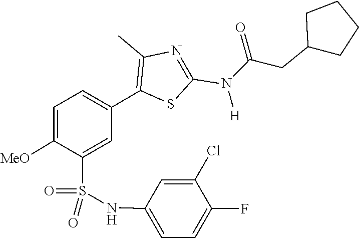 [SMILES: COc1ccc(-c2sc(NC(=O)CC3CCCC3)nc2C)cc1S(=O)(=O)Nc1ccc(F)c(Cl)c1] |
| | N-[5-[3-[(3-chloro-4-fluoro- |
| | phenyl)sulfamoyl]-4-methoxy-phenyl]-4- |
| | methyl-thiazol-2-yl]-2-cyclopentyl- |
| | acetamide |
| S-104 | 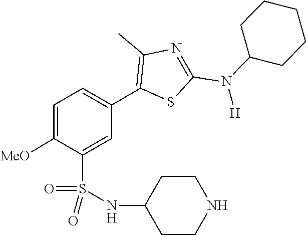 [SMILES: COc1ccc(-c2sc(NC3CCCCC3)nc2C)cc1S(=O)(=O)NC1CCNCC1] |
| | 5-[2-(cyclohexylamino)-4-methyl- |
| | thiazol-5-yl]-2-methoxy-N-(4- |
| | piperidyl)benzenesulfonamide |
| S-105 | 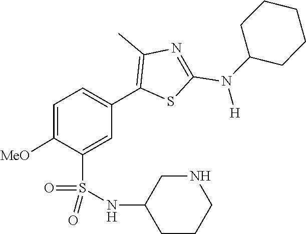 [SMILES: COc1ccc(-c2sc(NC3CCCCC3)nc2C)cc1S(=O)(=O)NC1CCCNC1] |
| | 5-[2-(cyclohexylamino)-4-methyl-thiazol- |
| | 5-yl]-2-methoxy-N-(3- |
| | piperidyl)benzenesulfonamide |
| S-106 | 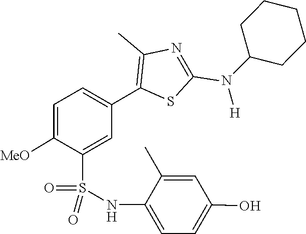 [SMILES: COc1ccc(-c2sc(NC3CCCCC3)nc2C)cc1S(=O)(=O)Nc1ccc(O)cc1C] |
| | 5-[2-(cyclohexylamino)-4-methyl- |
| | thiazol-5-yl]-N-(4-hydroxy-2-methyl- |
| | phenyl)-2-methoxy-benzenesulfonamide |
| S-107 | 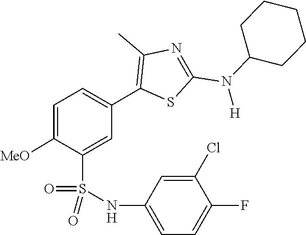 [SMILES: COc1ccc(-c2sc(NC3CCCCC3)nc2C)cc1S(=O)(=O)Nc1ccc(F)c(Cl)c1] |
| | N-(3-chloro-4-fluoro-phenyl)-5-[2- |
| | (cyclohexylamino)-4-methyl-thiazol-5-yl]- |
| | 2-methoxy-benzenesulfonamide |
| S-108 | 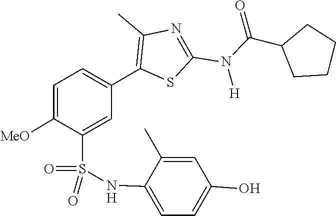 [SMILES: COc1ccc(-c2sc(NC(=O)C3CCCC3)nc2C)cc1S(=O)(=O)Nc1ccc(O)cc1C] |
| | N-[5-[3-[(4-hydroxy-2-methyl- |
| | phenyl)sulfamoyl]-4-methoxy-phenyl]- |
| | 4-methyl-thiazol-2- |
| | yl]cyclopentanecarboxamide |
| S-109 | 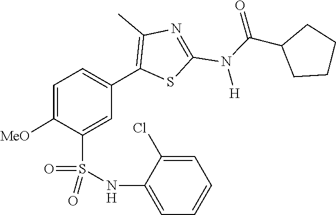 [SMILES: COc1ccc(-c2sc(NC(=O)C3CCCC3)nc2C)cc1S(=O)(=O)Nc1ccccc1Cl] |
| | N-[5-[3-[(2-chlorophenyl)sulfamoyl]-4- |
| | methoxy-phenyl]-4-methyl-thiazol-2- |
| | yl]cyclopentanecarboxamide |
| S-110 | 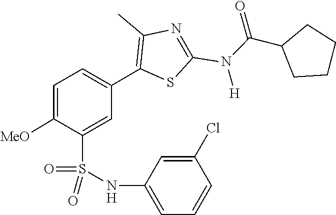 [SMILES: COc1ccc(-c2sc(NC(=O)C3CCCC3)nc2C)cc1S(=O)(=O)Nc1cccc(Cl)c1] |
| | N-[5-[3-[(3-chlorophenyl)sulfamoyl]-4- |
| | methoxy-phenyl]-4-methyl-thiazol-2- |
| | yl]cyclopentanecarboxamide |
| S-111 | 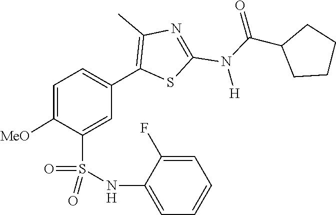 [SMILES: COc1ccc(-c2sc(NC(=O)C3CCCC3)nc2C)cc1S(=O)(=O)Nc1ccccc1F] |
| | N-[5-[3-[(2-fluorophenyl)sulfamoyl]-4- |
| | methoxy-phenyl]-4-methyl-thiazol-2- |
| | yl]cyclopentanecarboxamide |
| S-112 | 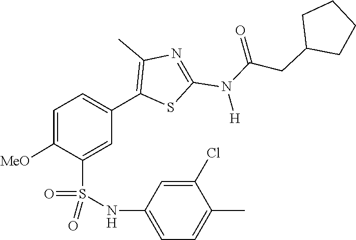 [SMILES: COc1ccc(-c2sc(NC(=O)CC3CCCC3)nc2C)cc1S(=O)(=O)Nc1ccc(C)c(Cl)c1] |
| | N-[5-[3-[(3-chloro-4-methyl- |
| | phenyl)sulfamoyl]-4-methoxy-phenyl]- |
| | 4-methyl-thiazol-2-yl]-2-cyclopentyl- |
| | acetamide |
| S-113 | 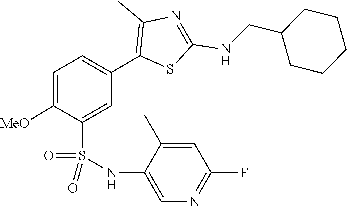 [SMILES: COc1ccc(-c2sc(NCC3CCCCC3)nc2C)cc1S(=O)(=O)Nc1cnc(F)cc1C] |
| | 5-[2-(cyclohexylmethylamino)-4-methyl- |
| | thiazol-5-yl]-N-(6-fluoro-4-methyl-3- |
| | pyridyl)-2-methoxy-benzenesulfonamide |
| S-114 | 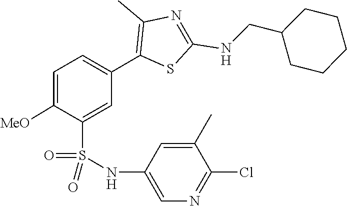 [SMILES: COc1ccc(-c2sc(NCC3CCCCC3)nc2C)cc1S(=O)(=O)Nc1cnc(Cl)c(C)c1] |
| | N-(6-chloro-5-methyl-3-pyridyl)-5-[2- |
| | (cyclohexylmethylamino)-4-methyl- |
| | thiazol-5-yl]-2-methoxy- |
| | benzenesulfonamide |
| S-115 | 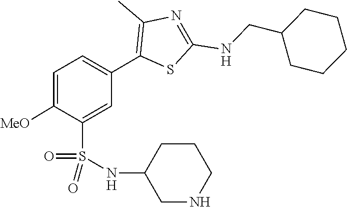 [SMILES: COc1ccc(-c2sc(NCC3CCCCC3)nc2C)cc1S(=O)(=O)NC1CCCNC1] |
| | 5-[2-(cyclohexylmethylamino)-4-methyl- |
| | thiazol-5-yl]-2-methoxy-N-(3- |
| | piperidyl)benzenesulfonamide |
| S-116 | 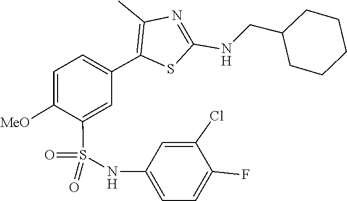 [SMILES: COc1ccc(-c2sc(NCC3CCCCC3)nc2C)cc1S(=O)(=O)Nc1ccc(F)c(Cl)c1] |
| | N-(3-chloro-4-fluoro-phenyl)-5-[2- |
| | (cyclohexylmethylamino)-4-methyl- |
| | thiazol-5-yl]-2-methoxy- |
| | benzenesulfonamide |
| S-117 | 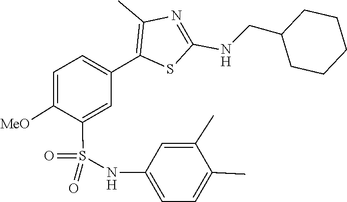 [SMILES: COc1ccc(-c2sc(NCC3CCCCC3)nc2C)cc1S(=O)(=O)Nc1ccc(C)c(C)c1] |
| | 5-[2-(cyclohexylmethylamino)-4-methyl- |
| | thiazol-5-yl]-N-(3,4-dimethylphenyl)-2- |
| | methoxy-benzenesulfonamide |
| S-118 | 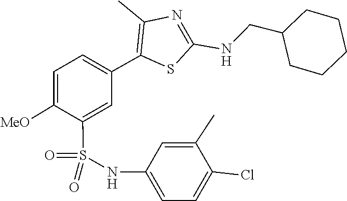 [SMILES: COc1ccc(-c2sc(NCC3CCCCC3)nc2C)cc1S(=O)(=O)Nc1ccc(Cl)c(C)c1] |
| | N-(4-chloro-3-methyl-phenyl)-5-[2- |
| | (cyclohexylmethylamino)-4-methyl- |
| | thiazol-5-yl]-2-methoxy- |
| | benzenesulfonamide |
| S-119 | 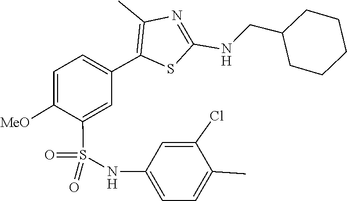 [SMILES: COc1ccc(-c2sc(NCC3CCCCC3)nc2C)cc1S(=O)(=O)Nc1ccc(C)c(Cl)c1] |
| | N-(3-chloro-4-methyl-phenyl)-5-[2- |
| | (cyclohexylmethylamino)-4-methyl- |
| | thiazol-5-yl]-2-methoxy- |
| | benzenesulfonamide |
| S-120 | 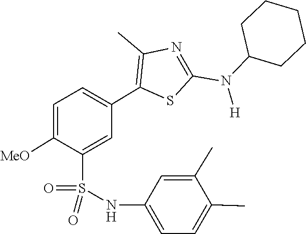 [SMILES: COc1ccc(-c2sc(NC3CCCCC3)nc2C)cc1S(=O)(=O)Nc1ccc(C)c(C)c1] |
| | 5-[2-(cyclohexylamino)-4-methyl- |
| | thiazol-5-yl]-N-(3,4-dimethylphenyl)-2- |
| | methoxy-benzenesulfonamide |
| S-121 | 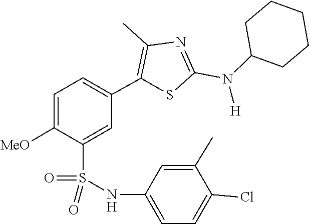 [SMILES: COc1ccc(-c2sc(NC3CCCCC3)nc2C)cc1S(=O)(=O)Nc1ccc(Cl)c(C)c1] |
| | N-(4-chloro-3-methyl-phenyl)-5-[2- |
| | (cyclohexylamino)-4-methyl-thiazol-5-yl]- |
| | 2-methoxy-benzenesulfonamide |
| S-122 | 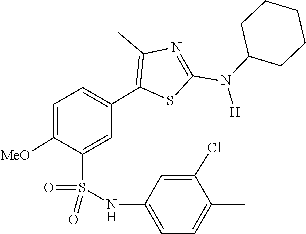 [SMILES: COc1ccc(-c2sc(NC3CCCCC3)nc2C)cc1S(=O)(=O)Nc1ccc(C)c(Cl)c1] |
| | N-(3-chloro-4-methyl-phenyl)-5-[2- |
| | (cyclohexylamino)-4-methyl-thiazol-5- |
| | yl]-2-methoxy-benzenesulfonamide |
| S-123 | 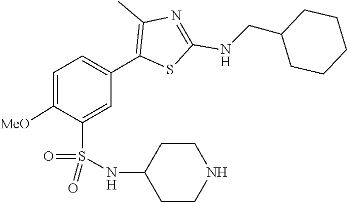 [SMILES: COc1ccc(-c2sc(NCC3CCCCC3)nc2C)cc1S(=O)(=O)NC1CCNCC1] |
| | 5-[2-(cyclohexylmethylamino)-4-methyl- |
| | thiazol-5-yl]-2-methoxy-N-(4- |
| | piperidyl)benzenesulfonamide |
| S-124 | 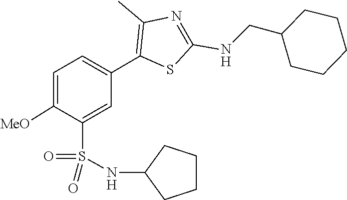 [SMILES: COc1ccc(-c2sc(NCC3CCCCC3)nc2C)cc1S(=O)(=O)NC1CCCC1] |
| | 5-[2-(cyclohexylmethylamino)-4- |
| | methyl-thiazol-5-yl]-N-cyclopentyl-2- |
| | methoxy-benzenesulfonamide |
| S-125 | 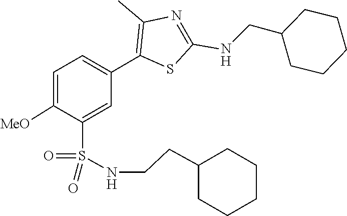 [SMILES: COc1ccc(-c2sc(NCC3CCCCC3)nc2C)cc1S(=O)(=O)NCCC1CCCCC1] |
| | N-(2-cyclohexylethyl)-5-[2- |
| | (cyclohexylmethylamino)-4-methyl- |
| | thiazol-5-yl]-2-methoxy- |
| | benzenesulfonamide |
| S-126 | 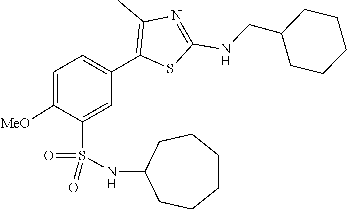 [SMILES: COc1ccc(-c2sc(NCC3CCCCC3)nc2C)cc1S(=O)(=O)NC1CCCCCC1] |
| | N-cycloheptyl-5-[2- |
| | (cyclohexylmethylamino)-4-methyl- |
| | thiazol-5-yl]-2-methoxy- |
| | benzenesulfonamide |
| S-127 | 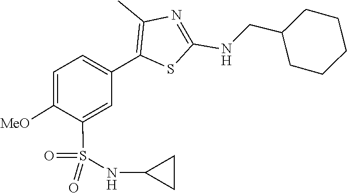 [SMILES: COc1ccc(-c2sc(NCC3CCCCC3)nc2C)cc1S(=O)(=O)NC1CC1] |
| | 5-[2-(cyclohexylmethylamino)-4-methyl- |
| | thiazol-5-yl]-N-cyclopropyl-2-methoxy- |
| | benzenesulfonamide |
| S-128 | 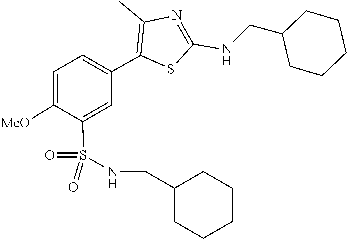 [SMILES: COc1ccc(-c2sc(NCC3CCCCC3)nc2C)cc1S(=O)(=O)NCC1CCCCC1] |
| | N-(cyclohexylmethyl)-5-[2- |
| | (cyclohexylmethylamino)-4-methyl- |
| | thiazol-5-yl]-2-methoxy- |
| | benzenesulfonamide |
| S-129 | 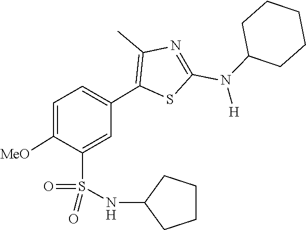 [SMILES: COc1ccc(-c2sc(NC3CCCCC3)nc2C)cc1S(=O)(=O)NC1CCCC1] |
| | 5-[2-(cyclohexylamino)-4-methyl-thiazol- |
| | 5-yl]-N-cyclopentyl-2-methoxy- |
| | benzenesulfonamide |
| S-130 | 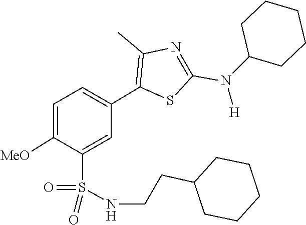 [SMILES: COc1ccc(-c2sc(NC3CCCCC3)nc2C)cc1S(=O)(=O)NCCC1CCCCC1] |
| | 5-[2-(cyclohexylamino)-4-methyl- |
| | thiazol-5-yl]-N-(2-cyclohexylethyl)-2- |
| | methoxy-benzenesulfonamide |
| S-131 | 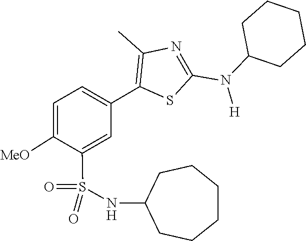 [SMILES: COc1ccc(-c2sc(NC3CCCCC3)nc2C)cc1S(=O)(=O)NC1CCCCCC1] |
| | N-cycloheptyl-5-[2-(cyclohexylamino)-4- |
| | methyl-thiazol-5-yl]-2-methoxy- |
| | benzenesulfonamide |
| S-132 | 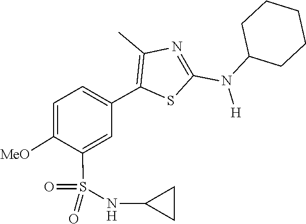 [SMILES: COc1ccc(-c2sc(NC3CCCCC3)nc2C)cc1S(=O)(=O)NC1CC1] |
| | 5-[2-(cyclohexylamino)-4-methyl- |
| | thiazol-5-yl]-N-cyclopropyl-2-methoxy- |
| | benzenesulfonamide |
| S-133 | 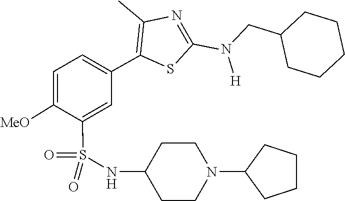 [SMILES: COc1ccc(-c2sc(NCC3CCCCC3)nc2C)cc1S(=O)(=O)NC1CCN(C2CCCC2)CC1] |
| | 5-[2-(cyclohexylmethylamino)-4-methyl- |
| | thiazol-5-yl]-N-(1-cyclopentyl-4- |
| | piperidyl)-2-methoxy-benzenesulfonamide |
| S-134 | 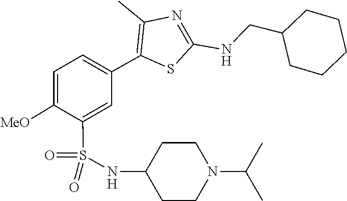 [SMILES: COc1ccc(-c2sc(NCC3CCCCC3)nc2C)cc1S(=O)(=O)NC1CCN(C(C)C)CC1] |
| | 5-[2-(cyclohexylmethylamino)-4- |
| | methyl-thiazol-5-yl]-N-(1-isopropyl-4- |
| | piperidyl)-2-methoxy- |
| | benzenesulfonamide |
| S-135 | 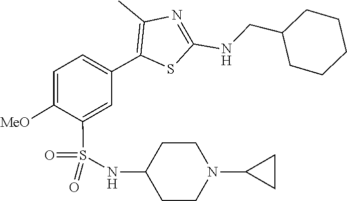 [SMILES: COc1ccc(-c2sc(NCC3CCCCC3)nc2C)cc1S(=O)(=O)NC1CCN(C2CC2)CC1] |
| | 5-[2-(cyclohexylmethylamino)-4-methyl- |
| | thiazol-5-yl]-N-(1-cyclopropyl-4- |
| | piperidyl)-2-methoxy-benzenesulfonamide |
| S-136 | 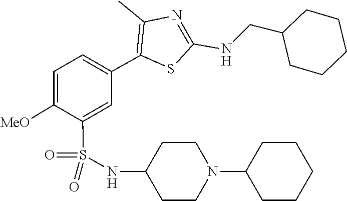 [SMILES: COc1ccc(-c2sc(NCC3CCCCC3)nc2C)cc1S(=O)(=O)NC1CCN(C2CCCCC2)CC1] |
| | 5-[2-(cyclohexylmethylamino)-4- |
| | methyl-thiazol-5-yl]-N-(1-cyclohexyl-4- |
| | piperidyl)-2-methoxy- |
| | benzenesulfonamide |
| S-137 | 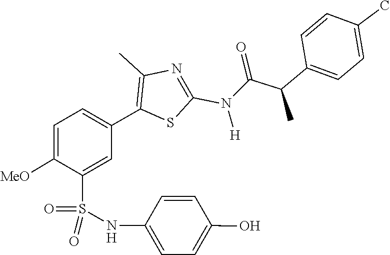 |
| | (2R)-2-(4-chlorophenye-N-[5-[3-[(4- |
| | hydroxyphenyl)sulfamoyl]-4-methoxy- |
| | phenyl]-4-methyl-thiazol-2- |
| | yl]propanamide |
| S-138 | 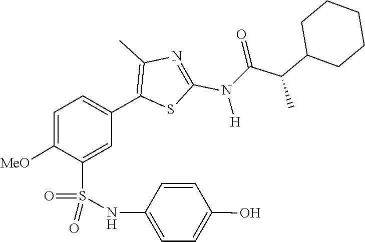 [SMILES: COc1ccc(-c2sc(NC(=O)[C@@H](C)C3CCCCC3)nc2C)cc1S(=O)(=O)Nc1ccc(O)cc1] |
| | (2S)-2-cyclohexyl-N-[5-[3-[(4- |
| | hydroxyphenyl)sulfamoyl]-4-methoxy- |
| | phenyl]-4-methyl-thiazol-2- |
| | yl]propanamide |
| S-139 | 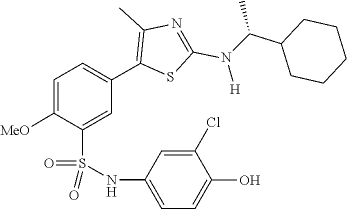 [SMILES: COc1ccc(-c2sc(N[C@H](C)C3CCCCC3)nc2C)cc1S(=O)(=O)Nc1ccc(O)c(Cl)c1] |
| | N-(3-chloro-4-fluoro-phenyl)-5-[2-[[(1R)- |
| | 1-cyclohexylethyl]amino]-4-methyl- |
| | thiazol-5-yl]-2-methoxy- |
| | benzenesulfonamide |
| S-140 | 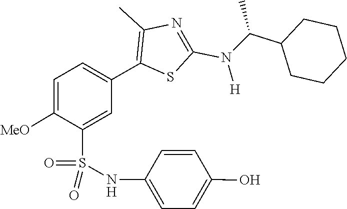 [SMILES: COc1ccc(-c2sc(N[C@H](C)C3CCCCC3)nc2C)cc1S(=O)(=O)Nc1ccc(O)cc1] |
| | 5-[2-[[(1R)-1-cyclohexylethyl]amino]-4- |
| | methyl-thiazol-5-yl]-N-(4- |
| | hydroxyphenyl)-2-methoxy- |
| | benzenesulfonamide |
| S-141 | 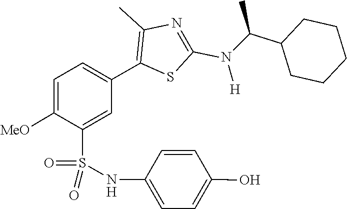 [SMILES: COc1ccc(-c2sc(N[C@@H](C)C3CCCCC3)nc2C)cc1S(=O)(=O)Nc1ccc(O)cc1] |
| | 5-[2-[[(1S)-1-cyclohexylethyl]amino]-4- |
| | methyl-thiazol-5-yl]-N-(4-hydroxyphenyl)- |
| | 2-methoxy-benzenesulfonamide |
| S-142 | 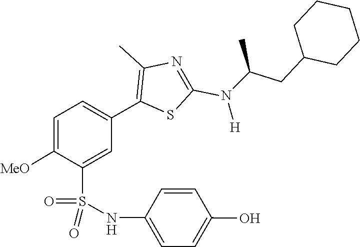 [SMILES: COc1ccc(-c2sc(N[C@@H](C)CC3CCCCC3)nc2C)cc1S(=O)(=O)Nc1ccc(O)cc1] |
| | 5-[2-[[(1S)-2-cyclohexyl-1-methyl- |
| | ethyl]amino]-4-methyl-thiazol-5-yl]-N- |
| | (4-hydroxyphenyl)-2-methoxy-benzenesulfonamide |
| S-143 | 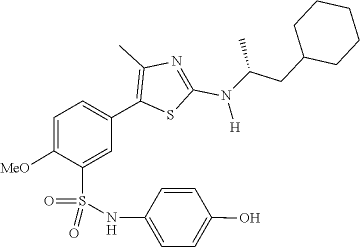 [SMILES: COc1ccc(-c2sc(N[C@H](C)CC3CCCCC3)nc2C)cc1S(=O)(=O)Nc1ccc(O)cc1] |
| | 5-[2-[[(1R)-2-cyclohexyl-1-methyl- |
| | ethyl]amino]-4-methyl-thiazol-5-yl]-N-(4- |
| | hydroxyphenyl)-2-methoxy- |
| | benzenesulfonamide |
| S-144 | 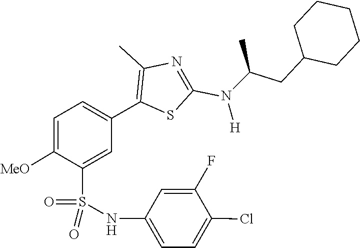 [SMILES: COc1ccc(-c2sc(N[C@@H](C)CC3CCCCC3)nc2C)cc1S(=O)(=O)Nc1ccc(Cl)c(F)c1] |
| | N-(4-chloro-3-fluoro-phenyl)-5-[2- |
| | [[(1S)-2-cyclohexyl-1-methyl- |
| | ethyl]amino]-4-methyl-thiazol-5-yl]-2- |
| | methoxy-benzenesulfonamide |
| S-145 | 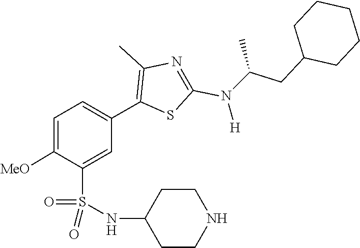 [SMILES: COc1ccc(-c2sc(N[C@H](C)CC3CCCCC3)nc2C)cc1S(=O)(=O)NC1CCNCC1] |
| | 5-[2-[[(1R)-2-cyclohexyl-1-methyl- |
| | ethyl]amino]-4-methyl-thiazol-5-yl]-2- |
| | methoxy-N-(4- |
| | piperidyl)benzenesulfonamide |
| S-146 | 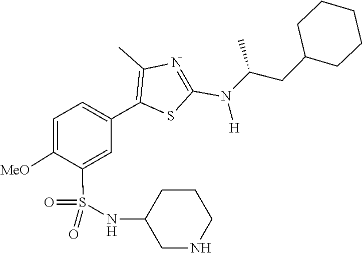 [SMILES: COc1ccc(-c2sc(N[C@H](C)CC3CCCCC3)nc2C)cc1S(=O)(=O)NC1CCCNC1] |
| | 5-[2-[[(1R)-2-cyclohexyl-1-methyl- |
| | ethyl]amino]-4-methyl-thiazol-5-yl]-2- |
| | methoxy-N-(3- |
| | piperidyl)benzenesulfonamide |
| S-147 | 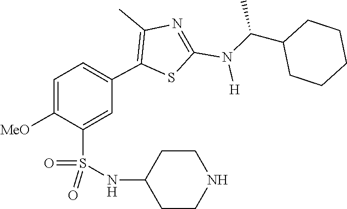 [SMILES: COc1ccc(-c2sc(N[C@H](C)C3CCCCC3)nc2C)cc1S(=O)(=O)NC1CCNCC1] |
| | 5-[2-[[(1R)-1-cyclohexylethyl]amino]-4- |
| | methyl-thiazol-5-yl]-2-methoxy-N-(4- |
| | piperidyl)benzenesulfonamide |
| S-148 | 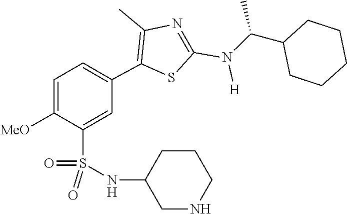 [SMILES: COc1ccc(-c2sc(N[C@H](C)C3CCCCC3)nc2C)cc1S(=O)(=O)NC1CCCNC1] |
| | 5-[2-[[(1R)-1-cyclohexylethyl]amino]-4- |
| | methyl-thiazol-5-yl]-2-methoxy-N-(3- |
| | piperidyl)benzenesulfonamide |
| S-149 | 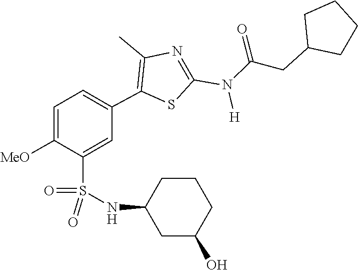 [SMILES: COc1ccc(-c2sc(NC(=O)CC3CCCC3)nc2C)cc1S(=O)(=O)N[C@H]1CCC[C@@H](O)C1] |
| | 2-cyclopentyl-N-[5-[3-[[(1S,3S)-3- |
| | hydroxycyclohexyl]sulfamoyl]-4-methoxy- |
| | phenyl]-4-methyl-thiazol-2-yl]acetamide |
| S-150 | 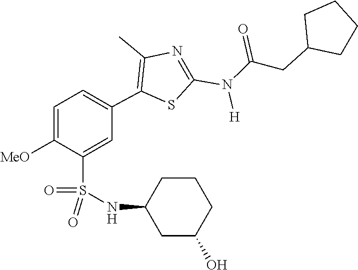 [SMILES: COc1ccc(-c2sc(NC(=O)CC3CCCC3)nc2C)cc1S(=O)(=O)N[C@H]1CCC[C@H](O)C1] |
| | 2-cyclopentyl-N-[5-[3-[[(1S,3R)-3- |
| | hydroxycyclohexyl]sulfamoyl]-4- |
| | methoxy-phenyl]-4-methyl-thiazol-2- |
| | yl]acetamide |
| S-151 | 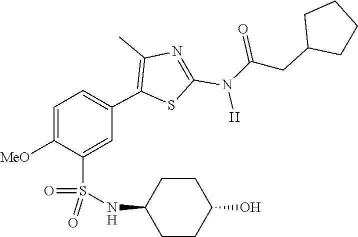 [SMILES: COc1ccc(-c2sc(NC(=O)CC3CCCC3)nc2C)cc1S(=O)(=O)N[C@H]1CC[C@H](O)CC1] |
| | 2-cyclopentyl-N-[5-[3-[(4- |
| | hydroxycyclohexyl)sulfamoyl]-4-methoxy- |
| | phenyl]-4-methyl-thiazol-2-yl]acetamide |
| S-152 | 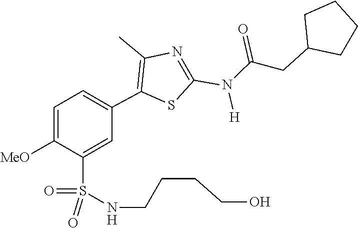 [SMILES: COc1ccc(-c2sc(NC(=O)CC3CCCC3)nc2C)cc1S(=O)(=O)NCCCCO] |
| | 2-cyclopentyl-N-[5-[3-(4- |
| | hydroxybutylsulfamoye-4-methoxy- |
| | phenyl]-4-methyl-thiazol-2-yl]acetamide |
| S-153 | 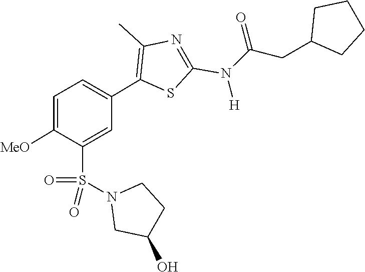 [SMILES: COc1ccc(-c2sc(NC(=O)CC3CCCC3)nc2C)cc1S(=O)(=O)N1CC[C@@H](O)C1] |
| | 2-cyclopentyl-N-[5-[3-[(3R)-3- |
| | hydroxypyrrolidin-1-yl]sulfonyl-4- |
| | methoxy-phenyl]-4-methyl-thiazol-2- |
| | yl]acetamide |
| S-154 | 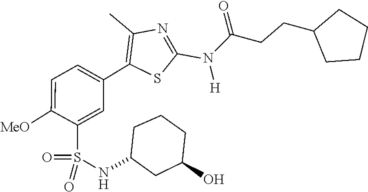 [SMILES: COc1ccc(-c2sc(NC(=O)CCC3CCCC3)nc2C)cc1S(=O)(=O)N[C@@H]1CCC[C@@H](O)C1] |
| | 3-cyclopentyl-N-[5-[3-[[(1R,3R)-3- |
| | hydroxycyclohexyl]sulfamoyl]-4- |
| | methoxy-phenyl]-4-methyl-thiazol-2- |
| | yl]propanamide |
| S-155 | 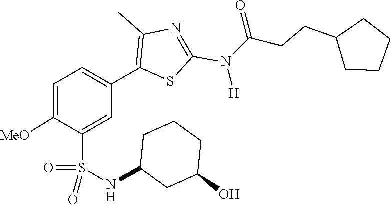 [SMILES: COc1ccc(-c2sc(NC(=O)CCC3CCCC3)nc2C)cc1S(=O)(=O)N[C@H]1CCC[C@@H](O)C1] |
| | 3-cyclopentyl-N-[5-[3-[[(1S,3R)-3- |
| | hydroxycyclohexyl]sulfamoyl]-4-methoxy- |
| | phenyl]-4-methyl-thiazol-2- |
| | yl]propanamide |
| S-156 | 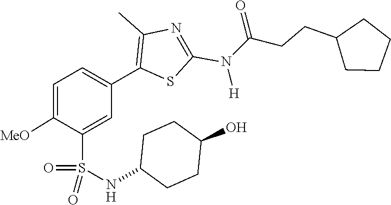 [SMILES: COc1ccc(-c2sc(NC(=O)CCC3CCCC3)nc2C)cc1S(=O)(=O)N[C@H]1CC[C@H](O)CC1] |
| | 3-cyclopentyl-N-[5-[3-[(4- |
| | hydroxycyclohexyl)sulfamoyl]-4- |
| | methoxy-phenyl]-4-methyl-thiazol-2- |
| | yl]propanamide |
| S-157 | 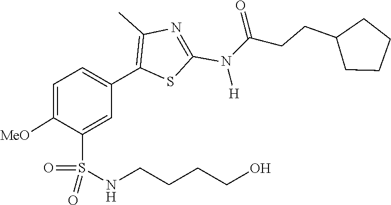 [SMILES: COc1ccc(-c2sc(NC(=O)CCC3CCCC3)nc2C)cc1S(=O)(=O)NCCCCO] |
| | 3-cyclopentyl-N-[5-[3-(4- |
| | hydroxybutylsulfamoyl)-4-methoxy- |
| | phenyl]-4-methyl-thiazol-2- |
| | yl]propanamide |
| S-158 | 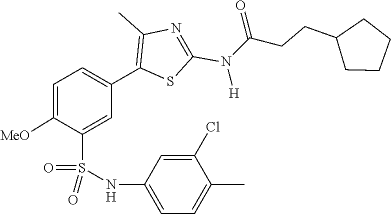 [SMILES: COc1ccc(-c2sc(NC(=O)CCC3CCCC3)nc2C)cc1S(=O)(=O)Nc1ccc(C)c(Cl)c1] |
| | 3-cyclopentyl-N-[5-[3-[(3-hydroxy-4- |
| | methyl-phenyl)sulfamoyl]-4-methoxy- |
| | phenyl]-4-methyl-thiazol-2- |
| | yl]propanamide |
| S-159 | 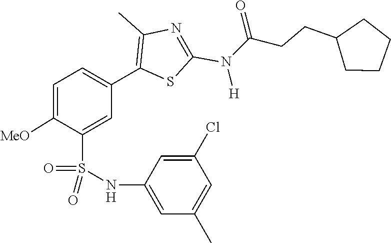 [SMILES: COc1ccc(-c2sc(NC(=O)CCC3CCCC3)nc2C)cc1S(=O)(=O)Nc1cc(C)cc(Cl)c1] |
| | 3-cyclopentyl-N-[5-[3-[(3-hydroxy-5- |
| | methyl-phenyl)sulfamoyl]-4-methoxy- |
| | phenyl]-4-methyl-thiazol-2- |
| | yl]propanamide |
| S-160 | 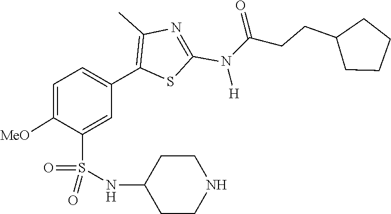 [SMILES: COc1ccc(-c2sc(NC(=O)CCC3CCCC3)nc2C)cc1S(=O)(=O)NC1CCNCC1] |
| | 3-cyclopentyl-N-[5-[4-methoxy-3-(4 |
| | piperidylsulfamoyl)phenyl]-4-methyl- |
| | thiazol-2-yl]propanamide |
| S-161 | 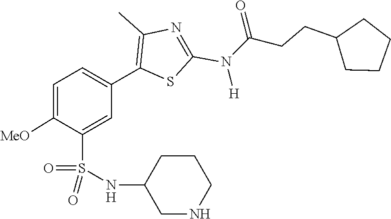 [SMILES: COc1ccc(-c2sc(NC(=O)CCC3CCCC3)nc2C)cc1S(=O)(=O)NC1CCCNC1] |
| | 3-cyclopentyl-N-[5-[4-methoxy-3-(3- |
| | piperidylsulfamoyl)phenyl]-4-methyl- |
| | thiazol-2-yl]propanamide |
| S-162 | 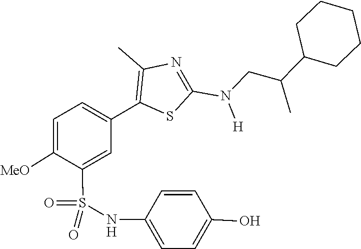 [SMILES: COc1ccc(-c2sc(NCC(C)C3CCCCC3)nc2C)cc1S(=O)(=O)Nc1ccc(O)cc1] |
| | 5-[2-(2-cyclohexylpropylamino)-4- |
| | methyl-thiazol-5-yl]-N-(4- |
| | hydroxyphenyl)-2-methoxy- |
| | benzenesulfonamide |
| S-163 | 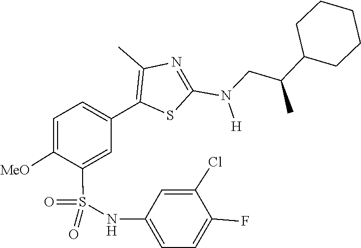 [SMILES: COc1ccc(-c2sc(NC[C@H](C)C3CCCCC3)nc2C)cc1S(=O)(=O)Nc1ccc(F)c(Cl)c1] |
| | N-(3-chloro-4-fluoro-phenyl)-5-[2-[[(2R)- |
| | 2-cyclohexylpropyl]amino]-4-methyl- |
| | thiazol-5-yl]-2-methoxy- |
| | benzenesulfonamide |
| S-164 | 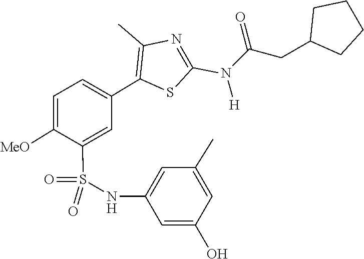 [SMILES: COc1ccc(-c2sc(NC(=O)CC3CCCC3)nc2C)cc1S(=O)(=O)Nc1cc(C)cc(O)c1] |
| | 2-cyclopentyl-N-[5-[3-[(3-hydroxy-5- |
| | methyl-phenyl)sulfamoyl]-4-methoxy- |
| | phenyl]-4-methyl-thiazol-2-yl]acetamide |
| S-165 | 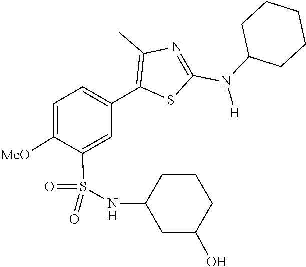 [SMILES: COc1ccc(-c2sc(NC3CCCCC3)nc2C)cc1S(=O)(=O)NC1CCCC(O)C1] |
| | 5-[2-(cyclohexylamino)-4-methyl-thiazol- |
| | 5-yl]-N-(3-hydroxycyclohexyl)-2- |
| | methoxy-benzenesulfonamide |
| S-166 | 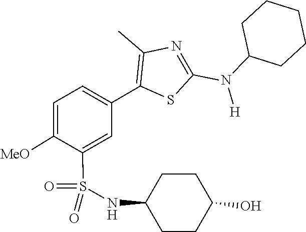 [SMILES: COc1ccc(-c2sc(NC3CCCCC3)nc2C)cc1S(=O)(=O)N[C@H]1CC[C@H](O)CC1] |
| | 5-[2-(cyclohexylamino)-4-methyl- |
| | thiazol-5-yl]-N-(4-hydroxycyclohexyl)- |
| | 2-methoxy-benzenesulfonamide |
| S-167 | 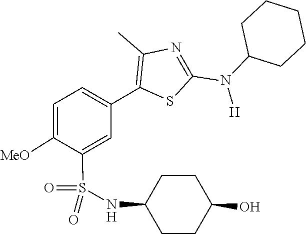 [SMILES: COc1ccc(-c2sc(NC3CCCCC3)nc2C)cc1S(=O)(=O)N[C@H]1CC[C@@H](O)CC1] |
| | 5-[2-(cyclohexylamino)-4-methyl-thiazol- |
| | 5-yl]-N-(4-hydroxycyclohexyl)-2- |
| | methoxy-benzenesulfonamide |
| S-168 | 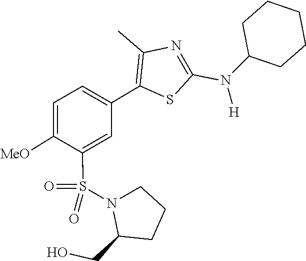 [SMILES: COc1ccc(-c2sc(NC3CCCCC3)nc2C)cc1S(=O)(=O)N1CCC[C@H]1CO] |
| | [(2S)-1-[5-[2-(cyclohexylamino)-4- |
| | methyl-thiazol-5-yl]-2-methoxy- |
| | phenyl]sulfonylpyrrolidin-2-yl]methanol |
| S-169 | 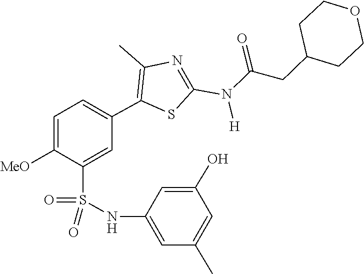 [SMILES: COc1ccc(-c2sc(NC(=O)CC3CCOCC3)nc2C)cc1S(=O)(=O)Nc1cc(C)cc(O)c1] |
| | N-[5-[3-[(3-hydroxy-5-methyl- |
| | phenyl)sulfamoyl]-4-methoxy-phenyl]-4- |
| | methyl-thiazol-2-yl]-2-tetrahydropyran-4- |
| | yl-acetamide |
| S-170 | 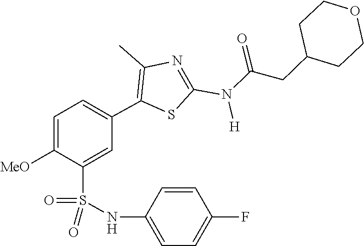 [SMILES: COc1ccc(-c2sc(NC(=O)CC3CCOCC3)nc2C)cc1S(=O)(=O)Nc1ccc(F)cc1] |
| | N-[5-[3-[(4-fluorophenyl)sulfamoyl]-4- |
| | methoxy-phenyl]-4-methyl-thiazol-2-yl]- |
| | 2-tetrahydropyran-4-yl-acetamide |
| S-171 | 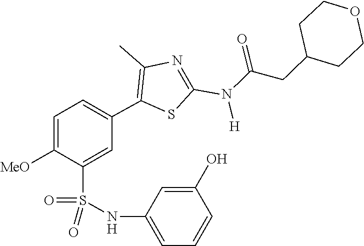 [SMILES: COc1ccc(-c2sc(NC(=O)CC3CCOCC3)nc2C)cc1S(=O)(=O)Nc1cccc(O)c1] |
| | N-[5-[3-[(3-hydroxyphenyl)sulfamoyl]-4- |
| | methoxy-phenyl]-4-methyl-thiazol-2-yl]-2- |
| | tetrahydropyran-4-yl-acetamide |
| S-172 | 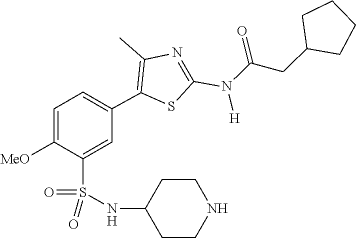 [SMILES: COc1ccc(-c2sc(NC(=O)CC3CCCC3)nc2C)cc1S(=O)(=O)NC1CCNCC1] |
| | 2-cyclopentyl-N-[5-[4-methoxy-3-(4- |
| | piperidylsulfamoyl)phenyl]-4-methyl- |
| | thiazol-2-yl]acetamide;2,2,2- |
| | trifluoroacetic acid |
| S-173 | 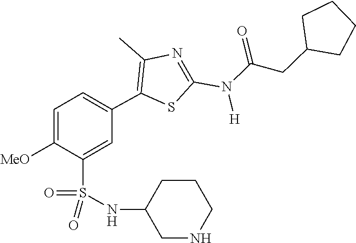 [SMILES: COc1ccc(-c2sc(NC(=O)CC3CCCC3)nc2C)cc1S(=O)(=O)NC1CCCNC1] |
| | 2-cyclopentyl-N-[5-[4-methoxy-3-(3 |
| | piperidylsulfamoyl)phenyl]-4-methyl- |
| | thiazol-2-yl]acetamide;2,2,2-trifluoroacetic |
| | acid |
| S-174 | 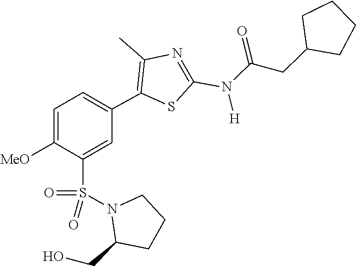 [SMILES: COc1ccc(-c2sc(NC(=O)CC3CCCC3)nc2C)cc1S(=O)(=O)N1CCC[C@H]1CO] |
| | 2-cyclopentyl-N-[5-[3-[(2S)-2- |
| | (hydroxymethyl)pyrrolidin-1- |
| | yl]sulfonyl-4-methoxy-phenyl]-4- |
| | methyl-thiazol-2-yl]acetamide |
| S-175 | 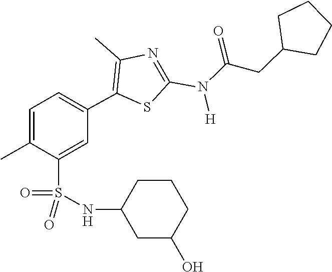 [SMILES: Cc1ccc(-c2sc(NC(=O)CC3CCCC3)nc2C)cc1S(=O)(=O)NC1CCCC(O)C1] |
| | 2-cyclopentyl-N-[5-[3-[(3- |
| | hydroxycyclohexyl)sulfamoyl]-4-methyl- |
| | phenyl]-4-methyl-thiazol-2-yl]acetamide |
| S-176 | 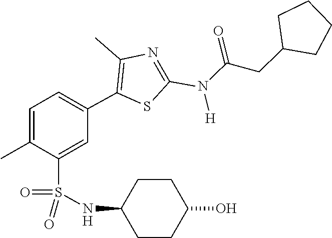 [SMILES: Cc1ccc(-c2sc(NC(=O)CC3CCCC3)nc2C)cc1S(=O)(=O)N[C@H]1CC[C@H](O)CC1] |
| | 2-cyclopentyl-N-[5-[3-[(4- |
| | hydroxycyclohexyl)sulfamoyl]-4- |
| | methyl-phenyl]-4-methyl-thiazol-2- |
| | yl]acetamide |
| S-177 | 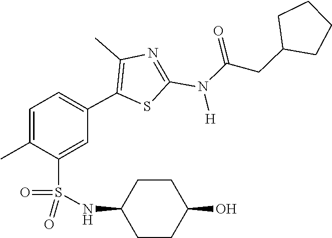 [SMILES: Cc1ccc(-c2sc(NC(=O)CC3CCCC3)nc2C)cc1S(=O)(=O)N[C@H]1CC[C@@H](O)CC1] |
| | 2-cyclopentyl-N-[5-[3-[(4- |
| | hydroxycyclohexyl)sulfamoyl]-4-methyl- |
| | phenyl]-4-methyl-thiazol-2-yl]acetamide |
| S-178 | 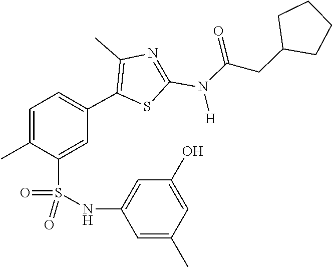 [SMILES: Cc1cc(O)cc(NS(=O)(=O)c2cc(-c3sc(NC(=O)CC4CCCC4)nc3C)ccc2C)c1] |
| | 2-cyclopentyl-N-[5-[3-[(3-hydroxy-5- |
| | methyl-phenyl)sulfamoyl]-4-methyl- |
| | phenyl]-4-methyl-thiazol-2-yl]acetamide |
| S-179 | 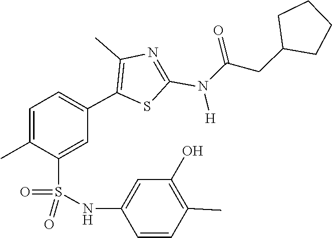 [SMILES: Cc1ccc(NS(=O)(=O)c2cc(-c3sc(NC(=O)CC4CCCC4)nc3C)ccc2C)cc1O] |
| | 2-cyclopentyl-N-[5-[3-[(3-hydroxy-4- |
| | methyl-phenyl)sulfamoyl]-4-methyl- |
| | phenyl]-4-methyl-thiazol-2-yl]acetamide |
| S-180 | 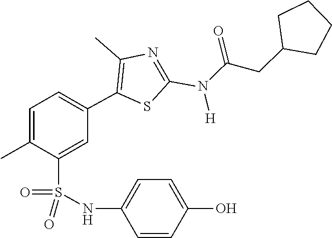 [SMILES: Cc1ccc(-c2sc(NC(=O)CC3CCCC3)nc2C)cc1S(=O)(=O)Nc1ccc(O)cc1] |
| | 2-cyclopentyl-N-[5-[3-[(4- |
| | hydroxyphenyl)sulfamoyl]-4-methyl- |
| | phenyl]-4-methyl-thiazol-2-yl]acetamide |
| S-181 | 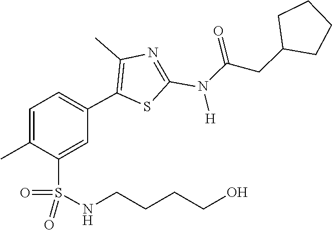 [SMILES: Cc1ccc(-c2sc(NC(=O)CC3CCCC3)nc2C)cc1S(=O)(=O)NCCCCO] |
| | 2-cyclopentyl-N-[5-[3-(4- |
| | hydroxybutylsulfamoyl)-4-methyl-phenyl]- |
| | 4-methyl-thiazol-2-yl]acetamide |
| S-182 | 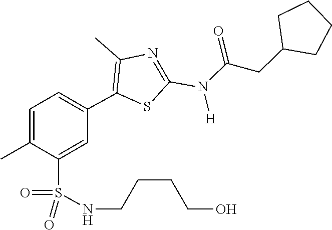 [SMILES: Cc1ccc(-c2sc(NC(=O)CC3CCCC3)nc2C)cc1S(=O)(=O)NCCCCO] |
| | 2-cyclopentyl-N-[5-[3- |
| | (cyclopentylsulfamoyl)-4-methyl- |
| | phenyl]-4-methyl-thiazol-2-yl]acetamide |
| S-183 | 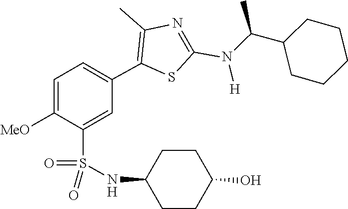 [SMILES: COc1ccc(-c2sc(N[C@@H](C)C3CCCCC3)nc2C)cc1S(=O)(=O)N[C@H]1CC[C@H](O)CC1] |
| | 5-[2-[[(1S)-1-cyclohexylethyl]amino]-4- |
| | methyl-thiazol-5-yl]-N-(4- |
| | hydroxycyclohexyl)-2-methoxy- |
| | benzenesulfonamide |
| S-184 | 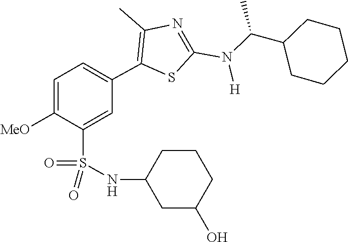 [SMILES: COc1ccc(-c2sc(N[C@H](C)C3CCCCC3)nc2C)cc1S(=O)(=O)NC1CCCC(O)C1] |
| | 5-[2-[[(1R)-1-cyclohexylethyl]amino]-4- |
| | methyl-thiazol-5-yl]-N-(3- |
| | hydroxycyclohexyl)-2-methoxy- |
| | benzenesulfonamide |
| S-185 | 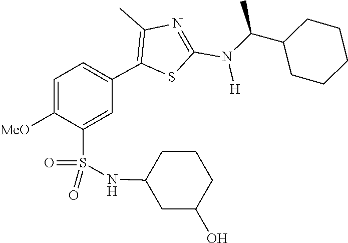 [SMILES: COc1ccc(-c2sc(N[C@@H](C)C3CCCCC3)nc2C)cc1S(=O)(=O)NC1CCCC(O)C1] |
| | 5-[2-[[(1S)-1-cyclohexylethyl]amino]-4- |
| | methyl-thiazol-5-yl]-N-(3- |
| | hydroxycyclohexyl)-2-methoxy- |
| | benzenesulfonamide |
| S-186 | 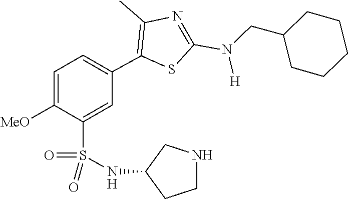 [SMILES: COc1ccc(-c2sc(NCC3CCCCC3)nc2C)cc1S(=O)(=O)N[C@H]1CCNC1] |
| | 5-[2-(cyclohexylmethylamino)-4- |
| | methyl-thiazol-5-yl]-2-methoxy-N- |
| | [(3S)-pyrrolidin-3- |
| | yl]benzenesulfonamide |
| S-187 | 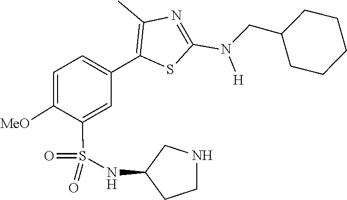 [SMILES: COc1ccc(-c2sc(NCC3CCCCC3)nc2C)cc1S(=O)(=O)N[C@@H]1CCNC1] |
| | 5-[2-(cyclohexylmethylamino)-4-methyl- |
| | thiazol-5-yl]-2-methoxy-N-[(3R)- |
| | pyrrolidin-3-yl]benzenesulfonamide;2,2,2- |
| | trifluoroacetic acid |
| S-188 | 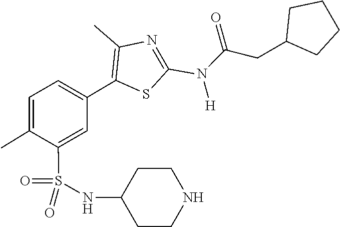 [SMILES: Cc1ccc(-c2sc(NC(=O)CC3CCCC3)nc2C)cc1S(=O)(=O)NC1CCNCC1] |
| | 2-cyclopentyl-N-[4-methyl-5-[4-methyl- |
| | 3-(4-piperidylsulfamoyl)phenyl]thiazol- |
| | 2-yl]acetamide |
| S-189 | 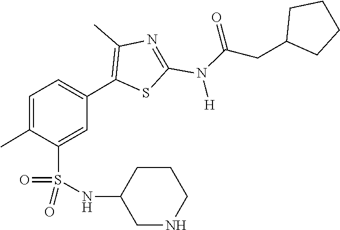 [SMILES: Cc1ccc(-c2sc(NC(=O)CC3CCCC3)nc2C)cc1S(=O)(=O)NC1CCCNC1] |
| | 2-cyclopentyl-N-[4-methyl-5-[4-methyl-3- |
| | (3-piperidylsulfamoyl)phenyl]thiazol-2- |
| | yl]acetamide |
| S-190 | 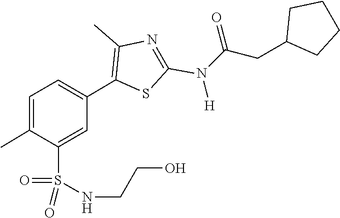 [SMILES: Cc1ccc(-c2sc(NC(=O)CC3CCCC3)nc2C)cc1S(=O)(=O)NCCO] |
| | 2-cyclopentyl-N-[5-[3-(2- |
| | hydroxyethylsulfamoyl)-4-methyl- |
| | phenyl]-4-methyl-thiazol-2-yl]acetamide |
| S-191 | 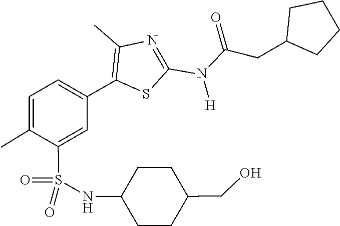 [SMILES: Cc1ccc(-c2sc(NC(=O)CC3CCCC3)nc2C)cc1S(=O)(=O)NC1CCC(CO)CC1] |
| | 2-cyclopentyl-N-[5-[3-[[4- |
| | (hydroxymethyl)cyclohexyl]sulfamoyl]-4- |
| | methyl-phenyl]-4-methyl-thiazol-2- |
| | yl]acetamide |
| S-192 | 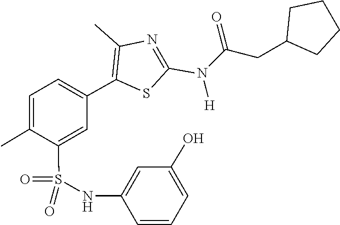 [SMILES: Cc1ccc(-c2sc(NC(=O)CC3CCCC3)nc2C)cc1S(=O)(=O)Nc1cccc(O)c1] |
| | 2-cyclopentyl-N-[5-[3-[(3- |
| | hydroxyphenyl)sulfamoyl]-4-methyl- |
| | phenyl]-4-methyl-thiazol-2-yl]acetamide |
| S-193 | 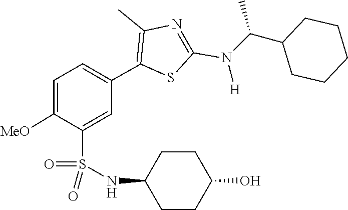 [SMILES: COc1ccc(-c2sc(N[C@H](C)C3CCCCC3)nc2C)cc1S(=O)(=O)N[C@H]1CC[C@H](O)CC1] |
| | 5-[2-[[(1R)-1-cyclohexylethyl]amino]-4- |
| | methyl-thiazol-5-yl]-N-(4- |
| | hydroxycyclohexyl)-2-methoxy- |
| | benzenesulfonamide |
| S-194 | 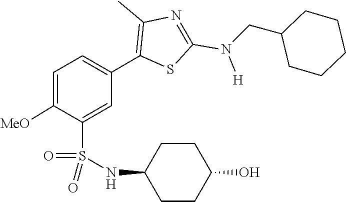 [SMILES: COc1ccc(-c2sc(NCC3CCCCC3)nc2C)cc1S(=O)(=O)N[C@H]1CC[C@H](O)CC1] |
| | 5-[2-(cyclohexylmethylamino)-4- |
| | methyl-thiazol-5-yl]-N-(4- |
| | hydroxycyclohexyl)-2-methoxy- |
| | benzenesulfonamide |
| S-195 | 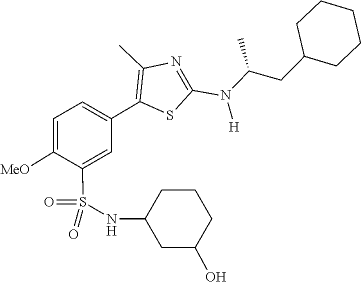 [SMILES: COc1ccc(-c2sc(N[C@H](C)CC3CCCCC3)nc2C)cc1S(=O)(=O)NC1CCCC(O)C1] |
| | 5-[2-[[(1R)-2-cyclohexyl-1-methyl- |
| | ethyl]amino]-4-methyl-thiazol-5-yl]-N-(3- |
| | hydroxycyclohexyl)-2-methoxy- |
| | benzenesulfonamide |
| S-196 | 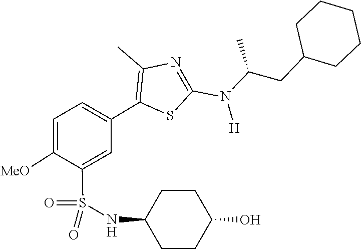 [SMILES: COc1ccc(-c2sc(N[C@H](C)CC3CCCCC3)nc2C)cc1S(=O)(=O)N[C@H]1CC[C@H](O)CC1] |
| | 5-[2-[[(1R)-2-cyclohexyl-1-methyl- |
| | ethyl]amino]-4-methyl-thiazol-5-yl]-N- |
| | (4-hydroxycyclohexyl)-2-methoxy- |
| | benzenesulfonamide |
| S-197 | 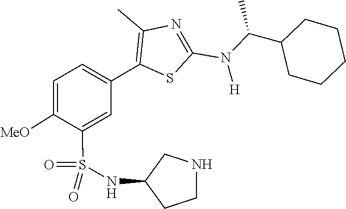 [SMILES: COc1ccc(-c2sc(N[C@H](C)C3CCCCC3)nc2C)cc1S(=O)(=O)N[C@@H]1CCNC1] |
| | 5-[2-[[(1R)-1-cyclohexylethyl]amino]-4- |
| | methyl-thiazol-5-yl]-2-methoxy-N-[(3R)- |
| | pyrrolidin-3-yl]benzenesulfonamide |
| S-198 | 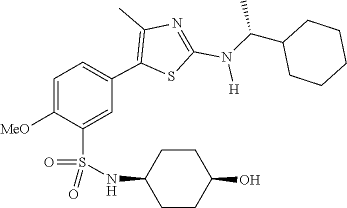 [SMILES: COc1ccc(-c2sc(N[C@H](C)C3CCCCC3)nc2C)cc1S(=O)(=O)N[C@H]1CC[C@@H](O)CC1] |
| | 5-[2-[[(1R)-1-cyclohexylethyl]amino]-4- |
| | methyl-thiazol-5-yl]-N-(4- |
| | hydroxycyclohexyl)-2-methoxy- |
| | benzenesulfonamide |
| S-199 | 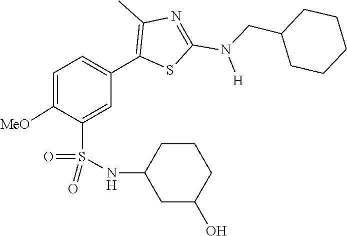 [SMILES: COc1ccc(-c2sc(NCC3CCCCC3)nc2C)cc1S(=O)(=O)NC1CCCC(O)C1] |
| | 5-[2-(cyclohexylmethylamino)-4-methyl- |
| | thiazol-5-yl]-N-(3-hydroxycyclohexyl)-2- |
| | methoxy-benzenesulfonamide |
| S-200 | 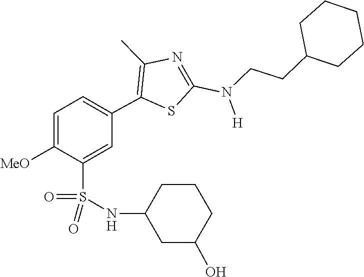 [SMILES: COc1ccc(-c2sc(NCCC3CCCCC3)nc2C)cc1S(=O)(=O)NC1CCCC(O)C1] |
| | 5-[2-(2-cyclohexylethylamino)-4- |
| | methyl-thiazol-5-yl]-N-(3- |
| | hydroxycyclohexyl)-2-methoxy- |
| | benzenesulfonamide |
| S-201 | 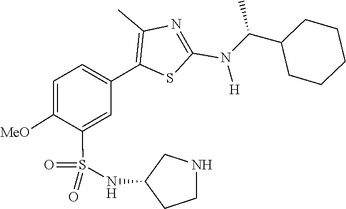 [SMILES: COc1ccc(-c2sc(N[C@H](C)C3CCCCC3)nc2C)cc1S(=O)(=O)N[C@H]1CCNC1] |
| | 5-[2-[[(1R)-1-cyclohexylethyl]amino]-4- |
| | methyl-thiazol-5-yl]-2-methoxy-N-[(3S)- |
| | pyrrolidin-3-yl]benzenesulfonamide |
| S-202 | 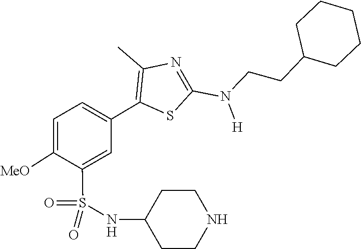 [SMILES: COc1ccc(-c2sc(NCCC3CCCCC3)nc2C)cc1S(=O)(=O)NC1CCNCC1] |
| | 5-[2-(2-cyclohexylethylamino)-4- |
| | methyl-thiazol-5-yl]-2-methoxy-N-(4- |
| | piperidyl)benzenesulfonamide |
| S-203 | 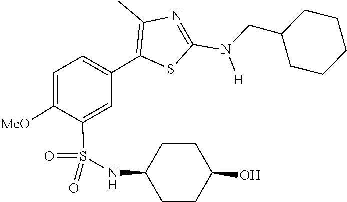 [SMILES: COc1ccc(-c2sc(NCC3CCCCC3)nc2C)cc1S(=O)(=O)N[C@H]1CC[C@@H](O)CC1] |
| | 5-[2-(cyclohexylmethylamino)-4-methyl- |
| | thiazol-5-yl]-N-(4-hydroxycyclohexyl)-2- |
| | methoxy-benzenesulfonamide |
| S-204 | 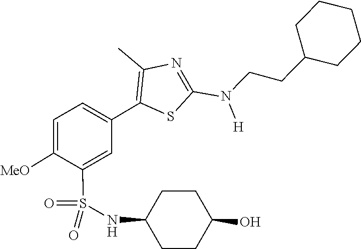 [SMILES: COc1ccc(-c2sc(NCCC3CCCCC3)nc2C)cc1S(=O)(=O)N[C@H]1CC[C@@H](O)CC1] |
| | 5-[2-(2-cyclohexylethylamino)-4- |
| | methyl-thiazol-5-yl]-N-(4- |
| | hydroxycyclohexyl)-2-methoxy- |
| | benzenesulfonamide |
| S-205 | 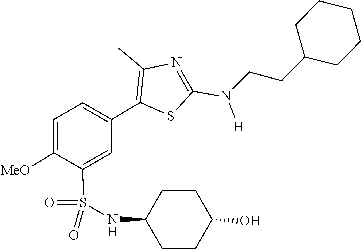 [SMILES: COc1ccc(-c2sc(NCCC3CCCCC3)nc2C)cc1S(=O)(=O)N[C@H]1CC[C@H](O)CC1] |
| | 5-[2-(2-cyclohexylethylamino)-4-methyl- |
| | thiazol-5-yl]-N-(4-hydroxycyclohexyl)-2- |
| | methoxy-benzenesulfonamide |
| S-206 | 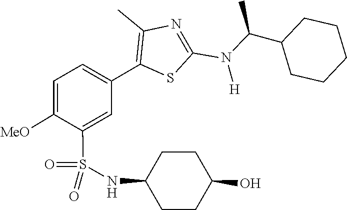 [SMILES: COc1ccc(-c2sc(N[C@@H](C)C3CCCCC3)nc2C)cc1S(=O)(=O)N[C@H]1CC[C@@H](O)CC1] |
| | 5-[2-[[(1S)-1-cyclohexylethyl]amino]-4- |
| | methyl-thiazol-5-yl]-N-(4- |
| | hydroxycyclohexyl)-2-methoxy- |
| | benzenesulfonamide |
| S-207 | 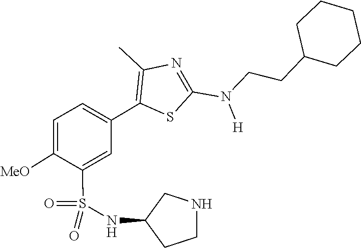 [SMILES: COc1ccc(-c2sc(NCCC3CCCCC3)nc2C)cc1S(=O)(=O)N[C@@H]1CCNC1] |
| | 5-[2-(2-cyclohexylethylamino)-4-methyl- |
| | thiazol-5-yl]-2-methoxy-N-[(3R)- |
| | pyrrolidin-3-yl]benzenesulfonamide |
| S-208 | 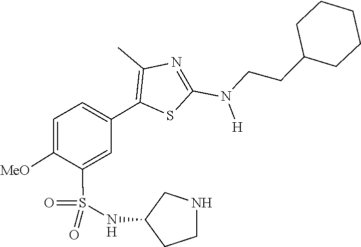 [SMILES: COc1ccc(-c2sc(NCCC3CCCCC3)nc2C)cc1S(=O)(=O)N[C@H]1CCNC1] |
| | 5-[2-(2-cyclohexylethylamino)-4- |
| | methyl-thiazol-5-yl]-2-methoxy-N- |
| | [(3S)-pyrrolidin-3- |
| | yl]benzenesulfonamide |
| S-209 | 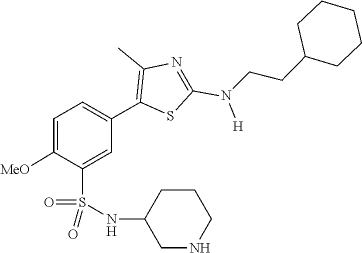 [SMILES: COc1ccc(-c2sc(NCCC3CCCCC3)nc2C)cc1S(=O)(=O)NC1CCCNC1] |
| | 5-[2-(2-cyclohexylethylamino)-4-methyl- |
| | thiazol-5-yl]-2-methoxy-N-(3- |
| | piperidyl)benzenesulfonamide |
| S-210 | 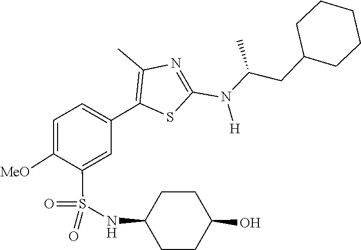 [SMILES: COc1ccc(-c2sc(N[C@H](C)CC3CCCCC3)nc2C)cc1S(=O)(=O)N[C@H]1CC[C@@H](O)CC1] |
| | 5-[2-[[(1R)-2-cyclohexyl-1-methyl- |
| | ethyl]amino]-4-methyl-thiazol-5-yl]-N- |
| | (4-hydroxycyclohexyl)-2-methoxy- |
| | benzenesulfonamide |
| S-211 | 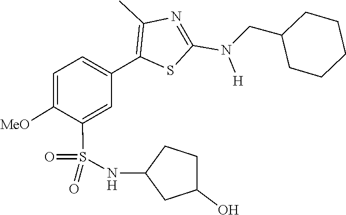 [SMILES: COc1ccc(-c2sc(NCC3CCCCC3)nc2C)cc1S(=O)(=O)NC1CCC(O)C1] |
| | 5-[2-(cyclohexylmethylamino)-4-methyl- |
| | thiazol-5-yl]-N-(3-hydroxycyclopentyl)-2- |
| | methoxy-benzenesulfonamide |
| S-212 | 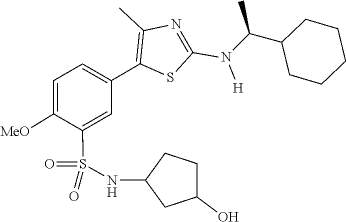 [SMILES: COc1ccc(-c2sc(N[C@@H](C)C3CCCCC3)nc2C)cc1S(=O)(=O)NC1CCC(O)C1] |
| | 5-[2-[[(1S)-1-cyclohexylethyl]amino]-4- |
| | methyl-thiazol-5-yl]-N-(3- |
| | hydroxycyclopentyl)-2-methoxy- |
| | benzenesulfonamide |
| S-213 | 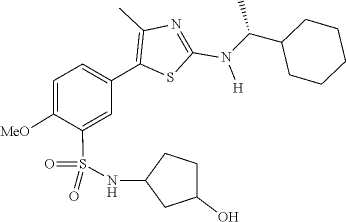 [SMILES: COc1ccc(-c2sc(N[C@H](C)C3CCCCC3)nc2C)cc1S(=O)(=O)NC1CCC(O)C1] |
| | 5-[2-[[(1R)-1-cyclohexylethyl]amino]-4- |
| | methyl-thiazol-5-yl]-N-(3- |
| | hydroxycyclopentyl)-2-methoxy- |
| | benzenesulfonamide |
| S-214 | 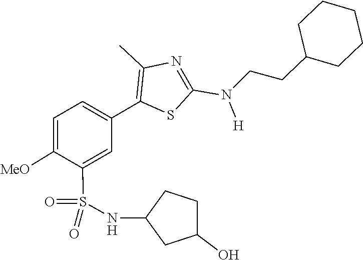 [SMILES: COc1ccc(-c2sc(NCCC3CCCCC3)nc2C)cc1S(=O)(=O)NC1CCC(O)C1] |
| | 5-[2-(2-cyclohexylethylamino)-4- |
| | methyl-thiazol-5-yl]-N-(3- |
| | hydroxycyclopentyl)-2-methoxy- |
| | benzenesulfonamide |
| S-215 | 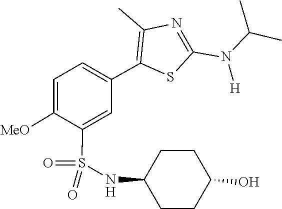 [SMILES: COc1ccc(-c2sc(NC(C)C)nc2C)cc1S(=O)(=O)N[C@H]1CC[C@H](O)CC1] |
| | N-(4-hydroxycyclohexyl)-5-[2- |
| | (isopropylamino)-4-methyl-thiazol-5-yl]-2- |
| | methoxy-benzenesulfonamide |
| S-216 | 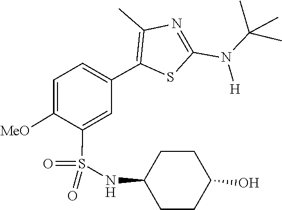 [SMILES: COc1ccc(-c2sc(NC(C)(C)C)nc2C)cc1S(=O)(=O)N[C@H]1CC[C@H](O)CC1] |
| | 5-[2-(tert-butylamino)-4-methyl-thiazol- |
| | 5-yl]-N-(4-hydroxycyclohexyl)-2- |
| | methoxy-benzenesulfonamide |
| S-217 | 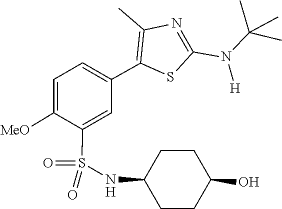 [SMILES: COc1ccc(-c2sc(NC(C)(C)C)nc2C)cc1S(=O)(=O)N[C@H]1CC[C@@H](O)CC1] |
| | 5-[2-(tert-butylamino)-4-methyl-thiazol-5- |
| | yl]-N-(4-hydroxycyclohexyl)-2-methoxy- |
| | benzenesulfonamide |
| S-218 | 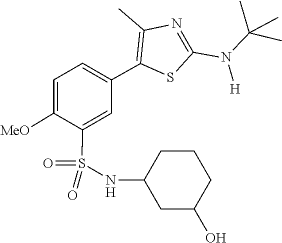 [SMILES: COc1ccc(-c2sc(NC(C)(C)C)nc2C)cc1S(=O)(=O)NC1CCCC(O)C1] |
| | 5-[2-(tert-butylamino)-4-methyl-thiazol- |
| | 5-yl]-N-(3-hydroxycyclohexyl)-2- |
| | methoxy-benzenesulfonamide |
| S-219 | 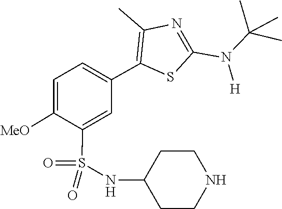 [SMILES: COc1ccc(-c2sc(NC(C)(C)C)nc2C)cc1S(=O)(=O)NC1CCNCC1] |
| | 5-[2-(tert-butylamino)-4-methyl-thiazol-5- |
| | yl]-2-methoxy-N-(4- |
| | piperidyl)benzenesulfonamide |
| S-220 | 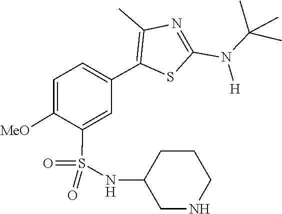 [SMILES: COc1ccc(-c2sc(NC(C)(C)C)nc2C)cc1S(=O)(=O)NC1CCCNC1] |
| | 5-[2-(tert-butylamino)-4-methyl-thiazol- |
| | 5-yl]-2-methoxy-N-(3- |
| | piperidyl)benzenesulfonamide |
| S-221 | 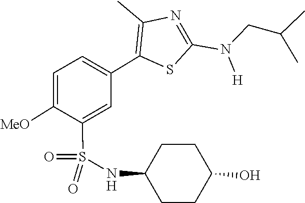 [SMILES: COc1ccc(-c2sc(NCC(C)C)nc2C)cc1S(=O)(=O)N[C@H]1CC[C@H](O)CC1] |
| | N-(4-hydroxycyclohexyl)-5-[2- |
| | (isobutylamino)-4-methyl-thiazol-5-yl]-2- |
| | methoxy-benzenesulfonamide |
| S-222 | 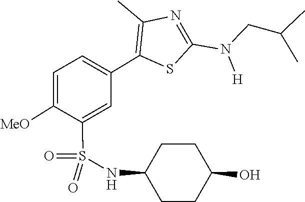 [SMILES: COc1ccc(-c2sc(NCC(C)C)nc2C)cc1S(=O)(=O)N[C@H]1CC[C@@H](O)CC1] |
| | N-(4-hydroxycyclohexyl)-5-[2- |
| | (isobutylamino)-4-methyl-thiazol-5-yl]- |
| | 2-methoxy-benzenesulfonamide |
| S-223 | 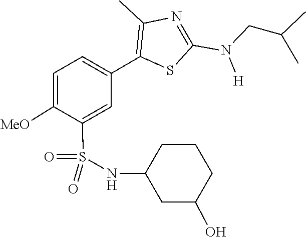 [SMILES: COc1ccc(-c2sc(NCC(C)C)nc2C)cc1S(=O)(=O)NC1CCCC(O)C1] |
| | N-(3-hydroxycyclohexyl)-5-[2- |
| | (isobutylamino)-4-methyl-thiazol-5-yl]-2- |
| | methoxy-benzenesulfonamide;2,2,2- |
| | trifluoroacetic acid |
| S-224 | 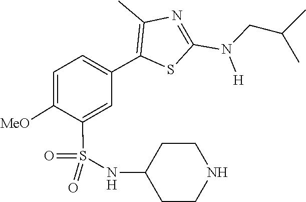 [SMILES: COc1ccc(-c2sc(NCC(C)C)nc2C)cc1S(=O)(=O)NC1CCNCC1] |
| | 5-[2-(isobutylamino)-4-methyl-thiazol- |
| | 5-yl]-2-methoxy-N-(4- |
| | piperidyl)benzenesulfonamide;2,2,2- |
| | trifluoroacetic acid |
| S-225 | 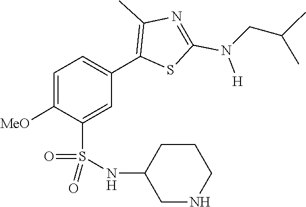 [SMILES: COc1ccc(-c2sc(NCC(C)C)nc2C)cc1S(=O)(=O)NC1CCCNC1] |
| | 5-[2-(isobutylamino)-4-methyl-thiazol-5- |
| | yl]-2-methoxy-N-(3- |
| | piperidyl)benzenesulfonamide;2,2,2- |
| | trifluoroacetic acid |
| S-226 | 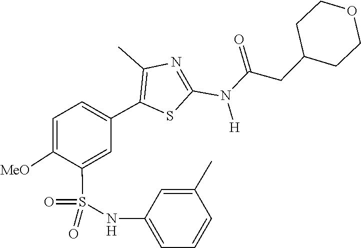 [SMILES: COc1ccc(-c2sc(NC(=O)CC3CCOCC3)nc2C)cc1S(=O)(=O)Nc1cccc(C)c1] |
| | N-[5-[4-methoxy-3-(m- |
| | tolylsulfamoyl)phenyl]-4-methyl- |
| | thiazol-2-yl]-2-tetrahydropyran-4-yl- |
| | acetamide |
| S-227 | 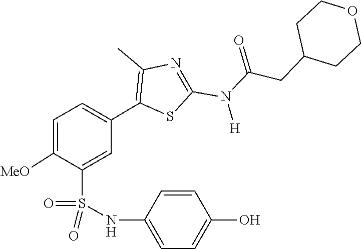 [SMILES: COc1ccc(-c2sc(NC(=O)CC3CCOCC3)nc2C)cc1S(=O)(=O)Nc1ccc(O)cc1] |
| | N-[5-[3-[(4-hydroxyphenyl)sulfamoyl]-4- |
| | methoxy-phenyl]-4-methyl-thiazol-2-yl]-2- |
| | tetrahydropyran-4-yl-acetamide |
| S-228 | 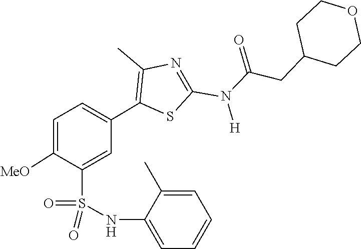 [SMILES: COc1ccc(-c2sc(NC(=O)CC3CCOCC3)nc2C)cc1S(=O)(=O)Nc1ccccc1C] |
| | N-[5-[4-methoxy-3-(o- |
| | tolylsulfamoyl)phenyl]-4-methyl- |
| | thiazol-2-yl]-2-tetrahydropyran-4-yl- |
| | acetamide |
| S-229 | 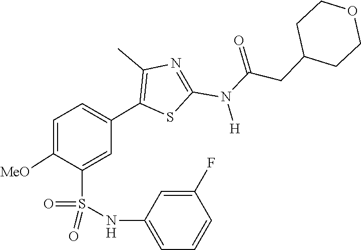 [SMILES: COc1ccc(-c2sc(NC(=O)CC3CCOCC3)nc2C)cc1S(=O)(=O)Nc1cccc(F)c1] |
| | N-[5-[3-[(3-fluorophenyl)sulfamoyl]-4- |
| | methoxy-phenyl]-4-methyl-thiazol-2-yl]-2- |
| | tetrahydropyran-4-yl-acetamide |
| S-230 | 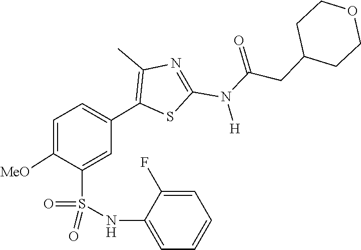 [SMILES: COc1ccc(-c2sc(NC(=O)CC3CCOCC3)nc2C)cc1S(=O)(=O)Nc1ccccc1F] |
| | N-[5-[3-[(2-fluorophenyl)sulfamoyl]-4- |
| | methoxy-phenyl]-4-methyl-thiazol-2-yl]- |
| | 2-tetrahydropyran-4-yl-acetamide |
| S-231 | 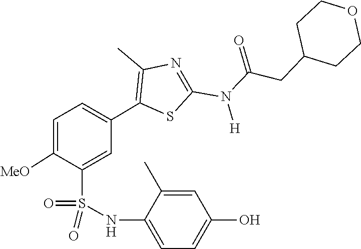 [SMILES: COc1ccc(-c2sc(NC(=O)CC3CCOCC3)nc2C)cc1S(=O)(=O)Nc1ccc(O)cc1C] |
| | N-[5-[3-[(4-hydroxy-2-methyl- |
| | phenyl)sulfamoyl]-4-methoxy-phenyl]-4- |
| | methyl-thiazol-2-yl]-2-tetrahydropyran-4- |
| | yl-acetamide |
| S-232 | 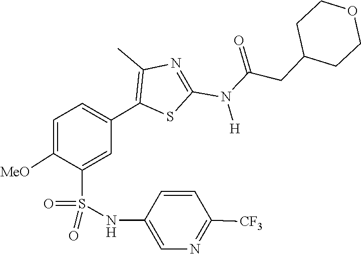 [SMILES: COc1ccc(-c2sc(NC(=O)CC3CCOCC3)nc2C)cc1S(=O)(=O)Nc1ccc(C(F)(F)F)nc1] |
| | N-[5-[4-methoxy-3-[[6- |
| | (trifluoromethyl)-3- |
| | pyridyl]sulfamoyl]phenyl]-4-methyl- |
| | thiazol-2-yl]-2-tetrahydropyran-4-yl |
| | acetamide |
| S-233 | 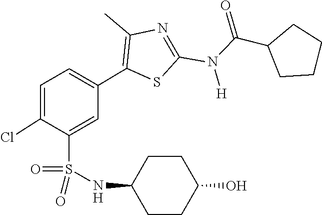 [SMILES: Cc1nc(NC(=O)C2CCCC2)sc1-c1ccc(Cl)c(S(=O)(=O)N[C@H]2CC[C@H](O)CC2)c1] |
| | N-[5-[4-chloro-3-[(4- |
| | hydroxycyclohexyl)sulfamoyl]phenyl]-4- |
| | methyl-thiazol-2- |
| | yl]cyclopentanecarboxamide |
| S-234 | 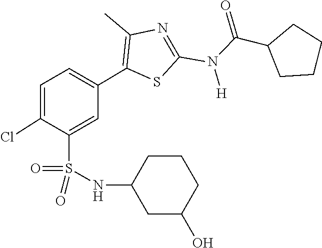 [SMILES: Cc1nc(NC(=O)C2CCCC2)sc1-c1ccc(Cl)c(S(=O)(=O)NC2CCCC(O)C2)c1] |
| | N-[5-[4-chloro-3-[(3- |
| | hydroxycyclohexyl)sulfamoyl]phenyl]- |
| | 4-methyl-thiazol-2- |
| | yl]cyclopentanecarboxamide |
| S-235 | 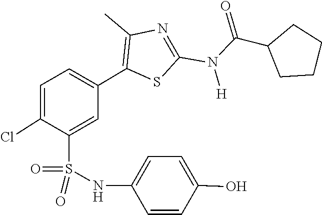 [SMILES: Cc1nc(NC(=O)C2CCCC2)sc1-c1ccc(Cl)c(S(=O)(=O)Nc2ccc(O)cc2)c1] |
| | N-[5-[4-chloro-3-[(4- |
| | hydroxyphenyl)sulfamoyl]phenyl]-4- |
| | methyl-thiazol-2- |
| | yl]cyclopentanecarboxamide |
| S-236 | 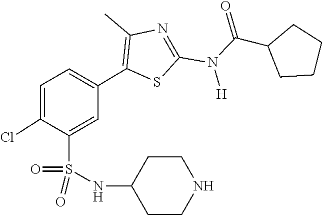 [SMILES: Cc1nc(NC(=O)C2CCCC2)sc1-c1ccc(Cl)c(S(=O)(=O)NC2CCNCC2)c1] |
| | N-[5-[4-chloro-3-(4- |
| | piperidylsulfamoyl)phenyl]-4-methyl- |
| | thiazol-2-yl]cyclopentanecarboxamide |
| S-237 | 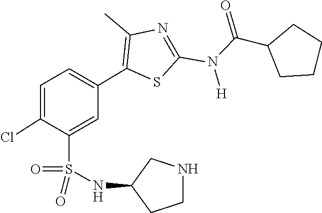 [SMILES: Cc1nc(NC(=O)C2CCCC2)sc1-c1ccc(Cl)c(S(=O)(=O)N[C@@H]2CCNC2)c1] |
| | N-[5-[4-chloro-3-[[(3R)-pyrrolidin-3- |
| | yl]sulfamoyl]phenyl]-4-methyl-thiazol-2- |
| | yl]cyclopentanecarboxamide |
| S-238 | 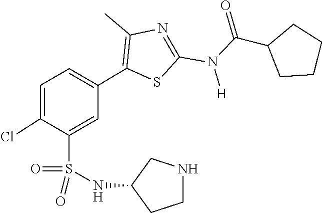 [SMILES: Cc1nc(NC(=O)C2CCCC2)sc1-c1ccc(Cl)c(S(=O)(=O)N[C@H]2CCNC2)c1] |
| | N-[5-[4-chloro-3-[[(3S)-pyrrolidin-3- |
| | yl]sulfamoyl]phenyl]-4-methyl-thiazol- |
| | 2-yl]cyclopentanecarboxamide |
| S-239 | 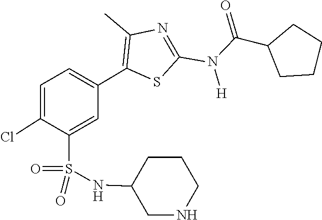 [SMILES: Cc1nc(NC(=O)C2CCCC2)sc1-c1ccc(Cl)c(S(=O)(=O)NC2CCCNC2)c1] |
| | N-[5-[4-chloro-3-(3- |
| | piperidylsulfamoyl)phenyl]-4-methyl- |
| | thiazol-2-yl]cyclopentanecarboxamide |
| S-240 | 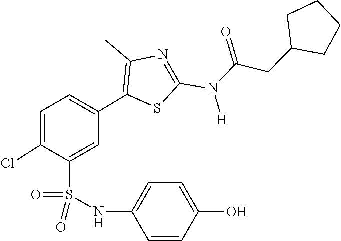 [SMILES: Cc1nc(NC(=O)CC2CCCC2)sc1-c1ccc(Cl)c(S(=O)(=O)Nc2ccc(O)cc2)c1] |
| | N-[5-[4-chloro-3-[(4- |
| | hydroxyphenyl)sulfamoyl]phenyl]-4- |
| | methyl-thiazol-2-yl]-2-cyclopentyl- |
| | acetamide |
| S-241 | 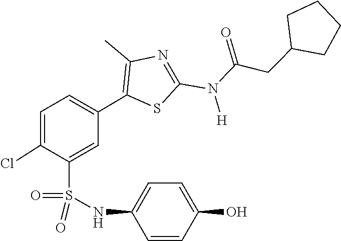 |
| | N-[5-[4-chloro-3-[(4- |
| | hydroxycyclohexyl)sulfamoyl]phenyl]-4- |
| | methyl-thiazol-2-yl]-2-cyclopentyl- |
| | acetamide |
| S-242 | 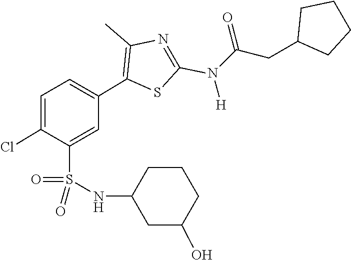 [SMILES: Cc1nc(NC(=O)CC2CCCC2)sc1-c1ccc(Cl)c(S(=O)(=O)NC2CCCC(O)C2)c1] |
| | N-[5-[4-chloro-3-[(3- |
| | hydroxycyclohexyl)sulfamoyl]phenyl]- |
| | 4-methyl-thiazol-2-yl]-2-cyclopentyl- |
| | acetamide |
| S-243 | 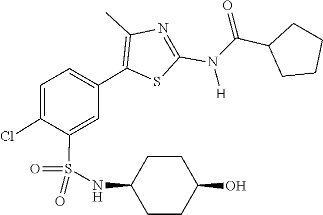 [SMILES: Cc1nc(NC(=O)C2CCCC2)sc1-c1ccc(Cl)c(S(=O)(=O)N[C@H]2CC[C@@H](O)CC2)c1] |
| | N-[5-[4-chloro-3-[(4- |
| | hydroxycyclohexyl)sulfamoyl]phenyl]-4- |
| | methyl-thiazol-2- |
| | yl]cyclopentanecarboxamide |
| S-244 | 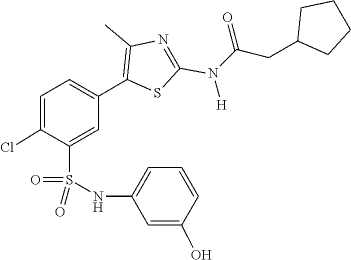 [SMILES: Cc1nc(NC(=O)CC2CCCC2)sc1-c1ccc(Cl)c(S(=O)(=O)Nc2cccc(O)c2)c1] |
| | N-[5-[4-chloro-3-[(3- |
| | hydroxyphenyl)sulfamoyl]phenyl]-4- |
| | methyl-thiazol-2-yl]-2-cyclopentyl- |
| | acetamide |
| S-245 | 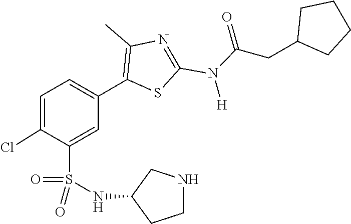 [SMILES: Cc1nc(NC(=O)CC2CCCC2)sc1-c1ccc(Cl)c(S(=O)(=O)N[C@H]2CCNC2)c1] |
| | N-[5-[4-chloro-3-[[(3S)-pyrrolidin-3- |
| | yl]sulfamoyl]phenyl]-4-methyl-thiazol-2- |
| | yl]-2-cyclopentyl-acetamide;2,2,2- |
| | trifluoroacetic acid |
| S-246 | 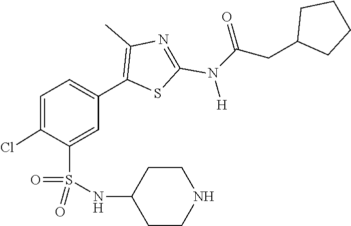 [SMILES: Cc1nc(NC(=O)CC2CCCC2)sc1-c1ccc(Cl)c(S(=O)(=O)NC2CCNCC2)c1] |
| | N-[5-[4-chloro-3-(4- |
| | piperidylsulfamoyl)phenyl]-4-methyl- |
| | thiazol-2-yl]-2-cyclopentyl-acetamide |
| S-247 | 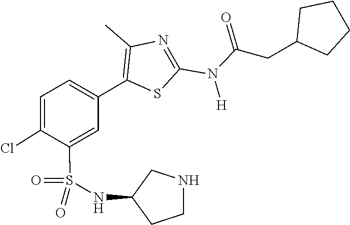 [SMILES: Cc1nc(NC(=O)CC2CCCC2)sc1-c1ccc(Cl)c(S(=O)(=O)N[C@@H]2CCNC2)c1] |
| | N-[5-[4-chloro-3-[[(3R)-pyrrolidin-3- |
| | yl]sulfamoyl]phenyl]-4-methyl-thiazol-2- |
| | yl]-2-cyclopentyl-acetamide |
| S-248 | 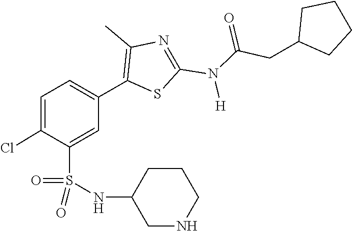 [SMILES: Cc1nc(NC(=O)CC2CCCC2)sc1-c1ccc(Cl)c(S(=O)(=O)NC2CCCNC2)c1] |
| | N-[5-[4-chloro-3-(3- |
| | piperidylsulfamoyl)phenyl]-4-methyl- |
| | thiazol-2-yl]-2-cyclopentyl-acetamide |
| S-249 | 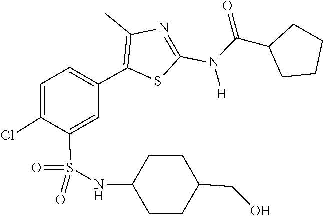 [SMILES: Cc1nc(NC(=O)C2CCCC2)sc1-c1ccc(Cl)c(S(=O)(=O)NC2CCC(CO)CC2)c1] |
| | N-[5-[4-chloro-3-[[4- |
| | (hydroxymethyl)cyclohexyl]sulfamoyl] |
| | phenyl]-4-methyl-thiazol-2- |
| | yl]cyclopentanecarboxamide |
| S-250 | 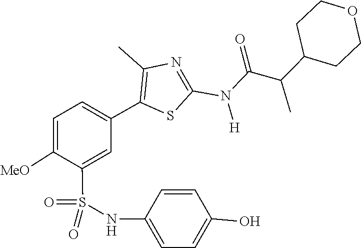 [SMILES: COc1ccc(-c2sc(NC(=O)C(C)C3CCOCC3)nc2C)cc1S(=O)(=O)Nc1ccc(O)cc1] |
| | N-[5-[3-[(4-hydroxyphenyl)sulfamoyl]- |
| | 4-methoxy-phenyl]-4-methyl-thiazol-2-yl]-2- |
| | tetrahydropyran-4-yl-propanamide |
| S-251 | 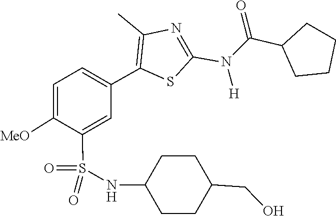 [SMILES: COc1ccc(-c2sc(NC(=O)C3CCCC3)nc2C)cc1S(=O)(=O)NC1CCC(CO)CC1] |
| | N-[5-[3-[[4- |
| | (hydroxymethyl)cyclohexyl]sulfamoyl]-4- |
| | methoxy-phenyl]-4-methyl-thiazol-2- |
| | yl]cyclopentanecarboxamide |
| S-252 | 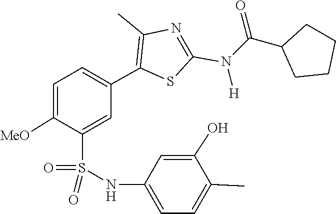 [SMILES: COc1ccc(-c2sc(NC(=O)C3CCCC3)nc2C)cc1S(=O)(=O)Nc1ccc(C)c(O)c1] |
| | N-[5-[3-[(3-hydroxy-4-methyl- |
| | phenyl)sulfamoyl]-4-methoxy-phenyl]- |
| | 4-methyl-thiazol-2- |
| | yl]cyclopentanecarboxamide |
| 2-253 | 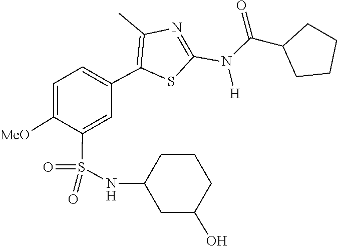 [SMILES: COc1ccc(-c2sc(NC(=O)C3CCCC3)nc2C)cc1S(=O)(=O)NC1CCCC(O)C1] |
| | N-[5-[3-[(3- |
| | hydroxycyclohexyl)sulfamoyl]-4-methoxy- |
| | phenyl]-4-methyl-thiazol-2- |
| | yl]cyclopentanecarboxamide |
| 2-254 | 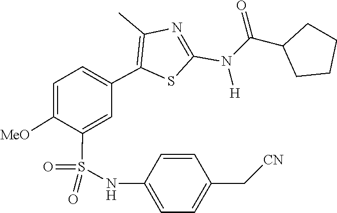 [SMILES: COc1ccc(-c2sc(NC(=O)C3CCCC3)nc2C)cc1S(=O)(=O)Nc1ccc(CC#N)cc1] |
| | N-[5-[3-[[4- |
| | (cyanomethyl)phenyl]sulfamoyl]-4- |
| | methoxy-phenyl]-4-methyl-thiazol-2- |
| | yl]cyclopentanecarboxamide |
| S-255 | 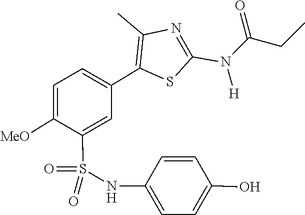 [SMILES: CCC(=O)Nc1nc(C)c(-c2ccc(OC)c(S(=O)(=O)Nc3ccc(O)cc3)c2)s1] |
| | N-[5-[3-[(4-hydroxyphenyl)sulfamoyl]-4- |
| | methoxy-phenyl]-4-methyl-thiazol-2- |
| | yl]propanamide |
| S-256 | 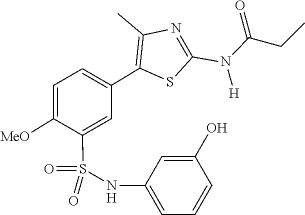 [SMILES: CCC(=O)Nc1nc(C)c(-c2ccc(OC)c(S(=O)(=O)Nc3cccc(O)c3)c2)s1] |
| | N-[5-[3-[(3-hydroxyphenyl)sulfamoyl]- |
| | 4-methoxy-phenyl]-4-methyl-thiazol-2- |
| | yl]propanamide |
| S-257 | 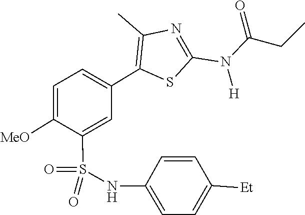 [SMILES: CCC(=O)Nc1nc(C)c(-c2ccc(OC)c(S(=O)(=O)Nc3ccc(CC)cc3)c2)s1] |
| | N-[5-[3-[(4-ethylphenyl)sulfamoyl]-4- |
| | methoxy-phenyl]-4-methyl-thiazol-2- |
| | yl]propanamide |
| S-258 | 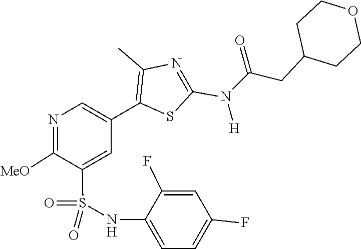 [SMILES: COc1ncc(-c2sc(NC(=O)CC3CCOCC3)nc2C)cc1S(=O)(=O)Nc1ccc(F)cc1F] |
| S-259 | 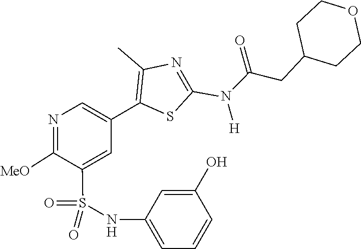 [SMILES: COc1ncc(-c2sc(NC(=O)CC3CCOCC3)nc2C)cc1S(=O)(=O)Nc1cccc(O)c1] |
| S-260 | 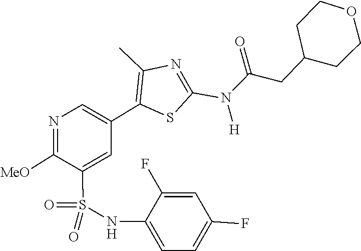 [SMILES: COc1ncc(-c2sc(NC(=O)CC3CCOCC3)nc2C)cc1S(=O)(=O)Nc1ccc(F)cc1F] |
| S-261 | 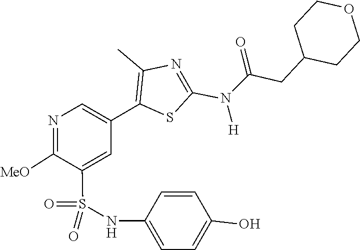 [SMILES: COc1ncc(-c2sc(NC(=O)CC3CCOCC3)nc2C)cc1S(=O)(=O)Nc1ccc(O)cc1] |
| S-262 | 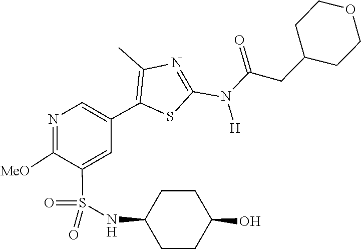 [SMILES: COc1ncc(-c2sc(NC(=O)CC3CCOCC3)nc2C)cc1S(=O)(=O)N[C@H]1CC[C@@H](O)CC1] |
| S-263 | 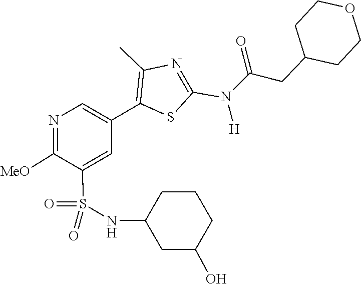 [SMILES: COc1ncc(-c2sc(NC(=O)CC3CCOCC3)nc2C)cc1S(=O)(=O)NC1CCCC(O)C1] |
| S-264 | 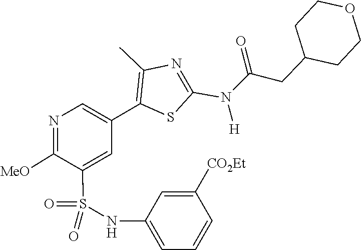 [SMILES: CCOC(=O)c1cccc(NS(=O)(=O)c2cc(-c3sc(NC(=O)CC4CCOCC4)nc3C)cnc2OC)c1] |
| S-265 | 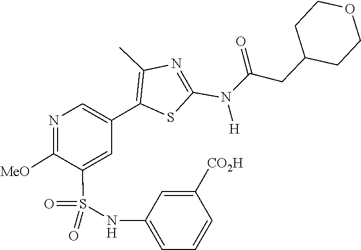 [SMILES: COc1ncc(-c2sc(NC(=O)CC3CCOCC3)nc2C)cc1S(=O)(=O)Nc1cccc(C(=O)O)c1] |
| S-266 | 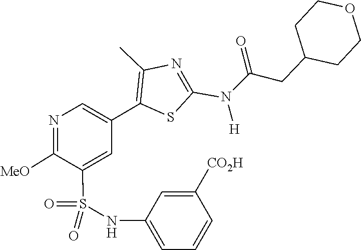 [SMILES: COc1ncc(-c2sc(NC(=O)CC3CCOCC3)nc2C)cc1S(=O)(=O)Nc1cccc(C(=O)O)c1] |
| S-267 | 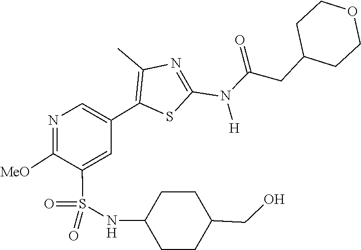 [SMILES: COc1ncc(-c2sc(NC(=O)CC3CCOCC3)nc2C)cc1S(=O)(=O)NC1CCC(CO)CC1] |
| S-268 | 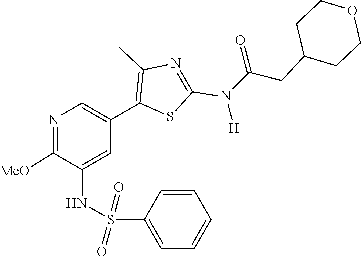 [SMILES: COc1ncc(-c2sc(NC(=O)CC3CCOCC3)nc2C)cc1NS(=O)(=O)c1ccccc1] |
| S-269 | 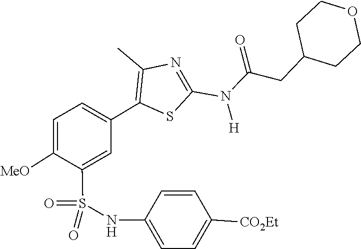 [SMILES: CCOC(=O)c1ccc(NS(=O)(=O)c2cc(-c3sc(NC(=O)CC4CCOCC4)nc3C)ccc2OC)cc1] |
| S-270 | 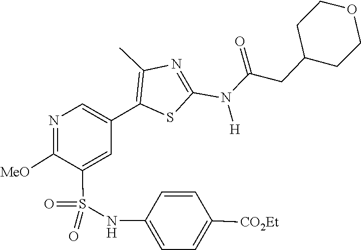 [SMILES: CCOC(=O)c1ccc(NS(=O)(=O)c2cc(-c3sc(NC(=O)CC4CCOCC4)nc3C)cnc2OC)cc1] |
| S-271 | 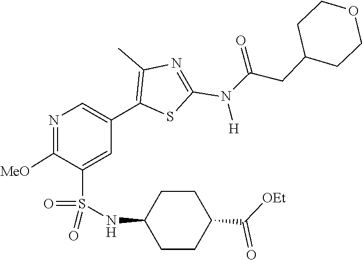 [SMILES: CCOC(=O)[C@H]1CC[C@H](NS(=O)(=O)c2cc(-c3sc(NC(=O)CC4CCOCC4)nc3C)cnc2OC)CC1] |
| S-272 | 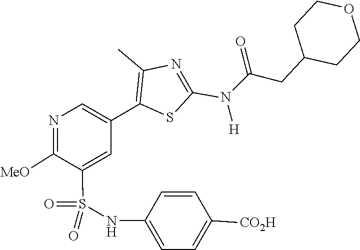 [SMILES: COc1ncc(-c2sc(NC(=O)CC3CCOCC3)nc2C)cc1S(=O)(=O)Nc1ccc(C(=O)O)cc1] |
| S-273 | 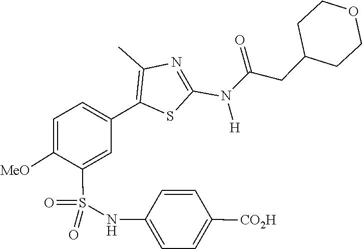 [SMILES: COc1ccc(-c2sc(NC(=O)CC3CCOCC3)nc2C)cc1S(=O)(=O)Nc1ccc(C(=O)O)cc1] |
| | 4-(2-methoxy-5-(4-methyl-2-(2- |
| | (tetrahydro-2H-pyran-4- |
| | yl)acetamido)thiazol-5- |
| | yl)phenylsulfonamido)benzoic acid |
| S-274 | 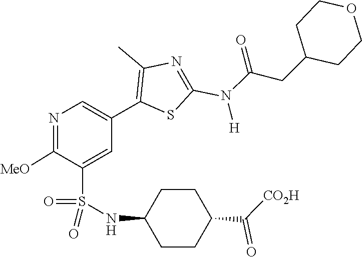 [SMILES: COc1ncc(-c2sc(NC(=O)CC3CCOCC3)nc2C)cc1S(=O)(=O)N[C@H]1CC[C@H](C(=O)C(=O)O)CC1] |
| | 2-((1r,4r)-4-(2-methoxy-5-(4-methyl-2- |
| | (2-(tetrahydro-2H-pyran-4- |
| | yl)acetamido)thiazol-5-yl)pyridine-3- |
| | sulfonamido)cyclohexyl)-2-oxoacetic |
| | acid |
| S-275 | 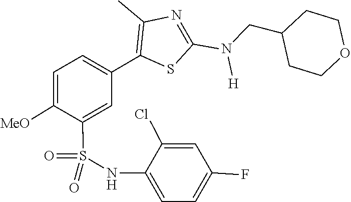 [SMILES: COc1ccc(-c2sc(NCC3CCOCC3)nc2C)cc1S(=O)(=O)Nc1ccc(F)cc1Cl] |
| | N-(2-chloro-4-fluoro-phenyl)-2-methoxy- |
| | 5-[4-methyl-2-(tetrahydropyran-4- |
| | ylmethylamino)thiazol-5- |
| | yl]benzenesulfonamide |
| S-276 | 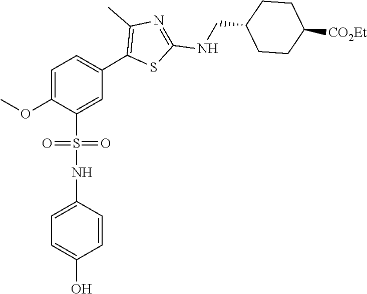 [SMILES: CCOC(=O)[C@H]1CC[C@H](CNc2nc(C)c(-c3ccc(OC)c(S(=O)(=O)Nc4ccc(O)cc4)c3)s2)CC1] |
| S-277 | 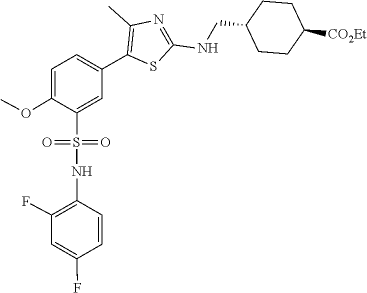 [SMILES: CCOC(=O)[C@H]1CC[C@H](CNc2nc(C)c(-c3ccc(OC)c(S(=O)(=O)Nc4ccc(F)cc4F)c3)s2)CC1] |
| S- 276b | 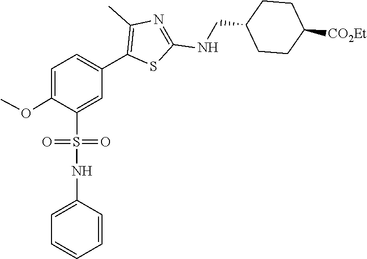 [SMILES: CCOC(=O)[C@H]1CC[C@H](CNc2nc(C)c(-c3ccc(OC)c(S(=O)(=O)Nc4ccccc4)c3)s2)CC1] |
| S-278 | 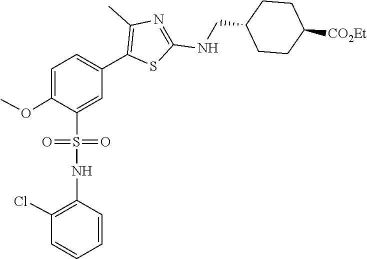 [SMILES: CCOC(=O)[C@H]1CC[C@H](CNc2nc(C)c(-c3ccc(OC)c(S(=O)(=O)Nc4ccccc4Cl)c3)s2)CC1] |
| S-279 | 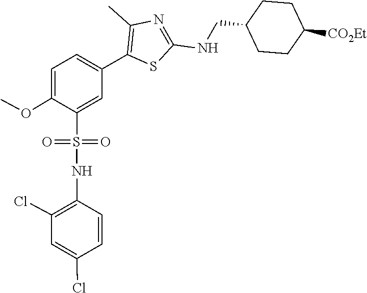 [SMILES: CCOC(=O)[C@H]1CC[C@H](CNc2nc(C)c(-c3ccc(OC)c(S(=O)(=O)Nc4ccc(Cl)cc4Cl)c3)s2)CC1] |
| S-280 | 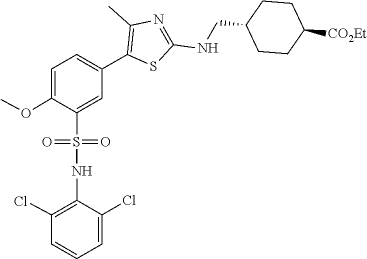 [SMILES: CCOC(=O)[C@H]1CC[C@H](CNc2nc(C)c(-c3ccc(OC)c(S(=O)(=O)Nc4c(Cl)cccc4Cl)c3)s2)CC1] |
| S-281 | 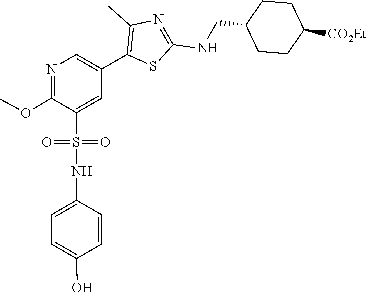 [SMILES: CCOC(=O)[C@H]1CC[C@H](CNc2nc(C)c(-c3cnc(OC)c(S(=O)(=O)Nc4ccc(O)cc4)c3)s2)CC1] |
| S-282 | 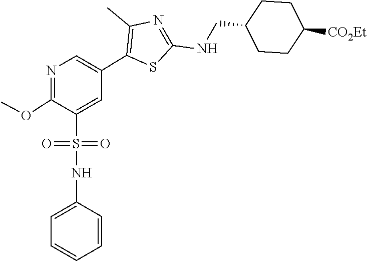 [SMILES: CCOC(=O)[C@H]1CC[C@H](CNc2nc(C)c(-c3cnc(OC)c(S(=O)(=O)Nc4ccccc4)c3)s2)CC1] |
| S-283 | 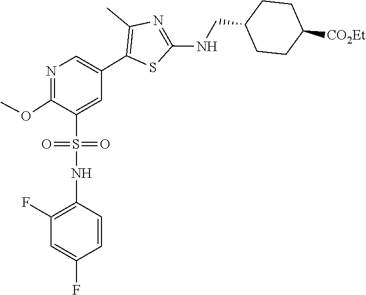 [SMILES: CCOC(=O)[C@H]1CC[C@H](CNc2nc(C)c(-c3cnc(OC)c(S(=O)(=O)Nc4ccc(F)cc4F)c3)s2)CC1] |
| S-284 | 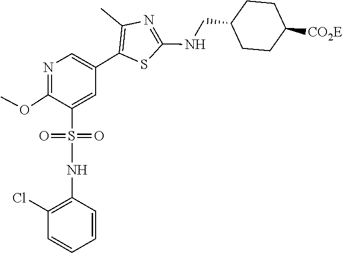 |
| S-285 | 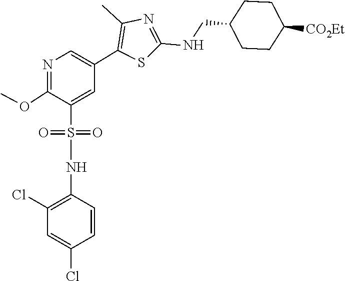 [SMILES: CCOC(=O)[C@H]1CC[C@H](CNc2nc(C)c(-c3cnc(OC)c(S(=O)(=O)Nc4ccc(Cl)cc4Cl)c3)s2)CC1] |
| S-286 | 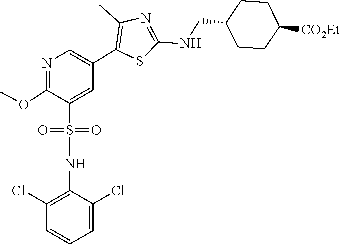 [SMILES: CCOC(=O)[C@H]1CC[C@H](CNc2nc(C)c(-c3cnc(OC)c(S(=O)(=O)Nc4c(Cl)cccc4Cl)c3)s2)CC1] |
| S-287 | 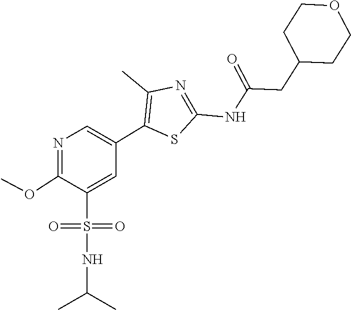 [SMILES: COc1ncc(-c2sc(NC(=O)CC3CCOCC3)nc2C)cc1S(=O)(=O)NC(C)C] |
| S-288 | 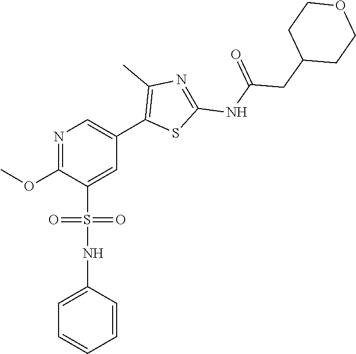 [SMILES: COc1ncc(-c2sc(NC(=O)CC3CCOCC3)nc2C)cc1S(=O)(=O)Nc1ccccc1] |
| S-289 | 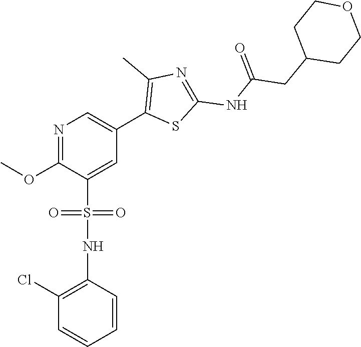 [SMILES: COc1ncc(-c2sc(NC(=O)CC3CCOCC3)nc2C)cc1S(=O)(=O)Nc1ccccc1Cl] |
| S-290 | 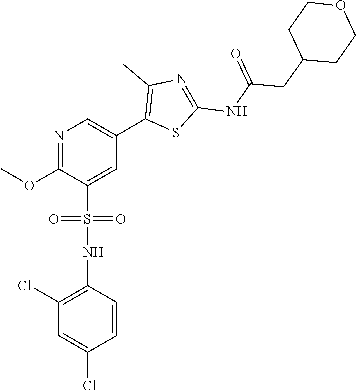 [SMILES: COc1ncc(-c2sc(NC(=O)CC3CCOCC3)nc2C)cc1S(=O)(=O)Nc1ccc(Cl)cc1Cl] |
| S-291 | 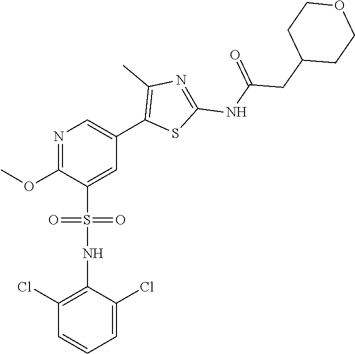 [SMILES: COc1ncc(-c2sc(NC(=O)CC3CCOCC3)nc2C)cc1S(=O)(=O)Nc1c(Cl)cccc1Cl] |
| S-292 | 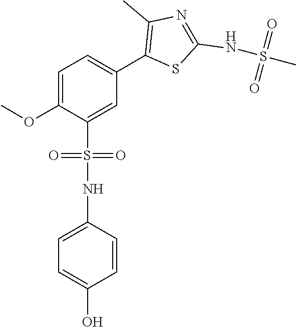 |
| S-293 | 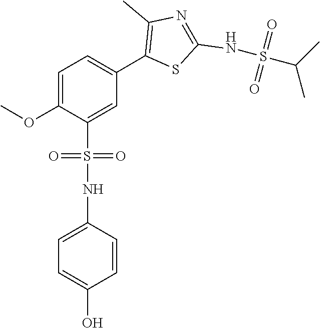 [SMILES: COc1ccc(-c2sc(NS(=O)(=O)C(C)C)nc2C)cc1S(=O)(=O)Nc1ccc(O)cc1] |
| S-294 | 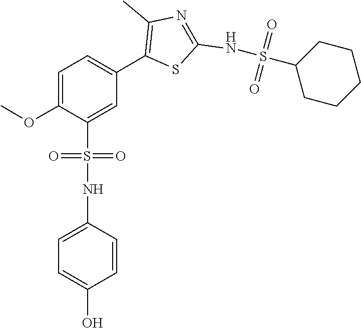 [SMILES: COc1ccc(-c2sc(NS(=O)(=O)C3CCCCC3)nc2C)cc1S(=O)(=O)Nc1ccc(O)cc1] |
| S- 294b | 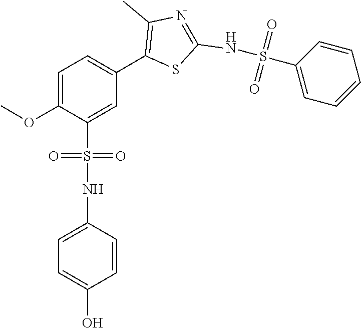 [SMILES: COc1ccc(-c2sc(NS(=O)(=O)c3ccccc3)nc2C)cc1S(=O)(=O)Nc1ccc(O)cc1] |
| S-295 | 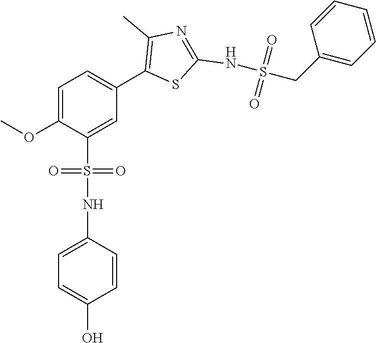 [SMILES: COc1ccc(-c2sc(NS(=O)(=O)Cc3ccccc3)nc2C)cc1S(=O)(=O)Nc1ccc(O)cc1] |
| S-296 | 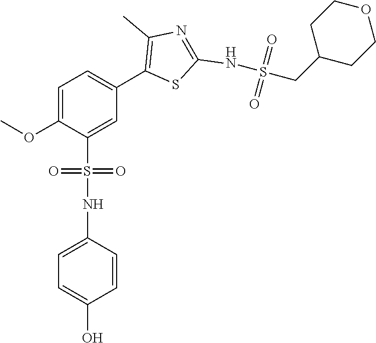 [SMILES: COc1ccc(-c2sc(NS(=O)(=O)CC3CCOCC3)nc2C)cc1S(=O)(=O)Nc1ccc(O)cc1] |
| S-297 | 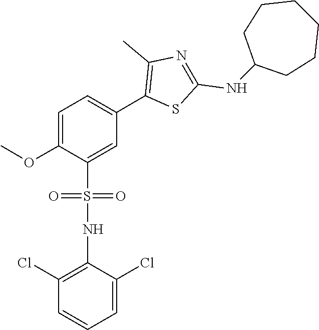 [SMILES: COc1ccc(-c2sc(NC3CCCCCC3)nc2C)cc1S(=O)(=O)Nc1c(Cl)cccc1Cl] |
| S-298 | 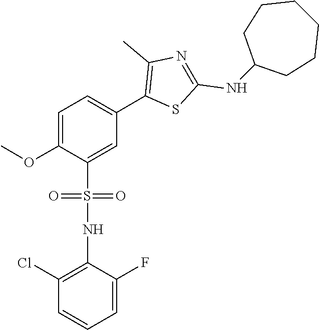 [SMILES: COc1ccc(-c2sc(NC3CCCCCC3)nc2C)cc1S(=O)(=O)Nc1c(F)cccc1Cl] |
| S-299 | 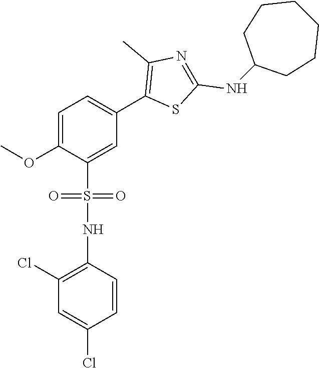 [SMILES: COc1ccc(-c2sc(NC3CCCCCC3)nc2C)cc1S(=O)(=O)Nc1ccc(Cl)cc1Cl] |
| S-300 | 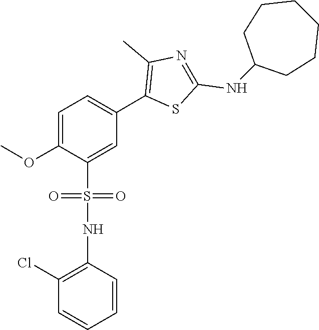 [SMILES: COc1ccc(-c2sc(NC3CCCCCC3)nc2C)cc1S(=O)(=O)Nc1ccccc1Cl] |
| S-301 | 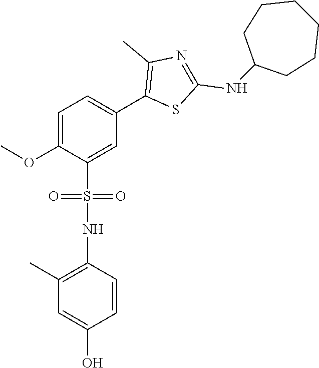 [SMILES: COc1ccc(-c2sc(NC3CCCCCC3)nc2C)cc1S(=O)(=O)Nc1ccc(O)cc1C] |
| S-302 | 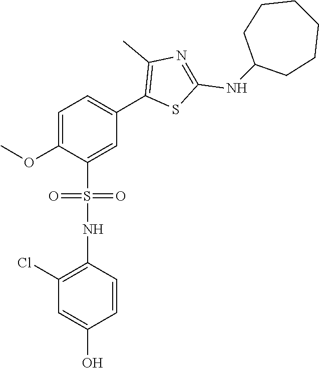 [SMILES: COc1ccc(-c2sc(NC3CCCCCC3)nc2C)cc1S(=O)(=O)Nc1ccc(O)cc1Cl] |
| S-303 | 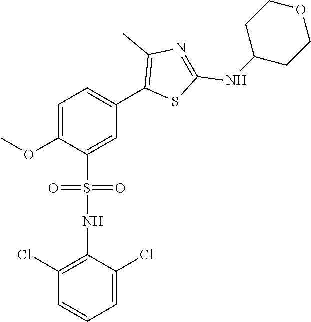 [SMILES: COc1ccc(-c2sc(NC3CCOCC3)nc2C)cc1S(=O)(=O)Nc1c(Cl)cccc1Cl] |
| S-304 | 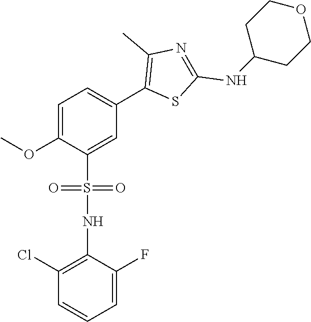 [SMILES: COc1ccc(-c2sc(NC3CCOCC3)nc2C)cc1S(=O)(=O)Nc1c(F)cccc1Cl] |
| S-305 | 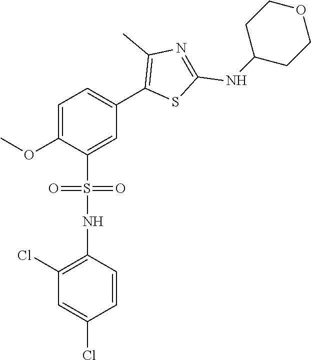 [SMILES: COc1ccc(-c2sc(NC3CCOCC3)nc2C)cc1S(=O)(=O)Nc1ccc(Cl)cc1Cl] |
| S-306 | 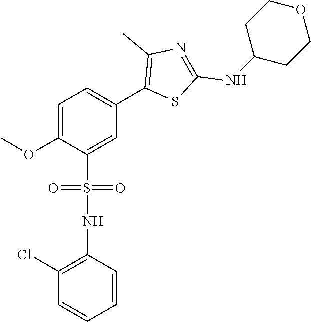 [SMILES: COc1ccc(-c2sc(NC3CCOCC3)nc2C)cc1S(=O)(=O)Nc1ccccc1Cl] |
| S-307 | 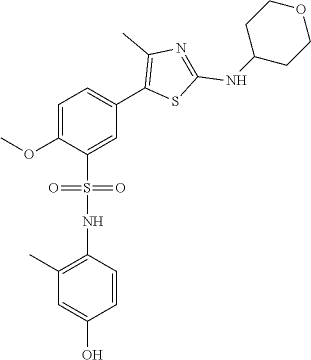 [SMILES: COc1ccc(-c2sc(NC3CCOCC3)nc2C)cc1S(=O)(=O)Nc1ccc(O)cc1C] |
| S-308 | 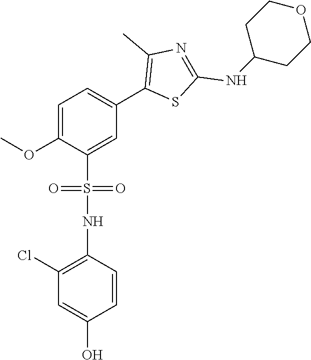 [SMILES: COc1ccc(-c2sc(NC3CCOCC3)nc2C)cc1S(=O)(=O)Nc1ccc(O)cc1Cl] |
| S-309 | 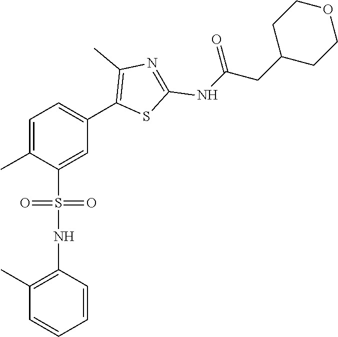 [SMILES: Cc1ccccc1NS(=O)(=O)c1cc(-c2sc(NC(=O)CC3CCOCC3)nc2C)ccc1C] |
| S-310 | 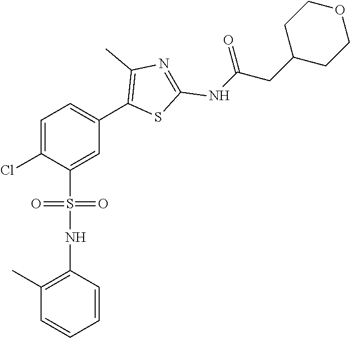 [SMILES: Cc1ccccc1NS(=O)(=O)c1cc(-c2sc(NC(=O)CC3CCOCC3)nc2C)ccc1Cl] |
| S-311 | 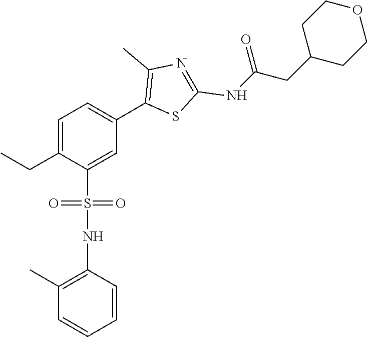 [SMILES: CCc1ccc(-c2sc(NC(=O)CC3CCOCC3)nc2C)cc1S(=O)(=O)Nc1ccccc1C] |
| S-312 | 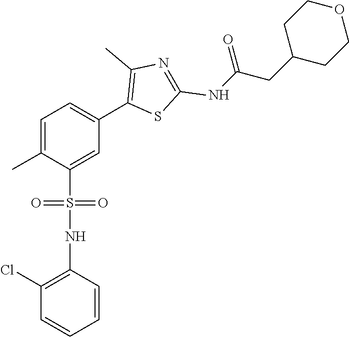 [SMILES: Cc1ccc(-c2sc(NC(=O)CC3CCOCC3)nc2C)cc1S(=O)(=O)Nc1ccccc1Cl] |
| S-313 | 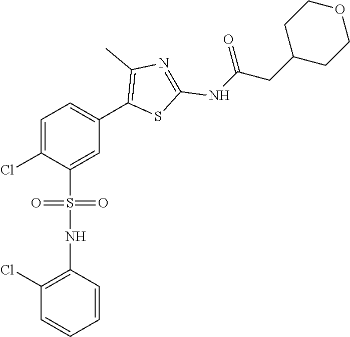 [SMILES: Cc1nc(NC(=O)CC2CCOCC2)sc1-c1ccc(Cl)c(S(=O)(=O)Nc2ccccc2Cl)c1] |
| S-314 | 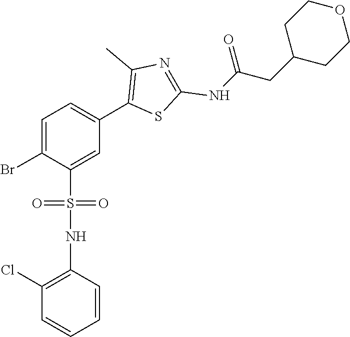 [SMILES: Cc1nc(NC(=O)CC2CCOCC2)sc1-c1ccc(Br)c(S(=O)(=O)Nc2ccccc2Cl)c1] |

TABLE 2
Compounds
Compounds

| Cmpd # | Structure |
|---|---|
| S-315 | 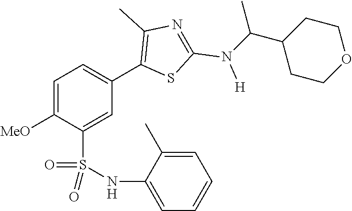 [SMILES: COc1ccc(-c2sc(NC(C)C3CCOCC3)nc2C)cc1S(=O)(=O)Nc1ccccc1C] |
| S-316 | 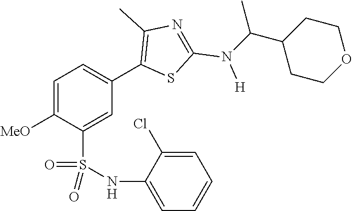 [SMILES: COc1ccc(-c2sc(NC(C)C3CCOCC3)nc2C)cc1S(=O)(=O)Nc1ccccc1Cl] |
| S-317 | 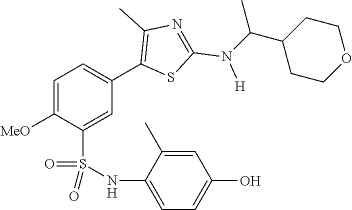 [SMILES: COc1ccc(-c2sc(NC(C)C3CCOCC3)nc2C)cc1S(=O)(=O)Nc1ccc(O)cc1C] |
| S-318 | 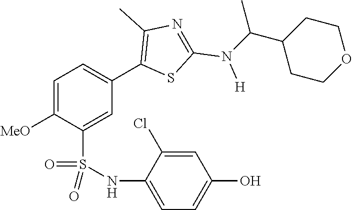 [SMILES: COc1ccc(-c2sc(NC(C)C3CCOCC3)nc2C)cc1S(=O)(=O)Nc1ccc(O)cc1Cl] |
| S-319 | 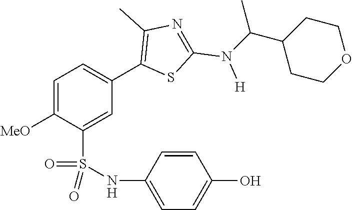 [SMILES: COc1ccc(-c2sc(NC(C)C3CCOCC3)nc2C)cc1S(=O)(=O)Nc1ccc(O)cc1] |
| S-320 | 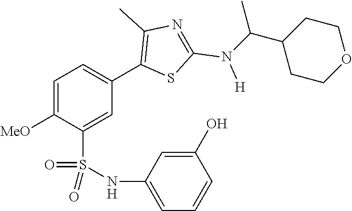 [SMILES: COc1ccc(-c2sc(NC(C)C3CCOCC3)nc2C)cc1S(=O)(=O)Nc1cccc(O)c1] |
| S-321 | 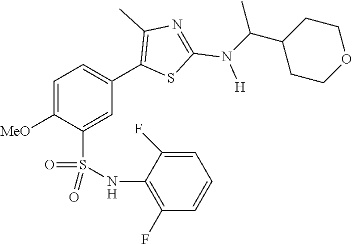 [SMILES: COc1ccc(-c2sc(NC(C)C3CCOCC3)nc2C)cc1S(=O)(=O)Nc1c(F)cccc1F] |
| S-322 | 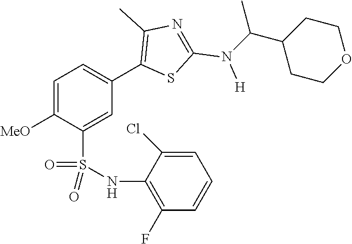 [SMILES: COc1ccc(-c2sc(NC(C)C3CCOCC3)nc2C)cc1S(=O)(=O)Nc1c(F)cccc1Cl] |
| S-323 | 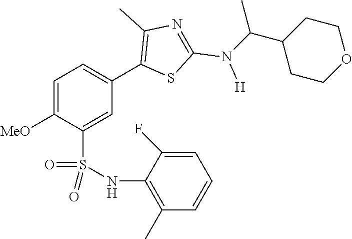 [SMILES: COc1ccc(-c2sc(NC(C)C3CCOCC3)nc2C)cc1S(=O)(=O)Nc1c(C)cccc1F] |
| S-324 | 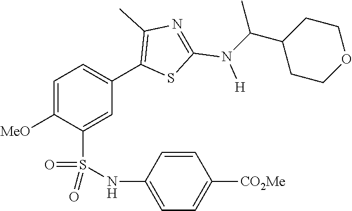 [SMILES: COC(=O)c1ccc(NS(=O)(=O)c2cc(-c3sc(NC(C)C4CCOCC4)nc3C)ccc2OC)cc1] |
| S-325 | 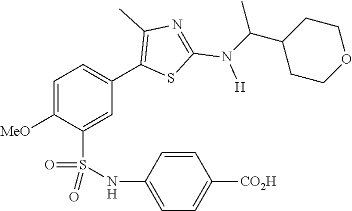 [SMILES: COc1ccc(-c2sc(NC(C)C3CCOCC3)nc2C)cc1S(=O)(=O)Nc1ccc(C(=O)O)cc1] |
| S-326 | 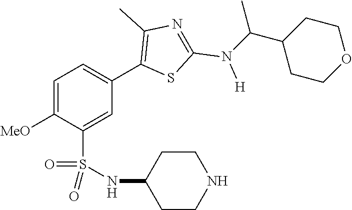 |
| S-327 | 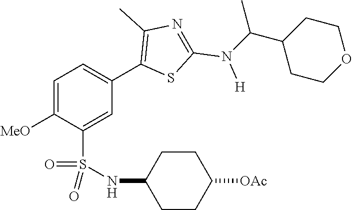 |
| S-328 | 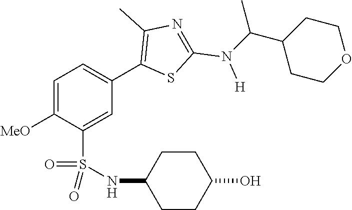 |
| S-329 | 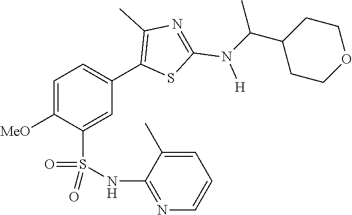 [SMILES: COc1ccc(-c2sc(NC(C)C3CCOCC3)nc2C)cc1S(=O)(=O)Nc1ncccc1C] |
| S-330 | 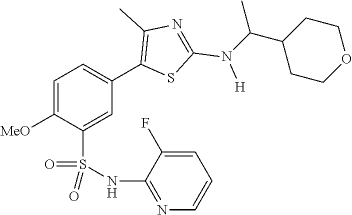 [SMILES: COc1ccc(-c2sc(NC(C)C3CCOCC3)nc2C)cc1S(=O)(=O)Nc1ncccc1F] |
| S-331 | 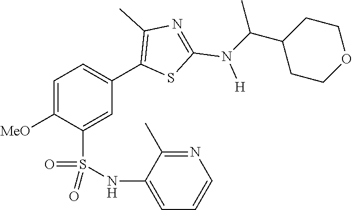 [SMILES: COc1ccc(-c2sc(NC(C)C3CCOCC3)nc2C)cc1S(=O)(=O)Nc1cccnc1C] |
| S-332 | 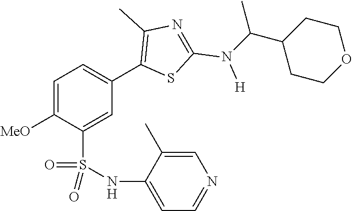 [SMILES: COc1ccc(-c2sc(NC(C)C3CCOCC3)nc2C)cc1S(=O)(=O)Nc1ccncc1C] |
| S-333 | 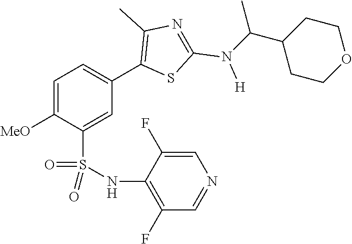 [SMILES: COc1ccc(-c2sc(NC(C)C3CCOCC3)nc2C)cc1S(=O)(=O)Nc1c(F)cncc1F] |
| S-334 | 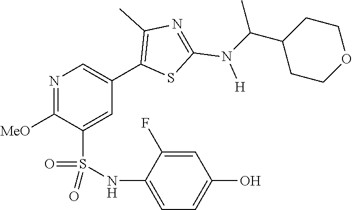 [SMILES: COc1ncc(-c2sc(NC(C)C3CCOCC3)nc2C)cc1S(=O)(=O)Nc1ccc(O)cc1F] |
| S-335 | 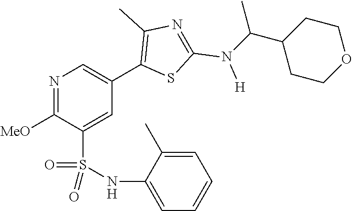 [SMILES: COc1ncc(-c2sc(NC(C)C3CCOCC3)nc2C)cc1S(=O)(=O)Nc1ccccc1C] |
| S-336 | 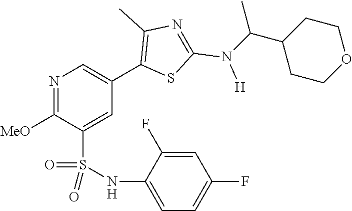 [SMILES: COc1ncc(-c2sc(NC(C)C3CCOCC3)nc2C)cc1S(=O)(=O)Nc1ccc(F)cc1F] |
| S-337 | 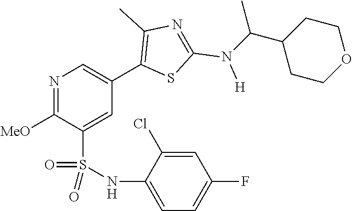 [SMILES: COc1ncc(-c2sc(NC(C)C3CCOCC3)nc2C)cc1S(=O)(=O)Nc1ccc(F)cc1Cl] |
| S-338 | 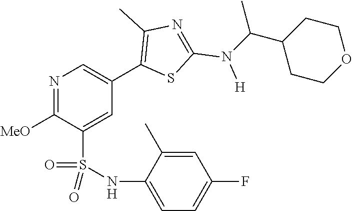 [SMILES: COc1ncc(-c2sc(NC(C)C3CCOCC3)nc2C)cc1S(=O)(=O)Nc1ccc(F)cc1C] |
| S-339 | 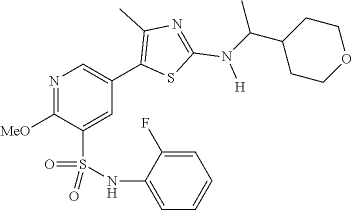 [SMILES: COc1ncc(-c2sc(NC(C)C3CCOCC3)nc2C)cc1S(=O)(=O)Nc1ccccc1F] |
| S-340 | 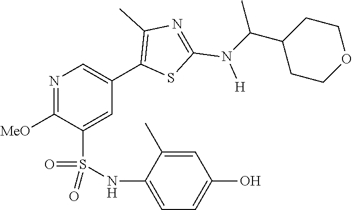 [SMILES: COc1ncc(-c2sc(NC(C)C3CCOCC3)nc2C)cc1S(=O)(=O)Nc1ccc(O)cc1C] |
| S-341 | 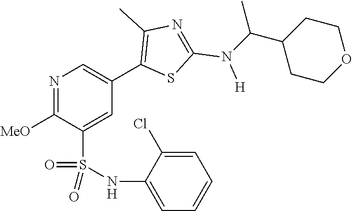 [SMILES: COc1ncc(-c2sc(NC(C)C3CCOCC3)nc2C)cc1S(=O)(=O)Nc1ccccc1Cl] |
| S-342 | 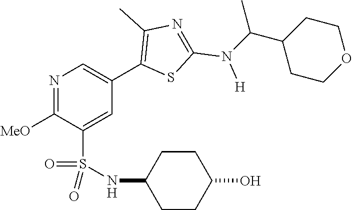 |
| S-343 | 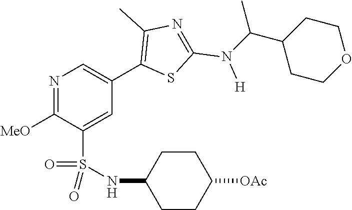 |
| S-344 | 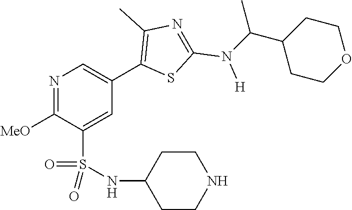 [SMILES: COc1ncc(-c2sc(NC(C)C3CCOCC3)nc2C)cc1S(=O)(=O)NC1CCNCC1] |
| S-345 | 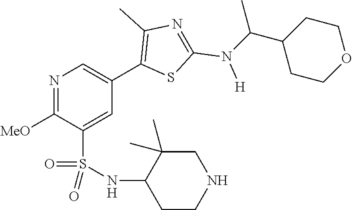 [SMILES: COc1ncc(-c2sc(NC(C)C3CCOCC3)nc2C)cc1S(=O)(=O)NC1CCNCC1(C)C] |
| S-346 | 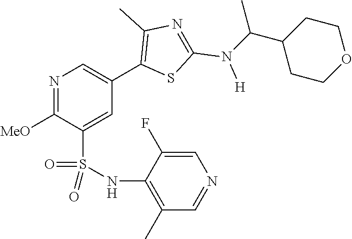 [SMILES: COc1ncc(-c2sc(NC(C)C3CCOCC3)nc2C)cc1S(=O)(=O)Nc1c(C)cncc1F] |
| S-347 | 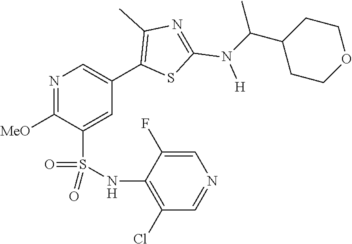 [SMILES: COc1ncc(-c2sc(NC(C)C3CCOCC3)nc2C)cc1S(=O)(=O)Nc1c(F)cncc1Cl] |
| S-348 | 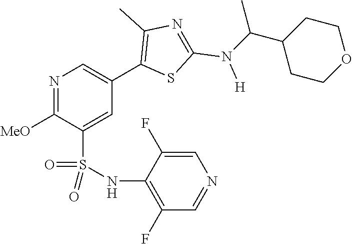 [SMILES: COc1ncc(-c2sc(NC(C)C3CCOCC3)nc2C)cc1S(=O)(=O)Nc1c(F)cncc1F] |
| S-349 | 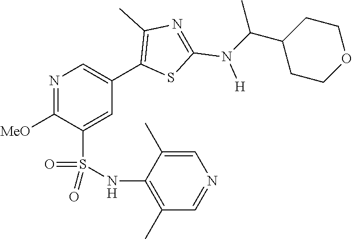 [SMILES: COc1ncc(-c2sc(NC(C)C3CCOCC3)nc2C)cc1S(=O)(=O)Nc1c(C)cncc1C] |
| S-350 | 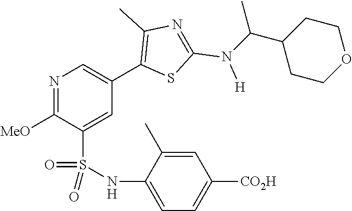 [SMILES: COc1ncc(-c2sc(NC(C)C3CCOCC3)nc2C)cc1S(=O)(=O)Nc1ccc(C(=O)O)cc1C] |
| S-351 | 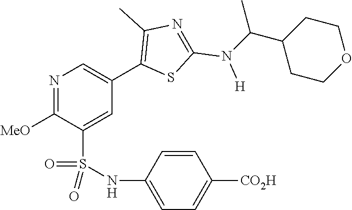 [SMILES: COc1ncc(-c2sc(NC(C)C3CCOCC3)nc2C)cc1S(=O)(=O)Nc1ccc(C(=O)O)cc1] |
| S-352 | 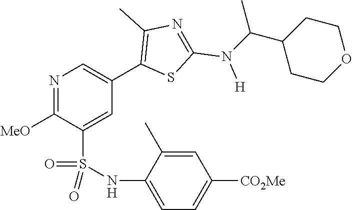 [SMILES: COC(=O)c1ccc(NS(=O)(=O)c2cc(-c3sc(NC(C)C4CCOCC4)nc3C)cnc2OC)c(C)c1] |
| S-353 | 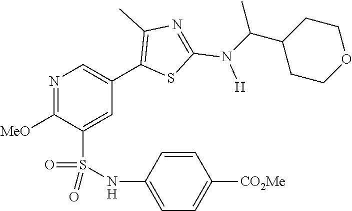 [SMILES: COC(=O)c1ccc(NS(=O)(=O)c2cc(-c3sc(NC(C)C4CCOCC4)nc3C)cnc2OC)cc1] |
| S-354 | 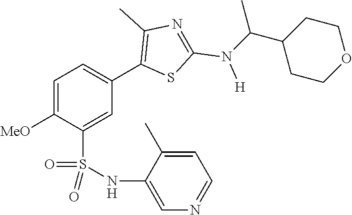 [SMILES: COc1ccc(-c2sc(NC(C)C3CCOCC3)nc2C)cc1S(=O)(=O)Nc1cnccc1C] |
| S-355 | 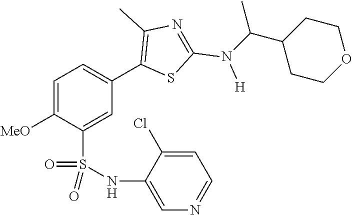 [SMILES: COc1ccc(-c2sc(NC(C)C3CCOCC3)nc2C)cc1S(=O)(=O)Nc1cnccc1Cl] |
| S-356 | 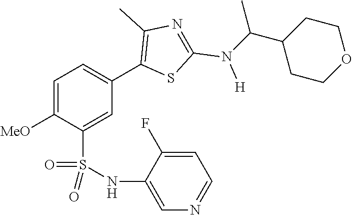 [SMILES: COc1ccc(-c2sc(NC(C)C3CCOCC3)nc2C)cc1S(=O)(=O)Nc1cnccc1F] |
| S-357 | 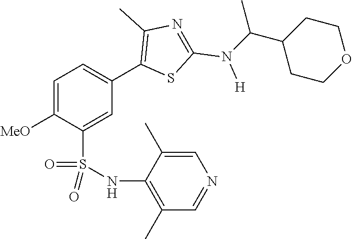 [SMILES: COc1ccc(-c2sc(NC(C)C3CCOCC3)nc2C)cc1S(=O)(=O)Nc1c(C)cncc1C] |
| S-358 | 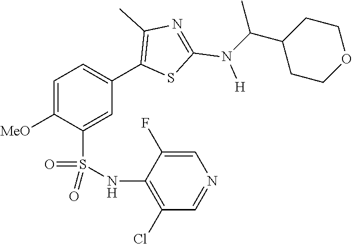 [SMILES: COc1ccc(-c2sc(NC(C)C3CCOCC3)nc2C)cc1S(=O)(=O)Nc1c(F)cncc1Cl] |
| S-359 | 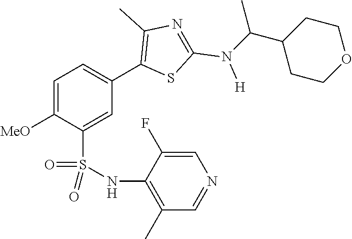 [SMILES: COc1ccc(-c2sc(NC(C)C3CCOCC3)nc2C)cc1S(=O)(=O)Nc1c(C)cncc1F] |
| S-360 | 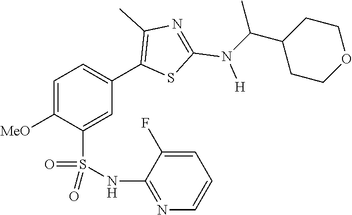 [SMILES: COc1ccc(-c2sc(NC(C)C3CCOCC3)nc2C)cc1S(=O)(=O)Nc1ncccc1F] |
| S-361 | 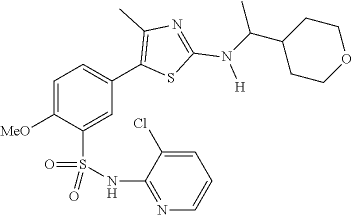 [SMILES: COc1ccc(-c2sc(NC(C)C3CCOCC3)nc2C)cc1S(=O)(=O)Nc1ncccc1Cl] |
| S-362 | 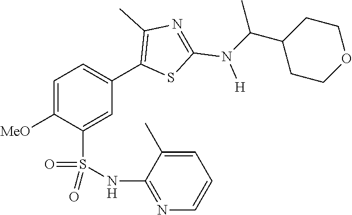 [SMILES: COc1ccc(-c2sc(NC(C)C3CCOCC3)nc2C)cc1S(=O)(=O)Nc1ncccc1C] |
| S-363 | 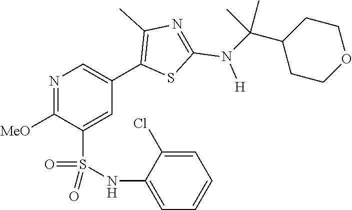 [SMILES: COc1ncc(-c2sc(NC(C)(C)C3CCOCC3)nc2C)cc1S(=O)(=O)Nc1ccccc1Cl] |
| S-364 | 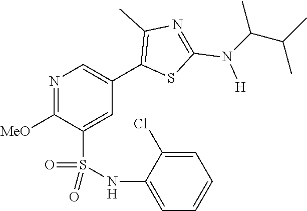 [SMILES: COc1ncc(-c2sc(NC(C)C(C)C)nc2C)cc1S(=O)(=O)Nc1ccccc1Cl] |
| S-365 | 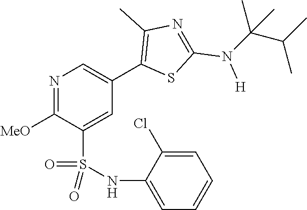 [SMILES: COc1ncc(-c2sc(NC(C)(C)C(C)C)nc2C)cc1S(=O)(=O)Nc1ccccc1Cl] |
| S-366 | 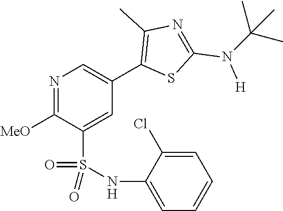 [SMILES: COc1ncc(-c2sc(NC(C)(C)C)nc2C)cc1S(=O)(=O)Nc1ccccc1Cl] |
| S-367 | 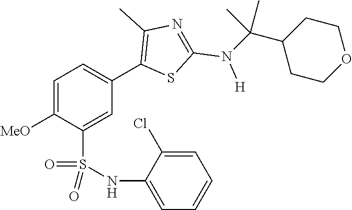 [SMILES: COc1ccc(-c2sc(NC(C)(C)C3CCOCC3)nc2C)cc1S(=O)(=O)Nc1ccccc1Cl] |
| S-368 | 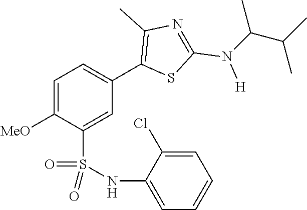 [SMILES: COc1ccc(-c2sc(NC(C)C(C)C)nc2C)cc1S(=O)(=O)Nc1ccccc1Cl] |
| S-369 | 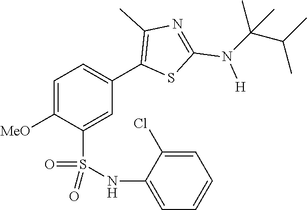 [SMILES: COc1ccc(-c2sc(NC(C)(C)C(C)C)nc2C)cc1S(=O)(=O)Nc1ccccc1Cl] |
| S-370 | 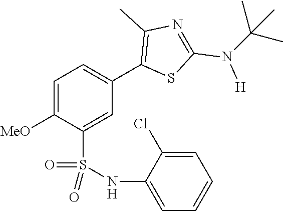 [SMILES: COc1ccc(-c2sc(NC(C)(C)C)nc2C)cc1S(=O)(=O)Nc1ccccc1Cl] |
| S-371 | 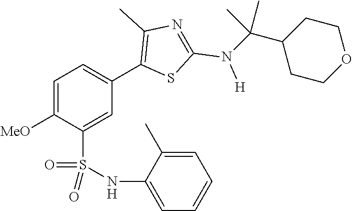 [SMILES: COc1ccc(-c2sc(NC(C)(C)C3CCOCC3)nc2C)cc1S(=O)(=O)Nc1ccccc1C] |
| S-372 | 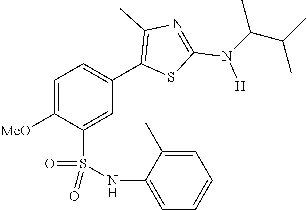 [SMILES: COc1ccc(-c2sc(NC(C)C(C)C)nc2C)cc1S(=O)(=O)Nc1ccccc1C] |
| S-373 | 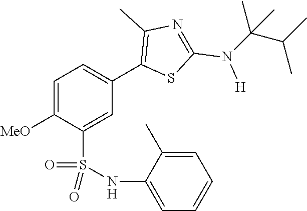 [SMILES: COc1ccc(-c2sc(NC(C)(C)C(C)C)nc2C)cc1S(=O)(=O)Nc1ccccc1C] |
| S-374 | 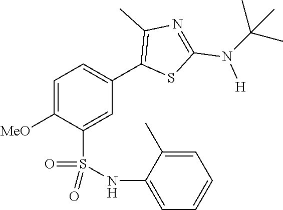 [SMILES: COc1ccc(-c2sc(NC(C)(C)C)nc2C)cc1S(=O)(=O)Nc1ccccc1C] |
| S-375 | 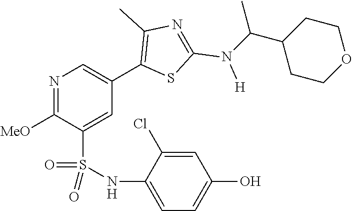 [SMILES: COc1ncc(-c2sc(NC(C)C3CCOCC3)nc2C)cc1S(=O)(=O)Nc1ccc(O)cc1Cl] |

In certain embodiments, the PI4-kinase inhibitor is described by the structure of one of the compounds of Table 1. In certain embodiments, the compound is described by the structure of one of the compounds of Table 2. It is understood that any of the compounds shown in Table 1 or 2 may be present in a salt form, such as a trifluoroacetate salt (e.g., CF₃COOH salt). In some cases, the salt form of the compound is a pharmaceutically acceptable salt.

In some cases, the subject compound is described by the structure of formula (Ia):

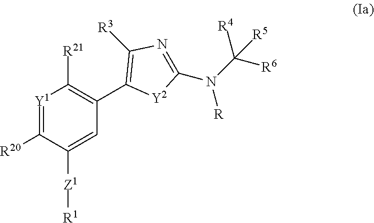

where:

Z¹ is a covalent bond or a linking functional group;

R is H, alkyl or alkyl (e.g., lower alkyl, such as methyl),

Y¹ is CR²² or N;

Y² is selected from S, O or NR¹⁹, wherein R¹⁹ is selected from hydrogen, alkyl, and substituted alkyl;

R¹ is selected from aryl, substituted aryl, heteroaryl, substituted heteroaryl, alkyl, substituted alkyl, cycloalkyl, substituted cycloalkyl, heterocycle and substituted heterocycle;

R³ is selected from hydrogen, lower alkyl and substituted lower alkyl;

R⁴ and R⁵ are each independently selected from lower alkyl and substituted lower alkyl; or R⁴ and R⁵ together with the carbon to which they are attached form a cyclic group selected from cycloalkyl, substituted cycloalkyl, heterocycle, substituted heterocycle, aryl, substituted aryl, heteroaryl and substituted heteroaryl; and

R⁶ is selected from substituted alkyl, cycloalkyl, substituted cycloalkyl, aryl, substituted aryl, heterocycle, substituted heterocycle, heteroaryl and substituted heteroaryl; or R⁴, R⁵ and R⁶ together with the carbon to which they are attached provide a bridged cyclic group selected from bridged cycloalkyl, substituted bridged cycloalkyl, bridged heterocycle and substituted bridged heterocycle; and

R²⁰, R²¹ and R²² are independently selected from hydrogen, an alkyl, a substituted alkyl, an aryl, a substituted aryl, a hydroxy, an alkoxy, a substituted alkoxy, an aryloxy, a substituted aryloxy, a heterocycle, a substituted heterocycle, a cyano, a halogen, an amino, a substituted amino, an acyl, an acyloxy, an amido, and a nitro.

In some cases, the subject compound is described by the structure of formula (Ib):

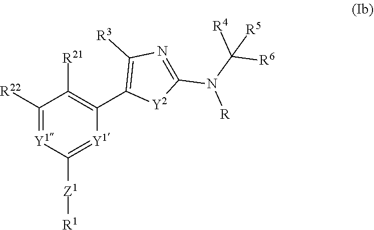

where:

Z¹ is a covalent bond or a linking functional group;

R is H, alkyl or alkyl (e.g., lower alkyl, such as methyl),

Y^{1′} and Y^{1″} are each independently CR²⁰ or N;

Y² is selected from S, O or NR¹⁹, wherein R¹⁹ is selected from hydrogen, alkyl, and substituted alkyl;

R¹ is selected from aryl, substituted aryl, heteroaryl, substituted heteroaryl, alkyl, substituted alkyl, cycloalkyl, substituted cycloalkyl, heterocycle and substituted heterocycle;

R³ is selected from hydrogen, lower alkyl and substituted lower alkyl;

R⁴ and R⁵ are each independently selected from lower alkyl and substituted lower alkyl; or R⁴ and R⁵ together with the carbon to which they are attached form a cyclic group selected from cycloalkyl, substituted cycloalkyl, heterocycle, substituted heterocycle, aryl, substituted aryl, heteroaryl and substituted heteroaryl; and

R⁶ is selected from substituted alkyl, cycloalkyl, substituted cycloalkyl, aryl, substituted aryl, heterocycle, substituted heterocycle, heteroaryl and substituted heteroaryl; or

R⁴, R⁵ and R⁶ together with the carbon to which they are attached provide a bridged cyclic group selected from bridged cycloalkyl, substituted bridged cycloalkyl, bridged heterocycle and substituted bridged heterocycle; and

R²⁰, R²¹ and R²² are independently selected from hydrogen, an alkyl, a substituted alkyl, an aryl, a substituted aryl, a hydroxy, an alkoxy, a substituted alkoxy, an aryloxy, a substituted aryloxy, a heterocycle, a substituted heterocycle, a cyano, a halogen, an amino, a substituted amino, an acyl, an acyloxy, an amido, and a nitro.

Further details regarding compounds of formulas 1(a) and 1(b) above are found in U.S. Provisional Patent Application Ser. No. 62/821,853; the disclosure of which is herein incorporated by reference.

Aspects of the present disclosure include use of PI4-kinase inhibiting compounds, salts thereof (e.g., pharmaceutically acceptable salts), and/or solvate, hydrate and/or prodrug forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

In some embodiments, the PI4-kinase inhibitors, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the PI4-kinase inhibitors are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)). In some cases, the promoiety is attached to a hydroxy or carboxylic acid group of the subject compounds. In certain cases, the promoiety is an acyl or substituted acyl group. In certain cases, the promoiety is an alkyl or substituted alkyl group, e.g., that forms an ester functional group when attached to a carboxylic acid group of the subject compounds.

In some embodiments, the PI4-kinase inhibitors, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the PI4-kinase inhibitors are provided by oral dosing and absorbed into the bloodstream. In some embodiments, the oral bioavailability of the subject compounds is 30% or more. Modifications may be made to the PI4-kinase inhibitors or their formulations using any convenient methods to increase absorption across the gut lumen or their bioavailability.

In some embodiments, the PI4-kinase inhibitors are metabolically stable (e.g., remain substantially intact in vivo during the half-life of the compound). In certain embodiments, the compounds have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

### Pi-Kinase Inhibition in Cancer Cells

As summarized above, aspects of the disclosure include use of PI4-kinase inhibiting compounds. The PI4-kinase inhibitors are compounds that inhibit the activity of a PI4-kinase in a cell, upon contact with a cell or components thereof. In some instances, the types of cells in which the compounds exhibit activity are cancer cells, as described herein. By inhibiting a PI4-kinase it is meant that the activity of the enzyme is decreased by a factor of 2 or more, such as 3 or more, 5 or more, 10 or more, 100 or more, or 1000 or more, relative to its normal activity (e.g., relative to a positive control).

The methods of the present disclosure can target cancer cells. The target cancer cells and their metastases can be considered “addicted” to increased PI4-kinase activity. The latter can result from amplification of chromosomal segments that harbor a PI4-kinase gene, such as PI4-III-kinase a or PI4-III-kinase β, or eukaryotic protein translation elongation factor 1 alpha 2 (eEF1A2). eEF1A2 is a translation factor that is involved in internal ribosome entry site (IRES) mediated translation. eEF1A2 also stimulates PI4-kinase activity and is overexpressed in many cancers. IRESs are often used by viruses as a means to ensure that viral translation is active when host translation is inhibited. IRES-mediated translation can contribute to the translation of certain cellular RNAs, particularly under abnormal cellular states. The target cancer cells can have the above chromosomal amplifications, or increased expression of eEF1A2 without chromosomal amplifications, any of which can lead to increased PI4 kinase activity. The inventors discovered that anti-viral PI4 kinase inhibitors that potently target IRES containing viruses were also effective in reducing proliferation of cancer cells and could find use in the treatment of cancer. In some embodiments, the cancer cells have normal levels of PI4-kinase activity, but are more sensitive to PI4-kinase activity than normal cells.

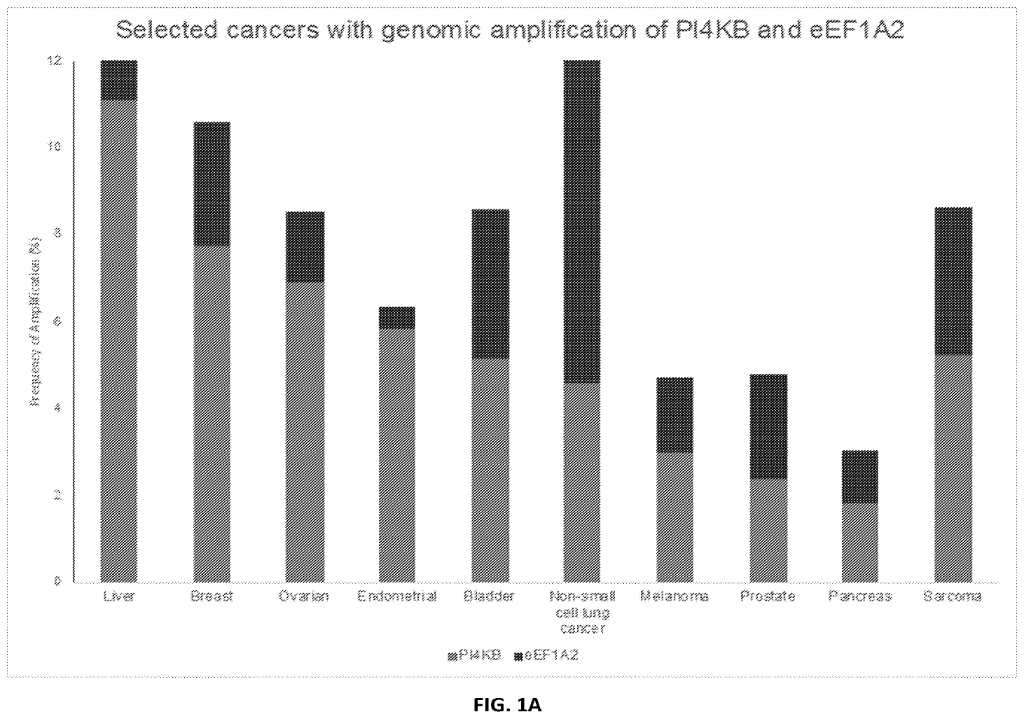

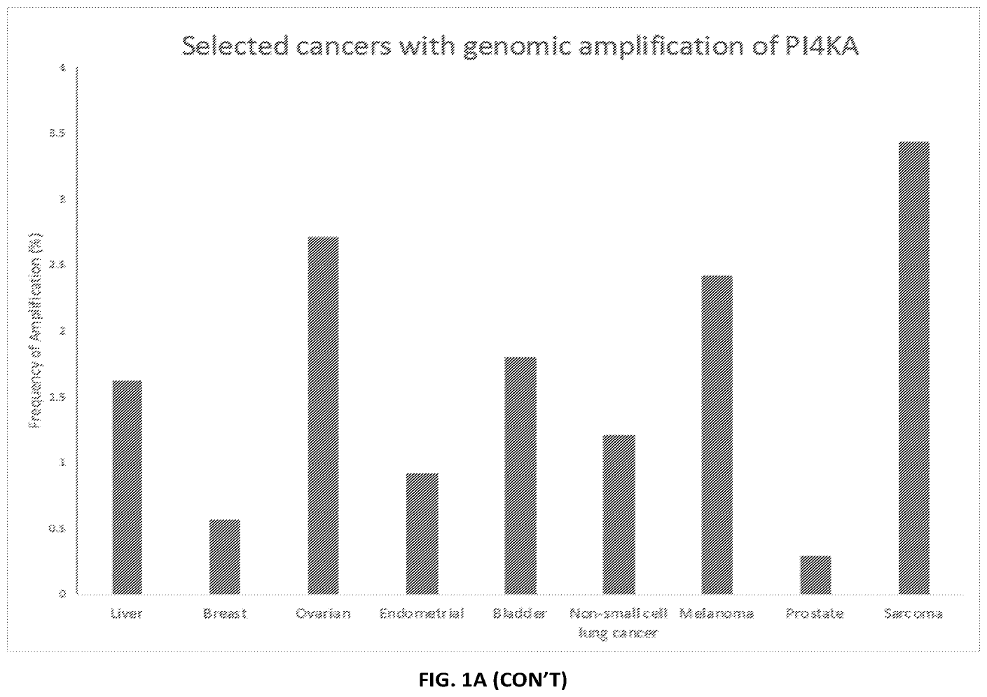

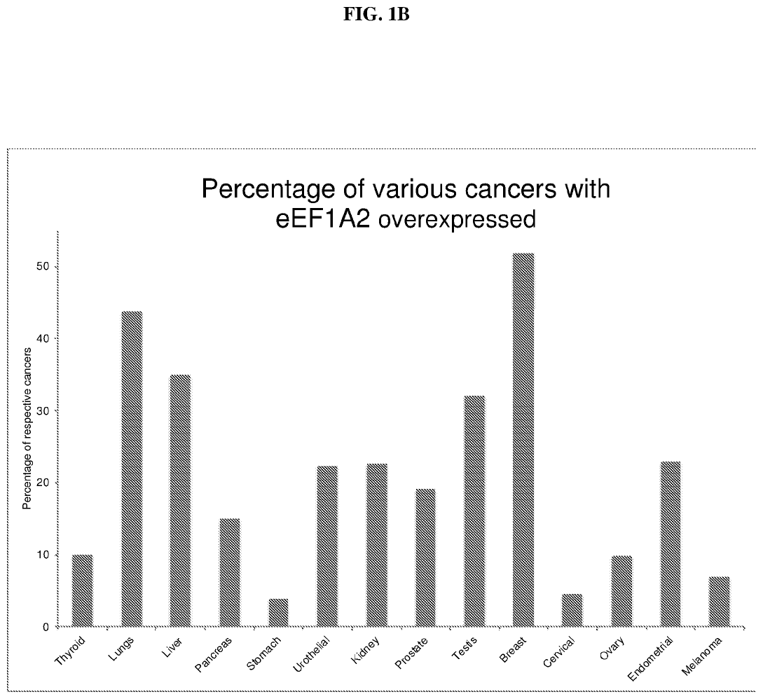

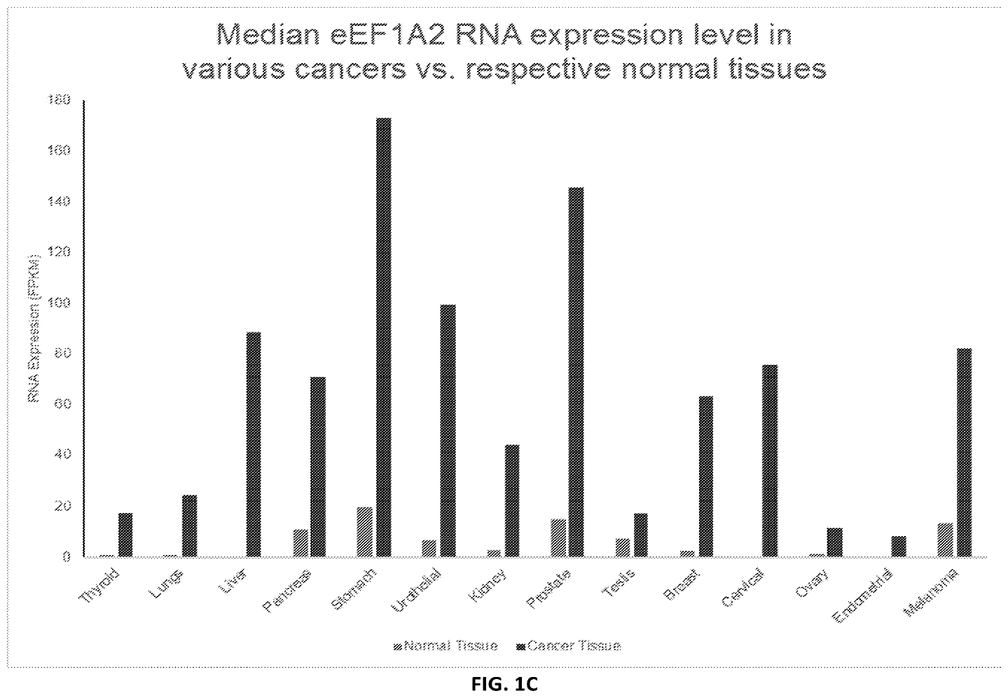

Cancer cells of interest which can be targeted according to the subject methods include those described in FIG. 1A-1C. In some instances, the cancer cell is selected from bladder, breast, colon, endometrial, liver, lung, non-small cell lung cancer (NSCLC), ovarian, prostate, pancreatic, melanoma and sarcoma cancer cells.

As such, aspects of this disclosure include assessing or measuring the level of expression of a PI4-kinase gene or a factor involved in IRES-mediated translation that stimulates PI4-kinase activity (e.g., eEF1A2 translation factor) in a target cell. In some cases, the assessing or measuring step includes determining whether the target cells have an elevated level of expression of a PI4-kinase gene or eEF1A2 translation factor. As used herein, the terms “elevated level of expression”, “overexpression” and “overexpressed” are used interchangeably and refer to a level of expression in a target cell that is 20% or more than the native or basal level of expression in a control cell, such as 30% or more, 40% or more, 40% or more, 40% or more, 40% or more, 40% or more, 40% or more, 2-fold greater or more, 5-fold greater or more, 10-fold greater or more, 30-fold greater or more, 100-fold greater or more or 1000-fold greater or more, as compared to the native or basal level of expression in a control cell. In some cases, the control cell is one or more control cells from a plurality of subjects. In certain cases, the control cell is one or more control cells from a plurality of cells of the same type as the target cell from a plurality of subjects. In some cases, the control cells are normal cells.

The methods that may be employed in measuring or determining levels of expression in a cell are numerous and include but are not limited to cellular assays in which a cellular phenotype is measured, e.g., gene expression assays. The methods can be qualitative or quantitative. Expression levels can be determined directly or indirectly. In some cases, the gene copy number for the gene of interest in the target cells is measured. In certain cases, the gene copy number of PI4 is determined, e.g., PI4KIIIβ or PI4KIIIα. In certain cases, the gene copy number of eEF1A2 is determined. In some cases, the eEF1A2 transcription level is determined. In some cases, the target cancer cells have a greater than diploid copy number of the PI4KIIIβ gene.

Aspects of this disclosure include assessing or measuring the level of activity of a PI4-kinase in a target cell. In some cases, the assessing or measuring step includes determining whether the target cells have an elevated level of activity of a PI4-kinase. The term “elevated level of activity” refers to a level of activity in a target cell that is 20% or more than the native or basal level of activity in a control cell, such as 30% or more, 40% or more, 40% or more, 40% or more, 40% or more, 40% or more, 40% or more, 2-fold greater or more, 5-fold greater or more, 10-fold greater or more, 30-fold greater or more, 100-fold greater or more or 1000-fold greater or more, as compared to the native or basal level of activity in a control cell. In some cases, the control cell is one or more control cells from a plurality of subjects. In certain cases, the control cell is one or more control cells from a plurality of cells of the same type as the target cell from a plurality of subjects. In some cases, the control cells are normal cells.

The methods that may be employed in determining PI4-kinase activity are numerous, and include but are not limited to cell-free assays, e.g., binding assays; assays using purified enzymes, measurements of PI4-P levels, cellular assays in which a cellular phenotype is measured, e.g., gene expression assays; and in vivo assays that involve a particular animal (which, in certain embodiments may be an animal model for a condition dependent on PI-kinase activity). In some cases, the target cancer cells have an elevated level of PI4KIIIβ activity. In some embodiments of the subject methods, the target cancer cells are cells that are sensitive to PI4KIIIβ inhibition. In certain cases, these PI4KIIIβ inhibition-sensitive cells do not exhibit an elevated level of expression or activity of PI4KIIIβ. In some embodiments, the PI4-kinase inhibitors are inhibitors of a PI4-III-kinase (e.g., PI4-IIIα or PI4-IIIβ). In some embodiments, the PI4-kinase inhibitors have a PI-kinase inhibition profile that reflects activity against two or more PI-kinases. In some embodiments, the PI4-kinase inhibitors specifically inhibit both a type II PI3-kinase, such as PI3-kinase IIβ, and a type III PI4-kinase, such as PI4K-IIIα and/or PI4K-IIIβ). In some embodiments, the PI4-kinase inhibitors specifically inhibit a PI4-kinase without undesired inhibition of other protein kinases. In some embodiments, the PI4-kinase inhibitors specifically inhibit a PI4-kinase without undesired inhibition of PI3-kinase. In some embodiments, the PI4-kinase inhibitors specifically inhibit a PI4-kinase and/or a specific PI3-kinase subclass without undesired inhibition of other PI3-kinase subclasses or protein kinases.

In some embodiments, the PI4-kinase inhibitors interfere with the interaction of a basic amino acid PIP-2 pincer (BAAPP) domain with phosphatidylinositol-4,5-bisphosphate PIP2 in a cell. See e.g., Glenn et al. US2011/0262565 and U.S. Pat. No. 9,926,309. For example, the subject compounds may act by decreasing the levels of PIP2 either directly or indirectly that bind specifically to the BAAPP domain.

PI4-kinase inhibition can be as determined by an inhibition assay, e.g., by an assay that determines the level of activity of the enzyme either in a cell-free system or in a cell after treatment with a subject compound, relative to a control, by measuring the IC₅₀ or EC₅₀ value, respectively. In certain embodiments, the subject compounds have an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower.

PI4-kinase inhibition can be determined by a kinase activity assay, e.g., by an assay that determines the level of incorporation of radiolabeled phosphate from [γ-³²P]-ATP into a substrate molecule after treatment with a subject compound, relative to a control, by measuring the beta-particle emission rate using a scintillation counter or phosphorimaging. In certain embodiments, the inhibitors have an IC₅₀ value for PI4K-IIIβ of less than about 1 μM, less than about 0.2 μM, less than about 0.1 μM, less than about 10 nM, less than about 1 nM, or even less, such as described in Table 3. In certain embodiments, the inhibitors have an IC₅₀ value for PI4K-IIIα of less than about 50 μM, less than about 10 μM, less than about 1 μM, less than about 0.1 μM, less than about 10 nM, less than about 1 nM, or even less, such as described in Tables 2-3. In certain further embodiments, the inhibitors have an IC50 value for PI4K-IIIβ of 50 μM or less, such as 10 nM or less, 6 nM or less, or even less, such as described in Tables 2-3. In certain embodiments, the inhibitors have an IC50 value for type II PI3-kinase alpha of less than 10 μM. In certain embodiments, the inhibitors have an IC50 value for type II PI3-kinase alpha of 1 μM or more, such as 10 μM or more. In certain further embodiments, more than one of the above criteria is independently satisfied by a particular compound.

In some embodiments, the anti-cancer potency of the PI4-kinase inhibitors track with anti-infective (e.g., antiviral) activity. In some cases, the enzymatic and anti-cancer activities of the subject compounds diverge. In some embodiments, the anti-cancer activity of the subject compounds depends on a combination of inhibition of both PI4KIIIα and PI4KIIIβ, or a combination of inhibition of class III PI4-kinases and/or class II PI3-kinases (especially class II PI3-kinase beta). The subject compound may have increased specificity for one isoform of these PI-kinase family members.

In certain embodiments, the PI4-kinase inhibitors have no significant effect on the viability of a normal mammalian cell, as determined by a cell cytotoxicity assay, e.g., as determined by administering a compound to primary human liver cells and determining the number of viable cells present. The compound may exhibit a % cell viability, as compared to a control (e.g., a DMSO control), of 15% or more, such as 20% or more, 30% or more, 40% or more, 50% or more, 60% or more, 70% or more, 80% or more, 90% or more, 100% or more, 120% or more, or even higher. The subject compounds may exhibit a CC₅₀ value (the concentration at which 50% of the cells remain viable) of 1 nM or higher, such as 100 nM or higher, 300 nM or higher, 1 μM or higher, 3 μM or higher, 5 nM or higher, 10 nM or higher, 20 nM or higher, 30 nM or higher, 50 nM or higher, or even higher.

In certain embodiments, the PI4-kinase inhibitors have a therapeutic index (e.g., the ratio of a compound's cytotoxicity (e.g., normal cell cytotoxicity, CC50) to bioactivity (e.g., anticancer activity, EC50—the concentration at which 50% of the cancer cells are inhibited)) that is 2 or more, such as 5 or more, such as 10 or more, such as 20 or more, 50 or more, 100 or more, 200 or more, 300 or more, 400 or more, 500 or more, or even more.

As summarized above, aspects of the disclosure include methods of inhibiting a PI4-kinase (e.g., a PI4-IIIα, and/or a PI4-IIIβ kinase) in a cell of interest. The compound (e.g., as described herein) may inhibit at least one activity of the PI4-kinase in the range of 10% to 100%, e.g., by 10% or more, 20% or more, 30% or more, 40% or more, 50% or more, 60% or more, 70% or more, 80% or more, or 90% or more. In certain assays, a PI4-kinase inhibitor may inhibit its target with an IC₅₀ (the concentration needed to inhibit 50% of the kinase activity) of 1×10⁻⁶M or less (e.g., 1×10⁻⁶M or less, 1×10⁻⁷ M or less, 1×10⁻⁸M or less, 1×10⁻⁹ M or less, 1×10⁻¹⁰ M or less, or 1×10⁻¹¹ M or less). The protocols that may be employed in determining PI-kinase activity are numerous, and include but are not limited to cell-free assays, e.g., binding assays; assays using purified enzymes, cellular assays in which a cellular phenotype is measured, e.g., gene expression assays; and in vivo assays that involve a particular animal (which, in certain embodiments may be an animal model for a condition dependent on PI-kinase activity).

In some embodiments, the subject method is an in vitro method that includes contacting a sample with a compound that specifically inhibits a target PI-kinase. In certain embodiments, the sample is suspected of containing the PI-kinase and the subject method further comprises evaluating whether the compound inhibits the PI-kinase, or a PI-kinase dependent function such as cancer cell growth. In certain embodiments, the PI-kinase is a PI4-kinase, e.g., a PI4-III kinase, such as a PI4-IIIβ kinase. In another embodiment of the subject method, the sample is known to contain the target PI-kinase.

In some embodiments, the subject method is an in vivo method that includes administering to a subject an effective amount of a compound that specifically inhibits a PI4-kinase. An “effective amount” is an amount of a compound that, when administered to an individual in one or more doses, in monotherapy or in combination therapy, is effective to inhibit a PI4-kinase by at least about 20% (20% inhibition), such as at least about 30% (30% inhibition), at least about 40% (40% inhibition), at least about 50% (50% inhibition), at least about 60% (60% inhibition), at least about 70% (70% inhibition), at least about 80% (80% inhibition), or at least about 90% (90% inhibition), compared to the PI4-kinase activity in the individual in the absence of treatment with the compound, or alternatively, compared to the PI4-kinase activity in the individual before or after treatment with the compound.

The subject may be one who has a cancer as described in FIG. 1A-1C. Cancers of interest which can be treated according to the subject methods include, but are not limited to, bladder, breast, colon, endometrial, liver, lung, non-small cell lung cancer (NSCLC), ovarian, prostate, pancreatic, melanoma and sarcoma cancer. In some embodiments, a “therapeutically effective amount” is an amount of a compound that, when administered to an individual in one or more doses, in monotherapy or in combination therapy, is effective to decrease tumor burden in the subject by at least about 20%, such as at least about 30%, at least about 40%, at least about 50%, at least about 60%, at least about 70%, at least about 80%, or at least about 90%, compared to tumor burden in the individual in the absence of treatment with the compound, or alternatively, compared to the tumor burden in the subject before or after treatment with the compound. As used herein the term “tumor burden” refers to the total mass of tumor tissue carried by a subject with cancer.

In some embodiments, a “therapeutically effective amount” is an amount of a subject compound that, when administered to an individual in one or more doses, in monotherapy or in combination therapy, is effective to reduce the dose of radiotherapy required to observe tumor shrinkage in the subject by at least about 20%, at least about 30%, at least about 40%, at least about 50%, at least about 60%, at least about 70%, at least about 80%, or at least about 90%, compared to the dose of radiotherapy required to observe tumor shrinkage in the individual in the absence of treatment with the compound.

In some embodiments, a “therapeutically effective amount” of a compound is an amount that, when administered in one or more doses to an individual having cancer, is effective to achieve a 1.5-log, a 2-log, a 2.5-log, a 3-log, a 3.5-log, a 4-log, a 4.5-log, or a 5-log reduction in tumor size.

In some embodiments, an effective amount of a compound is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of a compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of a compound is administered. In other embodiments, multiple doses are administered. Where multiple doses are administered over a period of time, the compound can be administered twice daily (bid), daily (qd), every other day (qod), every third day, three times per week (tiw), or twice per week (biw) over a period of time. For example, a compound is administered bid, qd, qod, tiw, or biw over a period of from one day to about 2 years or more. For example, a compound is administered at any of the aforementioned frequencies for one week, two weeks, one month, two months, six months, one year, or two years, or more, depending on various factors. In some embodiments, the compound may be administered orally, intravenously, subcutaneously, via inhalation, topically, or sublingually, among other routes of administration. In some embodiments, the compound is administered in combination with an inhibitor of its metabolism, such as an inhibitor of cytochrome P450 3A/4 (e.g. ritonavir or cobicistat). In some embodiments, the compound may be administered in courses wherein “drug holidays” are allowed that may last from 1-7 days.

Administration of a therapeutically effective amount of a subject compound to an individual with cancer can result in one or more of: 1) a reduction in tumor burden; 2) a reduction in the dose of radiotherapy required to effect tumor shrinkage; 3) a reduction in the spread of a cancer from one location to another in an individual; 4) a reduction of morbidity or mortality in clinical outcomes; 5) shortening the total length of treatment when combined with other anti-cancer agents; and 6) an improvement in an indicator of disease response (e.g., a reduction in one or more symptoms of cancer).

Any of a variety of methods can be used to determine whether a treatment method is effective. For example, a biological sample obtained from an individual who has been treated with a subject method can be assayed, or an imaging study may be performed.

Any of the PI4-kinase inhibitors described herein can be utilized in the subject methods of treatment. In certain instances, the PI4-kinase inhibitor is of one of formulae (Ia), (Ib), (IIa), (IIb) and (III)-(XLIX). In certain cases, the compound is one of the compounds of Table 1, 2 or 3.

In some embodiments, the compound specifically inhibits PI4IIIβ-kinase. In some embodiments, the compound modulates the activity of a cancer cell that includes an elevated expression of PI4-kinase or a factor involved in IRES-mediated translation that stimulates PI4-kinase activity (e.g. eEF1A2), or Golgi-mediated secretion. In some instances, the cancer cells include chromosome amplification of a PI4-kinase gene (such as PI4IIIβ or PI4IIIα), chromosome amplification of the eEF1A2 gene, or chromosome 1q amplification, i.e., a 1q-amplified cancer cell, which contains PI4IIIβ-kinase on the amplified segment. In some embodiments, the cancer cell has increased expression of eEF1A2 that is not a result of chromosome amplification of the eEF1A2 gene.

In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). The subject may be in need of treatment for cancer. In some instances, the subject methods include diagnosing cancer, including any one of the cancers described herein. In some embodiments, the compound is administered as a pharmaceutical preparation.

In certain embodiments, the PI4-kinase inhibitor is a modified compound that includes a label, and the method further includes detecting the label in the subject. The selection of the label depends on the means of detection. Any convenient labeling and detection systems may be used in the subject methods, see e.g., Baker, “The whole picture,” Nature, 463, 2010, p 977-980. In certain embodiments, the compound includes a fluorescent label suitable for optical detection. In certain embodiments, the compound includes a radiolabel for detection using positron emission tomography (PET) or single photon emission computed tomography (SPECT). In some cases, the compound includes a paramagnetic label suitable for tomographic detection. The subject compound may be labeled, as described above, although in some methods, the compound is unlabeled and a secondary labeling agent is used for imaging.

Co-administration with a Metabolizing Enzyme Inhibitor

In some aspects of the subject methods, the PI4-kinase inhibitors can be administered to a subject in combination with an additional or second agent, such as an agent that extends the half-life, and/or increases the plasma concentration of the PI4-kinase inhibitor that is co-administered. The additional agent can be a compound that is capable of inhibiting in situ an enzyme that is responsible for metabolizing the PI4-kinase inhibitor from an active form to a less or inactive form or derivative of the compound. In some cases, the metabolizing enzyme is a cytochrome P-450. Any convenient cytochrome P-450s can be targeted for inhibition by use of the additional agent in the subject methods. In certain cases, the cytochrome P-450 is CYP3A4.

Metabolizing enzyme inhibitors of interest include, but are not limited to, clarithromycin, cobicistat, telithromycin, nefazodone, itraconazole, ketoconazole, atazanavir, darunavir, indinavir, lopinavir, nelfinavir, ritonavir, saquinavir and tipranavir. For example, ritonavir is a potent inhibitor of CYP3A4 that itself finds use as a therapeutic HIV protease inhibitor. In some cases, the metabolizing enzyme inhibitor is co-administered at a dose effective to inhibit the metabolizing enzyme action on the PI4-kinase inhibitor, but which is a subtherapeutic dose relative to its therapeutic application, e.g., in treating HIV.

The terms “co-administration” and “in combination with” include the administration of two or more agents either simultaneously, concurrently or sequentially within no specific time limits. In one embodiment, the agents are present in the cell or in the subject's body at the same time or exert their biological or therapeutic effect at the same time. In one embodiment, the agents are in the same composition or unit dosage form. In other embodiments, the agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second agent. Routes of administration of the two agents may vary, where representative routes of administration are described in greater detail below. A person of ordinary skill in the art would have no difficulty determining the appropriate timing, sequence and dosages of administration for a PI4-kinase inhibitor and the additional agent.

### Combination Therapies

The PI4-kinase inhibitors can be administered to a subject alone or in combination with an additional, i.e., second, active agent. Combination therapeutic methods where the PI4-kinase inhibitors may be used in combination with a second active agent or an additional therapy, e.g., radiation therapy. The terms “agent,” “compound,” and “drug” are used interchangeably herein. For example, PI4-kinase inhibitors can be administered alone or in conjunction with one or more other drugs, such as drugs employed in the treatment of diseases of interest, including but not limited to, immunomodulatory diseases and conditions and cancer. In some embodiments, the subject method further includes coadministering concomitantly or in sequence a second agent, e.g., a small molecule, a chemotherapeutic, an antibody, an antibody fragment, an antibody-drug conjugate, an aptamer, a protein, or a checkpoint inhibitor. In some embodiments, the method further includes performing radiation therapy on the subject.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug or additional therapy with a pharmaceutical composition of the present disclosure means administration of the compound and second agent or additional therapy at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administration are described in greater detail below. A person of ordinary skill in the art would have no difficulty determining the appropriate timing, sequence and dosages of administration for particular drugs or therapies and compounds of the present disclosure.

In some embodiments, the compounds (e.g., a PI4-kinase inhibitor and the at least one additional compound or therapy) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

Also provided are pharmaceutical preparations of the PI4-kinase inhibitor and the second active agent. In pharmaceutical dosage forms, the compounds may be administered in the form of their pharmaceutically acceptable salts, or they may also be used alone or in appropriate association, as well as in combination, with other pharmaceutically active compounds.

In conjunction with any of the subject methods, the PI4-kinase inhibitors (e.g., as described herein) (or pharmaceutical compositions comprising such compounds) can be administered in combination with another drug designed to reduce or prevent inflammation, treat or prevent chronic inflammation or fibrosis, or treat cancer. In each case, the PI4-kinase inhibitor can be administered prior to, at the same time as, or after the administration of the other drug. In certain cases, the cancer is selected from adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioma, glioblastomas, melanoma and various head and neck tumors.

For the treatment of cancer, the PI4-kinase inhibitors can be administered in combination with a chemotherapeutic agent selected from the group consisting of alkylating agents, nitrosoureas, antimetabolites, antitumor antibiotics, plant (vinca) alkaloids, steroid hormones, taxanes, nucleoside analogs, steroids, anthracyclines, thyroid hormone replacement drugs, thymidylate-targeted drugs, Chimeric Antigen Receptor/T cell therapies, Chimeric Antigen Receptor/NK cell therapies, apoptosis regulator inhibitors (e.g., B cell CLL/lymphoma 2 (BCL-2) BCL-2-like 1 (BCL-XL) inhibitors), CARP-1/CCAR1 (Cell division cycle and apoptosis regulator 1) inhibitors, colony-stimulating factor-1 receptor (CSF1R) inhibitors, CD47 inhibitors, cancer vaccine (e.g., a Th17-inducing dendritic cell vaccine, or a genetically modified tyrosinase such as Oncept®) and other cell therapies.

Specific chemotherapeutic agents of interest include, but are not limited to, Gemcitabine, Docetaxel, Bleomycin, Erlotinib, Gefitinib, Lapatinib, Imatinib, Dasatinib, Nilotinib, Bosutinib, Crizotinib, Ceritinib, Trametinib, Bevacizumab, Sunitinib, Sorafenib, Trastuzumab, Ado-trastuzumab emtansine, Rituximab, Ipilimumab, Rapamycin, Temsirolimus, Everolimus, Methotrexate, Doxorubicin, Abraxane, Folfirinox, Cisplatin, Carboplatin, 5-fluorouracil, Teysumo, Paclitaxel, Prednisone, Levothyroxine, Pemetrexed, navitoclax, and ABT-199. Peptidic compounds can also be used. Cancer chemotherapeutic agents of interest include, but are not limited to, dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD).

In some embodiments, the PI4-kinase inhibitors can be administered in combination with a chemotherapeutic agent to treat cancer. In certain cases, the chemotherapeutic agent is Gemcitabine. In some cases, the chemotherapeutic agent is Docetaxel. In some cases, the chemotherapeutic agent is Abraxane.

For the treatment of cancer (e.g., solid tumor cancer), the PI4-kinase inhibitors can be administered in combination an immunotherapeutic agent. An immunotherapeutic agent is any convenient agent that finds use in the treatment of disease by inducing, enhancing, or suppressing an immune response. In some cases, the immunotherapeutic agent is an immune checkpoint inhibitor. Any convenient checkpoint inhibitors can be utilized, including but not limited to, cytotoxic T-lymphocyte-associated antigen 4 (CTLA-4) inhibitors, programmed death 1 (PD-1) inhibitors and PD-L1 inhibitors. In certain instances, the checkpoint inhibitor is selected from a cytotoxic T-lymphocyte-associated antigen 4 (CTLA-4) inhibitor, a programmed death 1 (PD-1) inhibitor and a PD-L1 inhibitor. Exemplary checkpoint inhibitors of interest include, but are not limited to, ipilimumab, pembrolizumab and nivolumab. In certain embodiments, for treatment of cancer and/or inflammatory disease, the immunomodulatory polypeptide(s) can be administered in combination with a colony-stimulating factor-1 receptor (CSF1R) inhibitor. CSF1R inhibitors of interest include, but are not limited to, emactuzumab.

Any convenient cancer vaccine therapies and agents can be used in combination with the PI4-kinase inhibitors, compositions and methods. For treatment of cancer, e.g., ovarian cancer, the PI4-kinase inhibitors can be administered in combination with a vaccination therapy, e.g., a dendritic cell (DC) vaccination agent that promotes Th1/Th17 immunity Th17 cell infiltration correlates with markedly prolonged overall survival among ovarian cancer patients. In some cases, the ENPP1 inhibitor compound finds use as adjuvant treatment in combination with Th17-inducing vaccination.

Also of interest are agents that are CARP-1/CCAR1 (Cell division cycle and apoptosis regulator 1) inhibitors, including but not limited to those described by Rishi et al., Journal of Biomedical Nanotechnology, Volume 11, Number 9, September 2015, pp. 1608-1627(20), and CD47 inhibitors, including, but not limited to, anti-CD47 antibody agents such as Hu5F9-G4.

In certain instances, the combination provides an enhanced effect relative to either component alone; in some cases, the combination provides a supra-additive or synergistic effect relative to the combined or additive effects of the components. A variety of combinations of the subject compounds and the chemotherapeutic agent may be employed, used either sequentially or simultaneously. For multiple dosages, the two agents may directly alternate, or two or more doses of one agent may be alternated with a single dose of the other agent, for example. Simultaneous administration of both agents may also be alternated or otherwise interspersed with dosages of the individual agents. In some cases, the time between dosages may be for a period from about 1-6 hours, to about 6-12 hours, to about 12-24 hours, to about 1-2 days, to about 1-2 week or longer following the initiation of treatment.

### Utility

The compounds and methods of the invention, e.g., as described herein, find use in a variety of applications. Applications of interest include, but are not limited to: research applications and therapeutic applications. Methods of the invention find use in a variety of different applications including any convenient application where inhibition of a PI4-kinase is desired.

The subject compounds and methods find use in a variety of research applications. The subject compounds and methods may be used in the optimization of the bioavailability and metabolic stability of compounds.

The subject compounds and methods find use in a variety of therapeutic applications. Therapeutic applications of interest include those applications in cancer treatment. As such, the subject compounds find use in the treatment of a variety of different conditions in which the inhibition and/or treatment of cancer in the host is desired. For example, the subject compounds and methods may find use in treating a solid tumor cancer (e.g., as described herein).

### Pharmaceutical Compositions

The herein-discussed compounds can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the PI4-kinase inhibitor is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In some embodiments, the PI4-kinase inhibitor and a second active agent (e.g., as described herein), e.g. a small molecule, a chemotherapeutic, an antibody, an antibody fragment, an antibody-drug conjugate, an aptamer, or a protein, etc. are administered to individuals in a formulation (e.g., in the same or in separate formulations) with a pharmaceutically acceptable excipient(s). In some embodiments, the second active agent is a checkpoint inhibitor, e.g., a cytotoxic T-lymphocyte-associated antigen 4 (CTLA-4) inhibitor, a programmed death 1 (PD-1) inhibitor, or a PD-L1 inhibitor.

In another aspect, a pharmaceutical composition is provided, comprising, or consisting essentially of, a PI4-kinase inhibitor, or a pharmaceutically acceptable salt, isomer, tautomer or prodrug thereof, and further comprising one or more additional anti-cancer agents of interest. Any convenient anti-cancer agents can be utilized in the subject methods in conjunction with the subject compounds. The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used. Examples of suitable solid carriers include lactose, sucrose, gelatin, agar and bulk powders.

Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

Although such may not be necessary, compounds and agents described herein can optionally be targeted to the site of cancer, using any known targeting means. The compounds of the disclosure may be formulated with a wide variety of compounds that have been demonstrated to target compounds to the site of cancer. The terms “targeting to the site of cancer” and “cancer targeted” refer to targeting of a compound to a site of cancer, such that at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, of the compound administered to the subject enters the site of cancer.

### Subjects Amenable to Treatment Using the Compounds of the Disclosure

Individuals who have been clinically diagnosed as having cancer are suitable for treatment with the methods of the present disclosure. In particular embodiments of interest, individuals of interest for treatment according to the disclosure have detectable cancer. Any convenient methods may be used to determine whether subjects who have cancer are suitable for treatment using the subject methods. The effectiveness of the anti-cancer treatment may be determined using any convenient method. For example, whether a subject method is effective in treating cancer can be determined by measuring amelioration of one or more symptoms, decrease in tumor or metastasis size on imaging, or by measuring cancer cells in a biological sample of the subject being treated.

Notwithstanding the appended claims, the disclosure set forth herein is also described by the following clauses.

Clause 1. An anti-cancer compound having the formula (XXI)

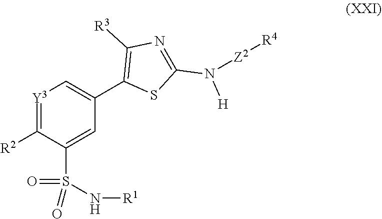

wherein: R² is an alkoxy (e.g., methoxy) or a substituted alkoxy; R³ is hydrogen, a lower alkyl (e.g., methyl) or a substituted lower alkyl; Y³ is CH or N; Z² is absent, CO or SO₂ (e.g., Z² is absent or CO); R¹ is an aryl, a substituted aryl (e.g., a substituted phenyl), a heteroaryl, a substituted heteroaryl, (e.g., a substituted pyridyl), an alkyl, a substituted alkyl, a cycloalkyl, a substituted cycloalkyl (e.g., a substituted cyclohexyl), a heterocycle (e.g., a tetrahydropyran or a piperidinyl) or a substituted heterocycle; and R⁴ is selected from alkyl, substituted alkyl, cycloalkyl, substituted cycloalkyl, alkyl-cycloalkyl, substituted alkyl-cycloalkyl, aryl, substituted aryl, heterocycle, substituted heterocycle, alkyl-heterocycle (e.g., —CH₂-(4-tetrahydropyran)) and substituted alkyl-heterocycle; or a prodrug thereof, or a pharmaceutically acceptable salt thereof.
2. The compound of clause 1, having the formula (XXII):

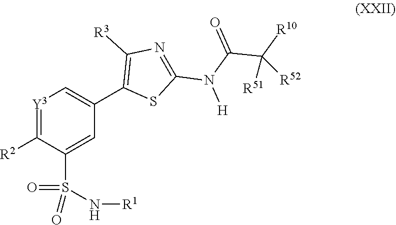

wherein: R¹⁰ is selected from cycloalkyl, substituted cycloalkyl, heterocycle (e.g., 4-tetrahydropyran) and substituted heterocycle; and R⁵¹ and R⁵² are independently selected from H, halogen (e.g., fluoro), alkyl (e.g., lower alkyl) and substituted alkyl.
3. The compound of clause 7 having the formula (XXIII):

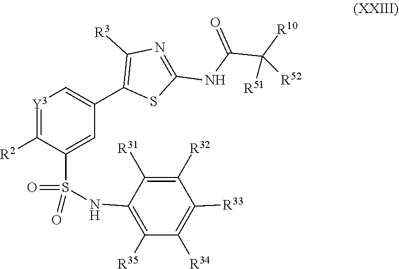

wherein: R³¹-R³⁵ are independently selected from hydrogen, halogen (e.g., fluoro), alkyl, substituted alkyl, hydroxy, alkoxy, substituted alkoxy, acyl, substituted acyl and —CO₂R, wherein R is H, alkyl or substituted alkyl.
4. The compound of clause 2 or 3, having the formula (XXIV) or formula (XXV):

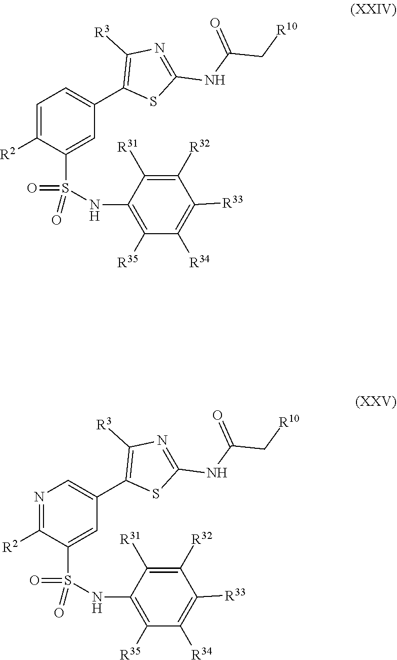

wherein one and only one of R³³ and R³⁴ is hydroxy.
5. The compound of clause 4, having any one of the formulae (XXVI)-(XXVIII):

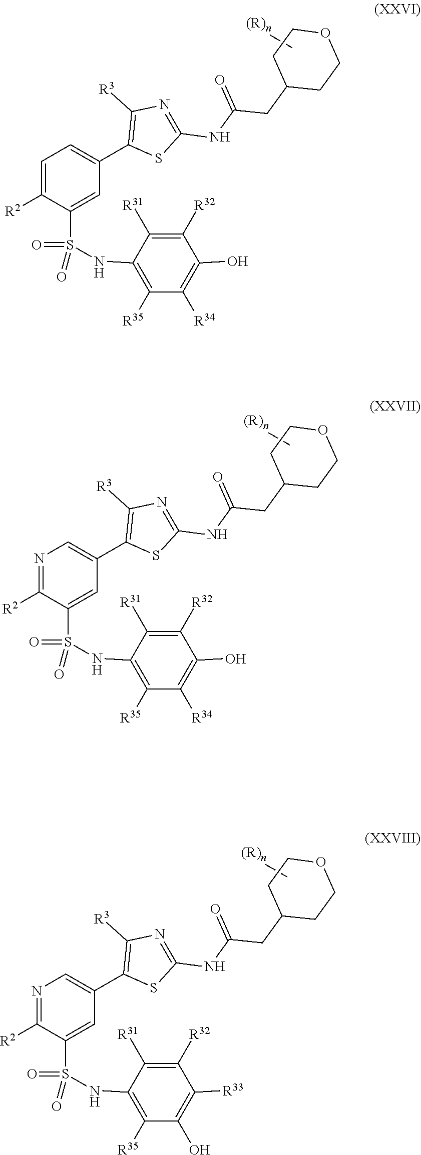

wherein: R³¹, R³², R³⁴ and R³⁵ are independently selected from hydrogen and halogen (e.g., fluoro); and (R)ₙ is one or more optional substituents each independently selected from alkyl, substituted alkyl, hydroxyl, alkoxy, substituted alkoxy, halogen (e.g., fluoro or chloro), and CO₂R″ wherein R″ is hydrogen, alkyl or substituted alkyl.
6. The compound of clause 5, having one of the following structures:

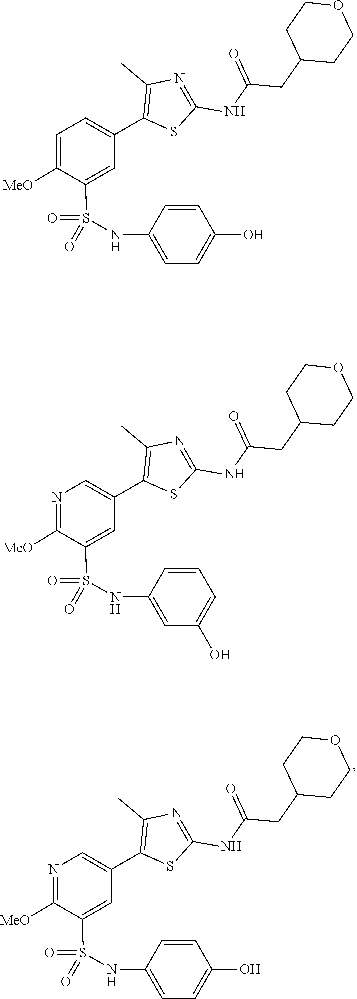 [SMILES: COc1ccc(-c2sc(NC(=O)CC3CCOCC3)nc2C)cc1S(=O)(=O)Nc1ccc(O)cc1 ; COc1ncc(-c2sc(NC(=O)CC3CCOCC3)nc2C)cc1S(=O)(=O)Nc1cccc(O)c1 ; COc1ncc(-c2sc(NC(=O)CC3CCOCC3)nc2C)cc1S(=O)(=O)Nc1ccc(O)cc1]

or a prodrug thereof, or a pharmaceutically acceptable salt thereof.
7. The compound of clause 3, having formula (XIX):

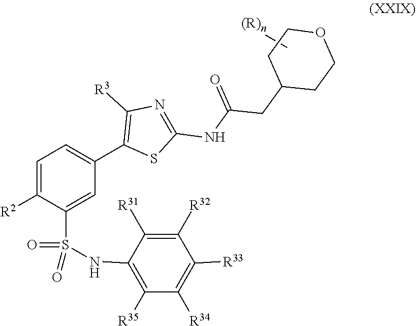

wherein: R³¹-R³⁵ are independently selected from hydrogen and halogen (e.g., fluoro or chloro), wherein 0, 1 or 2 of R³¹-R³⁵ are halogen; and (R)ₙ is one or more optional substituents each independently selected from alkyl, substituted alkyl, hydroxyl, alkoxy, substituted alkoxy, halogen (e.g., fluoro or chloro), and CO₂R″ wherein R″ is hydrogen, alkyl or substituted alkyl.
8. The compound of clause 2, having formula (XXX):

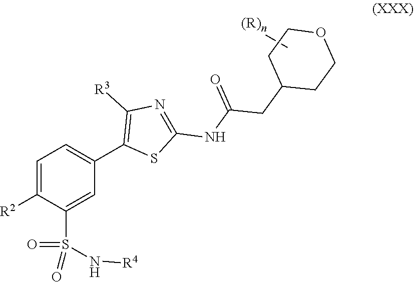

wherein: R⁴ is a lower alkyl or a substituted lower alkyl (e.g., an isopropyl); and (R)ₙ is one or more optional substituents each independently selected from alkyl, substituted alkyl, hydroxyl, alkoxy, substituted alkoxy, halogen (e.g., fluoro or chloro), and CO₂R″ wherein R″ is hydrogen, alkyl or substituted alkyl.
9. The compound of clause 2, having the formula (XXXI):

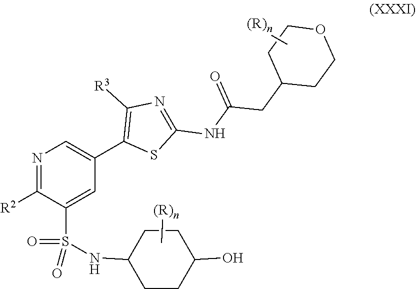

wherein: each (R)ₙ is one or more optional substituents each independently selected from alkyl, substituted alkyl, hydroxyl, alkoxy, substituted alkoxy, halogen (e.g., fluoro or chloro) and CO₂R″ wherein R″ is hydrogen, alkyl or substituted alkyl.
10. The compound of clause 9, having the structure:

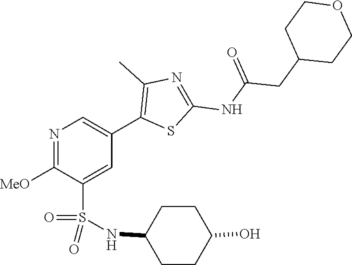

or a prodrug thereof, or a pharmaceutically acceptable salt thereof.
11. The compound of clause 3, having the formula (XXXII):

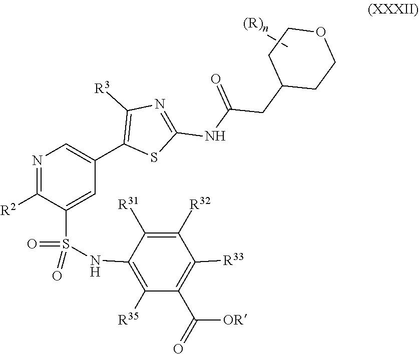

wherein R³¹-R³³ and R³⁵ are independently selected from hydrogen and halogen; (R)ₙ is one or more optional substituents each independently selected from alkyl, substituted alkyl, hydroxyl, alkoxy, substituted alkoxy, halogen and CO₂R″ wherein R″ is hydrogen, alkyl or substituted alkyl; and R′ is H, alkyl or substituted alkyl.
12. The compound of clause 11, having the structure:

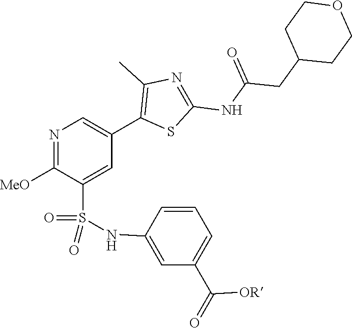

wherein R′ is H or a lower alkyl; or a prodrug thereof, or a pharmaceutically acceptable salt thereof.
13. The compound of clause 1, having the formula (XXXIII) or formula (XXXIV):

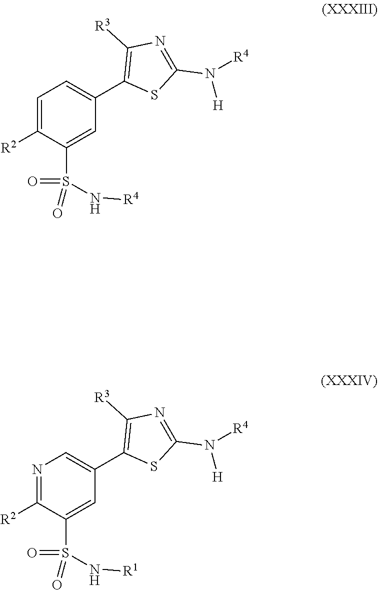

wherein: R² is an alkoxy (e.g., methoxy) or a substituted alkoxy; R³ is hydrogen, a lower alkyl (e.g., methyl) or a substituted lower alkyl; R¹ is an aryl, a substituted aryl, (e.g., a substituted phenyl), a heteroaryl, a substituted heteroaryl, (e.g., a substituted pyridyl), a cycloalkyl, a substituted cycloalkyl (e.g., a substituted cyclohexyl), a heterocycle (e.g., a tetrahydropyran or a piperidinyl) or a substituted heterocycle; and R⁴ is selected from cycloalkyl, substituted cycloalkyl, alkyl-cycloalkyl, substituted alkyl-cycloalkyl, heterocycle, substituted heterocycle, alkyl-heterocycle (e.g., —CH₂-(4-tetrahydropyran)) and substituted alkyl-heterocycle.
14. The compound of clause 13, having one of the formula (XXXV) and formula (XXXVI):

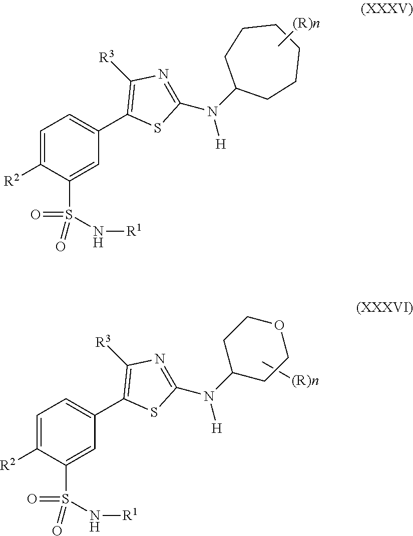

wherein: (R)ₙ is one or more optional substituents each independently selected from alkyl, substituted alkyl, hydroxyl, alkoxy, substituted alkoxy, halogen and CO₂R″ wherein R″ is hydrogen, alkyl or substituted alkyl.
15. The compound of clause 14, having one of the formula (XXXVII) and formula (XXXVIII):

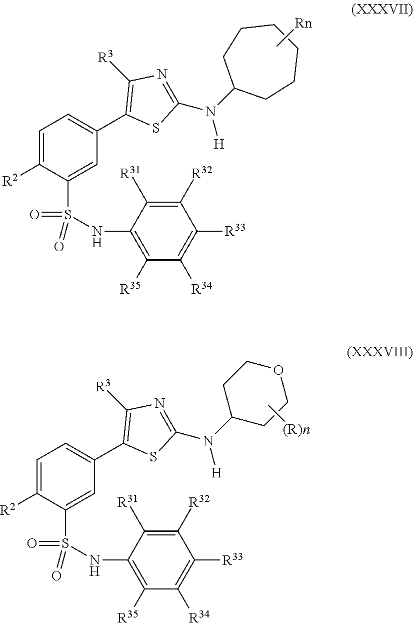

wherein: R³¹-R³⁵ are independently selected from hydrogen, halogen (e.g., fluoro), alkyl, substituted alkyl, hydroxy, alkoxy, substituted alkoxy, acyl, substituted acyl and —CO₂R, wherein R is H, alkyl or substituted alkyl.
16. The compound of clause 15, wherein R³¹-R³⁵ are independently selected from hydrogen, methyl, halogen (e.g., fluoro or chloro) and hydroxy.
17. The compound of clause 15 or 16, wherein R³¹ and R³⁵ are independently lower alkyl or substituted lower alkyl (e.g., methyl).
18. The compound of clause 17, having the structure:

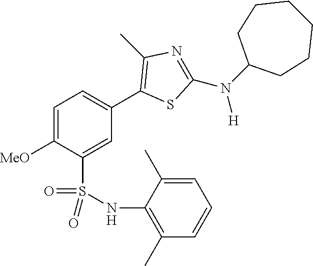 [SMILES: COc1ccc(-c2sc(NC3CCCCCC3)nc2C)cc1S(=O)(=O)Nc1c(C)cccc1C]

or a prodrug thereof, or a pharmaceutically acceptable salt thereof.
19. The compound of clause 1, having the formula (XXXIX):

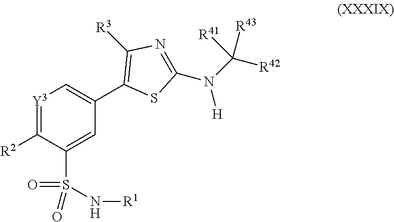

wherein R⁴¹ and R⁴³ are independently hydrogen, a lower alkyl or a substituted lower alkyl (e.g., methyl); and R⁴² is selected from cycloalkyl, substituted cycloalkyl, alkyl-cycloalkyl, substituted alkyl-cycloalkyl, heterocycle, substituted heterocycle, alkyl-heterocycle (e.g., —CH₂-(4-tetrahydropyran)) and substituted alkyl-heterocycle.
20. The compound of clause 19, having the formula (XL) or (XLI):

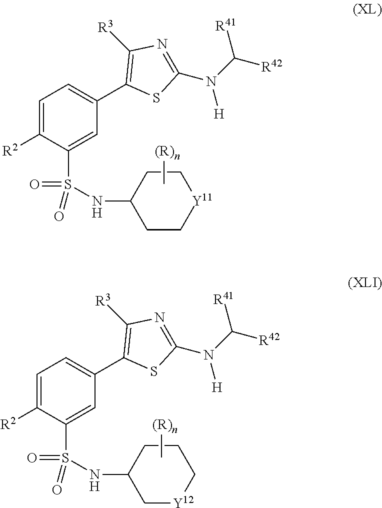

wherein: Y¹¹ and Y¹² are selected from CR″₂, NR″ and O, wherein each R″ is independently H, R, an acyl or a substituted acyl; each R is independently H, an alkyl, a substituted alkyl, an alkoxy or a halogen (e.g., a fluoro); and n is 0, 1, 2, 3 or 4.
21. The compound of clause 20, having the formula (XLII) or (XLIII):

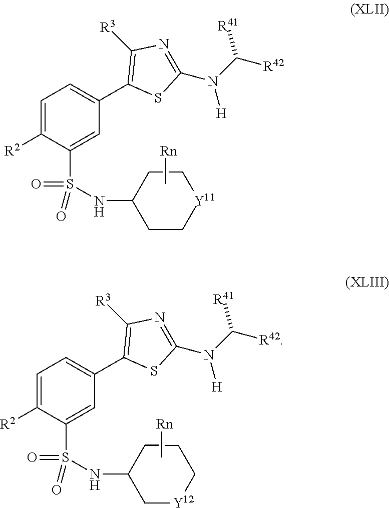

22. The compound of clause 21, wherein Y¹¹ and Y¹² are each NH.
23. The compound of clause 21, wherein n is 0.
24. The compound of clause 21, wherein (R)ₙ is 4-CO₂R′, wherein R′ is hydrogen or lower alkyl (e.g., ethyl).
25. The compound of clause 20, wherein: R⁴¹ is methyl; and R⁴² is selected from cyclohexyl, substituted cyclohexyl, —CH₂-cyclohexyl and substituted —CH₂-cyclohexyl.
26. The compound of clause 25, wherein the compound has one of the following structures:

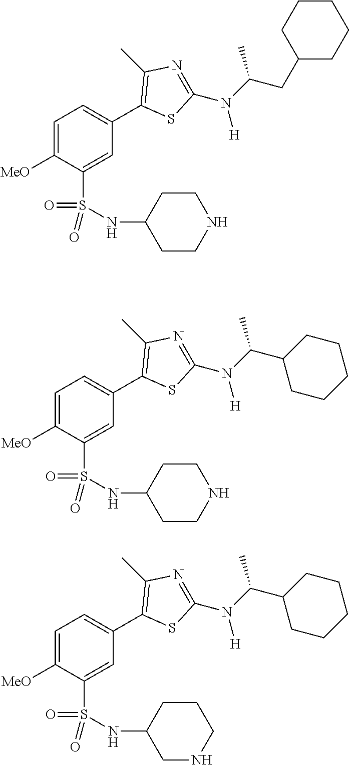 [SMILES: COc1ccc(-c2sc(N[C@H](C)CC3CCCCC3)nc2C)cc1S(=O)(=O)NC1CCNCC1 ; COc1ccc(-c2sc(N[C@H](C)C3CCCCC3)nc2C)cc1S(=O)(=O)NC1CCNCC1 ; COc1ccc(-c2sc(N[C@H](C)C3CCCCC3)nc2C)cc1S(=O)(=O)NC1CCCNC1]

or a prodrug thereof, or a pharmaceutically acceptable salt thereof.
27. The compound of clause 19, having one of the formulae (XLIVa)-(XLIVd) and (XLVa)-(XLVd):

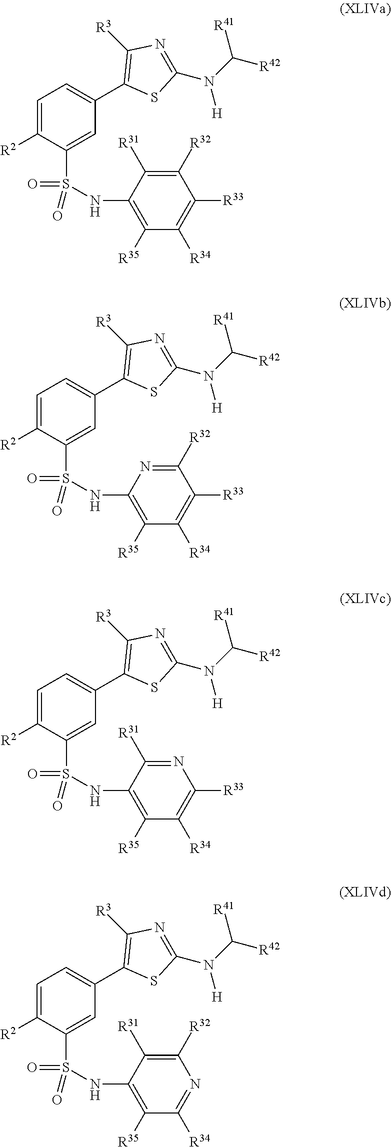

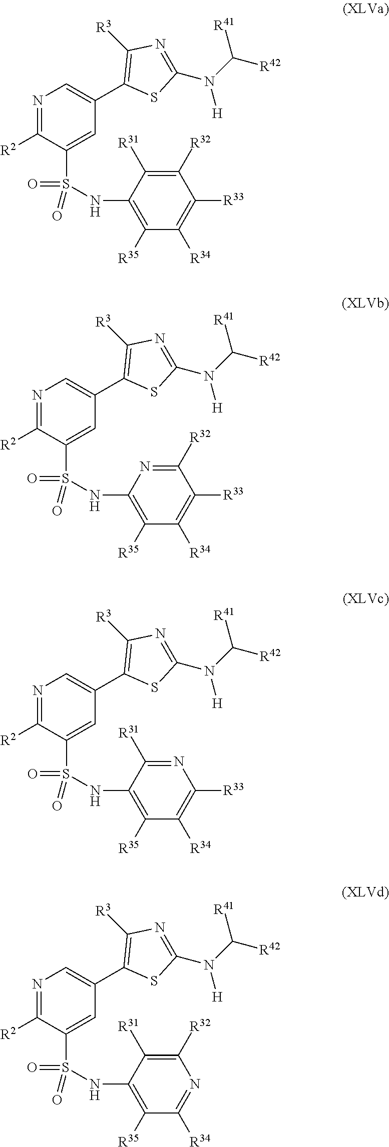

wherein: R³¹-R³⁵ are independently selected from hydrogen, halogen (e.g., fluoro or chloro), alkyl, substituted alkyl, hydroxy, alkoxy, substituted alkoxy, acyl, substituted acyl and —CO₂R, wherein R is H, alkyl or substituted alkyl.
28. The compound of clause 27, having one of the formulae (XLVIa)-(XLVId) and (XLVIIa)-(XLVIId):

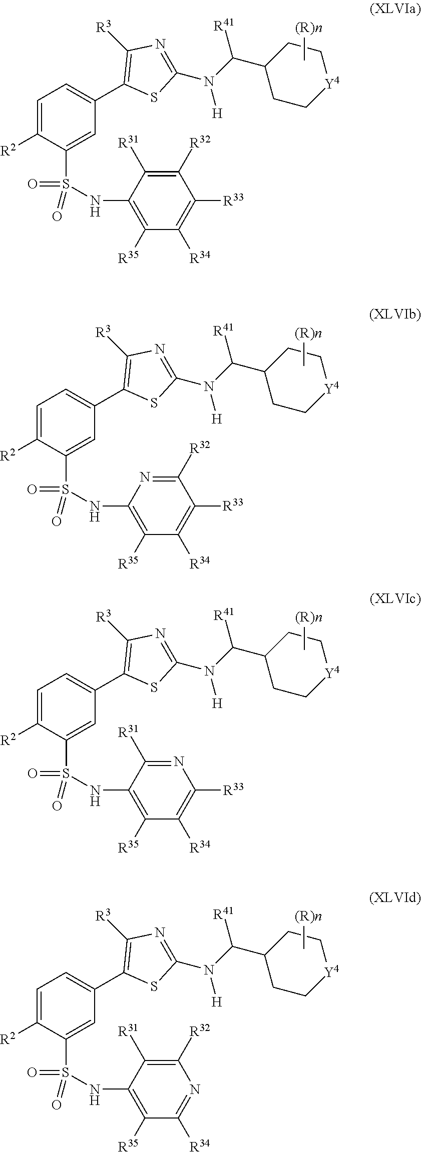

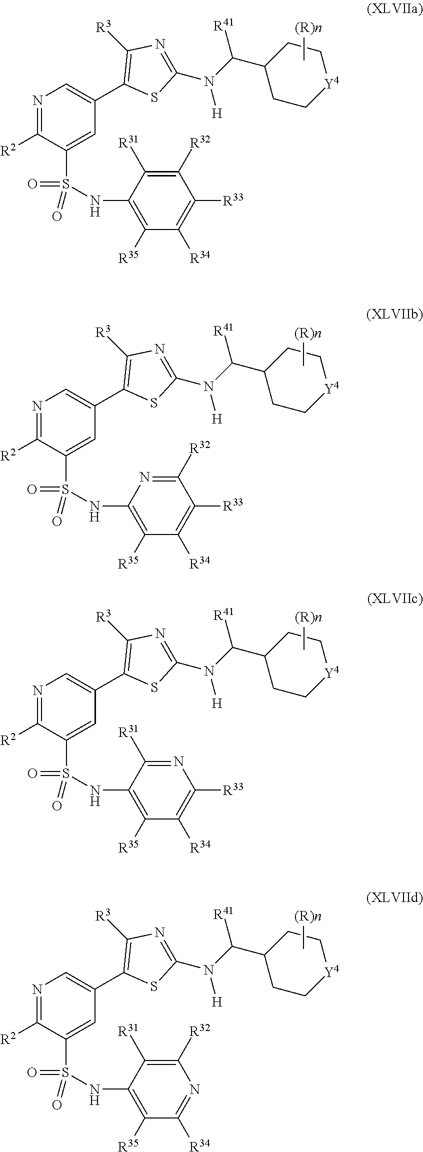

wherein: (R)ₙ is one or more optional substituents (i.e., n is 0, 1, 2, 3, 4 or 5) each independently selected from alkyl, substituted alkyl, hydroxyl, alkoxy, substituted alkoxy, halogen and CO₂R″ wherein R″ is hydrogen, alkyl or substituted alkyl; Y⁴ is CH, CR or O; and R⁴¹ is H, lower alkyl or substituted lower alkyl.
29. The compound of clause 28, wherein R³¹-R³⁵ are independently selected from hydrogen, methyl, halogen and hydroxy, R⁴¹ is lower alkyl and Y⁴ is O.
30. The compound of clause 1, having one of the formula (XLVIII) or formula (XLIX):

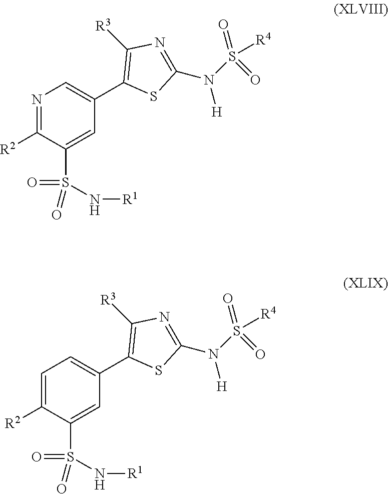

wherein R¹ is selected from aryl, substituted aryl, heteroaryl and substituted heteroaryl.
31. The compound of clause 30, wherein R⁴ is methyl, isopropyl, cyclohexyl, substituted cyclohexyl, phenyl, substituted phenyl, benzyl, substituted benzyl or —CH₂-4-tetrahydropyran.
32. The compound of clause 30 or 31, wherein R¹ is phenyl, a substituted phenyl, a pyridyl or a substituted pyridyl.
33. The compound of any one of clauses 1-32, wherein the compound is selected from the compounds of Table 2, 4, 5 and 6, or a prodrug thereof, or a pharmaceutically acceptable salt thereof.
34. The compound of any one of clauses 1-32, wherein the compound is selected from the compounds of Table 1, or a prodrug thereof, or a pharmaceutically acceptable salt thereof.
35. An anti-cancer pharmaceutical composition comprising: the compound of any one of clauses 1-34; and a pharmaceutically acceptable excipient.
36. A method of inhibiting a PI4-kinase in a cancer cell, the method comprising contacting a sample comprising the PI4-kinase with PI4-kinase inhibitor.
37. The method of clause 37, wherein the cancer cell is derived from an epithelial cell.
37. The method of clause 36, wherein the PI4-kinase inhibitor is a 5-aryl or heteroaryl-thiazole compound.
38. The method of clause 37, wherein the PI4-kinase inhibitor is a substituted 2-amino-5-phenylthiazole or a substituted 2-amino-5-pyridylthiazole compound.
39. The method of clause 38, wherein the PI4-kinase inhibitor is a compound of any one of clauses 1-34.
40. The method of any one of clauses 36-39, wherein the PI4-kinase is a PI4-III kinase.
41. The method of clause 40, wherein the PI4411 kinase is a PI4KIIIα- or PI4KIIIβ-kinase.
42. A method of treating a subject for cancer, the method comprising administering to the subject a pharmaceutical composition comprising an effective amount of a PI4-kinase inhibitor.
43. The method of clause 42, wherein the PI4-kinase inhibitor is a 5-aryl or heteroaryl-thiazole compound.
44. The method of clause 43, wherein the PI4-kinase inhibitor is a substituted 2-amino-5-phenylthiazole or a substituted 2-amino-5-pyridylthiazole compound.
45. The method of clause 43, wherein the compound of any one of clauses 1-34 or the pharmaceutical composition of clause 35, or a pharmaceutically acceptable salt thereof.
46. The method of any one of clauses 42-45, wherein the compound is one of the compounds of Table 1 or Table 2.
47. The method of any one of clauses 42-46, wherein the PI4-kinase is a PI4-III kinase.
48. The method of clause 47, wherein the PI4-III kinase is a PI4KIIIα- or PI4KIIIβ-kinase.

### EXAMPLES

The following examples are put forth so as to provide those of ordinary skill in the art with a complete disclosure and description of how to make and use embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present disclosure. All such modifications are intended to be within the scope of the claims appended hereto.

### Example 1: Synthesis and PI4 Kinase Activity of Selected Compounds

The preparation of exemplary 5-aryl or heteroaryl-thiazole compounds and their activity as PI4 kinase inhibitors are described by Glenn et al. in the experimental section of WO 2017/147526, the disclosure of which is herein incorporated by reference.

PI-kinase assay: Compounds are tested in C.1.1. PI kinase assays as described by Shokat et al., “A membrane capture assay for lipid kinase activity.” Nat. Protoc. 2007; 2(10):2459-66. Selected compounds of Table 1 and 2 were prepared and tested for inhibition activity in a variety of kinase assays.

TABLE 3
Comparison of PI4K and PI3K Kinase activity of select compounds

| Com- | | | PI3K | PI3K | | |
|---|---|---|---|---|---|---|
| pound | PI4KIIIα | PI4KIIIβ | p110a | p110g | PI3KC2B | PI3KC2G |
| S-1 | | A | D | D | D | A |
| S-8 | | B | | | | |
| S-9 | | A | | | | |
| S-25 | | B | | | | |
| S-28 | | C | | | | |
| S-29 | | B | | | | |
| S-37 | | B | | | | |
| S-41 | | A | | | | |
| S-43 | | B | | | | |
| S-46 | | B | | | | |
| S-49 | | A | | | | |
| S-50 | | B | | | | |
| S-52 | | B | | | | |
| S-53 | | B | | | | |
| S-54 | | C | | | | |
| S-78 | | C | | | | |
| S-81 | | B | | | | |
| S-91 | | B | | | | |
| S-92 | | C | | | | |
| S-94 | | C | | | | |
| S-95 | | B | | | | |
| S-98 | D | A | D | D | | |
| S-100 | D | B | | D | | C |
| S-101 | | C | | | | |
| S-102 | | B | | | | |
| S-103 | | C | | | | |
| S-107 | | C | | | | |
| S-109 | | B | | | | |
| S-111 | | B | | | | |
| S-115 | | A | | | | |
| S-118 | | B | | | | |
| S-119 | | B | | | | |
| S-121 | | C | | | | |
| S-122 | | B | | | | |
| S-123 | | A | | | | |
| S-127 | D | B | | D | | D |
| S-128 | D | B | | D | | D |
| S-129 | D | B | | D | | D |
| S-131 | D | B | | | | D |
| S-133 | | A | | | | |
| S-134 | | A | | | | |
| S-135 | | A | | | | |
| S-136 | | A | | | | |
| S-138 | | B | | | | |
| S-145 | B | A | D | D | | |
| S-146 | | B | | | | |
| S-147 | B | A | D | D | | |
| S-148 | B | A | D | D | | |
| S-149 | | A | | | | |
| S-150 | | B | | | | |
| S-151 | | A | | | | |
| S-152 | | A | | | | |
| S-153 | | A | | | | |
| S-154 | | B | | | | |
| S-155 | | B | | | | |
| S-156 | | A | | | | |
| S-157 | | B | | | | |
| S-159 | | B | | | | |
| S-160 | | A | | D | D | B |
| S-161 | | B | | | | |
| S-185 | | A | | | | |
| S-166 | | A | | | | |
| S-167 | | A | | | | |
| S-169 | A | A | D | D | | D |
| S-170 | A | A | D | D | | D |
| S-173 | | A | | | | |
| S-178 | | B | | | | |
| S-183 | | A | | | | |
| S-184 | | B | | | | |
| S-185 | | C | | | | |
| S-188 | | A | | | | |
| S-189 | | A | | | | |
| S-192 | | B | | | | |
| S-193 | | A | | | D | D |
| S-194 | | B | | | | |
| S-195 | | B | | | | |
| S-196 | | B | | | | |
| S-197 | | A | | | | |
| S-198 | | B | | | | |
| S-199 | | A | | | | |
| S-200 | | B | | | | |
| S-201 | | A | | | | |
| S-202 | | A | | | | |
| S-203 | | A | | | | |
| S-204 | | B | | | | |
| S-205 | | B | | | | |
| S-206 | | C | | | | |
| S-207 | | A | | | | |
| S-208 | | B | | | | |
| S-209 | | B | | | | |
| S-210 | | C | | | | |
| S-214 | D | B | | D | | D |
| S-215 | C | A | | D | D | C |
| S-216 | | A | | D | D | C |
| S-218 | | A | | | | |
| S-219 | | A | | | | |
| S-220 | | A | | | | |
| S-221 | | A | | | | |
| S-222 | | A | | | | |
| S-223 | | A | | | | |
| S-226 | | A | | | | |
| S-227 | | A | D | D | D | C |
| S-228 | | B | | | | |
| S-229 | | B | | | | |
| S-230 | | A | | | | |
| S-231 | | A | | D | D | B |
| S-234 | | B | | | | |
| S-235 | | B | | | | |
| S-253 | | B | | | | |
| S-258 | | C | | | | |
| S-259 | A | A | C | C | | C |
| S-260 | A | A | D | D | | D |
| S-261 | A | A | B | B | | B |
| S-262 | A | A | D | C | | B |
| S-263 | A | A | C | C | | B |
| S-265 | A | A | D | D | | C |

A = <100 nM; B = 100 nM-1 uM; C = 1-10 uM; D = >10 uM

Exemplary 5-aryl-thiazole PI4KIIIβ inhibitor compounds including IN-9, IN-10 and compounds A, B and C were obtained and tested for anti-cancer activity as described below. PI4KIIIβ inhibitors IN-9 and IN-10 are described by Rutaganira et al. (J Med Chem. 2016 Mar. 10; 59(5):1830-9) and are commercially available.

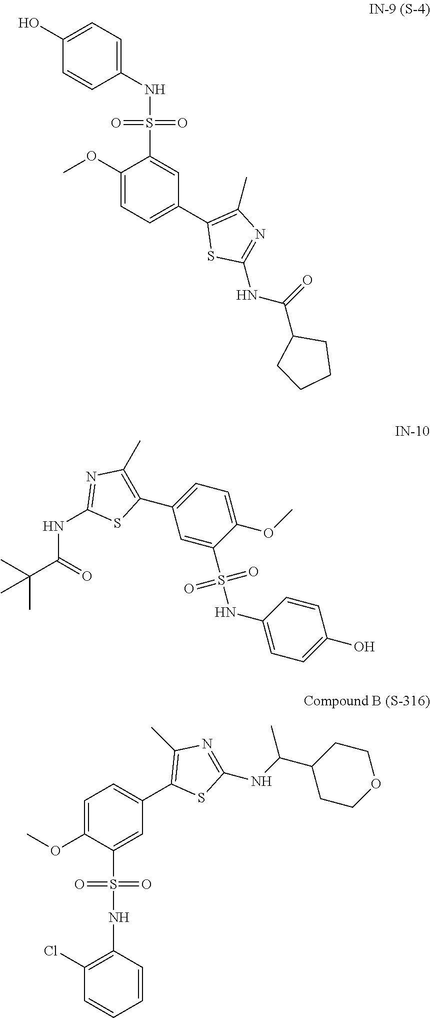 [SMILES: COc1ccc(-c2sc(NC(=O)C3CCCC3)nc2C)cc1S(=O)(=O)Nc1ccc(O)cc1 ; COc1ccc(-c2sc(NC(=O)C(C)(C)C)nc2C)cc1S(=O)(=O)Nc1ccc(O)cc1 ; COc1ccc(-c2sc(NC(C)C3CCOCC3)nc2C)cc1S(=O)(=O)Nc1ccccc1Cl]

PI4KIIIbeta-IN-9 is a PI4KIIIβ inhibitor with an IC50 of 7 nM. PI4KIIIbeta-IN-9 also inhibits PI3Kδ and PI3Kγ with IC50s of 152 nM and 1046 nM, respectively. PI4KIIIbeta-IN-10 is a PI4KIIIβ inhibitor with very minor off-target inhibition of PI4KIIIβ related lipid kinases. PI4KIIIbeta-IN-10 shows weak inhibition of PI3KC2γ (IC50˜1 μM), PI3Kα (˜10 μM), and PI4KIIIα (˜3 μM), and <20% inhibition at concentrations up to 20 μM for PI4K2α, PI4K2β, and PI3Kβ.

### Example 2: Anti-Cancer Activity of Exemplary Compounds

### General Methods

Mice received standard care and were euthanized according to the standards set forth by the Institutional Animal Care and Use Committee. To generate orthotopic lung tumors using human lung adenocarcinoma cell lines, nu/nu mice (n=10 mice per cohort) were subjected to intrathoracic injection with 10⁶ human tumor cells, necropsied after a week or more of treatment, and primary tumor size and the number of metastases on the contralateral lung surface measured.

To treat mice bearing human orthotopic lung tumors with the PI4KIIIβ inhibitor compound B, nu/nu mice were injected with 10⁶ human lung adenocarcinoma cells by the intra-thoracic approach and treatment initiated with compound B (20 or 40 mg/kg each plus 20 mg/kg ritonavir) or vehicle (5% DMSO, 20% HPBCD, 2% Poly 80 and 10% PEG300) one week after tumor cell injection. Drugs were administered subcutaneously twice daily for three weeks. On the last day of treatment, mice were subjected to micro-computed tomography to measure primary tumor size. The following day, mice were necropsied to measure primary tumor size, count metastases to the contralateral lung, and obtain lung tissues for analysis.

To treat mice bearing human orthotopic lung tumors with the PI4KIIIβ inhibitor compound A, nu/nu mice were injected with 10⁶ human lung adenocarcinoma cells by the intra-thoracic approach and treatment initiated with compound A (100 mg/kg plus 20 mg/kg ritonavir) or vehicle (5% DMSO, 20% HPBCD, 2% Poly 80 and 10% PEG300) one week after tumor cell injection. Drugs were administered subcutaneously twice daily for 8 days. On the last day of treatment, mice were subjected to micro-computed tomography to measure primary tumor size. The following day, mice were necropsied to measure primary tumor size, count metastases to the contralateral lung, and obtain lung tissues

Human lung cancer cells (A549, H1299, H460, H23, H2122, and H3122) were cultured in RPMI 1640 containing 10% FBS. Cells were maintained at 37° C. in an incubator with a humidified atmosphere containing 5% CO₂.

### Results: Anti-Cancer Activity of PI4K Antagonists

Small-molecule PI4K antagonists have been used as antiviral agents against single stranded RNA viruses that require PI4KIIIβ for replication (Rutaganira, F. U., et al. Design and Structural Characterization of Potent and Selective Inhibitors of Phosphatidylinositol 4 Kinase IIIbeta. J. Med. Chem. 59, 1830-1839, 2016). Applicants understood that PI4K inhibitors could find use in anti-cancer applications.

To assess the anti-cancer activity of exemplary PI4K antagonists, a panel of lung cancer cell lines annotated for the presence or absence of PI4K amplifications were treated with PI4K inhibitors (IN-9, IN-10, or compound B) that have greater than 1000-fold selectivity against PI4KIIIβ over class I and class III PI3K family members. PI4K inhibitor treatment decreased PI4P levels in a dose-dependent fashion (FIG. 2A) and reduced cell proliferation in monolayer culture, migration and invasion in Boyden chambers, and colony formation in soft agar and on plastic (FIG. 2B-2E). IC50 values were even lower with the presence of PI4K amplifications (FIG. 2B).

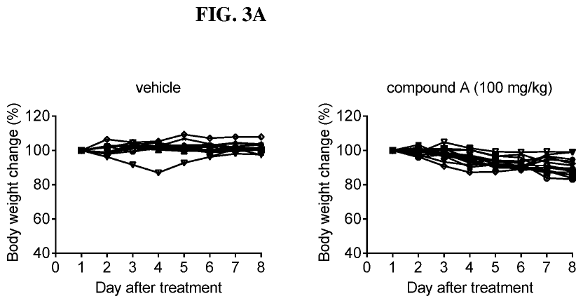

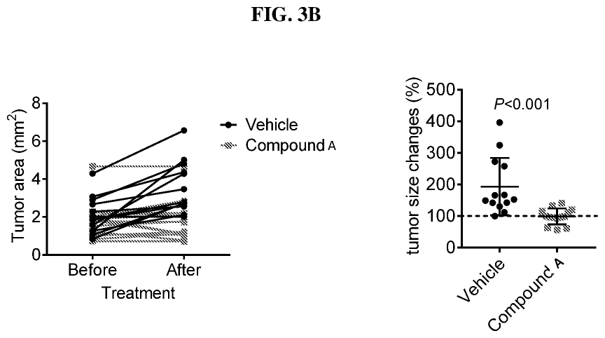

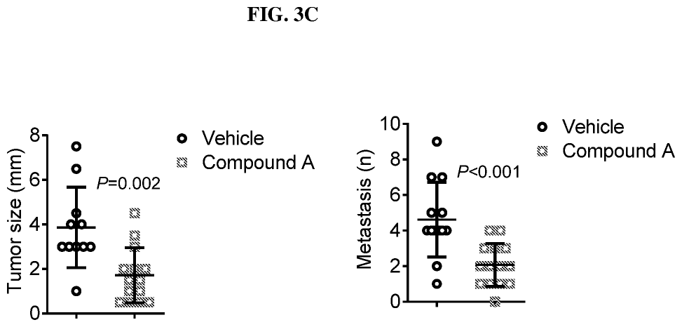

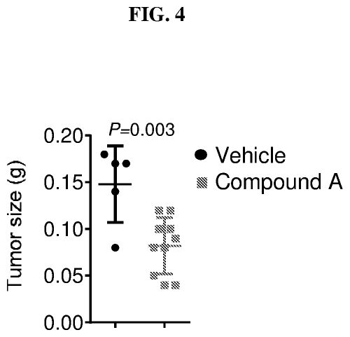

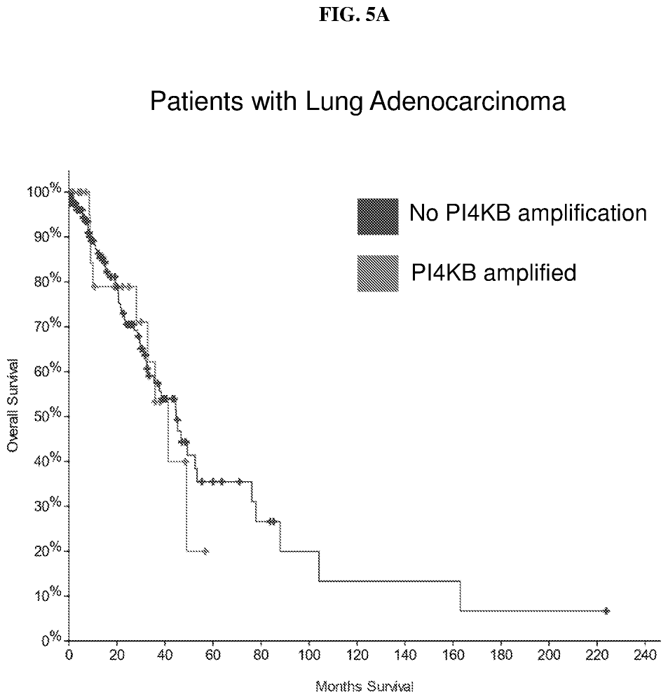

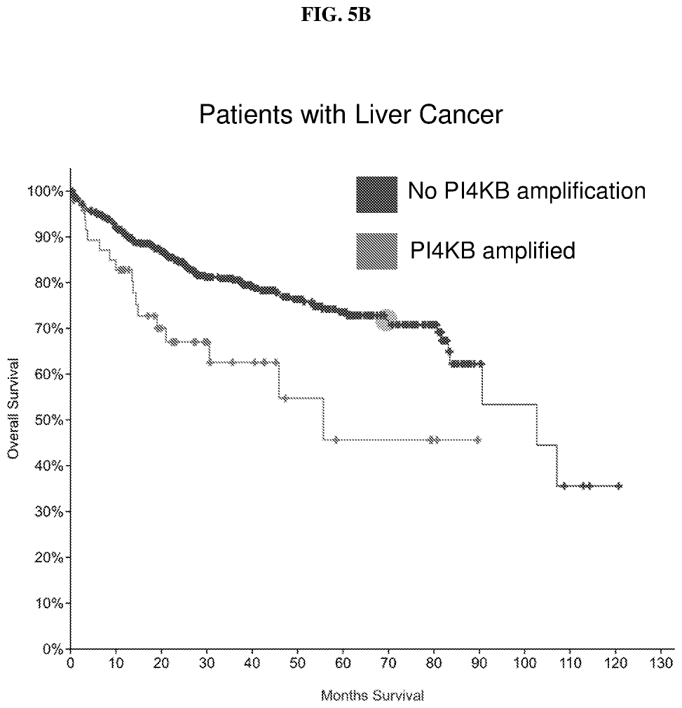

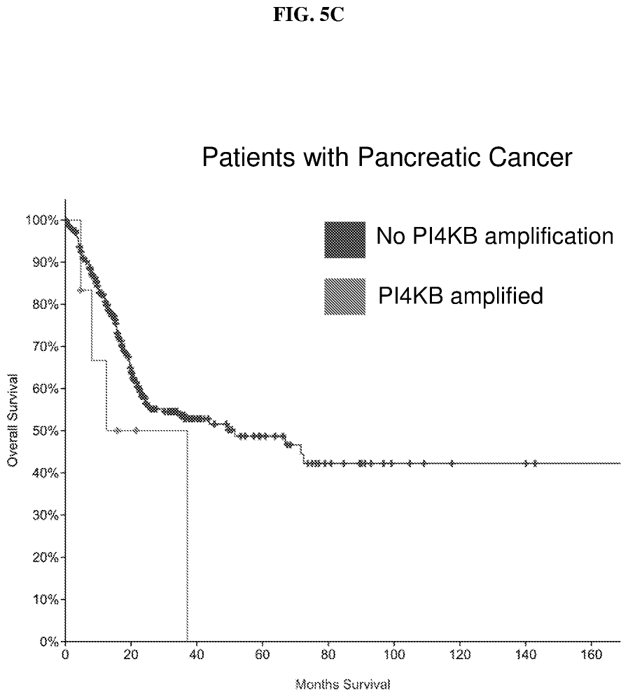

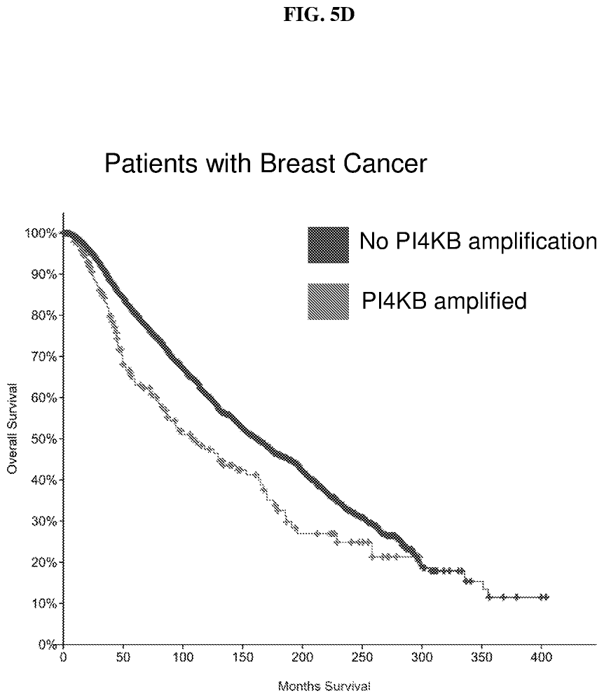

PI4K inhibition leads to decreased PI-4P dependent processes including PI-4P mediated membrane association and intracellular trafficking. Moreover, PI4K antagonists demonstrated robust anti-tumor activity in nu/nu mice bearing H2122 human orthotopic lung tumors (FIG. 2F-G and FIG. 3B-3C)

FIG. 1A-1C, 2A-2G, and 3B-C illustrate that PI4KIIIβ is a target in human cancers, including lung adenocarcinoma. (FIG. 2A). PI4P concentrations in H2122 cells (dots) treated in triplicate (dots) with Compound B or vehicle dimethyl sulfoxide (DMSO). (FIG. 2B) Relative densities of PI4-kinase-amplified (red) and diploid (black) human lung adenocarcinoma cell lines determined by WST-1 assays after 5 days of Compound B treatment. Results expressed relative to the lowest dose, which was set at 100%. (FIG. 2B, right panel) Half maximal inhibitory (IC50) concentrations of compound B determined from FIG. 2B, left panel. (FIG. 2C) Migrated and invaded H23 human lung adenocarcinoma cells in Transwell chambers were photographed (images) and counted (bar graphs) after treatment with compound B. Results expressed relative to DMSO-treated cells, which were set at 1.0. (FIG. 2C, right panel). Colonies formed by H2122 human lung cancer cells in soft agarose (FIG. 2D) and on plastic (FIG. 2E) were photographed (images) and counted (bar graphs) after 7 days of treatment with the indicated doses of compound B or vehicle DMSO (0 μM). Results expressed relative to DMSO control, which were set at 1.0. PI4-kinase inhibition leads to selective cytotoxicity for cancer cells (FIG. 2F).

FIG. 2A. Intracellular PI4P concentrations in H2122 lung cancer cells treated with compound B (PI4-kinase inhibitor) or vehicle DMSO. FIG. 2B Left panel: Relative densities of PI4KIIIβ-amplified (red) and diploid (black) human lung adenocarcinoma cell lines by WST-1 assays after 5 days of compound B treatment. Results expressed relative to the lowest dose, which was set at 100%. Right panel: Half maximal inhibitory (IC50) concentrations of compound B determined from left panel data. FIG. 2C. Migrated and invaded H23 human lung cancer cells in Transwell chambers were photographed (images) and counted (bar graphs) after treatment with compound B. Results expressed relative to DMSO-treated cells, which were set at 1.0. (FIG. 2D-2E). Colonies formed by H2122 human lung cancer cells in soft agarose (FIG. 2D) and on plastic (FIG. 2E) were photographed (images) and counted (bar graphs) after 7 days of treatment with the indicated doses of compound B or vehicle DMSO (0 μM). Results expressed relative to DMSO control, which were set at 1.0.

PI4-kinase inhibition leads to significant cytotoxicity for cancer cells (Table 4).

TABLE 4
CC50 of cancer cells in response to
treatment with PI4-kinase inhibitors

| | | Tumor type: | |
|---|---|---|---|
| | | Glioblastoma | Melanoma |
| | | Cancer cell line: | |
| | | A172 | A375 |
| Compound/CC50 (M): | Compound A | 2⁻⁷ | 6⁻⁷ |
| | Compound B | 5⁻⁷ | 7⁻⁶ |
| | Compound C | 1⁻⁷ | 4⁻⁶ |
| | Erlontinib | 1⁻⁴ | 1⁻⁵ |

FIGS. 2F and 2G. Schema of compound B treatment: Day 0, H2122 human lung cancer cell injection; day 7-27 compound B treatment; tumor imaging day 26 and necropsy day 27. (FIG. 2F) Mice subjected to micro-computed tomography after 19 days of treatment to determine tumor areas (left dot plot). Tumor diameters determined at necropsy (right dot plot). (FIG. 2G) Mice grouped on the basis of lung tumor measurements determined at necropsy, which showed a shift toward smaller tumor diameters in compound B-treated mice. No metastases were detectable following treatment with Compound B, and the sizes of the primary tumors following Compound B treatment were smaller than in those mice receiving treatment with vehicle alone.

Schema of compound A treatment: Day 0, H2122 human lung cancer cell injection; day 7-15 compound A treatment; tumor imaging day 14 and necropsy day 15. (FIG. 3A) Mouse body weight changes after 8 days treatment with vehicle (left panel) or vehicle plus 100 mg/kg/day compound A (right panel). (FIG. 3B) Mice subjected to micro-computed tomography before and after treatment to determine tumor areas after 7 days treatment with vehicle or vehicle plus 100 mg/kg/day compound A. Left panel: tumor area as measured before and after treatment. Right panel: tumor area expressed as percent of baseline measurement. (FIG. 3C) Tumor diameters determined at necropsy (left panel), and number of tumor metastases (right panel). Whereas the primary tumors increased in size in mice receiving treatment with vehicle alone, the primary tumors in Compound A-treated mice did not (FIG. 3B). Moreover, even though this treatment was quite short, consisting of just one week, the number of metastases in Compound A-treated mice was significantly lower than in the mice treated with vehicle alone (FIG. 3C).

PI4K antagonists are shown to induce apoptosis and impair metastatic properties in cancers, as well as preferentially in cancers with increased PI4K activity as a result of gene amplification (e.g. PI4K, eEF1A2) or increased expression of PI4K stimulating factors (e.g. eEF1A2). These findings have therapeutic implications spanning different cancer types.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of treating cancer, the method comprising:
administering to a subject with cancer a therapeutically effective amount of a PI4-kinase antagonist or a pro-drug thereof, a pharmaceutically acceptable salt or a solvate thereof.

2. The method of claim 1, wherein the cancer is a carcinoma.

3. The method of claim 1 or 2, wherein the cancer is a solid tumor cancer.

4. The method of claim 3, wherein the compound inhibits metastasis of the solid tumor.

5. The method of any one of claims 1-4, wherein the cancer is selected from bladder, breast, colon, endometrial, liver, lung, non-small cell lung cancer (NSCLC), ovarian, prostate, pancreatic, melanoma and sarcoma.

6. The method of claim 5, wherein the cancer is lung cancer.

7. The method of claim 6, wherein the cancer is a lung adenocarcinoma.

8. The method of any one of claims 1-7, wherein cancer cells of the subject comprise an elevated level of PI4KIIIβ expression (e.g., relative to a basal level in one or more normal or control cells).

9. The method of any one of claims 1-7, wherein cancer cells of the subject comprise an elevated expression level of a factor involved in IRES-mediated translation that stimulates PI4-kinase activity (e.g. eEF1A2).

10. The method of any one of claims 1-9, wherein cancer cells of the subject comprise an elevated level of PI4KIIIβ activity.

11. The method of any one of claims 1-10, wherein cancer cells of the subject are sensitive to PI4KIIIβ inhibition.

12. The method of any one of claims 1-11, further comprising:
measuring the expression level or activity level of PI4KIIIβ in cancer cells of a biological sample obtained from the subject; and
determining whether the expression level or activity level of PI4KIIIβ in the cancer cells is elevated relative to one or more control cells.

13. The method of claims 1-12, wherein the cancer cells of the subject have a greater than diploid copy number of the PI4KIIIβ gene.

14. The method of any one of claims 1-13, wherein the PI4-kinase antagonist is selective for PI4-kinase over PI3-kinase.

15. The method of any one of claims 1-14, wherein the PI4-kinase antagonist is a PI4KIIIβ inhibitor.

16. The method of any one of claims 1-14, wherein the PI4-kinase antagonist is a PI4KIIIα inhibitor.

17. The method of any one of claims 1-16, further comprising co-administering an effective amount of an additional agent to the subject.

18. The method of claim 16, wherein the additional agent is a chemotherapeutic agent or an immunotherapeutic agent.

19. The method of claim 16, wherein the additional agent is an inhibitor of a compound-metabolizing enzyme.

20. The method of claim 19, wherein the metabolizing enzyme is a cytochrome P-450 (e.g., CYP3A4).

21. The method of claim 19 or 20, wherein the additional agent is selected from clarithromycin, cobicistat, telithromycin, nefazodone, itraconazole, ketoconazole, atazanavir, darunavir, indinavir, lopinavir, nelfinavir, ritonavir, saquinavir and tipranavir (e.g., ritonavir or cobicistat).

22. The method of any one of claims 1-21, wherein the PI4-kinase antagonist is selected from pyrazolopyridines (e.g., KDU731), aminoimidazoles, aminoquinolines, quinazolinones, imidazo[1,2-a]pyrazines, quercetin, wortmannin, pyrazolo[1,5-a]pyrimidines (e.g., T-00127-HEV1), 2-(4-morpholinyl)-8-phenyl-1(4H)-benzopyran-4-one (LY-294,002), and 4-anilino-quinazolines (e.g., AL-9).

23. The method of any one of claims 1-21, wherein the PI4-kinase antagonist is a 5-aryl or heteroaryl-thiazole compound.

24. The method of claim 23, wherein the PI4-kinase antagonist is a substituted 2-amino-5-phenylthiazole or a substituted 2-amino-5-pyridylthiazole compound.

25. The method of claim 24, wherein the PI4-kinase antagonist is of formula (XXI): 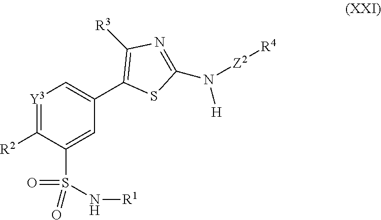
wherein:
R² is an alkoxy or a substituted alkoxy;
R³ is hydrogen, a lower alkyl or a substituted lower alkyl;
Y³ is CH or N;
Z² is absent or CO;
R¹ is an aryl, a substituted aryl, a heteroaryl, a substituted heteroaryl, an alkyl, a substituted alkyl, a cycloalkyl, a substituted cycloalkyl, a heterocycle or a substituted heterocycle; and
R⁴ is selected from alkyl, substituted alkyl, cycloalkyl, substituted cycloalkyl, alkyl-cycloalkyl, substituted alkyl-cycloalkyl, aryl, substituted aryl, heterocycle, substituted heterocycle, alkyl-heterocycle and substituted alkyl-heterocycle;
or a prodrug thereof, or a pharmaceutically acceptable salt thereof.

26. The method of any one of claims 23-25, wherein the PI4-kinase inhibitor is selected from a compound of Table 1-3.

27. A method of inhibiting proliferation of a cancer cell, the method comprising:
contacting a cancer cell with an effective amount of a PI4-kinase inhibitor or a pharmaceutically acceptable salt thereof.

28. The method of claim 27, wherein the cancer cell is selected from bladder, breast, colon, endometrial, liver, lung, non-small cell lung cancer (NSCLC), ovarian, prostate, pancreatic, melanoma and sarcoma cancer cells.

29. The method of any one of claims 27-28, wherein the cancer cell expresses PI4KIIIβ at elevated levels (e.g., relative to a basal level in one or more normal or control cells).

30. The method of any one of claims 27-28, wherein the cancer cell comprises an elevated expression level of a factor involved in IRES-mediated translation that stimulates PI4-kinase activity (e.g. eEF1A2).

31. The method of any one of claims 27-30, wherein cancer cell comprises an elevated level of PI4KIIIβ activity.

32. The method of any one of claims 27-31, wherein cancer cell is sensitive to PI4KIIIβ inhibition.

33. The method of any one of claims 27-32, wherein the PI4-kinase antagonist is selected from pyrazolopyridines (e.g., KDU731), aminoimidazoles, aminoquinolines, quinazolinones, imidazo[1,2-a]pyrazines, quercetin, wortmannin, pyrazolo[1,5-a]pyrimidines (e.g., T-00127-HEV1), 2-(4-morpholinyl)-8-phenyl-1(4H)-benzopyran-4-one (LY-294,002), and 4-anilino-quinazolines (e.g., AL-9).

34. The method of any one of claims 27-32, wherein the PI4-kinase inhibitor is a 5-aryl or heteroaryl-thiazole compound.

35. The method of claim 34, wherein the PI4-kinase inhibitor is a substituted 2-amino-5-phenylthiazole or a substituted 2-amino-5-pyridylthiazole compound.

36. The method of claim 35, wherein the compound is of formula (XXI): 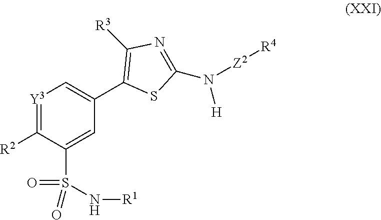
wherein:
R² is an alkoxy or a substituted alkoxy;
R³ is hydrogen, a lower alkyl or a substituted lower alkyl;
Y³ is CH or N;
Z² is absent or CO;
R¹ is an aryl, a substituted aryl, a heteroaryl, a substituted heteroaryl, an alkyl, a substituted alkyl, a cycloalkyl, a substituted cycloalkyl, a heterocycle or a substituted heterocycle; and
R⁴ is selected from alkyl, substituted alkyl, cycloalkyl, substituted cycloalkyl, alkyl-cycloalkyl, substituted alkyl-cycloalkyl, aryl, substituted aryl, heterocycle, substituted heterocycle, alkyl-heterocycle and substituted alkyl-heterocycle;
or a prodrug thereof, or a pharmaceutically acceptable salt thereof.

37. The method of claim 36, wherein the compound is selected from the compounds of Tables 1-3, or a prodrug thereof, or a pharmaceutically acceptable salt thereof.

38. An anti-cancer kit, comprising:
an effective dose of a PI4 kinase inhibitor;
an effective dose of an additional anticancer agent; and
instructions for use in treating cancer.

39. The kit of claim 38, wherein the PI4-kinase inhibitor is a 5-aryl or heteroaryl-thiazole compound.

40. The kit of claim 39, wherein the PI4-kinase inhibitor is a substituted 2-amino-5-phenylthiazole or a substituted 2-amino-5-pyridylthiazole compound.

41. The kit of claim 40, wherein the compound is of formula (XXI): 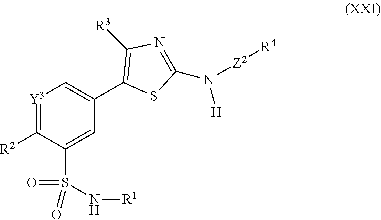
wherein:
R² is an alkoxy or a substituted alkoxy;
R³ is hydrogen, a lower alkyl or a substituted lower alkyl;
Y³ is CH or N;
Z² is absent or CO;
R¹ is an aryl, a substituted aryl, a heteroaryl, a substituted heteroaryl, an alkyl, a substituted alkyl, a cycloalkyl, a substituted cycloalkyl, a heterocycle or a substituted heterocycle; and
R⁴ is selected from alkyl, substituted alkyl, cycloalkyl, substituted cycloalkyl, alkyl-cycloalkyl, substituted alkyl-cycloalkyl, aryl, substituted aryl, heterocycle, substituted heterocycle, alkyl-heterocycle and substituted alkyl-heterocycle;
or a prodrug thereof, or a pharmaceutically acceptable salt thereof.

42. The kit of claim 41, wherein the compound is selected from the compounds of Tables 1-2, or a prodrug thereof, or a pharmaceutically acceptable salt thereof.
